(19) United States
(12) Patent Application Publication
(10) Pub. No.: US 2025/0144032 A1
(43) Pub. Date: May 8, 2025

(54) POLY(BETA-AMINO ESTER) MICROPARTICLES FOR MICRONUTRIENT FORTIFICATION

(71) Applicant: Massachusetts Institute of Technology, Cambridge, MA (US)

(72) Inventors: Robert S. Langer, Newton, MA (US); Ana Jaklenec, Lexington, MA (US); Ruiqing Xiao, Brookline, MA (US); Linzixuan Zhang, Cambridge, MA (US)

(73) Assignee: Massachusetts Institute of Technology, Cambridge, MA (US)

(21) Appl. No.: 18/714,142

(22) PCT Filed: Nov. 30, 2022
(86) PCT No.: PCT/US2022/051366

§ 371 Date: May 29, 2024

Related U.S. Application Data
(60) Provisional application No. 63/284,519, filed on Nov. 30, 2021.

(51) Int. Cl. A61K 9/48; A61K 31/07; A61K 31/122; A61K 31/355; A61K 31/375; A61K 31/455; A61K 31/519; A61K 31/525; A61K 31/593; A61K 31/714; A61K 33/18; A61K 33/26; A61K 33/30; C08G 73/06

(52) U.S. Cl. CPC A61K 9/4816; A61K 9/4858; A61K 9/4866; A61K 31/07; A61K 31/122; A61K 31/355; A61K 31/375; A61K 31/455; A61K 31/519; A61K 31/525; A61K 31/593; A61K 31/714; A61K 33/18; A61K 33/26; A61K 33/30; C08G 73/0627; C08G 73/0633

(57) ABSTRACT

Provided herein are compounds, such as compounds of Formulae (I) and (III), and compositions, methods, uses, and kits thereof. The compounds provided herein are poly(β-amino esters) useful for delivery of agents, such as vitamins and minerals, for the treatment and/or prevention of various diseases and conditions (e.g., micronutrient deficiency).

### CROSS-REFERENCE TO RELATED APPLICATIONS

This application claims the benefit of and priority under 35 U.S.C. § 119(e) to U.S. Provisional Application No. 63/284,519, filed Nov. 30, 2021, titled POLY(β-AMINO ESTER) MICROPARTICLES FOR MICRONUTRIENT FORTIFICATION, the contents of which are incorporated herewith by reference in their entirety.

### BACKGROUND

Micronutrient (MN) deficiencies are globally prevalent among approximately 372 million preschool-aged children and 1.2 billion non-pregnant women of reproductive age. Nearly two billion people in the developing world are plagued by micronutrient deficiencies, which cause up to two million childhood deaths per year. Micronutrient deficiencies contribute to many disabilities and diseases including cognitive and physical disorders, anemia, blindness, birth defects, and impaired growth in children, and chronic lack of micronutrients can result in severe morbidity and mortality. Adolescent girls, women of reproductive age, as well as pregnant and lactating women are especially susceptible to these consequences.

Large-scale human trials have established that fortification of food products, such as staple food, condiment (e.g., table salt) and sauce or soup, can effectively treat micronutrient deficiencies. Many of these findings did not impact the vast population suffering from micronutrient deficiencies because of implementation challenges in target countries and technical challenges related to micronutrient stability during storage and cooking. Populations in developing world countries consume food products that often require extensive cooking, which introduces heat, moisture, and oxidation. This results in either degradation of vitamins, which render them biologically inactive and impair delivery, or chemical changes to minerals that make the food unpalatable. Thus, a major limitation of food fortification is the requirement to effectively maintain micronutrient integrity during the food storage and cooking conditions.

In particular, vitamin A deficiency remains a significant challenge in Low and Middle Income Countries (LMIC), and is a leading cause of preventable childhood blindness and related to common childhood infections. Food fortification is one of the most promising interventions for improving population intakes of Vitamin A. While downstream issues of fortification mandates, regulatory enforcement, and technical assistance have complicated the ability to ameliorate Vitamin A deficiency (VAD), upstream challenges have also existed. Vitamin A is highly unstable, particularly in dry foods. Commercially available encapsulated Vitamin A are an improvement over oily forms, but significant overages have still been required to ensure active Vitamin A at the point of consumption. For instance, in fortified bouillon cubes, the average lifespan was 10 months and an overage of approximately 2.5× times the original level was required to meet the target.

Certain formulations of basic methacrylate copolymer (BMC) microencapsulated various micronutrients, which enabled exceptional stability of these micronutrients (11 of 11 tested) during storage and cooking. BMC is a pH-responsive polymer that is hydrophobic and can form stable particles in neutral pH, but quickly dissolves in acidic conditions. This property of BMC protected micronutrients from heat degradation during cooking and released them when they reached the stomach. The European Chemical Agency (ECHA) proposed a general ban on the use of microplastics in foods (defined as solids ranging from 1 nm-5 mm) used in the European Union (EU), and BMC was listed as one of many plastics in the proposed ban. Uncertainty regarding the use of BMC for food fortification created a need to develop stable micronutrient microparticles with natural materials and degradation products.

As such, the development of technologies to address specific micronutrient stability issues that can facilitate implementation of food products fortification and impact global health by treating worldwide deficiencies is needed.

### SUMMARY OF THE INVENTION

Microparticles (MPs) encapsulating micronutrients can serve as a protective layer between the outer environment and the unstable cargo as well as present as free-flowing powders to be easily handled and incorporated into food matrix processing. Microencapsulation may also ensure efficient dissociation between the delivery platform and the protected micronutrients in the digestive system to render high bioavailability for human absorption. Without wishing to be bound by theory, MPs fabricated from biodegradable, natural product-based polymers can reduce the regulatory barriers from the safety perspective and mitigate the long-term environmental risks associated with its large-scale implementation.

The present disclosure relates in part to new compounds (e.g., compounds of Formula (I)) that can deliver agents to a subject, and compositions and methods of using and preparing such compounds.

Provided herein are compounds (e.g., compounds of Formula (I)), and pharmaceutically acceptable salts, stereoisomers, and isotopically labeled derivatives thereof, and pharmaceutical compositions thereof. In certain embodiments, the compounds provided herein can form particles for delivery of various agents and can therefore be useful for the treatment and/or prevention of diseases (e.g., micronutrient deficiency).

The present disclosure also provides methods of using the compounds and compositions provided herein, e.g., for delivering an agent to a subject. Also provided herein are methods of preparing compounds provided herein (e.g., compounds of Formula (I)), and pharmaceutically acceptable salts, stereoisomers, and isotopically labeled derivatives thereof.

The present disclosure also provides kits comprising a compound provided herein (e.g., a compound of Formula (I)), or a pharmaceutically acceptable salt, stereoisomer, or isotopically labeled derivative thereof, or a pharmaceutical composition thereof.

In one aspect, the disclosure provides a compound of Formula (I):

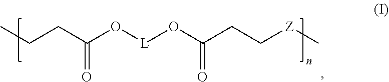

or a pharmaceutically acceptable salt, stereoisomer, or isotopically labeled derivative thereof, wherein L, Z, and n are as defined herein.

In another aspect, the disclosure provides a compound of Formula (III):

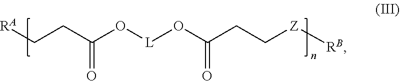

or a pharmaceutically acceptable salt, stereoisomer, or isotopically labeled derivative thereof, wherein R^{A}, R^{B}, L, Z, and n are as defined herein.

In certain embodiments, L is

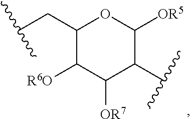

the compound of Formula (I) is of Formula (I-A):

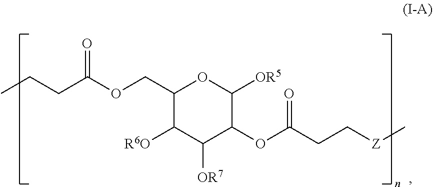

or a pharmaceutically acceptable salt, stereoisomer, or isotopically labeled derivative thereof, wherein R⁵, R⁶, R⁷, Z, and n are as defined herein.

In certain embodiments, L is

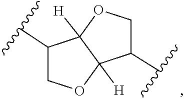 [SMILES: *C1COC2C(*)COC12]

the compound of Formula (I) is of Formula (I-B):

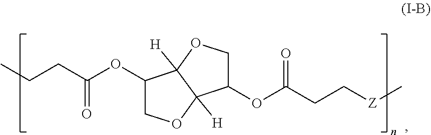

or a pharmaceutically acceptable salt, stereoisomer, or isotopically labeled derivative thereof, wherein Z and n are as defined herein.

In another aspect, the disclosure provides a compound prepared by reacting one or more compounds of Formula (II):

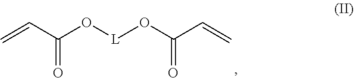

or a salt, isotope, or stereoisomer thereof, with one or more compounds selected from

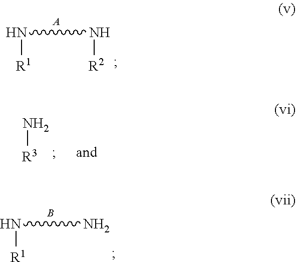

or a salt, isotope, or stereoisomer thereof, wherein L, R¹, R², R³, R⁴, linker A, and linker B are as defined herein.

In another aspect, the present disclosure provides compositions comprising a compound provided herein (e.g., a compound of Formula (I)), or a pharmaceutically acceptable salt, stereoisomer, or isotopically labeled derivative thereof, and an agent. In some embodiments, the agent is a vitamin, mineral, micronutrient, biologic, small molecule, probiotic, or polynucleotide. In certain embodiments, the agent is a vitamin or mineral. In some embodiments, degradation of the composition produces one or more natural byproducts (e.g., a monosaccharide, isosorbide, or a β-amino acid).

In another aspect, the present disclosure provides a pharmaceutical composition comprising a compound, or a pharmaceutically acceptable salt, stereoisomer, or isotopically labeled derivative thereof, or composition provided herein.

In another aspect, the present disclosure provides a nutraceutical composition comprising a compound, or a pharmaceutically acceptable salt, stereoisomer, or isotopically labeled derivative thereof, or composition provided herein.

In another aspect, the present disclosure provides a food product comprising a compound, or a pharmaceutically acceptable salt, stereoisomer, or isotopically labeled derivative thereof, or composition provided herein.

In another aspect, the present disclosure provides a beverage comprising a compound, or a pharmaceutically acceptable salt, stereoisomer, or isotopically labeled derivative thereof, or composition provided herein.

In another aspect, the present disclosure provides a nutritional supplement comprising a compound, or a pharmaceutically acceptable salt, stereoisomer, or isotopically labeled derivative thereof, or composition provided herein.

In another aspect, the present disclosure provides methods for treating and/or preventing a disease, disorder, or condition (e.g., micronutrient deficiency) in a subject, comprising administering to the subject a composition provided herein. In some embodiments, the disease, disorder, or condition is a micronutrient deficiency, genetic disease, proliferative disease, hematological disease, neurological disease, liver disease, spleen disease, lung disease, painful condition, psychiatric disorder, musculoskeletal disease, metabolic disorder, inflammatory disease, or autoimmune disease. In certain embodiments, the disease, disorder, or condition is a micronutrient deficiency. In some embodiments, the micronutrient deficiency is vitamin A deficiency.

In yet another aspect, the present disclosure provides methods of delivering an agent to a subject, comprising administering to the subject a composition provided herein.

In another aspect, the present disclosure provides kits comprising a compound provided herein (e.g., a compound of Formula (I)), or a pharmaceutically acceptable salt, stereoisomer, or isotopically labeled derivative thereof; or a pharmaceutical composition thereof; and instructions for using the compound, or pharmaceutically acceptable salt, stereoisomer, or isotopically labeled derivative thereof, or pharmaceutical composition thereof (e.g., for treating and/or preventing a disease or condition in a subject or delivering an agent to a subject).

In another aspect, the disclosure provides a method of preparing a compound of Formula (I), the method comprising reacting a compound of Formula (II):

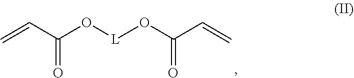

or a salt, isotope, or stereoisomer thereof, with one or more compounds selected from

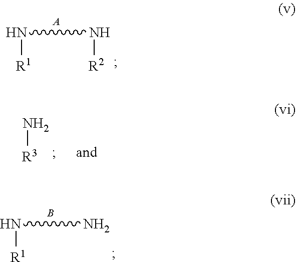

or a salt, isotope, or stereoisomer thereof, wherein L, R¹, R², R³, R⁴, linker A, and linker B are as defined herein.

The details of certain embodiments of the invention are set forth in the Detailed Description of Certain Embodiments, as described below. Other features, objects, and advantages of the invention will be apparent from the Definitions, Examples, Figures, and Claims.

### BRIEF DESCRIPTION OF THE DRAWINGS

The accompanying drawings, which are incorporated in and constitute a part of this specification, illustrate several embodiments of the invention and together with the description, provide non-limiting examples of the invention.

FIG. 1. PAEs_A-E are synthesized by isosorbide diacrylate, 4,4′-trimethylenedipiperidine (TDP), and piperazine (PP), varying the ratio of TDP and PP. The table presents five compositions of attempted PAEs for VA encapsulation. PAE_C, D, and E produced MP-form solid material after emulsion process. Under boiling conditions, PAEs degrade into isosorbide and one or two di-amino acids.

FIGS. 2A-2B. FIG. 2A, Among PAE_C, D, and E, PAE_E provides the highest protection efficiency for VA after two-hour boiling in water with over 80% recovery. FIG. 2B, Over 99% of VA is released from PAE_E MP upon 30 minutes of simulated gastric fluid (SGF) treatment under 37° C.

FIG. 3. PAE degradation profiles under water-boiling condition are established by NMR (left axis) for blank PAE_E MP and PAE_E MP with VA. For both formulations, over 90% of polymer degrades in boiled products after two hours, and no polymer residue is detected after four-hour boiling. Molecular weight (right axis) was monitored by gel permeation chromatography (GPC) as a function of boiling time for blank PAE_E MP.

FIG. 4. Long-term storage stability data for VA are compared between PAE_E-VA MP and BMC-VA MP formulations. At 4-week and 8-week time points, both formulations showed over 50% recovery.

FIGS. 5A-5B. FIG. 5A, Chemical structures of PAE. FIG. 5B, SEM images of PAE_E-VA MPs.

FIGS. 6A-6C. FIG. 6A. Effect of PAE composition on VA protection (100° C., water, 120 minutes). FIG. 6B. Recovery of VA in PAE_0:100 MPs in 100° C. water at different times. FIG. 6C, VA per mg MP versus boiling time.

FIGS. 7A-7G. Boiling stability of multiple micronutrients (vitamin A (VA), vitamin D (VD), vitamin E (VE), vitamin C (VC)). FIGS. 7A-7D. Protection provided by PAE_E for hydrophobic micronutrients (VA, VD, VE, VC) by individual encapsulation. FIG. 7A. Recovery of VA, VD, or VE in PAE_0:100 MPs in 100° C. water at different times. FIG. 7B, VA, VD, or VE per mg MP versus boiling time. FIG. 7C. Recovery of VC in PAE_0:100 MPs in 100° C. water at different times. FIG. 7E, VC per mg MP versus boiling time. FIG. 7E, Protection provided by PAE_E for hydrophobic micronutrients (VA, VD, VE) by co-encapsulation. FIG. 7F, micronutrient per mg MP versus boiling time for co-encapsulated micronutrients. FIG. 7G, micronutrient release of co-encapsulated VA, VD, or VE. Co-encapsulated VA, VD, and VE afforded approximately 100% release at 0.5, 1.0, and 2.0 hours.

FIGS. 8A-8D. Protection provided by PAE_E for hydrophilic micronutrients (iron and zinc) by individual encapsulation. FIG. 8A, Recovery of iron (ferrous sulfate) in PAE_0:100 MPs in 100° C. water at different times. FIG. 8B, iron per mg MP versus boiling time. FIG. 8C, Recovery of zinc (zinc sulfate) in PAE_0:100 MPs in 100° C. water at different times. FIG. 8D, zinc per mg MP versus boiling time.

FIGS. 9A-9E. Long-term storage stability of VA. VA stability in PAE_E MP was evaluated up to 6-month storage in accelerated conditions. Recovery percentage was calculated relative to recovery from the untreated (no boiling, no storage). FIGS. 9A-9D, Incubation condition was 40° C., 75% humidity. FIG. 9A, PAE_E-10% VA MP. FIG. 9B, PAE_E-10% VA MP with 0.5% butylated hydroxytoluene (BHT). FIG. 9C, PAE_E-15% VA MP. FIG. 9D, PAE_E-10% VA with 10% VE MP. FIGS. 9E, Incubation condition was 25° C., 40% humidity, PAE_E-10% VA MP.

FIG. 10. Release profile of multiple micronutrients (VA, VD, VE, iron, zinc). With treatment of simulated gastric fluid under 37° C., micronutrients are efficiently released from the PAE_E MP for subject absorption. PAE-VA and PAE-VD in SGF afforded similar release profiles. PAE-Dex-Zn and PAE-PVA-Zn in SGF afforded similar release profiles.

FIG. 11. Synthesis of poly(beta-amino ester) for VA Encapsulation. Hydrophobicity increases with the ratio of TDP:PP in PAE.

FIG. 12. Under cooking and acidic conditions, PAE degrades into isosorbide (a sugar derivative) and a di-β-amino acid.

FIG. 13. MPs are soluble in SGF after boiling.

FIGS. 14A-14B. Stability of free-form micronutrients. FIG. 14A, Vit-A Recovery of unencapsulated free form VA after various boiling times. FIG. 14B, Recovery of unencapsulated free form VA, VD, or VE after various boiling times.

FIG. 15 Recovery results for BMC-VA MP after 2 hours of boiling.

FIGS. 16A-16B. PAE_C-VA MP Cooking Stability. FIG. 16A, Samples fully dissolved in boiling water after 30 minutes. FIG. 16B, VA per mg MP versus boiling time.

FIG. 17. PAE_C MP SEM images.

FIGS. 18A-18B. PAE_D-VA MP Cooking Stability. FIG. 18A, Samples fully dissolved in boiling water after 60 minutes. FIG. 18B, VA per mg MP versus boiling time.

FIG. 19. PAE_D MP SEM images.

FIGS. 20A-20B. Degradation of PAE_E Blank MP. FIG. 20A, After 120 minutes in boiling water, the isosorbide peak in polymer was 7.4% of the total isosorbide peak (polymer+degradation product). FIG. 20B, After 60 minutes in SGF at 37° C. Degradation of PAE_E MP was estimated by comparing the ratio of isosorbide proton NMR peak in the polymer (5.18 parts per million) and in the monomer (4.69 parts per million).

FIG. 21. NMR spectra showing degradation of PAE_E Blank MP after boiling in water for various times.

FIGS. 22A-22B. Degradation of PAE_C & PAE_D Blank MP in boiling water as determined by NMR spectroscopy. FIG. 22A, After 120 minutes in boiling water, the polymer isosorbide peak was 6.1% of the total isosorbide peak (in polymer and in degradation product) for PAE_C. FIG. 22B, After 120 minutes in boiling water, the polymer isosorbide peak was 7.7% of the total isosorbide peak (in polymer and in degradation product) from PAE_D.

FIG. 23. VA recovery from PAE_E with or without BHT in boiling water (100° C.), 2 hours.

FIGS. 24A-24C. FIG. 24A, PAE_E MP yield versus VA loading ratio after boiling in water (100° C.) for 2 hours. FIG. 24B, VA cooking stability in boiling water (100° C.) for two hours versus loading ratio of VA encapsulated in PAE_E. FIG. 24C, VA cooking stability in boiling water (100° C.) for two hours versus loading ratio of VA encapsulated in PAE_E.

FIG. 25. Synthesis of monosaccharide-derived diacrylate and PAE. The monosaccharide is first reacted with a protecting group, followed by synthesis with acryloyl chloride to obtain the diacrylate. This compound is then polymerized with amine comonomers.

FIGS. 26A-26C. SEM images of blank PAE microparticles and PAE microparticles with encapsulated VA. FIG. 26A, PAE_E MPs. FIG. 26B, PAE_D MPs. FIG. 26C, PAE_C MPs.

FIGS. 27A-27B. PAE microparticle formulation. FIG. 27A, Encapsulation of VA or other hydrophobic micronutrients in PAE microparticles via emulsion of DCM or other organic phase in water. FIG. 27B, Encapsulation of hydrophilic micronutrients in PAE microparticles.

FIG. 28 Fluorescence images of PAE_E-10% VA MP.

FIGS. 29A-29B. Frequency distribution of MPs. FIG. 29A, PAE blank MP. FIG. 29B, PAE-10% VAMP.

FIG. 30. Water contact angle measurement of VA recovery from PAE_C, PAE_D, and PAE_E.

FIG. 31. SEM images of PAE MPs throughout water boiling process.

FIGS. 32A-32C. GPC data showing degradation in boiling water. FIG. 32A, LS 90° C. versus retention time for various boiling times. FIG. 32B, molecular weight versus boiling time. FIG. 32C, effect of molecular weight on VA protection efficacy.

FIGS. 33A-33E. Recovery of VA from PAE_E (P5) MP after long-term storage. FIG. 33A, Recovery of VA (10% wt) from PAE_E (P5) MP compared to the free-form VA. FIG. 33B, Recovery of VA (10% wt) from PAE_E (P5) MP with and without BHT. FIG. 33C, Recovery of VA (10% wt and 15% wt) from PAE_E (P5) MP. FIG. 33D, Recovery of VA (10% wt) from PAE_E (P5) MP compared to co-encapsulation with VE (10% wt). FIG. 33E, Recovery of VA (10% wt) from PAE_E (P5) MP stored at accelerated conditions (40° C. and 75% humidity) compared to room temperature conditions (25° C. and 45% humidity). *p<0.05, ***p<0.001, ****p<0.0001 as determined by the two-tailed Student t test. Error bars represent SD.

FIGS. 34A-34E. Recovery of VA from PAE_E (P5) MP after long-term storage followed by 2-hour boiling in water. FIG. 34A, Recovery of VA (10% wt) from PAE_E (P5) MP compared to the free-form VA. FIG. 34B, Recovery of VA (10% wt) from PAE_E (P5) MP with and without BHT. FIG. 34C, Recovery of VA (10% wt and 15% wt) from PAE_E (P5) MP. FIG. 34D, Recovery of VA (10% wt) from PAE_E (P5) MP compared to co-encapsulation with VE (10% wt). FIG. 34E, Recovery of VA (10% wt) from PAE_E (P5) MP stored at accelerated conditions (40° C. and 75% humidity) compared to room temperature conditions (25° C. and 45% humidity). **p<0.01,***p<0.001, ****p<0.0001 as determined by the two-tailed Student t test. Error bars represent SD.

FIG. 35. Long-term stability of unencapsulated free form VA under 40 C, 75% humidity.

FIG. 36. LC-MS of PAE_E degradation byproducts under boiling conditions.

FIGS. 37A-37D. Comparison with basic methacrylate copolymer (BMC)-VAMP. FIG. 37A, 10% VA loading of PAE_E or BMC stored at 40° C., 75% relative humidity. FIG. 37B, loading capacity of PAE_E versus BMC. FIG. 37C, VA loading versus initial VA concentration for PAE_E and BMC. FIG. 37D, VA recovery versus initial VA concentration.

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

The term “isotopes” refers to variants of a particular chemical element such that, while all isotopes of a given element share the same number of protons in each atom of the element, those isotopes differ in the number of neutrons.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

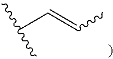

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is optionally substituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆-14 aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is optionally substituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is optionally substituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being optionally substituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is optionally substituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(=NR^{bb})OR^{aa}, —OC(=NR^{bb})R^{aa}, —OC(=NR^{bb})OR^{aa}, —C(=NR^{bb})N(R^{bb})₂, —OC(═NR^{bb})N(R^{bb})₂, —NR^{bb}C(=NR^{bb})N(R^{bb})₂, —C(═O)NR^{bb}SO₂R^{aa}, —NR^{bb}SO₂R^{aa}, —SO₂N(R^{bb})₂, —SO₂R′, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R′)₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X, —P(OR^{cc})₃X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃X⁻, —OP(R^{cc})₄, —OP(OR^{cc})₄, —B(R^{aa})₂, —B(OR^{cc})₂, —BR((OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

wherein:
each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R′, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ee})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or =S; wherein X⁻ is a counterion;
each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂₀C₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R⁹⁹ substituents can be joined to form ═O or =S; and
each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂ wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{aa})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂—NR^{bb}C(═NR^{bb})N(R^{bb})₂, —NR^{bb}SO₂R^{aa}, NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; optionally substituted hydroxyl; optionally substituted thiol; optionally substituted amino; optionally substituted acyl, cyclic or acyclic, optionally substituted, branched or unbranched aliphatic; cyclic or acyclic, optionally substituted, branched or unbranched heteroaliphatic; cyclic or acyclic, optionally substituted, branched or unbranched alkyl; cyclic or acyclic, optionally substituted, branched or unbranched alkenyl; optionally substituted alkynyl; optionally substituted aryl, optionally substituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

Nitrogen atoms can be optionally substituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NRC)N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{a} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R′, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R′, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{cc})₂, —P(═O)(OR)₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methoxy, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms.

The term “polymer” refers to a compound comprising eleven or more covalently connected repeating units. In certain embodiments, a polymer is naturally occurring. In certain embodiments, a polymer is synthetic (i.e., not naturally occurring).

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

These and other exemplary substituents are described in more detail in the Detailed Description, Examples, and Claims. The invention is not limited in any manner by the above exemplary listing of substituents.

### Other Definitions

The following definitions are more general terms used throughout the present application.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “particle” refers to a small object, fragment, or piece of a substance that may be a single element, inorganic material, organic material, or mixture thereof. Examples of particles include polymeric particles, single-emulsion particles, double-emulsion particles, coacervates, liposomes, microparticles, nanoparticles, macroscopic particles, pellets, crystals, aggregates, composites, pulverized, milled or otherwise disrupted matrices, and cross-linked protein or polysaccharide particles, each of which have an average characteristic dimension of about less than about 1 mm and at least 1 nm, where the characteristic dimension, or “critical dimension,” of the particle is the smallest cross-sectional dimension of the particle. A particle may be composed of a single substance or multiple substances. In certain embodiments, the particle is not a viral particle. In other embodiments, the particle is not a liposome. In certain embodiments, the particle is not a micelle. In certain embodiments, the particle is substantially solid throughout. In certain embodiments, the particle is a microparticle or nanoparticle. In certain embodiments, the particle is a nanoparticle. In certain embodiments, the particle is a microparticle.

The term “nanoparticle” refers to a particle having an average (e.g., mean) dimension (e.g., diameter) of between about 1 nanometer (nm) and about 1 micrometer (μm) (e.g., between about 1 nm and about 300 nm, between about 1 nm and about 100 nm, between about 1 nm and about 30 nm, between about 1 nm and about 10 nm, or between about 1 nm and about 3 nm), inclusive.

The term “microparticle” refers to a particle having an average (e.g., mean) dimension (e.g., diameter) of between about 1 micrometer (μm) and about 1 millimeter (mm) (e.g., between about 1 μm and about 100 μm, between about 1 μm and about 30 μm, between about 1 μm and about 10 μm, or between about 1 μm and about 3 μm), inclusive.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “administer,” “administering,” or “administration” refers to implanting, absorbing, ingesting, injecting, inhaling, or otherwise introducing a compound described herein, or a composition thereof, in or on a subject.

The terms “condition,” “disease,” and “disorder” are used interchangeably.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

An “effective amount” of a compound or agent provided herein refers to an amount sufficient to elicit the desired biological response. An effective amount of a compound or agent provided herein may vary depending on such factors as the desired biological endpoint, severeity of side effects, disease, or disorder, the identity, pharmacokinetics, and pharmacodynamics of the particular compound or agent, the condition being treated, the mode, route, and desired or required frequency of administration, the species, age and health or general condition of the subject. In certain embodiments, an effective amount is a therapeutically effective amount. In certain embodiments, an effective amount is a prophylactically effective amount. In certain embodiments, an effective amount is the amount of a compound or agent provided herein in a single dose. In certain embodiments, an effective amount is the combined amounts of a compound or agent provided herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 10000 mg, about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit.

In certain embodiments, the compounds of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound or agent described herein is an amount sufficient to provide a therapeutic benefit in the treatment of a condition or to delay or minimize one or more symptoms associated with the condition. A therapeutically effective amount of a compound or agent means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for delivering an agent to a subject. In certain embodiments, a therapeutically effective amount is an amount sufficient for treating a micronutrient deficiency. In certain embodiments, a therapeutically effective amount is an amount sufficient for delivering an agent to a subject and treating a micronutrient deficiency.

A “prophylactically effective amount” of a compound or agent described herein is an amount sufficient to prevent a condition, or one or more symptoms associated with the condition or prevent its recurrence. A prophylactically effective amount of a compound or agent means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for delivering an agent to a subject. In certain embodiments, a prophylactically effective amount is an amount sufficient for preventing a micronutrient deficiency. In certain embodiments, a prophylactically effective amount is an amount sufficient for delivering an agent to a subject and preventing a micronutrient deficiency.

The term “genetic disease” refers to a disease caused by one or more abnormalities in the genome of a subject, such as a disease that is present from birth of the subject. Genetic diseases may be heritable and may be passed down from the parents' genes. A genetic disease may also be caused by mutations or changes of the DNAs and/or RNAs of the subject. In such cases, the genetic disease will be heritable if it occurs in the germline. Exemplary genetic diseases include, but are not limited to, Aarskog-Scott syndrome, Aase syndrome, achondroplasia, acrodysostosis, addiction, adreno-leukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia (e.g., hemophilias A and B), hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum, and Zellweger syndrome.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

As used herein, the term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrinetumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

As used herein, the term “inflammatory disease” or “inflammation” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

As used herein, an “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid, arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigusvulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme arthritis, Guillain-Barre syndrome, Hashimoto's thyroiditis, and cardiomyopathy.

The term “liver disease” or “hepatic disease” refers to damage to or a disease of the liver. Non-limiting examples of liver disease include intrahepatic cholestasis (e.g., alagille syndrome, biliary liver cirrhosis), fatty liver (e.g., alcoholic fatty liver, Reye's syndrome), hepatic vein thrombosis, hepatolenticular degeneration (i.e., Wilson's disease), hepatomegaly, liver abscess (e.g., amebic liver abscess), liver cirrhosis (e.g., alcoholic, biliary, and experimental liver cirrhosis), alcoholic liver diseases (e.g., fatty liver, hepatitis, cirrhosis), parasitic liver disease (e.g., hepatic echinococcosis, fascioliasis, amebic liver abscess), jaundice (e.g., hemolytic, hepatocellular, cholestatic jaundice), cholestasis, portal hypertension, liver enlargement, ascites, hepatitis (e.g., alcoholic hepatitis, animal hepatitis, chronic hepatitis (e.g., autoimmune, hepatitis B, hepatitis C, hepatitis D, drug induced chronic hepatitis), toxic hepatitis, viral human hepatitis (e.g., hepatitis A, hepatitis B, hepatitis C, hepatitis D, hepatitis E), granulomatous hepatitis, secondary biliary cirrhosis, hepatic encephalopathy, varices, primary biliary cirrhosis, primary sclerosing cholangitis, hepatocellular adenoma, hemangiomas, bile stones, liver failure (e.g., hepatic encephalopathy, acute liver failure), angiomyolipoma, calcified liver metastases, cystic liver metastases, fibrolamellar hepatocarcinoma, hepatic adenoma, hepatoma, hepatic cysts (e.g., Simple cysts, Polycystic liver disease, hepatobiliary cystadenoma, choledochal cyst), mesenchymal tumors (mesenchymal hamartoma, infantile hemangioendothelioma, hemangioma, peliosis hepatis, lipomas, inflammatory pseudotumor), epithelial tumors (e.g., bile duct hamartoma, bile duct adenoma), focal nodular hyperplasia, nodular regenerative hyperplasia, hepatoblastoma, hepatocellular carcinoma, cholangiocarcinoma, cystadenocarcinoma, tumors of blood vessels, angiosarcoma, Karposi's sarcoma, hemangioendothelioma, embryonal sarcoma, fibrosarcoma, leiomyosarcoma, rhabdomyosarcoma, carcinosarcoma, teratoma, carcinoid, squamous carcinoma, primary lymphoma, peliosis hepatis, erythrohepatic porphyria, hepatic porphyria (e.g., acute intermittent porphyria, porphyria cutanea tarda), and Zellweger syndrome.

The term “spleen disease” refers to a disease of the spleen. Example of spleen diseases include, but are not limited to, splenomegaly, spleen cancer, asplenia, spleen trauma, idiopathic purpura, Felty's syndrome, Hodgkin's disease, and immune-mediated destruction of the spleen.

The term “lung disease” or “pulmonary disease” refers to a disease of the lung. Examples of lung diseases include, but are not limited to, bronchiectasis, bronchitis, bronchopulmonary dysplasia, interstitial lung disease, occupational lung disease, emphysema, cystic fibrosis, acute respiratory distress syndrome (ARDS), severe acute respiratory syndrome (SARS), asthma (e.g., intermittent asthma, mild persistent asthma, moderate persistent asthma, severe persistent asthma), chronic bronchitis, chronic obstructive pulmonary disease (COPD), emphysema, interstitial lung disease, sarcoidosis, asbestosis, aspergilloma, aspergillosis, pneumonia (e.g., lobar pneumonia, multilobar pneumonia, bronchial pneumonia, interstitial pneumonia), pulmonary fibrosis, pulmonary tuberculosis, rheumatoid lung disease, pulmonary embolism, and lung cancer (e.g., non-small-cell lung carcinoma (e.g., adenocarcinoma, squamous-cell lung carcinoma, large-cell lung carcinoma), small-cell lung carcinoma).

As used herein, a “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as Pernicious Anemia, Hemorrhagic Anemia, Hemolytic Anemia, Aplastic Anemia, Sickle Cell Anemia, Sideroblastic Anemia, Anemia associated with chronic infections such as Malaria, Trypanosomiasis, HTV, Hepatitis virus or other viruses, Myelophthisic Anemias caused by marrow deficiencies, renal failure resulting from Anemia, Anemia, Polycethemia, Infectious Mononucleosis (EVI), Acute Non-Lymphocytic Leukemia (ANLL), Acute Myeloid Leukemia (AML), Acute Promyelocytic Leukemia (APL), Acute Myelomonocytic Leukemia (AMMoL), Polycethemia Vera, Lymphoma, Acute Lymphocytic Leukemia (ALL), Chronic Lymphocytic Leukemia, Wilm's Tumor, Ewing's Sarcoma, Retinoblastoma, Hemophilia, disorders associated with an increased risk of Thrombosis, Herpes, Thalessemia, antibody-mediated disorders such as transfusion reactions and Erythroblastosis, mechanical trauma to red blood cells such as micro-angiopathic hemolytic anemias, Thrombotic Thrombocytopenic Purpura and disseminated intravascular coagulation, infections by parasites such as Plasmodium, chemical injuries from, e.g., lead poisoning, and Hypersplenism.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases also refer to a type of neurological disease marked by the loss of nerve cells, including, but not limited to, Alzheimer's disease, Parkinson's disease, amyotrophic lateral sclerosis, tauopathies (including fronto-temporal dementia), and Huntington's disease. Examples of neurological diseases include, but are not limited to, headache, stupor and coma, dementia, seizure, sleep disorders, trauma, infections, neoplasms, neuroophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include Acquired Epileptiform Aphasia; Acute Disseminated Encephalomyelitis; Adrenoleukodystrophy; agenesis of the corpus callosum; Agnosia; Aicardi syndrome; Alexander disease; Alpers' disease; Alternating hemiplegia; Alzheimer's disease; Amyotrophic lateral sclerosis; anencephaly; Angelman syndrome; Angiomatosis; Anoxia; aphasia; apraxia; Arachnoid Cysts; Arachnoiditis; Arnold-Chiari malformation; Arteriovenous malformation; Asperger syndrome; Ataxia Telangiectasia; Attention Deficit Hyperactivity Disorder; autism; autonomic dysfunction; Back Pain; Batten disease; Behcet's disease; Bell's palsy; Benign Essential Blepharospasm; Benign Focal; Amyotrophy; Benign Intracranial Hypertension; Binswanger's disease; Blepharospasm; Bloch Sulzberger syndrome; Brachial plexus injury; Brain abscess; Brain injury; Brain tumors (including Glioblastoma multiforme); Spinal tumor; Brown-Sequard syndrome; Canavan disease; Carpal tunnel syndrome (CTS); Causalgia; Central pain syndrome; Central pontine myelinolysis; Cephalic disorder; Cerebral aneurysm; Cerebral arteriosclerosis; Cerebral atrophy; Cerebral gigantism; Cerebral palsy; Charcot-Marie-Tooth disease; Chemotherapy-induced neuropathy and neuropathic pain; Chiari malformation; Chorea; Chronic inflammatory demyelinating polyneuropathy (CIDP); Chronic pain; Chronic regional pain syndrome; Coffin Lowry syndrome; Coma, including Persistent Vegetative State; Congenital facial diplegia; Corticobasal degeneration; Cranial arteritis; Craniosynostosis; Creutzfeldt-Jakob disease; Cumulative trauma disorders; Cushing's syndrome; Cytomegalic inclusion body disease (CIBD); Cytomegalovirus Infection; Dancing eyes-dancing feet syndrome; Dandy-Walker syndrome; Dawson disease; De Morsier's syndrome; Dejerine-Klumpke palsy; Dementia; Dermatomyositis; Diabetic neuropathy; Diffuse sclerosis; Dysautonomia; Dysgraphia; Dyslexia; Dystonias; Early infantile epileptic encephalopathy; Empty sella syndrome; Encephalitis; Encephaloceles; Encephalotrigeminal angiomatosis; Epilepsy; Erb's palsy; Essential tremor; Fabry's disease; Fahr's syndrome; Fainting; Familial spastic paralysis; Febrile seizures; Fisher syndrome; Friedreich's ataxia; Fronto-Temporal Dementia and other “Tauopathies”; Gaucher's disease; Gerstmann's syndrome; Giant cell arteritis; Giant cell inclusion disease; Globoid cell Leukodystrophy; Guillain-Barre syndrome; HTLV-1 associated myelopathy; Hallervorden-Spatz disease; Head injury; Headache; Hemifacial Spasm; Hereditary Spastic Paraplegia; Heredopathia atactica polyneuritiformis; Herpes zoster oticus; Herpes zoster; Hirayama syndrome; HIV-Associated Dementia and Neuropathy (see also Neurological manifestations of AIDS); Holoprosencephaly; Huntington's disease and other polyglutamine repeat diseases; Hydranencephaly; Hydrocephalus; Hypercortisolism; Hypoxia; Immune-Mediated encephalomyelitis; Inclusion body myositis; Incontinentia pigmenti; Infantile; phytanic acid storage disease; Infantile Refsum disease; Infantile spasms; Inflammatory myopathy; Intracranial cyst; Intracranial hypertension; Joubert syndrome; Kearns-Sayre syndrome; Kennedy disease; Kinsbourne syndrome; Klippel Feil syndrome; Krabbe disease; Kugelberg-Welander disease; Kuru; Lafora disease; Lambert-Eaton myasthenic syndrome; Landau-Kleffner syndrome; Lateral medullary (Wallenberg) syndrome; Learning disabilities; Leigh's disease; Lennox-Gastaut syndrome; Lesch-Nyhan syndrome; Leukodystrophy; Lewy body dementia; Lissencephaly; Locked-In syndrome; Lou Gehrig's disease (aka Motor Neuron Disease or Amyotrophic Lateral Sclerosis); Lumbar disc disease; Lyme disease-Neurological Sequelae; Machado-Joseph disease; Macrencephaly; Megalencephaly; Melkersson-Rosenthal syndrome; Menieres disease; Meningitis; Menkes disease; Metachromatic leukodystrophy; Microcephaly; Migraine; Miller Fisher syndrome; Mini-Strokes; Mitochondrial Myopathies; Mobius syndrome; Monomelic amyotrophy; Motor Neurone Disease; Moyamoya disease; Mucopolysaccharidoses; Multi-Infarct Dementia; Multifocal motor neuropathy; Multiple sclerosis and other demyelinating disorders; Multiple system atrophy with postural hypotension; Muscular dystrophy; Myasthenia gravis; Myelinoclastic diffuse sclerosis; Myoclonic encephalopathy of infants; Myoclonus; Myopathy; Myotonia congenital; Narcolepsy; Neurofibromatosis; Neuroleptic malignant syndrome; Neurological manifestations of AIDS; Neurological sequelae of lupus; Neuromyotonia; Neuronal ceroid lipofuscinosis; Neuronal migration disorders; Niemann-Pick disease; O'Sullivan-McLeod syndrome; Occipital Neuralgia; Occult Spinal Dysraphism Sequence; Ohtahara syndrome; Olivopontocerebellar Atrophy; Opsoclonus Myoclonus; Optic neuritis; Orthostatic Hypotension; Overuse syndrome; Paresthesia; Parkinson's disease; Paramyotonia Congenita; Paraneoplastic diseases; Paroxysmal attacks; Parry Romberg syndrome; Pelizaeus-Merzbacher disease; Periodic Paralyses; Peripheral Neuropathy; Painful Neuropathy and Neuropathic Pain; Persistent Vegetative State; Pervasive developmental disorders; Photic sneeze reflex; Phytanic Acid Storage disease; Pick's disease; Pinched Nerve; Pituitary Tumors; Polymyositis; Porencephaly; Post-Polio syndrome; Postherpetic Neuralgia (PHN); Postinfectious Encephalomyelitis; Postural Hypotension; Prader-Willi syndrome; Primary Lateral Sclerosis; Prion diseases; Progressive; Hemifacial Atrophy; Progressive multifocal leukoencephalopathy; Progressive Sclerosing Poliodystrophy; Progressive Supranuclear Palsy; Pseudotumor cerebri; Ramsay-Hunt syndrome (Type I and Type II); Rasmussen's Encephalitis; Reflex Sympathetic Dystrophy syndrome; Refsum disease; Repetitive Motion Disorders; Repetitive Stress Injuries; Restless Legs syndrome; Retrovirus-Associated Myelopathy; Rett syndrome; Reye's syndrome; Saint Vitus Dance; Sandhoff disease; Schilder's disease; Schizencephaly; Septo-Optic Dysplasia; Shaken Baby syndrome; Shingles; Shy-Drager syndrome; Sjogren's syndrome; Sleep Apnea; Soto's syndrome; Spasticity; Spina bifida; Spinal cord injury; Spinal cord tumors; Spinal Muscular Atrophy; Stiff-Person syndrome; Stroke; Sturge-Weber syndrome; Subacute Sclerosing Panencephalitis; Subarachnoid Hemorrhage; Subcortical Arteriosclerotic Encephalopathy; Sydenham Chorea; Syncope; Syringomyelia; Tardive dyskinesia; Tay-Sachs disease; Temporal arteritis; Tethered Spinal Cord syndrome; Thomsen disease; Thoracic Outlet syndrome; Tic Douloureux; Todd's Paralysis; Tourette syndrome; Transient ischemic attack; Transmissible Spongiform Encephalopathies; Transverse myelitis; Traumatic Brain injury; Tremor; Trigeminal Neuralgia; Tropical Spastic Paraparesis; Tuberous Sclerosis; Vascular Dementia (Multi-Infarct Dementia); Vasculitis including Temporal Arteritis; Von Hippel-Lindau Disease (VHL); Wallenberg's syndrome; Werdnig-Hoffman disease; West syndrome; Whiplash; Williams syndrome; Wilson's disease; and Zellweger syndrome.

A “painful condition” includes, but is not limited to, neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g. nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

The term “psychiatric disorder” refers to a disease of the mind and includes diseases and disorders listed in the Diagnostic and Statistical Manual of Mental Disorders—Fourth Edition (DSM-IV), published by the American Psychiatric Association, Washington D. C. (1994). Psychiatric disorders include, but are not limited to, anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., type 1 diabetes, type 2 diabetes, gestational diabetes), hyperglycemia, hyperinsulinemia, insulin resistance, and obesity.

The term “musculoskeletal disease” or “MSD” refers to an injury and/or pain in a subject's joints, ligaments, muscles, nerves, tendons, and structures that support limbs, neck, and back. In certain embodiments, an MSD is a degenerative disease. In certain embodiments, an MSD includes an inflammatory condition. Body parts of a subject that may be associated with MSDs include upper and lower back, neck, shoulders, and extremities (arms, legs, feet, and hands). In certain embodiments, an MSD is a bone disease, such as achondroplasia, acromegaly, bone callus, bone demineralization, bone fracture, bone marrow disease, bone marrow neoplasm, dyskeratosis congenita, leukemia (e.g., hairy cell leukemia, lymphocytic leukemia, myeloid leukemia, Philadelphia chromosome-positive leukemia, plasma cell leukemia, stem cell leukemia), systemic mastocytosis, myelodysplastic syndromes, paroxysmal nocturnal hemoglobinuria, myeloid sarcoma, myeloproliferative disorders, multiple myeloma, polycythemia vera, pearson marrow-pancreas syndrome, bone neoplasm, bone marrow neoplasm, Ewing sarcoma, osteochondroma, osteoclastoma, osteosarcoma, brachydactyly, Camurati-Engelmann syndrome, Craniosynostosis, Crouzon craniofacial dysostosis, dwarfism, achondroplasia, bloom syndrome, Cockayne syndrome, Ellis-van Creveld syndrome, Seckel syndrome, spondyloepiphyseal dysplasia, spondyloepiphyseal dysplasia congenita, Werner syndrome, hyperostosis, osteophyte, Klippel-Trenaunay-Weber syndrome, Marfan syndrome, McCune-Albright syndrome, osteitis, osteoarthritis, osteochondritis, osteochondrodysplasia, Kashin-Beck disease, Leri-Weill dyschondrosteosis, osteochondrosis, osteodystrophy, osteogenesis imperfecta, osteolysis, Gorham-Stout syndrome, osteomalacia, osteomyelitis, osteonecrosis, osteopenia, osteopetrosis, osteoporosis, osteosclerosis, otospondylomegaepiphyseal dysplasia, pachydermoperiostosis, Paget disease of bone, Polydactyly, Meckel syndrome, rickets, Rothmund-Thomson syndrome, Sotos syndrome, spondyloepiphyseal dysplasia, spondyloepiphyseal dysplasia congenita, syndactyly, Apert syndrome, syndactyly type II, or Werner syndrome. In certain embodiments, an MSD is a cartilage disease, such as cartilage neoplasm, osteochondritis, osteochondrodysplasia, Kashin-Beck disease, or Leri-Weill dyschondrosteosis. In certain embodiments, an MSD is hernia, such as intervertebral disk hernia. In certain embodiments, an MSD is a joint disease, such as arthralgia, arthritis (e.g., gout (e.g., Kelley-Seegmiller syndrome, Lesch-Nyhan syndrome), Lyme disease, osteoarthritis, psoriatic arthritis, reactive arthritis, rheumatic fever, rheumatoid arthritis, Felty syndrome, synovitis, Blau syndrome, nail-patella syndrome, spondyloarthropathy, reactive arthritis, Stickler syndrome, synovial membrane disease, synovitis, or Blau syndrome. In certain embodiments, an MSD is Langer-Giedion syndrome. In certain embodiments, an MSD is a muscle disease, such as Barth syndrome, mitochondrial encephalomyopathy, MELAS syndrome, MERRF syndrome, MNGIE syndrome, mitochondrial myopathy, Kearns-Sayre syndrome, myalgia, fibromyalgia, polymyalgia rheumatica, myoma, myositis, dermatomyositis, neuromuscular disease, Kearns-Sayre syndrome, muscular dystrophy, myasthenia, congenital myasthenic syndrome, Lambert-Eaton myasthenic syndrome, myasthenia gravis, myotonia, myotonia congenita, spinal muscular atrophy, tetany, ophthalmoplegia, or rhabdomyolysis. In certain embodiments, an MSD is Proteus syndrome. In certain embodiments, an MSD is a rheumatic diseases, such as arthritis (e.g., gout (e.g., Kelley-Seegmiller syndrome, Lesch-Nyhan lyme disease)), osteoarthritis, psoriatic arthritis, reactive arthritis, rheumatic fever, rheumatoid arthritis, Felty syndrome, synovitis, Blau syndrome, gout (e.g., Kelley-Seegmiller syndrome, Lesch-Nyhan syndrome), polymyalgia rheumatica, rheumatic fever, rheumatic heart disease, or Sjogren syndrome. In certain embodiments, an MSD is Schwartz-Jampel syndrome. In certain embodiments, an MSD is a skeleton disease, such as Leri-Weill dyschondrosteosis, skeleton malformations, Melnick-Needles syndrome, pachydermoperiostosis, Rieger syndrome, spinal column disease, intervertebral disk hernia, scoliosis, spina bifida, spondylitis, ankylosing spondylitis, spondyloarthropathy, reactive arthritis, spondyloepiphyseal dysplasia, spondyloepiphyseal dysplasia congenita, or spondylosis.

The term “micronutrient deficiency” refers to the lack of sufficient micronutrients required for optimal health. In humans and other animals they include both vitamin deficiencies and mineral deficiencies. In certain embodiments, micronutrient deficiency causes or exacerbates disease. In some embodiments, micronutrients include iodine, iron, zinc, calcium, selenium, fluorine, and vitamins A, B6, B12, B1, B2, B3, B9 and C. In certain embodiments, micronutrient deficiencies are caused by long-term shortages of nutritious food or by infections. In some embodiments, micronutrient deficiencies are caused or exacerbated by illnesses that cause rapid loss of nutrients through feces or vomit (e.g., diarrhea or malaria).

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

Unless otherwise required by context, singular terms shall include pluralities, and plural terms shall include the singular.

### DETAILED DESCRIPTION OF CERTAIN EMBODIMENTS

Provided herein are compounds (e.g., compounds of Formula (I)), and pharmaceutically acceptable salts, stereoisomers, and isotopically labeled derivatives thereof, and compositions and kits thereof. The compounds provided herein can form particles and may therefore be used to deliver agents (e.g., a vitamin or mineral) to a subject. Also provided herein are methods of delivery and methods of treating a disease, disorder, or condition, comprising administering to the subject a composition provided herein. Methods of synthesis of a compound provided herein (e.g., a compound of Formula (I)), or a pharmaceutically acceptable salt, stereoisomer, or isotopically labeled derivative thereof, are also provided.

### Compounds

Provided herein are compounds of Formula (I):

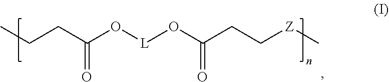

and pharmaceutically acceptable salts, stereoisomers, or isotopically labeled derivatives thereof, wherein:
L is a heterocycle comprising at least one oxygen atom;
each Z is independently of Formula (i), (ii), (iii), or (iv):

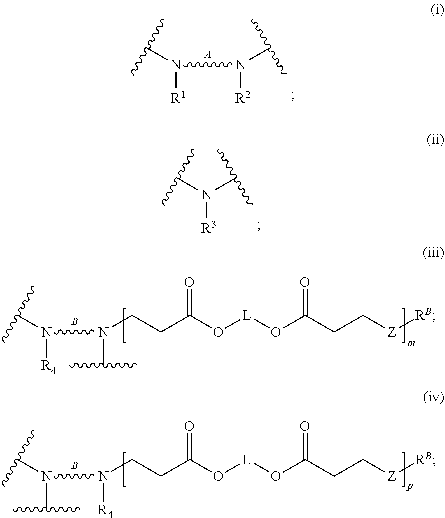

linker A is branched or unbranched, optionally substituted, cyclic or acyclic aliphatic; branched or unbranched, optionally substituted, cyclic or acyclic heteroaliphatic; optionally substituted arylene; or optionally substituted heteroarylene;
R¹ and R² are each independently hydrogen; branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group; or
one or both R¹ or R² are each optionally independently joined to linker A together with the intervening atoms to form a linker comprising one or more N-containing heterocycles; or
R¹ and R² are joined together with the intervening atoms to form a N-containing heterocycle;
R³ is branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group;
linker B is branched or unbranched, optionally substituted, cyclic or acyclic aliphatic; branched or unbranched, optionally substituted, cyclic or acyclic heteroaliphatic; optionally substituted arylene; or optionally substituted heteroarylene;
R⁴ is hydrogen, branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group; or
R⁴ is joined to linker B together with the intervening atoms to form a linker comprising a N-containing heterocycle; and
m, n, and p are each independently an integer between 1 and 10,000.

Also provided herein are compounds comprising Formula (I) (e.g., compounds of Formula (III)). Stability of PAE polymer may be enhanced with capped end groups, as carbon-carbon double bonds may be unstable and might transform to a radical and further react (e.g. with an agent, such as VA molecules, over long-term storage). Therefore, an additional step in synthesis caps this end group and eliminates occurrence of radicals, without significant change in the overall polymer structure.

Provided herein are compounds of Formula (III):

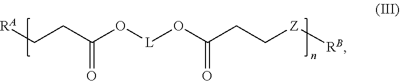

and pharmaceutically acceptable salts, stereoisomers, or isotopically labeled derivatives thereof, wherein:
L is a heterocycle comprising at least one oxygen atom;
each Z is independently of Formula (i), (ii), (iii), or (iv):

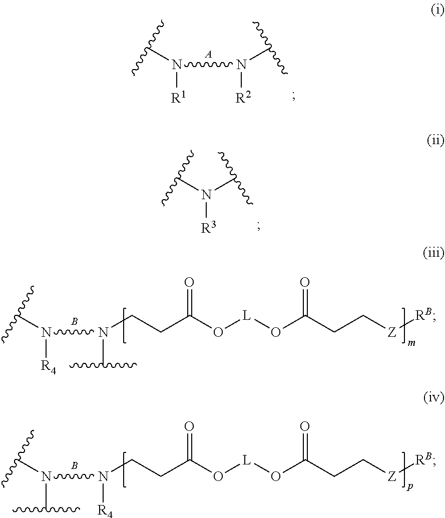

linker A is branched or unbranched, optionally substituted, cyclic or acyclic aliphatic; branched or unbranched, optionally substituted, cyclic or acyclic heteroaliphatic; optionally substituted arylene; or optionally substituted heteroarylene;
R¹ and R² are each independently hydrogen; branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group; or
one or both R¹ or R² are each optionally independently joined to linker A together with the intervening atoms to form a linker comprising one or more N-containing heterocycles; or
R¹ and R² are joined together with the intervening atoms to form a N-containing heterocycle;
R³ is branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group;
linker B is branched or unbranched, optionally substituted, cyclic or acyclic aliphatic; branched or unbranched, optionally substituted, cyclic or acyclic heteroaliphatic; optionally substituted arylene; or optionally substituted heteroarylene;
R⁴ is hydrogen, branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group; or
R⁴ is joined to linker B together with the intervening atoms to form a linker comprising a N-containing heterocycle;
m, n, and p are each independently an integer between 1 and 10,000;
each instance of R^{A} is independently —OR^{C}, —SR^{C}, —N(R^{C})₂, —ZR^{C}, or

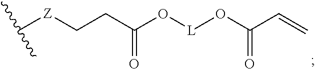

each instance of R^{B} is independently

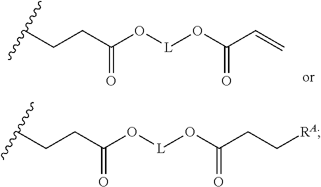

and
each instance of R^{C} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, a nitrogen protecting group when attached to a nitrogen atom, or two instances of R^{C} attached to the same intervening atom are joined together with the intervening atom to form optionally substituted heterocyclyl or optionally substituted heteroaryl.

As defined herein, each instance of R^{A} is independently —OR^{C}, —SR^{C}, —N(R^{C})₂, —ZR^{C}, or

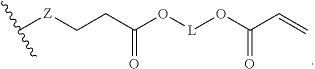

In some embodiments, each instance of R^{A} is independently —OR^{C}, —SR^{C}, —N(R^{C})₂, or —ZR^{C}. In some embodiments, each instance of R^{A} is independently —OR^{C}, —SR^{C}, or —N(R^{C})₂. In some embodiments, each instance of R^{A} is independently —N(R^{C})₂, —ZR^{C}, or

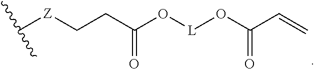

In some embodiments, each instance of R^{A} is independently —ZR^{C}, or

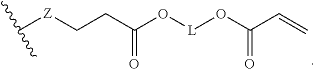

In some embodiments, at least one instance of R^{A} is —OR^{C}. In some embodiments, at least one instance of R^{A} is —SR^{C}. In some embodiments, at least one instance of R^{A} is —N(R^{C})₂. In some embodiments, at least one instance of R^{A} is —ZR^{C}. In some embodiments, at least one instance of R^{A} is

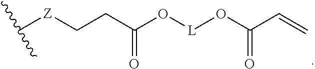

As defined herein each instance of R^{B} is independently or

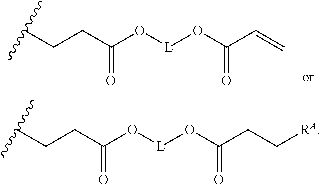

In some embodiments, at least one instance of R^{B} is

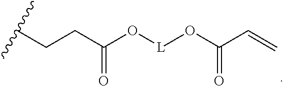

In some embodiments, at least one instance of R^{B} is

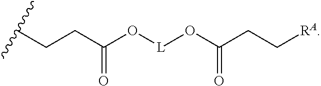

In some embodiments, each instance of R^{A} is independently —OR^{C}, —SR^{C}, —N(R^{C})₂, or —ZR^{C}, and R^{B} is

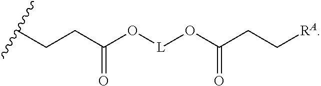

In some embodiments, each instance of R^{A} is independently —N(R^{c})₂ or —ZR^{C}, and R^{B} is

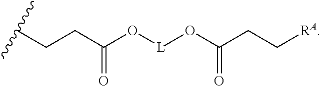

In some embodiments, R^{A} is —ZR^{C}, and R^{B} is

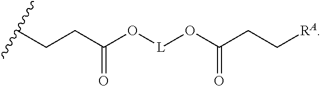

In some embodiments, R^{A} is

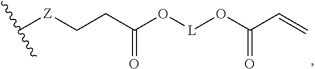

### and R^{B} is

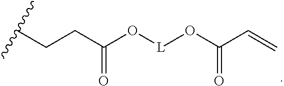

In some embodiments, R^{A} is

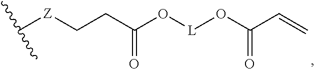

### and R^{B} is

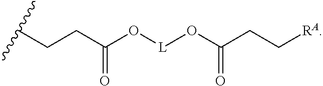

In some embodiments, each instance of R^{A} is independently —OR^{C}, —SR^{C}, —N(R^{c})₂, or —ZR^{C}, and R^{B} is

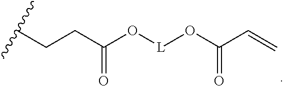

In some embodiments, each instance of R^{A} is independently —N(R^{C})₂ or —ZR^{C}, and R^{B} is

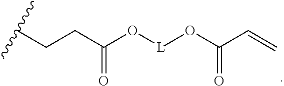

In some embodiments, R^{A} is —ZR^{C}, and R^{B} is

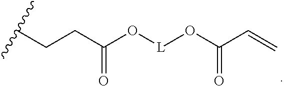

As defined herein, each instance of R^{C} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, a nitrogen protecting group when attached to a nitrogen atom, or two instances of R^{C} attached to the same intervening atom are joined together with the intervening atom to form optionally substituted heterocyclyl or optionally substituted heteroaryl. In some embodiments, at least one instance of R^{C} is independently hydrogen, optionally substituted alkyl, optionally substituted alkenyl, optionally substituted alkynyl, optionally substituted heteroalkyl, optionally substituted heteroalkenyl, or optionally substituted heteroalkynyl. In some embodiments, at least one instance of R^{C} is optionally substituted carbocyclyl, optionally substituted heterocyclyl, optionally substituted aryl, or optionally substituted heteroaryl. In some embodiments, at least one instance of R^{C} is independently hydrogen, optionally substituted C₁₋₁₀ alkyl, optionally substituted C₁₋₁₀ alkenyl, optionally substituted C₁₋₁₀ alkynyl, optionally substituted C₃₋₁₄ carbocyclyl, or optionally substituted C₆₋₁₄ aryl. In some embodiments, at least one instance of R^{C} is independently hydrogen, optionally substituted C₁₋₁₀ alkyl, or optionally substituted phenyl. In some embodiments, at least one instance of R^{C} is hydrogen, a nitrogen protecting group when attached to a nitrogen atom, an oxygen protecting group when attached to an oxygen atom, or a sulfur protecting group when attached to a sulfur atom. In some embodiments, at least one instance of R^{C} is a nitrogen protecting group when attached to a nitrogen atom, an oxygen protecting group when attached to an oxygen atom, or a sulfur protecting group when attached to a sulfur atom. In some embodiments, two instances of R^{C} attached to the same intervening atom are joined together with the intervening atom to form an optionally substituted, monocyclic, heterocyclic or heteroaryl ring.

As defined herein, L is a heterocycle comprising at least one oxygen atom. In certain embodiments, L comprises one oxygen atom. In some embodiments, L comprises at least two oxygen atoms. In certain embodiments, L comprises two oxygen atoms. In some embodiments, L comprises at least three oxygen atoms. In certain embodiments, L comprises three oxygen atoms. In certain embodiments, L comprises a pyran. In certain embodiments, L comprises a furan.

In some embodiments, L is derived from a monosaccharide. In certain embodiments, the monosaccharide is arabinose, lyxose, ribose, xylose, ribulofuranose, xylulose, allose, altrose, galactose, glucose, gulose, idose, mannose, talose, fructose, psicose, sorbose, tagatose, mannoheptulose, and sedoheptulose. In some embodiments, the monosaccharide is allose, altrose, galactose, glucose, gulose, idose, mannose, talose, fructose, psicose, sorbose, tagatose. In certain embodiments, the monosaccharide is glucose or galactose. In some embodiments, the monosaccharide is galactose. In certain embodiments, the monosaccharide is glucose.

In some embodiments, L is an unsubstituted 3-14 membered heterocycle comprising at least one oxygen atom. In certain embodiments, L is a substituted 3-14 membered heterocycle comprising at least one oxygen atom. In certain embodiments, L is optionally substituted, 3- to 7-membered, monocyclic heterocycle, comprising at least one oxygen atom. In certain embodiments, L is substituted, 3- to 7-membered, monocyclic heterocycle, comprising at least one oxygen atom. In certain embodiments, L is unsubstituted, 3- to 7-membered, monocyclic heterocycle, comprising at least one oxygen atom. In certain embodiments, L is optionally substituted, 5- to 6-membered, monocyclic heterocycle, comprising at least one oxygen atom. In certain embodiments, L is substituted, 5- to 5-membered, monocyclic heterocycle, comprising at least one oxygen atom. In certain embodiments, L is unsubstituted, 5- to 6-membered, monocyclic heterocycle, comprising at least one oxygen atom. In certain embodiments, L is optionally substituted, 8- to 10-membered, bicyclic heterocycle, comprising at least one oxygen atom. In certain embodiments, L is substituted, 8- to 10-membered, bicyclic heterocycle, comprising at least one oxygen atom. In certain embodiments, L is unsubstituted, 8- to 10-membered, bicyclic heterocycle, comprising at least one oxygen atom.

In some embodiments, L is substituted with at least one hydroxy or alkoxy substituent. In certain embodiments, L is substituted with —OH, —OR⁵, —OR⁶, or —OR⁷, wherein R⁵, R⁶, and R⁷ are each independently an oxygen protecting group, or wherein R⁵, R⁶, and R⁷ may be joined together with the intervening atoms to form optionally substituted heterocyclyl. In some embodiments, L is substituted with —OH. In certain embodiments, L is substituted with —OR⁵, —OR⁶, or —OR⁷. In some embodiments, L is substituted with —OR⁵. In certain embodiments, L is substituted with —OR⁶. In some embodiments, L is substituted with —OR⁷.

In certain embodiments, L is

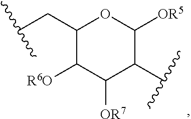

and the compound of Formula (I) is of Formula (I-A)

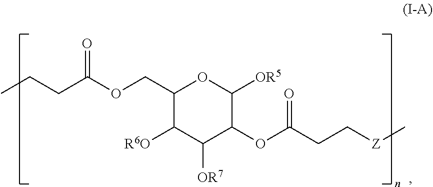

wherein R⁵, R⁶, and R⁷ are each independently hydrogen, C₁₋₆ aliphatic, an oxygen protecting group, or wherein R⁶ and R⁷ may be joined together with the intervening atoms to form optionally substituted heterocyclyl.

As defined herein, R⁵, R⁶, and R⁷ are each independently hydrogen, C₁₋₆, aliphatic, an oxygen protecting group, or wherein R⁶ and R⁷ may be joined together with the intervening atoms to form optionally substituted heterocyclyl. In some embodiments, R⁵, R⁶, and R⁷ are each independently an oxygen protecting group or wherein R⁶ and R⁷ may be joined together with the intervening atoms to form optionally substituted heterocyclyl. In some embodiments, R⁵ is an oxygen protecting group. In some embodiments, R⁵ is C₁₋₆ aliphatic. In some embodiments, R⁵ is hydrogen. In certain embodiments, R⁶ is an oxygen protecting group. In some embodiments, R⁶ is C₁₋₆ aliphatic. In some embodiments, R⁶ is hydrogen. In some embodiments, R⁷ is an oxygen protecting group. In some embodiments, R⁷ is C₁₋₆ aliphatic. In some embodiments, R⁷ is hydrogen. In certain embodiments, the oxygen protecting group is unsubstituted C₁-C₆ alkyl, silyl, TBDPS, TBDMS, TIPS, TES, TMS, MOM, THP, t-Bu, Bn, allyl, acetyl, pivaloyl, or benzoyl. In some embodiments, the oxygen protecting group is unsubstituted C₁-C₆ alkyl.

In certain embodiments, R⁶ and R⁷ are joined together with the intervening atoms to form optionally substituted heterocyclyl. In certain embodiments, R⁶ and R⁷ are joined together with the intervening atoms to form optionally substituted 5-6-membered heterocyclyl. In certain embodiments, R⁶ and R⁷ are joined together with the intervening atoms to form unsubstituted 5-6-membered heterocyclyl. In certain embodiments, R⁶ and R⁷ are joined together with the intervening atoms to form substituted 5-6-membered heterocyclyl. In certain embodiments, R⁶ and R⁷ are joined together with the intervening atoms to form 5-6-membered heterocyclyl substituted with one or more C₁-C₆ alkyl substituents. In some embodiments, R⁶ and R⁷ are joined together with the intervening atoms to form a cyclic ketal. In certain embodiments, R⁶ and R⁷ are joined together with the intervening atoms to form a 1,3-dioxolane. In some embodiments, R⁶ and R⁷ are joined together with the intervening atoms to form 2,2-dimethyl-1,3-dioxolane.

In certain embodiments, L is

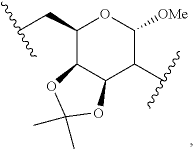 [SMILES: *C[C@H]1O[C@H](OC)C(*)[C@H]2OC(C)(C)O[C@H]21]

and
the compound of Formula (I-A) is of Formula (I-A-i)

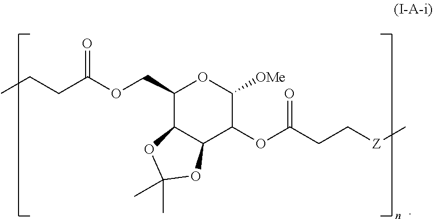

In certain embodiments, L is

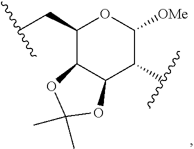 [SMILES: *C[C@H]1O[C@H](OC)[C@H](*)[C@H]2OC(C)(C)O[C@H]21]

and
the compound of Formula (I-A) is of Formula (I-A-ii)

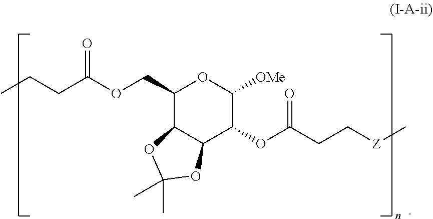

In certain embodiments, L is

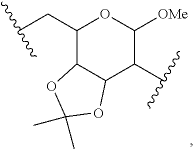 [SMILES: *CC1OC(OC)C(*)C2OC(C)(C)OC12]

and
the compound of Formula (I-A) is of Formula (I-A-iii)

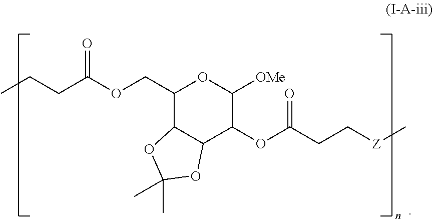

In certain embodiments, L is

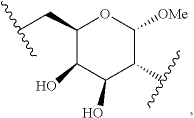 [SMILES: *C[C@H]1O[C@H](OC)[C@H](*)[C@@H](O)[C@H]1O]

and
the compound of Formula (I-A) is of Formula (I-A-iv)

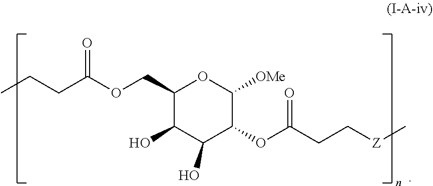

In certain embodiments, L is

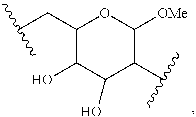 [SMILES: *CC1OC(OC)C(*)C(O)C1O]

and
the compound of Formula (I-A) is of Formula (I-A-v)

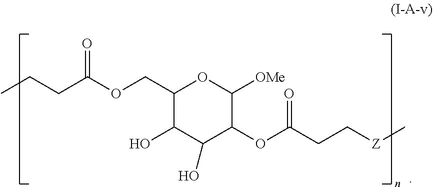

In certain embodiments, L is

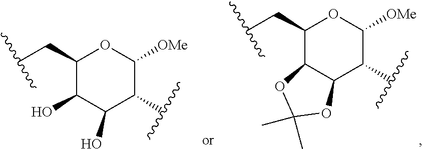 [SMILES: *C[C@H]1O[C@H](OC)[C@H](*)[C@@H](O)[C@H]1O ; *C[C@H]1O[C@H](OC)[C@H](*)[C@H]2OC(C)(C)O[C@H]21]

and
the compound of Formula (I-A) is of Formula (I-A-vi)
wherein the sum of n1 and n2 is n.

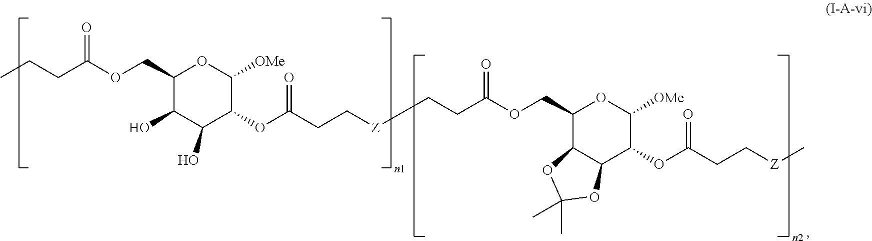

In certain embodiments, L is

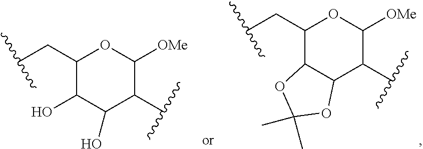 [SMILES: *CC1OC(OC)C(*)C(O)C1O ; *CC1OC(OC)C(*)C2OC(C)(C)OC12]

and
the compound of Formula (I-A) is of Formula (I-A-vii)

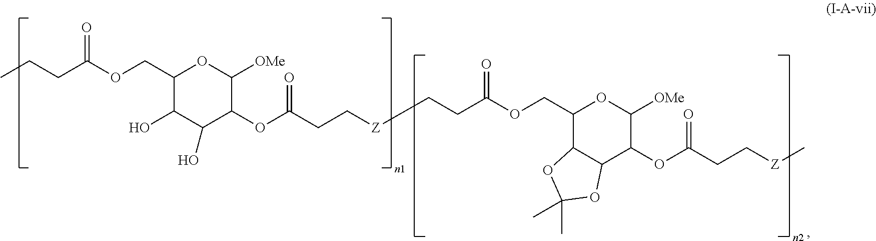

wherein the sum of n1 and n2 is n.

In certain embodiments, L is

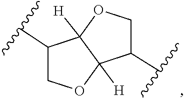 [SMILES: *C1COC2C(*)COC12]

and
the compound of Formula (I) is of Formula (I-B):

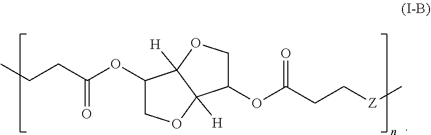

In certain embodiments, the compound of Formula (I) is of Formula (I-B-i) or Formula (I-B-ii):

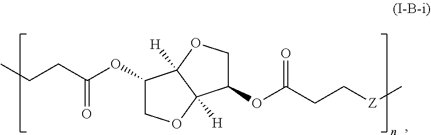

wherein L is

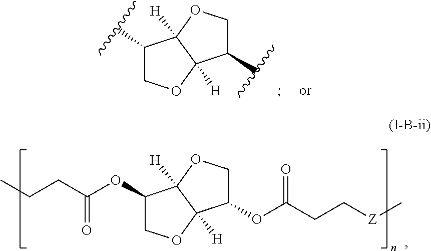

wherein L is

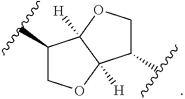 [SMILES: *[C@@H]1CO[C@H]2[C@@H]1OC[C@@H]2*]

In certain embodiments, the compound of Formula (I) is of Formula (I-B-i):

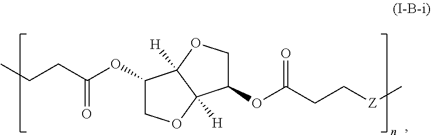

wherein L is

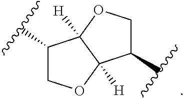 [SMILES: *[C@@H]1CO[C@H]2[C@@H]1OC[C@@H]2*]

In certain embodiments, the compound of Formula (I) is of Formula (I-B-ii):

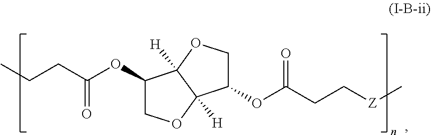

wherein L is

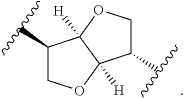 [SMILES: *[C@@H]1CO[C@H]2[C@@H]1OC[C@@H]2*]

In some embodiments, L is

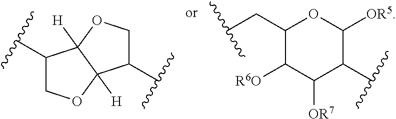

In some embodiments, L is

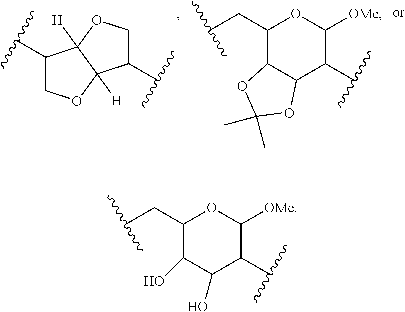 [SMILES: *C1COC2C(*)COC12 ; "," ; *CC1OC(OC)C(*)C2OC(C)(C)OC12 ; "or" ; *CC1OC(OC)C(*)C(O)C1O]

In some embodiments, L is

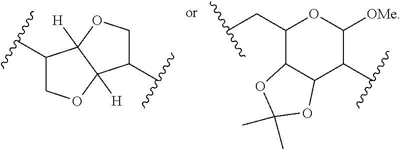 [SMILES: *C1COC2C(*)COC12 ; "or" ; *CC1OC(OC)C(*)C2OC(C)(C)OC12]

In some embodiments, L is

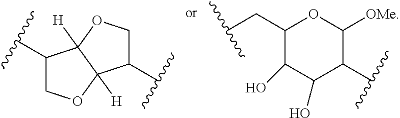 [SMILES: *C1COC2C(*)COC12 ; "or" ; *CC1OC(OC)C(*)C(O)C1O]

In some embodiments, L is or

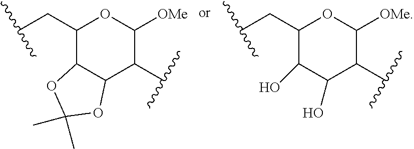 [SMILES: *CC1OC(OC)C(*)C2OC(C)(C)OC12 ; "or" ; *CC1OC(OC)C(*)C(O)C1O]

In some embodiments, L is

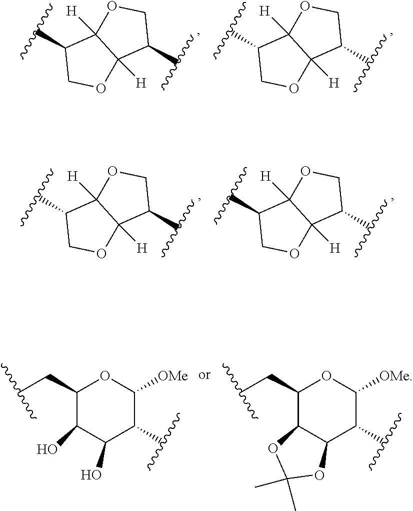

In some embodiments, L is

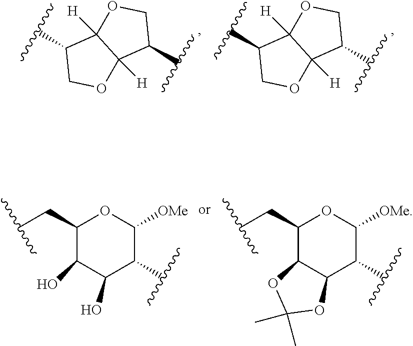

In some embodiments, L is

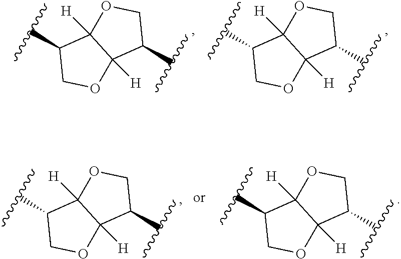

In some embodiments, L is

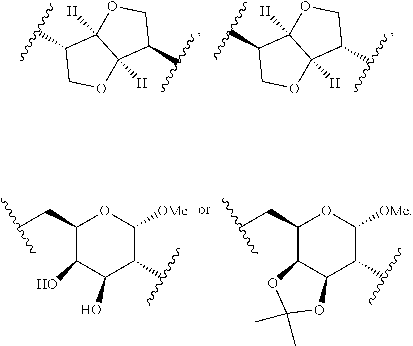

In some embodiments, L is

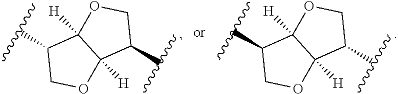 [SMILES: *[C@@H]1CO[C@H]2[C@@H]1OC[C@@H]2* ; "," ; "or" ; *[C@@H]1CO[C@H]2[C@@H]1OC[C@@H]2* ; "."]

In some embodiments, L is

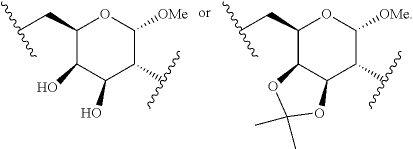 [SMILES: *C[C@H]1O[C@H](OC)[C@H](*)[C@@H](O)[C@H]1O ; "or" ; *C[C@H]1O[C@H](OC)[C@H](*)[C@H]2OC(C)(C)O[C@H]21]

As defined herein, n is an integer between 1 and 10,000. In certain embodiments, n is an integer between 3 and 10,000. In some embodiments, n is an integer between 3 and 10. In certain embodiments, n is an integer between 10 and 10,000. In some embodiments, n is an integer between 10 and 7,500. In certain embodiments, n is an integer between 10 and 5,000. In some embodiments, n is an integer between 10 and 2,000. In certain embodiments, n is an integer between 10 and 1,500. In some embodiments, n is an integer between 10 and 1,000. In certain embodiments, n is an integer between 10 and 500.

As defined herein, m is an integer between 1 and 10,000. In certain embodiments, m is an integer between 3 and 10,000. In some embodiments, m is an integer between 3 and 10. In certain embodiments, m is an integer between 10 and 10,000. In some embodiments, m is an integer between 10 and 7,500. In certain embodiments, m is an integer between 10 and 5,000. In some embodiments, m is an integer between 10 and 2,000. In certain embodiments, m is an integer between 10 and 1,500. In some embodiments, m is an integer between 10 and 1,000. In certain embodiments, m is an integer between 10 and 500.

As defined herein, p is an integer between 1 and 10,000. In certain embodiments, p is an integer between 3 and 10,000. In some embodiments, p is an integer between 3 and 10. In certain embodiments, p is an integer between 10 and 10,000. In some embodiments, p is an integer between 10 and 7,500. In certain embodiments, p is an integer between 10 and 5,000. In some embodiments, p is an integer between 10 and 2,000. In certain embodiments, p is an integer between 10 and 1,500. In some embodiments, p is an integer between 10 and 1,000. In certain embodiments, p is an integer between 10 and 500.

As defined herein, each Z is independently of Formula (i), (ii), (iii), or (iv):

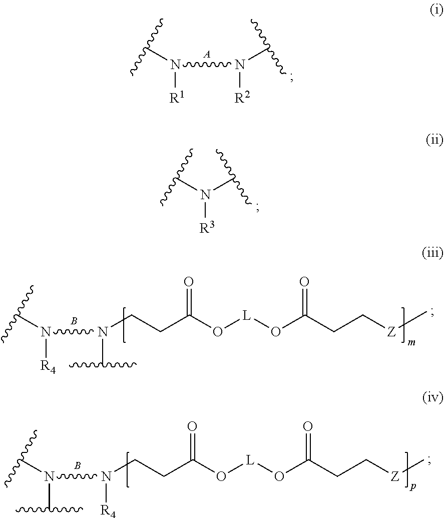

In certain embodiments, each Z is independently of Formula (i), (ii), or (iii). In some embodiments, each Z is independently of Formula (i), (ii), or (iv). In certain embodiments, each Z is independently of Formula (i), (iii), or (iv). In some embodiments, each Z is independently of Formula (ii), (iii), or (iv). In certain embodiments, each Z is independently of Formula (i) or (ii). In some embodiments, each Z is independently of Formula (iii) or (iv). In certain embodiments, each Z is independently of Formula (i). In certain embodiments, each Z is independently of Formula (ii). In certain embodiments, each Z is independently of Formula (iii). In certain embodiments, each Z is independently of Formula (iv).

In some embodiments, Formula (I) comprises at least one Z of Formula (i). In certain embodiments, Formula (I) comprises at least one Z of Formula (ii). In some embodiments, Formula (I) comprises at least one Z of Formula (iii). In certain embodiments, Formula (I) comprises at least one Z of Formula (iv).

In some embodiments, Formula (I) comprises at least one Z of Formula (i) and at least one Z of Formula (ii). In certain embodiments, Formula (I) comprises at least one Z of Formula (i) and at least one Z of Formula (iii). In some embodiments, Formula (I) comprises at least one Z of Formula (i) and at least one Z of Formula (iv). In certain embodiments, Formula (I) comprises at least one Z of Formula (ii) and at least one Z of Formula (iii). In some embodiments, Formula (I) comprises at least one Z of Formula (ii) and at least one Z of Formula (iv). In certain embodiments, Formula (I) comprises at least one Z of Formula (iii) and at least one Z of Formula (iv).

In some embodiments, Formula (I) comprises at least one Z of Formula (i), at least one Z of Formula (ii), and at least one Z of Formula (iii). In some embodiments, Formula (I) comprises at least one Z of Formula (i), at least one Z of Formula (ii), and at least one Z of Formula (iv). In some embodiments, Formula (I) comprises at least one Z of Formula (i), at least one Z of Formula (iii), and at least one Z of Formula (iv). In some embodiments, Formula (I) comprises at least one Z of Formula (ii), at least one Z of Formula (iii), and at least one Z of Formula (iv).

As defined herein, R¹ and R² are each independently hydrogen; branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group; or one or both R¹ or R² are each optionally independently joined to linker A together with the intervening atoms to form a linker comprising one or more N-containing heterocycles; or R¹ and R² are joined together with the intervening atoms to form a N-containing heterocycle.

In certain embodiments, R¹ and R² are each independently hydrogen; branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group.

In some embodiments, R¹ is hydrogen.

In certain embodiments, R¹ is branched or unbranched, optionally substituted, cyclic or acyclic aliphatic. In some embodiments, R¹ is branched or unbranched, optionally substituted, cyclic aliphatic. In certain embodiments, R¹ is substituted cyclic aliphatic. In some embodiments, linker is unsubstituted cyclic aliphatic. In certain embodiments, R¹ is branched or unbranched, optionally substituted carbocyclylene. In some embodiments, R¹ is substituted carbocyclylene. In certain embodiments, R¹ is unsubstituted carbocyclylene. In certain embodiments, R¹ is branched or unbranched, optionally substituted C₃-C₈ carbocyclylene. In some embodiments, R¹ is substituted C₃-C₈ carbocyclylene. In certain embodiments, R¹ is unsubstituted C₃-C₈ carbocyclylene. In some embodiments, R¹ is branched or unbranched, optionally substituted, acyclic aliphatic. In some embodiments, R¹ is substituted acyclic aliphatic. In certain embodiments, R¹ is unsubstituted acyclic aliphatic. In some embodiments, R¹ is branched or unbranched, optionally substituted, alkylene. In some embodiments, R¹ is substituted alkylene. In some embodiments, R¹ is unsubstituted alkylene. In some embodiments, R¹ is branched or unbranched, optionally substituted, C₁-C₁₀ alkylene. In some embodiments, R¹ is substituted C₁-C₁₀ alkylene. In some embodiments, R¹ is unsubstituted C₁-C₁₀ alkylene. In some embodiments, R¹ is branched or unbranched, optionally substituted, alkenylene. In some embodiments, R¹ is substituted alkenylene. In some embodiments, R¹ is unsubstituted alkenylene. In some embodiments, R¹ is branched or unbranched, optionally substituted, C₂-C₁₀ alkenylene. In some embodiments, R¹ is substituted C₂-C₁₀ alkenylene. In some embodiments, R¹ is unsubstituted C₂-C₁₀ alkenylene. In some embodiments, R¹ is branched or unbranched, optionally substituted, alkynylene. In some embodiments, R¹ is substituted alkynylene. In some embodiments, R¹ is unsubstituted alkynylene. In some embodiments, R¹ is branched or unbranched, optionally substituted, C₂-C₁₀ alkynylene. In some embodiments, R¹ is substituted C₂-C₁₀ alkynylene. In some embodiments, R¹ is unsubstituted C₂-C₁₀ alkynylene.

In certain embodiments, R¹ is branched or unbranched, optionally substituted, cyclic or acyclic heteroaliphatic. In some embodiments, R¹ is branched or unbranched, optionally substituted, cyclic heteroaliphatic. In certain embodiments, R¹ is substituted cyclic heteroaliphatic. In some embodiments, linker is unsubstituted cyclic heteroaliphatic. In certain embodiments, R¹ is branched or unbranched, optionally substituted heterocyclylene. In some embodiments, R¹ is substituted heterocyclylene. In certain embodiments, R¹ is unsubstituted heterocyclylene. In certain embodiments, R¹ is branched or unbranched, optionally substituted 3-8-membered heterocyclylene. In some embodiments, R¹ is substituted 3-8-membered heterocyclylene. In certain embodiments, R¹ is unsubstituted 3-8-membered heterocyclylene. In some embodiments, R¹ is branched or unbranched, optionally substituted, acyclic heteroaliphatic. In some embodiments, R¹ is substituted acyclic heteroaliphatic. In certain embodiments, R¹ is unsubstituted acyclic heteroaliphatic. In some embodiments, R¹ is branched or unbranched, optionally substituted, heteroalkylene. In some embodiments, R¹ is substituted heteroalkylene. In some embodiments, R¹ is unsubstituted heteroalkylene. In some embodiments, R¹ is branched or unbranched, optionally substituted, C₁-C₁₀ heteroalkylene. In some embodiments, R¹ is substituted C₁-C₁₀ heteroalkylene. In some embodiments, R¹ is unsubstituted C₁-C₁₀ heteroalkylene. In some embodiments, R¹ is branched or unbranched, optionally substituted, heteroalkenylene. In some embodiments, R¹ is substituted heteroalkenylene. In some embodiments, R¹ is unsubstituted heteroalkenylene. In some embodiments, R¹ is branched or unbranched, optionally substituted, C₂-C₁₀ heteroalkenylene. In some embodiments, R¹ is substituted C₂-C₁₀ heteroalkenylene. In some embodiments, R¹ is unsubstituted C₂-C₁₀ heteroalkenylene. In some embodiments, R¹ is branched or unbranched, optionally substituted, heteroalkynylene. In some embodiments, R¹ is substituted heteroalkynylene. In some embodiments, R¹ is unsubstituted heteroalkynylene. In some embodiments, R¹ is branched or unbranched, optionally substituted, C₂-C₁₀ heteroalkynylene. In some embodiments, R¹ is substituted C₂-C₁₀ heteroalkynylene. In some embodiments, R¹ is unsubstituted C₂-C₁₀ heteroalkynylene.

In certain embodiments, R¹ is branched or unbranched, optionally substituted, cyclic or acyclic acyl. In some embodiments, R¹ is substituted cyclic or acyclic acyl. In some embodiments, R¹ is unsubstituted cyclic or acyclic acyl. In certain embodiments, R¹ is branched or unbranched, optionally substituted, cyclic acyl. In some embodiments, R¹ is substituted cyclic acyl. In some embodiments, R¹ is unsubstituted cyclic acyl. In certain embodiments, R¹ is branched or unbranched, optionally substituted, C₅-C₁₀ cyclic acyl. In some embodiments, R¹ is substituted C₅-C₁₀ cyclic acyl. In some embodiments, R¹ is unsubstituted C₅-C₁₀ cyclic acyl. In certain embodiments, R¹ is branched or unbranched, optionally substituted, acyclic acyl. In some embodiments, R¹ is substituted acyclic acyl. In some embodiments, R¹ is unsubstituted acyclic acyl. In certain embodiments, R¹ is branched or unbranched, optionally substituted, C₁-C₁₀ acyclic acyl. In some embodiments, R¹ is substituted C₁-C₁₀ acyclic acyl. In some embodiments, R¹ is unsubstituted C₁-C₁₀ acyclic acyl.

In certain embodiments, R¹ is optionally substituted aryl. In some embodiments, R¹ is substituted aryl. In certain embodiments, R¹ is unsubstituted aryl. In some embodiments, R¹ is optionally substituted C₆-C₁₄ aryl. In certain embodiments, R¹ is substituted C₆-C₁₄ aryl. In some embodiments, R¹ is unsubstituted C₆-C₁₄ aryl.

In certain embodiments, R¹ is optionally substituted heteroaryl. In some embodiments, R¹ is substituted heteroaryl. In certain embodiments, R¹ is unsubstituted heteroaryl. In certain embodiments, R¹ is optionally substituted 5-14-membered heteroaryl. In some embodiments, R¹ is substituted 5-14-membered heteroaryl. In certain embodiments, R¹ is unsubstituted 5-14-membered heteroaryl.

In certain embodiments, R¹ is a nitrogen protecting group. In some embodiments, the nitrogen protecting group is Bn, Boc, Cbz, Fmoc, trifluoroacetyl, triphenylmethyl, acetyl, or Ts.

In some embodiments, R² is hydrogen.

In certain embodiments, R² is branched or unbranched, optionally substituted, cyclic or acyclic aliphatic. In some embodiments, R² is branched or unbranched, optionally substituted, cyclic aliphatic. In certain embodiments, R² is substituted cyclic aliphatic. In some embodiments, linker is unsubstituted cyclic aliphatic. In certain embodiments, R² is branched or unbranched, optionally substituted carbocyclylene. In some embodiments, R² is substituted carbocyclylene. In certain embodiments, R² is unsubstituted carbocyclylene. In certain embodiments, R² is branched or unbranched, optionally substituted C₃-C₈ carbocyclylene. In some embodiments, R² is substituted C₃-C₈ carbocyclylene. In certain embodiments, R² is unsubstituted C₃-C₈ carbocyclylene. In some embodiments, R² is branched or unbranched, optionally substituted, acyclic aliphatic. In some embodiments, R² is substituted acyclic aliphatic. In certain embodiments, R² is unsubstituted acyclic aliphatic. In some embodiments, R² is branched or unbranched, optionally substituted, alkylene. In some embodiments, R² is substituted alkylene. In some embodiments, R² is unsubstituted alkylene. In some embodiments, R² is branched or unbranched, optionally substituted, C₁-C₁₀ alkylene. In some embodiments, R² is substituted C₁-C₁₀ alkylene. In some embodiments, R² is unsubstituted C₁-C₁₀ alkylene. In some embodiments, R² is branched or unbranched, optionally substituted, alkenylene. In some embodiments, R² is substituted alkenylene. In some embodiments, R² is unsubstituted alkenylene. In some embodiments, R² is branched or unbranched, optionally substituted, C₂-C₁₀ alkenylene. In some embodiments, R² is substituted C₂-C₁₀ alkenylene. In some embodiments, R² is unsubstituted C₂-C₁₀ alkenylene. In some embodiments, R² is branched or unbranched, optionally substituted, alkynylene. In some embodiments, R² is substituted alkynylene. In some embodiments, R² is unsubstituted alkynylene. In some embodiments, R² is branched or unbranched, optionally substituted, C₂-C₁₀ alkynylene. In some embodiments, R² is substituted C₂-C₁₀ alkynylene. In some embodiments, R² is unsubstituted C₂-C₁₀ alkynylene. In certain embodiments, R² is branched or unbranched, optionally substituted, cyclic or acyclic heteroaliphatic. In some embodiments, R² is branched or unbranched, optionally substituted, cyclic heteroaliphatic. In certain embodiments, R² is substituted cyclic heteroaliphatic. In some embodiments, linker is unsubstituted cyclic heteroaliphatic. In certain embodiments, R² is branched or unbranched, optionally substituted heterocyclylene. In some embodiments, R² is substituted heterocyclylene. In certain embodiments, R² is unsubstituted heterocyclylene. In certain embodiments, R² is branched or unbranched, optionally substituted 3-8-membered heterocyclylene. In some embodiments, R² is substituted 3-8-membered heterocyclylene. In certain embodiments, R² is unsubstituted 3-8-membered heterocyclylene. In some embodiments, R² is branched or unbranched, optionally substituted, acyclic heteroaliphatic. In some embodiments, R² is substituted acyclic heteroaliphatic. In certain embodiments, R² is unsubstituted acyclic heteroaliphatic. In some embodiments, R² is branched or unbranched, optionally substituted, heteroalkylene. In some embodiments, R² is substituted heteroalkylene. In some embodiments, R² is unsubstituted heteroalkylene. In some embodiments, R² is branched or unbranched, optionally substituted, C₁-C₁₀ heteroalkylene. In some embodiments, R² is substituted C₁-C₁₀ heteroalkylene. In some embodiments, R² is unsubstituted C₁-C₁₀ heteroalkylene. In some embodiments, R² is branched or unbranched, optionally substituted, heteroalkenylene. In some embodiments, R² is substituted heteroalkenylene. In some embodiments, R² is unsubstituted heteroalkenylene. In some embodiments, R² is branched or unbranched, optionally substituted, C₂-C₁₀ heteroalkenylene. In some embodiments, R² is substituted C₂-C₁₀ heteroalkenylene. In some embodiments, R² is unsubstituted C₂-C₁₀ heteroalkenylene. In some embodiments, R² is branched or unbranched, optionally substituted, heteroalkynylene. In some embodiments, R² is substituted heteroalkynylene. In some embodiments, R² is unsubstituted heteroalkynylene. In some embodiments, R² is branched or unbranched, optionally substituted, C₂-C₁₀ heteroalkynylene. In some embodiments, R² is substituted C₂-C₁₀ heteroalkynylene. In some embodiments, R² is unsubstituted C₂-C₁₀ heteroalkynylene.

In certain embodiments, R² is branched or unbranched, optionally substituted, cyclic or acyclic acyl. In some embodiments, R² is substituted cyclic or acyclic acyl. In some embodiments, R² is unsubstituted cyclic or acyclic acyl. In certain embodiments, R² is branched or unbranched, optionally substituted, cyclic acyl. In some embodiments, R² is substituted cyclic acyl. In some embodiments, R² is unsubstituted cyclic acyl. In certain embodiments, R² is branched or unbranched, optionally substituted, C₅-C₁₀ cyclic acyl. In some embodiments, R² is substituted C₅-C₁₀ cyclic acyl. In some embodiments, R² is unsubstituted C₅-C₁₀ cyclic acyl. In certain embodiments, R² is branched or unbranched, optionally substituted, acyclic acyl. In some embodiments, R² is substituted acyclic acyl. In some embodiments, R² is unsubstituted acyclic acyl. In certain embodiments, R² is branched or unbranched, optionally substituted, C₁-C₁₀ acyclic acyl. In some embodiments, R² is substituted C₁-C₁₀ acyclic acyl. In some embodiments, R² is unsubstituted C₁-C₁₀ acyclic acyl.

In certain embodiments, R² is optionally substituted aryl. In some embodiments, R² is substituted aryl. In certain embodiments, R² is unsubstituted aryl. In some embodiments, R² is optionally substituted C₆-C₁₄ aryl. In certain embodiments, R² is substituted C₆-C₁₄ aryl. In some embodiments, R² is unsubstituted C₆-C₁₄ aryl.

In certain embodiments, R² is optionally substituted heteroaryl. In some embodiments, R² is substituted heteroaryl. In certain embodiments, R² is unsubstituted heteroaryl. In certain embodiments, R² is optionally substituted 5-14-membered heteroaryl. In some embodiments, R² is substituted 5-14-membered heteroaryl. In certain embodiments, R² is unsubstituted 5-14-membered heteroaryl.

In certain embodiments, R² is a nitrogen protecting group. In some embodiments, the nitrogen protecting group is Bn, Boc, Cbz, Fmoc, trifluoroacetyl, triphenylmethyl, acetyl, or Ts.

In some embodiments, one or both R¹ or R² are each independently joined to linker A together with the intervening atoms to form a linker comprising one or more N-containing heterocycles; or R¹ and R² are joined together with the intervening atoms to form a N-containing heterocycle. In certain embodiments, one or both R¹ or R² are each optionally independently joined to linker A together with the intervening atoms to form a linker comprising one or more N-containing heterocycles. In some embodiments, R¹ is joined to linker A together with the intervening atoms to form a linker comprising at least one N-containing heterocycle. In some embodiments, R^{B} is joined to linker A together with the intervening atoms to form a linker comprising at least one N-containing heterocycle. In some embodiments, R¹ and R² are joined together with the intervening atoms to form a N-containing heterocycle. In some embodiments, the N-containing heterocycle is a 3-8-membered N-containing heterocycle. In some embodiments, the N-containing heterocycle is a 5-7-membered N-containing heterocycle. In some embodiments, the N-containing heterocycle is a 5-membered N-containing heterocycle. In some embodiments, the N-containing heterocycle is a 6-membered N-containing heterocycle. In some embodiments, the N-containing heterocycle is a 7-membered N-containing heterocycle.

As defined herein, linker A comprises branched or unbranched, optionally substituted, cyclic or acyclic aliphatic; branched or unbranched, optionally substituted, cyclic or acyclic heteroaliphatic; optionally substituted arylene; or optionally substituted heteroarylene. As defined herein, linker A is branched or unbranched, optionally substituted, cyclic or acyclic aliphatic; branched or unbranched, optionally substituted, cyclic or acyclic heteroaliphatic; optionally substituted arylene; or optionally substituted heteroarylene.

In certain embodiments, linker A is branched or unbranched, optionally substituted, cyclic or acyclic aliphatic. In some embodiments, linker A is branched or unbranched, optionally substituted, cyclic aliphatic. In certain embodiments, linker A is substituted cyclic aliphatic. In some embodiments, linker is unsubstituted cyclic aliphatic. In certain embodiments, linker A is branched or unbranched, optionally substituted carbocyclylene. In some embodiments, linker A is substituted carbocyclylene. In certain embodiments, linker A is unsubstituted carbocyclylene. In certain embodiments, linker A is branched or unbranched, optionally substituted C₃-C₈ carbocyclylene. In some embodiments, linker A is substituted C₃-C₈ carbocyclylene. In certain embodiments, linker A is unsubstituted C₃-C₈ carbocyclylene.

In some embodiments, linker A is branched or unbranched, optionally substituted, acyclic aliphatic. In some embodiments, linker A is substituted acyclic aliphatic. In certain embodiments, linker A is unsubstituted acyclic aliphatic. In some embodiments, linker A is branched or unbranched, optionally substituted, alkylene. In some embodiments, linker A is substituted alkylene. In some embodiments, linker A is unsubstituted alkylene. In some embodiments, linker A is branched or unbranched, optionally substituted, C₁-C₁₀ alkylene. In some embodiments, linker A is substituted C₁-C₁₀ alkylene. In some embodiments, linker A is unsubstituted C₁-C₁₀ alkylene. In some embodiments, linker A is branched or unbranched, optionally substituted, alkenylene. In some embodiments, linker A is substituted alkenylene. In some embodiments, linker A is unsubstituted alkenylene. In some embodiments, linker A is branched or unbranched, optionally substituted, C₂-C₁₀ alkenylene. In some embodiments, linker A is substituted C₂-C₁₀ alkenylene. In some embodiments, linker A is unsubstituted C₂-C₁₀ alkenylene. In some embodiments, linker A is branched or unbranched, optionally substituted, alkynylene. In some embodiments, linker A is substituted alkynylene. In some embodiments, linker A is unsubstituted alkynylene. In some embodiments, linker A is branched or unbranched, optionally substituted, C₂-C₁₀ alkynylene. In some embodiments, linker A is substituted C₂-C₁₀ alkynylene. In some embodiments, linker A is unsubstituted C₂-C₁₀ alkynylene.

In certain embodiments, linker A is branched or unbranched, optionally substituted, cyclic or acyclic heteroaliphatic. In some embodiments, linker A is branched or unbranched, optionally substituted, cyclic heteroaliphatic. In certain embodiments, linker A is substituted cyclic heteroaliphatic. In some embodiments, linker is unsubstituted cyclic heteroaliphatic. In certain embodiments, linker A is branched or unbranched, optionally substituted heterocyclylene. In some embodiments, linker A is substituted heterocyclylene. In certain embodiments, linker A is unsubstituted heterocyclylene. In certain embodiments, linker A is branched or unbranched, optionally substituted 3-8-membered heterocyclylene. In some embodiments, linker A is substituted 3-8-membered heterocyclylene. In certain embodiments, linker A is unsubstituted 3-8-membered heterocyclylene.

In some embodiments, linker A is branched or unbranched, optionally substituted, acyclic heteroaliphatic. In some embodiments, linker A is substituted acyclic heteroaliphatic. In certain embodiments, linker A is unsubstituted acyclic heteroaliphatic. In some embodiments, linker A is branched or unbranched, optionally substituted, heteroalkylene. In some embodiments, linker A is substituted heteroalkylene. In some embodiments, linker A is unsubstituted heteroalkylene. In some embodiments, linker A is branched or unbranched, optionally substituted, C₁-C₁₀ heteroalkylene. In some embodiments, linker A is substituted C₁-C₁₀ heteroalkylene. In some embodiments, linker A is unsubstituted C₁-C₁₀ heteroalkylene. In some embodiments, linker A is branched or unbranched, optionally substituted, heteroalkenylene. In some embodiments, linker A is substituted heteroalkenylene. In some embodiments, linker A is unsubstituted heteroalkenylene. In some embodiments, linker A is branched or unbranched, optionally substituted, C₂-C₁₀ heteroalkenylene. In some embodiments, linker A is substituted C₂-C₁₀ heteroalkenylene. In some embodiments, linker A is unsubstituted C₂-C₁₀ heteroalkenylene. In some embodiments, linker A is branched or unbranched, optionally substituted, heteroalkynylene. In some embodiments, linker A is substituted heteroalkynylene. In some embodiments, linker A is unsubstituted heteroalkynylene. In some embodiments, linker A is branched or unbranched, optionally substituted, C₂-C₁₀ heteroalkynylene. In some embodiments, linker A is substituted C₂-C₁₀ heteroalkynylene. In some embodiments, linker A is unsubstituted C₂-C₁₀ heteroalkynylene.

In certain embodiments, linker A is optionally substituted arylene. In some embodiments, linker A is substituted arylene. In certain embodiments, linker A is unsubstituted arylene. In some embodiments, linker A is optionally substituted C₆-C₁₄ arylene. In certain embodiments, linker A is substituted C₆-C₁₄ arylene. In some embodiments, linker A is unsubstituted C₆-C₁₄ arylene.

In certain embodiments, linker A is optionally substituted heteroarylene. In some embodiments, linker A is substituted heteroarylene. In certain embodiments, linker A is unsubstituted heteroarylene. In certain embodiments, linker A is optionally substituted 5-14-membered heteroarylene. In some embodiments, linker A is substituted 5-14-membered heteroarylene. In certain embodiments, linker A is unsubstituted 5-14-membered heteroarylene.

In certain embodiments,

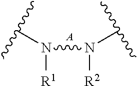

is selected from

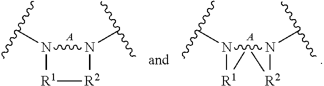

In some embodiments,

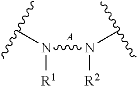

is

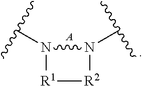

In certain embodiments,

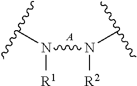

is

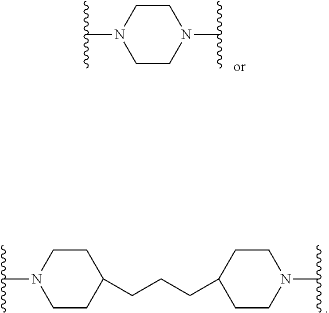 [SMILES: *N1CCN(*)CC1 ; "or" ; *N1CCC(CCCC2CCN(*)CC2)CC1 ; "."]

In certain embodiments,

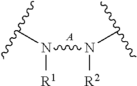

is

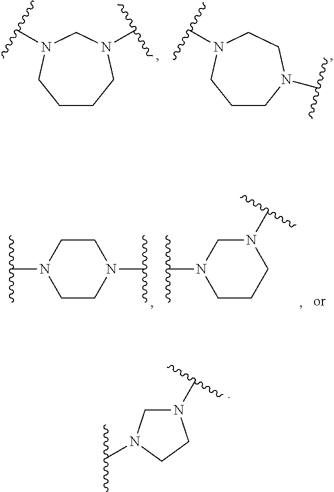

In some embodiments,

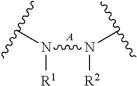

is

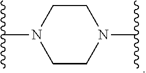 [SMILES: *N1CCN(*)CC1 ; "."]

In certain embodiments,

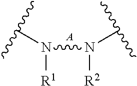

is

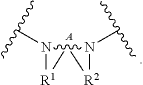

In some embodiments,

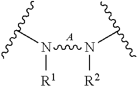

is

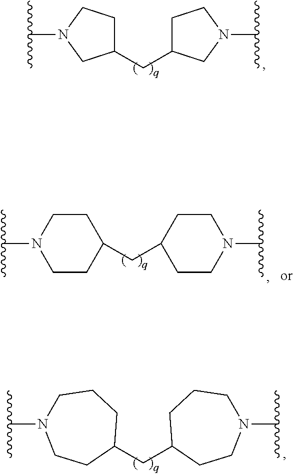

wherein each q is independently 1-10. In certain embodiments, q is 1-6. In some embodiments, q is 1-4. In certain embodiments, q is 1. In some embodiments, q is 2. In certain embodiments, q is 3. In some embodiments, q is 4. In certain embodiments, q is 5. In some embodiments, q is 6. In certain embodiments, q is 7. In some embodiments, q is 8. In certain embodiments, q is 9. In some embodiments, q is 10.

In certain embodiments,

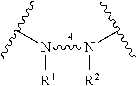

is

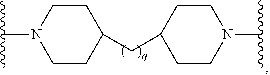

wherein each q is independently 1-10. In certain embodiments, q is 1-6. In some embodiments, q is 1-4. In certain embodiments, q is 1. In some embodiments, q is 2. In certain embodiments, q is 3. In some embodiments, q is 4. In certain embodiments, q is 5. In some embodiments, q is 6. In certain embodiments, q is 7. In some embodiments, q is 8. In certain embodiments, q is 9. In some embodiments, q is 10. In certain embodiments,

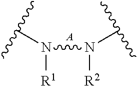

is

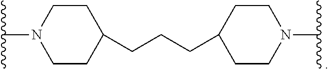 [SMILES: *N1CCC(CCCC2CCN(*)CC2)CC1 ; "."]

In certain embodiments, wherein L is

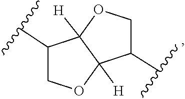 [SMILES: *C1COC2C(*)COC12 ; ","]

and the compound of Formula (I) is of Formula (I-B-iii):

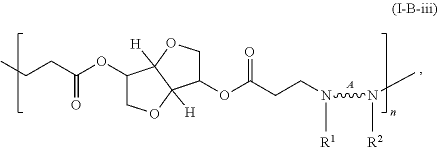

wherein each

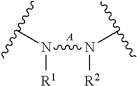

is independently selected from

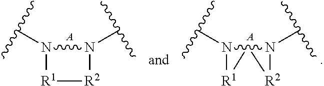

In certain embodiments, approximately 0-10%, approximately 5-15%, approximately 10-20%, approximately 15-25%, approximately 20-30%, approximately 25-35%, approximately 30-40%, approximately 35-45%, approximately 40-50%, approximately 45-55%, approximately 50-60%, approximately 55-65%, approximately 60-70%, approximately 65-75%, approximately 70-80%, approximately 75-85%, approximately 80-90%, approximately 85-95%, or approximately 90-100% of Z

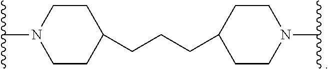 [SMILES: *N1CCC(CCCC2CCN(*)CC2)CC1 ; "."]

In some embodiments, approximately 0-10%, approximately 5-15%, approximately 10-20%, approximately 15-25%, approximately 20-30%, approximately 25-35%, approximately 30-40%, approximately 35-45%, approximately 40-50%, approximately 45-55%, approximately 50-60%, approximately 55-65%, approximately 60-70%, approximately 65-75%, approximately 70-80%, approximately 75-85%, approximately 80-90%, approximately 85-95%, or approximately 90-100% of Z is

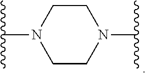 [SMILES: *N1CCN(*)CC1 ; "."]

In certain embodiments, Z is

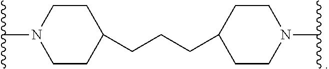 [SMILES: *N1CCC(CCCC2CCN(*)CC2)CC1 ; "."]

In some embodiments, approximately 15-30% of Z is

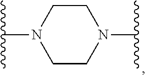 [SMILES: *N1CCN(*)CC1 ; ","]

and approximately 70-85% of Z is

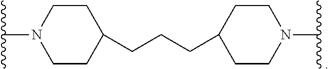 [SMILES: *N1CCC(CCCC2CCN(*)CC2)CC1 ; "."]

In certain embodiments, approximately 40-55% of Z is

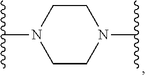 [SMILES: *N1CCN(*)CC1 ; ","]

and approximately 45-60% of Z is

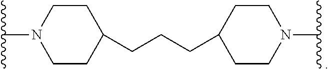 [SMILES: *N1CCC(CCCC2CCN(*)CC2)CC1 ; "."]

In some embodiments, approximately 70-85% of Z is

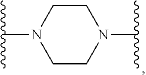 [SMILES: *N1CCN(*)CC1 ; ","]

and approximately 15-30% of Z is

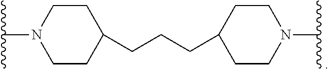 [SMILES: *N1CCC(CCCC2CCN(*)CC2)CC1 ; "."]

In certain embodiments, Z is

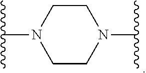 [SMILES: *N1CCN(*)CC1 ; "."]

As defined herein, R³ is branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group.

In certain embodiments, R³ is branched or unbranched, optionally substituted, cyclic or acyclic aliphatic. In some embodiments, R³ is branched or unbranched, optionally substituted, cyclic aliphatic. In certain embodiments, R³ is substituted cyclic aliphatic. In some embodiments, linker is unsubstituted cyclic aliphatic. In certain embodiments, R³ is branched or unbranched, optionally substituted carbocyclylene. In some embodiments, R³ is substituted carbocyclylene. In certain embodiments, R³ is unsubstituted carbocyclylene. In certain embodiments, R³ is branched or unbranched, optionally substituted C₃-C₈ carbocyclylene. In some embodiments, R³ is substituted C₃-C₈ carbocyclylene. In certain embodiments, R³ is unsubstituted C₃-C₈ carbocyclylene. In some embodiments, R³ is branched or unbranched, optionally substituted, acyclic aliphatic. In some embodiments, R³ is substituted acyclic aliphatic. In certain embodiments, R³ is unsubstituted acyclic aliphatic. In some embodiments, R³ is branched or unbranched, optionally substituted, alkylene. In some embodiments, R³ is substituted alkylene. In some embodiments, R³ is unsubstituted alkylene. In some embodiments, R³ is branched or unbranched, optionally substituted, C₁-C₁₀ alkylene. In some embodiments, R³ is substituted C₁-C₁₀ alkylene. In some embodiments, R³ is unsubstituted C₁-C₁₀ alkylene. In some embodiments, R³ is branched or unbranched, optionally substituted, alkenylene. In some embodiments, R³ is substituted alkenylene. In some embodiments, R³ is unsubstituted alkenylene. In some embodiments, R³ is branched or unbranched, optionally substituted, C₂-C₁₀ alkenylene. In some embodiments, R³ is substituted C₂-C₁₀ alkenylene. In some embodiments, R³ is unsubstituted C₂-C₁₀ alkenylene. In some embodiments, R³ is branched or unbranched, optionally substituted, alkynylene. In some embodiments, R³ is substituted alkynylene. In some embodiments, R³ is unsubstituted alkynylene. In some embodiments, R³ is branched or unbranched, optionally substituted, C₂-C₁₀ alkynylene. In some embodiments, R³ is substituted C₂-C₁₀ alkynylene. In some embodiments, R³ is unsubstituted C₂-C₁₀ alkynylene.

In certain embodiments, R³ is branched or unbranched, optionally substituted, cyclic or acyclic heteroaliphatic. In some embodiments, R³ is branched or unbranched, optionally substituted, cyclic heteroaliphatic. In certain embodiments, R³ is substituted cyclic heteroaliphatic. In some embodiments, linker is unsubstituted cyclic heteroaliphatic. In certain embodiments, R³ is branched or unbranched, optionally substituted heterocyclylene. In some embodiments, R³ is substituted heterocyclylene. In certain embodiments, R³ is unsubstituted heterocyclylene. In certain embodiments, R³ is branched or unbranched, optionally substituted 3-8-membered heterocyclylene. In some embodiments, R³ is substituted 3-8-membered heterocyclylene. In certain embodiments, R³ is unsubstituted 3-8-membered heterocyclylene. In some embodiments, R³ is branched or unbranched, optionally substituted, acyclic heteroaliphatic. In some embodiments, R³ is substituted acyclic heteroaliphatic. In certain embodiments, R³ is unsubstituted acyclic heteroaliphatic. In some embodiments, R³ is branched or unbranched, optionally substituted, heteroalkylene. In some embodiments, R³ is substituted heteroalkylene. In some embodiments, R³ is unsubstituted heteroalkylene. In some embodiments, R³ is branched or unbranched, optionally substituted, C₁-C₁₀ heteroalkylene. In some embodiments, R³ is substituted C₁-C₁₀ heteroalkylene. In some embodiments, R³ is unsubstituted C₁-C₁₀ heteroalkylene. In some embodiments, R³ is branched or unbranched, optionally substituted, heteroalkenylene. In some embodiments, R³ is substituted heteroalkenylene. In some embodiments, R³ is unsubstituted heteroalkenylene. In some embodiments, R³ is branched or unbranched, optionally substituted, C₂-C₁₀ heteroalkenylene. In some embodiments, R³ is substituted C₂-C₁₀ heteroalkenylene. In some embodiments, R³ is unsubstituted C₂-C₁₀ heteroalkenylene. In some embodiments, R³ is branched or unbranched, optionally substituted, heteroalkynylene. In some embodiments, R³ is substituted heteroalkynylene. In some embodiments, R³ is unsubstituted heteroalkynylene. In some embodiments, R³ is branched or unbranched, optionally substituted, C₂-C₁₀ heteroalkynylene. In some embodiments, R³ is substituted C₂-C₁₀ heteroalkynylene. In some embodiments, R³ is unsubstituted C₂-C₁₀ heteroalkynylene.

In certain embodiments, R³ is branched or unbranched, optionally substituted, cyclic or acyclic acyl. In some embodiments, R³ is substituted cyclic or acyclic acyl. In some embodiments, R³ is unsubstituted cyclic or acyclic acyl. In certain embodiments, R³ is branched or unbranched, optionally substituted, cyclic acyl. In some embodiments, R³ is substituted cyclic acyl. In some embodiments, R³ is unsubstituted cyclic acyl. In certain embodiments, R³ is branched or unbranched, optionally substituted, C₅-C₁₀ cyclic acyl. In some embodiments, R³ is substituted C₅-C₁₀ cyclic acyl. In some embodiments, R³ is unsubstituted C₅-C₁₀ cyclic acyl. In certain embodiments, R³ is branched or unbranched, optionally substituted, acyclic acyl. In some embodiments, R³ is substituted acyclic acyl. In some embodiments, R³ is unsubstituted acyclic acyl. In certain embodiments, R³ is branched or unbranched, optionally substituted, C₁-C₁₀ acyclic acyl. In some embodiments, R³ is substituted C₁-C₁₀ acyclic acyl. In some embodiments, R³ is unsubstituted C₁-C₁₀ acyclic acyl.

In certain embodiments, R³ is optionally substituted aryl. In some embodiments, R³ is substituted aryl. In certain embodiments, R³ is unsubstituted aryl. In some embodiments, R³ is optionally substituted C₆-C₁₄ aryl. In certain embodiments, R³ is substituted C₆-C₁₄ aryl. In some embodiments, R³ is unsubstituted C₆-C₁₄ aryl.

In certain embodiments, R³ is optionally substituted heteroaryl. In some embodiments, R³ is substituted heteroaryl. In certain embodiments, R³ is unsubstituted heteroaryl. In certain embodiments, R³ is optionally substituted 5-14-membered heteroaryl. In some embodiments, R³ is substituted 5-14-membered heteroaryl. In certain embodiments, R³ is unsubstituted 5-14-membered heteroaryl.

In certain embodiments, R³ is a nitrogen protecting group. In some embodiments, the nitrogen protecting group is Bn, Boc, Cbz, Fmoc, trifluoroacetyl, triphenylmethyl, acetyl, or Ts.

As defined herein, linker B comprises branched or unbranched, optionally substituted, cyclic or acyclic aliphatic; branched or unbranched, optionally substituted, cyclic or acyclic heteroaliphatic; optionally substituted arylene; or optionally substituted heteroarylene. As defined herein, linker B is branched or unbranched, optionally substituted, cyclic or acyclic aliphatic; branched or unbranched, optionally substituted, cyclic or acyclic heteroaliphatic; optionally substituted arylene; or optionally substituted heteroarylene.

In certain embodiments, linker B is branched or unbranched, optionally substituted, cyclic or acyclic aliphatic. In some embodiments, linker B is branched or unbranched, optionally substituted, cyclic aliphatic. In certain embodiments, linker B is substituted cyclic aliphatic. In some embodiments, linker is unsubstituted cyclic aliphatic. In certain embodiments, linker B is branched or unbranched, optionally substituted carbocyclylene. In some embodiments, linker B is substituted carbocyclylene. In certain embodiments, linker B is unsubstituted carbocyclylene. In certain embodiments, linker B is branched or unbranched, optionally substituted C₃-C₈ carbocyclylene. In some embodiments, linker B is substituted C₃-C₈ carbocyclylene. In certain embodiments, linker B is unsubstituted C₃-C₈ carbocyclylene.

In some embodiments, linker B is branched or unbranched, optionally substituted, acyclic aliphatic. In some embodiments, linker B is substituted acyclic aliphatic. In certain embodiments, linker B is unsubstituted acyclic aliphatic. In some embodiments, linker B is branched or unbranched, optionally substituted, alkylene. In some embodiments, linker B is substituted alkylene. In some embodiments, linker B is unsubstituted alkylene. In some embodiments, linker B is branched or unbranched, optionally substituted, C₁-C₁₀ alkylene. In some embodiments, linker B is substituted C₁-C₁₀ alkylene. In some embodiments, linker B is unsubstituted C₁-C₁₀ alkylene. In some embodiments, linker B is branched or unbranched, optionally substituted, alkenylene. In some embodiments, linker B is substituted alkenylene. In some embodiments, linker B is unsubstituted alkenylene. In some embodiments, linker B is branched or unbranched, optionally substituted, C₂-C₁₀ alkenylene. In some embodiments, linker B is substituted C₂-C₁₀ alkenylene. In some embodiments, linker B is unsubstituted C₂-C₁₀ alkenylene. In some embodiments, linker B is branched or unbranched, optionally substituted, alkynylene. In some embodiments, linker B is substituted alkynylene. In some embodiments, linker B is unsubstituted alkynylene. In some embodiments, linker B is branched or unbranched, optionally substituted, C₂-C₁₀ alkynylene. In some embodiments, linker B is substituted C₂-C₁₀ alkynylene. In some embodiments, linker B is unsubstituted C₂-C₁₀ alkynylene.

In certain embodiments, linker B is branched or unbranched, optionally substituted, cyclic or acyclic heteroaliphatic. In some embodiments, linker B is branched or unbranched, optionally substituted, cyclic heteroaliphatic. In certain embodiments, linker B is substituted cyclic heteroaliphatic. In some embodiments, linker is unsubstituted cyclic heteroaliphatic. In certain embodiments, linker B is branched or unbranched, optionally substituted heterocyclylene. In some embodiments, linker B is substituted heterocyclylene. In certain embodiments, linker B is unsubstituted heterocyclylene. In certain embodiments, linker B is branched or unbranched, optionally substituted 3-8-membered heterocyclylene. In some embodiments, linker B is substituted 3-8-membered heterocyclylene. In certain embodiments, linker B is unsubstituted 3-8-membered heterocyclylene.

In some embodiments, linker B is branched or unbranched, optionally substituted, acyclic heteroaliphatic. In some embodiments, linker B is substituted acyclic heteroaliphatic. In certain embodiments, linker B is unsubstituted acyclic heteroaliphatic. In some embodiments, linker B is branched or unbranched, optionally substituted, heteroalkylene. In some embodiments, linker B is substituted heteroalkylene. In some embodiments, linker B is unsubstituted heteroalkylene. In some embodiments, linker B is branched or unbranched, optionally substituted, C₁-C₁₀ heteroalkylene. In some embodiments, linker B is substituted C₁-C₁₀ heteroalkylene. In some embodiments, linker B is unsubstituted C₁-C₁₀ heteroalkylene. In some embodiments, linker B is branched or unbranched, optionally substituted, heteroalkenylene. In some embodiments, linker B is substituted heteroalkenylene. In some embodiments, linker B is unsubstituted heteroalkenylene. In some embodiments, linker B is branched or unbranched, optionally substituted, C₂-C₁₀ heteroalkenylene. In some embodiments, linker B is substituted C₂-C₁₀ heteroalkenylene. In some embodiments, linker B is unsubstituted C₂-C₁₀ heteroalkenylene. In some embodiments, linker B is branched or unbranched, optionally substituted, heteroalkynylene. In some embodiments, linker B is substituted heteroalkynylene. In some embodiments, linker B is unsubstituted heteroalkynylene. In some embodiments, linker B is branched or unbranched, optionally substituted, C₂-C₁₀ heteroalkynylene. In some embodiments, linker B is substituted C₂-C₁₀ heteroalkynylene. In some embodiments, linker B is unsubstituted C₂-C₁₀ heteroalkynylene.

In certain embodiments, linker B is optionally substituted arylene. In some embodiments, linker B is substituted arylene. In certain embodiments, linker B is unsubstituted arylene. In some embodiments, linker B is optionally substituted C₆-C₁₄ arylene. In certain embodiments, linker B is substituted C₆-C₁₄ arylene. In some embodiments, linker B is unsubstituted C₆-C₁₄ arylene.

In certain embodiments, linker B is optionally substituted heteroarylene. In some embodiments, linker B is substituted heteroarylene. In certain embodiments, linker B is unsubstituted heteroarylene. In certain embodiments, linker B is optionally substituted 5-14-membered heteroarylene. In some embodiments, linker B is substituted 5-14-membered heteroarylene. In certain embodiments, linker B is unsubstituted 5-14-membered heteroarylene.

As defined herein, R⁴ is hydrogen, branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group; or R⁴ is joined to linker B together with the intervening atoms to form a linker comprising a N-containing heterocycle. In certain embodiments, R⁴ is hydrogen, branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group.

In some embodiments, R⁴ is hydrogen.

In certain embodiments, R⁴ is branched or unbranched, optionally substituted, cyclic or acyclic aliphatic. In some embodiments, R⁴ is branched or unbranched, optionally substituted, cyclic aliphatic. In certain embodiments, R⁴ is substituted cyclic aliphatic. In some embodiments, linker is unsubstituted cyclic aliphatic. In certain embodiments, R⁴ is branched or unbranched, optionally substituted carbocyclylene. In some embodiments, R⁴ is substituted carbocyclylene. In certain embodiments, R⁴ is unsubstituted carbocyclylene. In certain embodiments, R⁴ is branched or unbranched, optionally substituted C₃-C₈ carbocyclylene. In some embodiments, R⁴ is substituted C₃-C₈ carbocyclylene. In certain embodiments, R⁴ is unsubstituted C₃-C₈ carbocyclylene. In some embodiments, R⁴ is branched or unbranched, optionally substituted, acyclic aliphatic. In some embodiments, R⁴ is substituted acyclic aliphatic. In certain embodiments, R⁴ is unsubstituted acyclic aliphatic. In some embodiments, R⁴ is branched or unbranched, optionally substituted, alkylene. In some embodiments, R⁴ is substituted alkylene. In some embodiments, R⁴ is unsubstituted alkylene. In some embodiments, R⁴ is branched or unbranched, optionally substituted, C₁-C₁₀ alkylene. In some embodiments, R⁴ is substituted C₁-C₁₀ alkylene. In some embodiments, R⁴ is unsubstituted C₁-C₁₀ alkylene. In some embodiments, R⁴ is branched or unbranched, optionally substituted, alkenylene. In some embodiments, R⁴ is substituted alkenylene. In some embodiments, R⁴ is unsubstituted alkenylene. In some embodiments, R⁴ is branched or unbranched, optionally substituted, C₂-C₁₀ alkenylene. In some embodiments, R⁴ is substituted C₂-C₁₀ alkenylene. In some embodiments, R⁴ is unsubstituted C₂-C₁₀ alkenylene. In some embodiments, R⁴ is branched or unbranched, optionally substituted, alkynylene. In some embodiments, R⁴ is substituted alkynylene. In some embodiments, R⁴ is unsubstituted alkynylene. In some embodiments, R⁴ is branched or unbranched, optionally substituted, C₂-C₁₀ alkynylene. In some embodiments, R⁴ is substituted C₂-C₁₀ alkynylene. In some embodiments, R⁴ is unsubstituted C₂-C₁₀ alkynylene.

In certain embodiments, R⁴ is branched or unbranched, optionally substituted, cyclic or acyclic heteroaliphatic. In some embodiments, R⁴ is branched or unbranched, optionally substituted, cyclic heteroaliphatic. In certain embodiments, R⁴ is substituted cyclic heteroaliphatic. In some embodiments, linker is unsubstituted cyclic heteroaliphatic. In certain embodiments, R⁴ is branched or unbranched, optionally substituted heterocyclylene. In some embodiments, R⁴ is substituted heterocyclylene. In certain embodiments, R⁴ is unsubstituted heterocyclylene. In certain embodiments, R⁴ is branched or unbranched, optionally substituted 3-8-membered heterocyclylene. In some embodiments, R⁴ is substituted 3-8-membered heterocyclylene. In certain embodiments, R⁴ is unsubstituted 3-8-membered heterocyclylene. In some embodiments, R⁴ is branched or unbranched, optionally substituted, acyclic heteroaliphatic. In some embodiments, R⁴ is substituted acyclic heteroaliphatic. In certain embodiments, R⁴ is unsubstituted acyclic heteroaliphatic. In some embodiments, R⁴ is branched or unbranched, optionally substituted, heteroalkylene. In some embodiments, R⁴ is substituted heteroalkylene. In some embodiments, R⁴ is unsubstituted heteroalkylene. In some embodiments, R⁴ is branched or unbranched, optionally substituted, C₁-C₁₀ heteroalkylene. In some embodiments, R⁴ is substituted C₁-C₁₀ heteroalkylene. In some embodiments, R⁴ is unsubstituted C₁-C₁₀ heteroalkylene. In some embodiments, R⁴ is branched or unbranched, optionally substituted, heteroalkenylene. In some embodiments, R⁴ is substituted heteroalkenylene. In some embodiments, R⁴ is unsubstituted heteroalkenylene. In some embodiments, R⁴ is branched or unbranched, optionally substituted, C₂-C₁₀ heteroalkenylene. In some embodiments, R⁴ is substituted C₂-C₁₀ heteroalkenylene. In some embodiments, R⁴ is unsubstituted C₂-C₁₀ heteroalkenylene. In some embodiments, R⁴ is branched or unbranched, optionally substituted, heteroalkynylene. In some embodiments, R⁴ is substituted heteroalkynylene. In some embodiments, R⁴ is unsubstituted heteroalkynylene. In some embodiments, R⁴ is branched or unbranched, optionally substituted, C₂-C₁₀ heteroalkynylene. In some embodiments, R⁴ is substituted C₂-C₁₀ heteroalkynylene. In some embodiments, R⁴ is unsubstituted C₂-C₁₀ heteroalkynylene.

In certain embodiments, R⁴ is branched or unbranched, optionally substituted, cyclic or acyclic acyl. In some embodiments, R⁴ is substituted cyclic or acyclic acyl. In some embodiments, R⁴ is unsubstituted cyclic or acyclic acyl. In certain embodiments, R⁴ is branched or unbranched, optionally substituted, cyclic acyl. In some embodiments, R⁴ is substituted cyclic acyl. In some embodiments, R⁴ is unsubstituted cyclic acyl. In certain embodiments, R⁴ is branched or unbranched, optionally substituted, C₅-C₁₀ cyclic acyl. In some embodiments, R⁴ is substituted C₅-C₁₀ cyclic acyl. In some embodiments, R⁴ is unsubstituted C₅-C₁₀ cyclic acyl. In certain embodiments, R⁴ is branched or unbranched, optionally substituted, acyclic acyl. In some embodiments, R⁴ is substituted acyclic acyl. In some embodiments, R⁴ is unsubstituted acyclic acyl. In certain embodiments, R⁴ is branched or unbranched, optionally substituted, C₁-C₁₀ acyclic acyl. In some embodiments, R⁴ is substituted C₁-C₁₀ acyclic acyl. In some embodiments, R⁴ is unsubstituted C₁-C₁₀ acyclic acyl.

In certain embodiments, R⁴ is optionally substituted aryl. In some embodiments, R⁴ is substituted aryl. In certain embodiments, R⁴ is unsubstituted aryl. In some embodiments, R⁴ is optionally substituted C₆-C₁₄ aryl. In certain embodiments, R⁴ is substituted C₆-C₁₄ aryl. In some embodiments, R⁴ is unsubstituted C₆-C₁₄ aryl.

In certain embodiments, R⁴ is optionally substituted heteroaryl. In some embodiments, R⁴ is substituted heteroaryl. In certain embodiments, R⁴ is unsubstituted heteroaryl. In certain embodiments, R⁴ is optionally substituted 5-14-membered heteroaryl. In some embodiments, R⁴ is substituted 5-14-membered heteroaryl. In certain embodiments, R⁴ is unsubstituted 5-14-membered heteroaryl.

In certain embodiments, R⁴ is a nitrogen protecting group. In some embodiments, the nitrogen protecting group is Bn, Boc, Cbz, Fmoc, trifluoroacetyl, triphenylmethyl, acetyl, or Ts.

In some embodiments, R⁴ is joined to linker B together with the intervening atoms to form a linker comprising a N-containing heterocycle. In some embodiments, the N-containing heterocycle is a 3-8-membered N-containing heterocycle. In some embodiments, the N-containing heterocycle is a 5-7-membered N-containing heterocycle. In some embodiments, the N-containing heterocycle is a 5-membered N-containing heterocycle. In some embodiments, the N-containing heterocycle is a 6-membered N-containing heterocycle. In some embodiments, the N-containing heterocycle is a 7-membered N-containing heterocycle.

In another aspect, the disclosure provides a compound prepared by reacting one or more compounds of Formula (II):

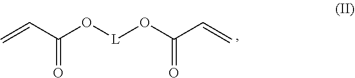

or a salt, isotope, or stereoisomer thereof, with one or more compounds selected from

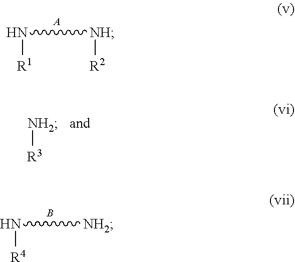

or a salt, isotope, or stereoisomer thereof, wherein:
L is a heterocycle comprising at least one oxygen atom;
linker A is branched or unbranched, optionally substituted, cyclic or acyclic aliphatic; branched or unbranched, optionally substituted, cyclic or acyclic heteroaliphatic; optionally substituted arylene; or optionally substituted heteroarylene;
R¹ and R² are each independently hydrogen; branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group; or
one or both R¹ or R² are each optionally independently joined to linker A together with the intervening atoms to form a linker comprising one or more N-containing heterocycles; or
R¹ and R² are joined together with the intervening atoms to form a N-containing heterocycle;
R³ is optionally substituted aliphatic, optionally substituted heteroaliphatic, optionally substituted carbocyclyl, optionally substituted heterocyclyl, optionally substituted optionally substituted aryl, or optionally substituted heteroaryl;
linker B is branched or unbranched, optionally substituted, cyclic or acyclic aliphatic; branched or unbranched, optionally substituted, cyclic or acyclic heteroaliphatic; optionally substituted arylene; or optionally substituted heteroarylene; and
R⁴ is optionally substituted aliphatic, optionally substituted heteroaliphatic, optionally substituted carbocyclyl, optionally substituted heterocyclyl, optionally substituted aryl, or optionally substituted heteroaryl; or
R⁴ is joined to linker B together with the intervening atoms to form a linker comprising a N-containing heterocycle.

As defined herein, L is a heterocycle comprising at least one oxygen atom. In certain embodiments, L comprises one oxygen atom. In some embodiments, L comprises at least two oxygen atoms. In certain embodiments, L comprises two oxygen atoms. In some embodiments, L comprises at least three oxygen atoms. In certain embodiments, L comprises three oxygen atoms. In certain embodiments, L comprises a pyran. In certain embodiments, L comprises a furan.

In some embodiments, L is derived from a monosaccharide. In certain embodiments, the monosaccharide is arabinose, lyxose, ribose, xylose, ribulofuranose, xylulose, allose, altrose, galactose, glucose, gulose, idose, mannose, talose, fructose, psicose, sorbose, tagatose, mannoheptulose, and sedoheptulose. In some embodiments, the monosaccharide is allose, altrose, galactose, glucose, gulose, idose, mannose, talose, fructose, psicose, sorbose, tagatose. In certain embodiments, the monosaccharide is glucose or galactose. In some embodiments, the monosaccharide is galactose. In certain embodiments, the monosaccharide is glucose.

In some embodiments, L is an unsubstituted 3-14 membered heterocycle comprising at least one oxygen atom. In certain embodiments, L is a substituted 3-14 membered heterocycle comprising at least one oxygen atom. In certain embodiments, L is optionally substituted, 3- to 7-membered, monocyclic heterocycle, comprising at least one oxygen atom. In certain embodiments, L is substituted, 3- to 7-membered, monocyclic heterocycle, comprising at least one oxygen atom. In certain embodiments, L is unsubstituted, 3- to 7-membered, monocyclic heterocycle, comprising at least one oxygen atom. In certain embodiments, L is optionally substituted, 5- to 6-membered, monocyclic heterocycle, comprising at least one oxygen atom. In certain embodiments, L is substituted, 5- to 5-membered, monocyclic heterocycle, comprising at least one oxygen atom. In certain embodiments, L is unsubstituted, 5- to 6-membered, monocyclic heterocycle, comprising at least one oxygen atom. In certain embodiments, L is optionally substituted, 8- to 10-membered, bicyclic heterocycle, comprising at least one oxygen atom. In certain embodiments, L is substituted, 8- to 10-membered, bicyclic heterocycle, comprising at least one oxygen atom. In certain embodiments, L is unsubstituted, 8- to 10-membered, bicyclic heterocycle, comprising at least one oxygen atom.

In some embodiments, L is substituted with at least one hydroxy or alkoxy substituent. In certain embodiments, L is substituted with —OH, —OR⁵, —OR⁶, or —OR⁷, wherein R⁵, R⁶, and R⁷ are each independently an oxygen protecting group, or wherein R⁵, R⁶, and R⁷ may be joined together with the intervening atoms to form optionally substituted heterocyclyl. In some embodiments, L is substituted with —OH. In certain embodiments, L is substituted with —OR⁵, —OR⁶, or —OR⁷. In some embodiments, L is substituted with —OR⁵. In certain embodiments, L is substituted with —OR⁶. In some embodiments, L is substituted with —OR⁷.

In certain embodiments, L is

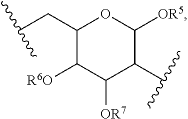

wherein R⁵, R⁶, and R⁷ are each independently hydrogen, C₁₋₆ aliphatic, an oxygen protecting group, or wherein R⁶ and R⁷ may be joined together with the intervening atoms to form optionally substituted heterocyclyl.

In certain embodiments, the compound of Formula (II) is of the formula:

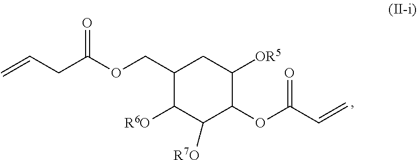

wherein R⁵, R⁶, and R are each independently hydrogen, C₁₋₆ aliphatic, an oxygen protecting group, or wherein R⁶ and R⁷ may be joined together with the intervening atoms to form optionally substituted heterocyclyl.

As defined herein, R⁵, R⁶, and R⁷ are each independently hydrogen, C₁₋₆ aliphatic, an oxygen protecting group, or wherein R⁶ and R⁷ may be joined together with the intervening atoms to form optionally substituted heterocyclyl. In some embodiments, R⁵, R⁶, and R⁷ are each independently an oxygen protecting group, or wherein R⁶ and R⁷ may be joined together with the intervening atoms to form optionally substituted heterocyclyl. In some embodiments, R⁵, R⁶, and R⁷ are each independently an oxygen protecting group or wherein R⁶ and R⁷ may be joined together with the intervening atoms to form optionally substituted heterocyclyl. In some embodiments, R⁵ is an oxygen protecting group. In some embodiments, R⁵ is C₁₋₆ aliphatic. In some embodiments, R⁵ is hydrogen. In certain embodiments, R⁶ is an oxygen protecting group. In some embodiments, R⁶ is C₁₋₆ aliphatic. In some embodiments, R⁶ is hydrogen. In some embodiments, R⁷ is an oxygen protecting group. In some embodiments, R⁷ is C₁₋₆ aliphatic. In some embodiments, R⁷ is hydrogen. In certain embodiments, the oxygen protecting group is unsubstituted C₁-C₆ alkyl, silyl, TBDPS, TBDMS, TIPS, TES, TMS, MOM, THP, t-Bu, Bn, allyl, acetyl, pivaloyl, or benzoyl. In some embodiments, the oxygen protecting group is unsubstituted C₁-C₆ alkyl.

In certain embodiments, R⁶ and R⁷ are joined together with the intervening atoms to form optionally substituted heterocyclyl. In certain embodiments, R⁶ and R⁷ are joined together with the intervening atoms to form optionally substituted 5-6-membered heterocyclyl. In certain embodiments, R⁶ and R⁷ are joined together with the intervening atoms to form unsubstituted 5-6-membered heterocyclyl. In certain embodiments, R⁶ and R⁷ are joined together with the intervening atoms to form substituted 5-6-membered heterocyclyl. In certain embodiments, R⁶ and R⁷ are joined together with the intervening atoms to form 5-6-membered heterocyclyl substituted with one or more C₁-C₆ alkyl substituents. In some embodiments, R⁶ and R⁷ are joined together with the intervening atoms to form a cyclic ketal. In certain embodiments, R⁶ and R⁷ are joined together with the intervening atoms to form a 1,3-dioxolane. In some embodiments, R⁶ and R⁷ are joined together with the intervening atoms to form 2,2-dimethyl-1,3-dioxolane.

In some embodiments, L is

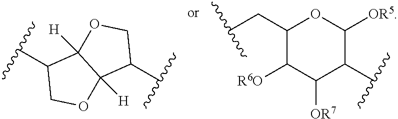

In some embodiments, L is

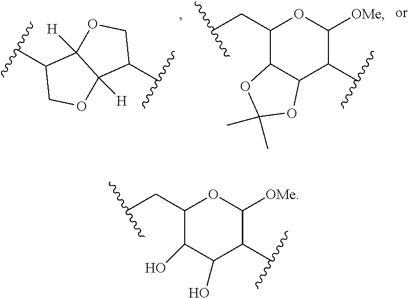

In some embodiments, L is

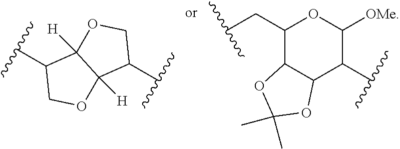 [SMILES: *C1COC2C(*)COC12 ; "or" ; *CC1OC(OC)C(*)C2OC(C)(C)OC12]

In some embodiments, L is

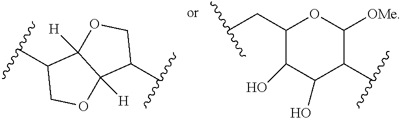 [SMILES: *C1COC2C(*)COC12 ; "or" ; *CC1OC(OC)C(*)C(O)C1O]

In some embodiments, L is

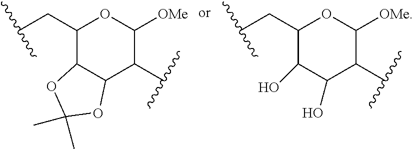 [SMILES: *CC1OC(OC)C(*)C2OC(C)(C)OC12 ; "or" ; *CC1OC(OC)C(*)C(O)C1O]

In some embodiments, L is

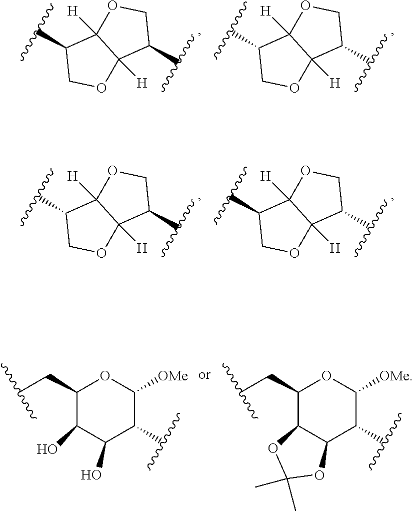

In some embodiments, L is

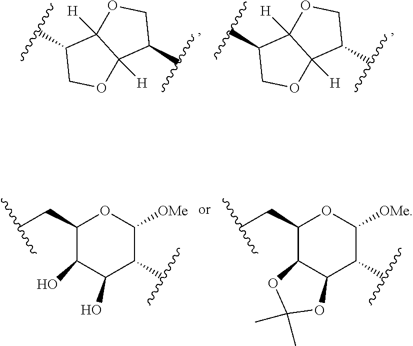

In some embodiments, L is

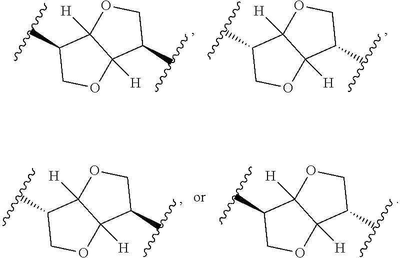

In some embodiments, L is

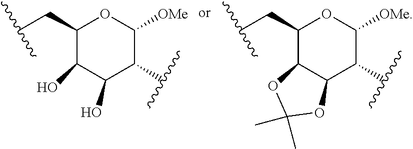 [SMILES: *C[C@H]1O[C@H](OC)[C@H](*)[C@@H](O)[C@H]1O ; "or" ; *C[C@H]1O[C@H](OC)[C@H](*)[C@H]2OC(C)(C)O[C@H]21]

In certain embodiments, L is

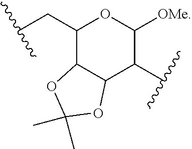 [SMILES: *CC1OC(OC)C(*)C2OC(C)(C)OC12]

In certain embodiments, the compound of Formula (II-i) is

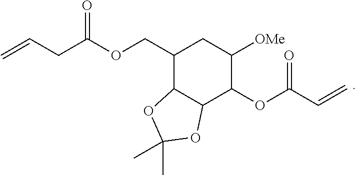 [SMILES: C=CCC(=O)OCC1CC(OC)C(OC(=O)C=C)C2OC(C)(C)OC12 ; "."]

In certain embodiments, L is

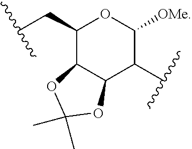 [SMILES: *C[C@H]1O[C@H](OC)C(*)[C@H]2OC(C)(C)O[C@H]21]

In certain embodiments, the compound of Formula (II-i) is

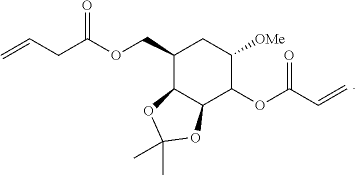 [SMILES: C=CCC(=O)OC[C@H]1C[C@H](OC)C(OC(=O)C=C)[C@H]2OC(C)(C)O[C@@H]12 ; "."]

In certain embodiments, L is

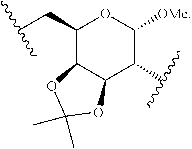 [SMILES: *C[C@H]1O[C@H](OC)[C@H](*)[C@H]2OC(C)(C)O[C@H]21]

In certain embodiments, the compound of Formula (II-i) is

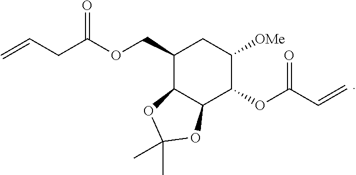 [SMILES: C=CCC(=O)OC[C@H]1C[C@H](OC)[C@H](OC(=O)C=C)[C@H]2OC(C)(C)O[C@@H]12 ; "."]

In certain embodiments, L is

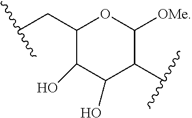 [SMILES: *CC1OC(OC)C(*)C(O)C1O]

In certain embodiments, the compound of Formula (II-i) is

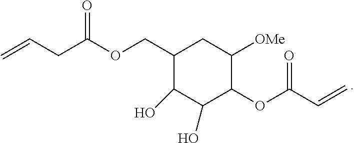 [SMILES: C=CCC(=O)OCC1CC(OC)C(OC(=O)C=C)C(O)C1O ; "."]

In certain embodiments, L is

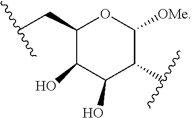 [SMILES: *C[C@H]1O[C@H](OC)[C@H](*)[C@@H](O)[C@H]1O]

In certain embodiments, the compound of Formula (II-i) is

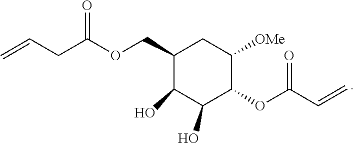 [SMILES: C=CCC(=O)OC[C@H]1C[C@H](OC)[C@H](OC(=O)C=C)[C@@H](O)[C@H]1O ; "."]

In certain embodiments, L is

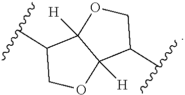 [SMILES: *C1COC2C(*)COC12 ; "."]

In certain embodiments, L is

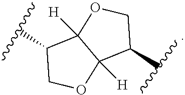 [SMILES: *[C@@H]1COC2C1OC[C@@H]2* ; "."]

In some embodiments, L is

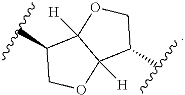 [SMILES: *[C@@H]1COC2C1OC[C@@H]2* ; "."]

In some embodiments, L is

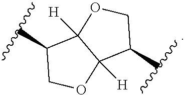 [SMILES: *[C@@H]1COC2C1OC[C@H]2* ; "."]

In some embodiments, L is

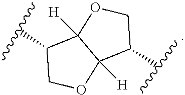 [SMILES: *[C@H]1COC2C1OC[C@@H]2* ; "."]

In certain embodiments, L is

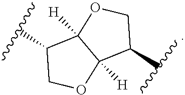 [SMILES: *[C@@H]1CO[C@H]2[C@@H]1OC[C@@H]2* ; "."]

In some embodiments, L is

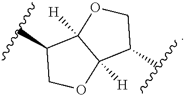 [SMILES: *[C@@H]1CO[C@H]2[C@@H]1OC[C@@H]2* ; "."]

In some embodiments, L is

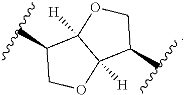 [SMILES: *[C@@H]1CO[C@H]2[C@@H]1OC[C@H]2* ; "."]

In some embodiments, L is

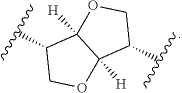 [SMILES: *[C@H]1CO[C@H]2[C@@H]1OC[C@@H]2*]

In certain embodiments, the compound of Formula (II) is

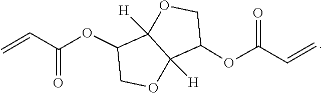 [SMILES: C=CC(=O)OC1COC2C(OC(=O)C=C)COC12]

In certain embodiments, the compound of Formula (II) is

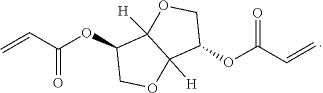 [SMILES: C=CC(=O)O[C@H]1COC2C1OC[C@H]2OC(=O)C=C]

In certain embodiments, the compound of Formula (II) is

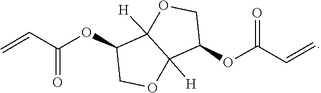 [SMILES: C=CC(=O)O[C@@H]1COC2C1OC[C@H]2OC(=O)C=C]

In certain embodiments, the compound of Formula (II) is

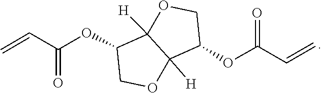 [SMILES: C=CC(=O)O[C@H]1COC2C1OC[C@@H]2OC(=O)C=C]

In certain embodiments, the compound of Formula (II) is

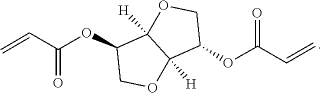 [SMILES: C=CC(=O)O[C@H]1CO[C@H]2[C@@H]1OC[C@H]2OC(=O)C=C]

In certain embodiments, the compound of Formula (II) is

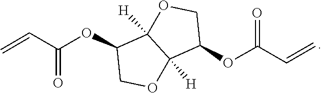 [SMILES: C=CC(=O)O[C@@H]1CO[C@H]2[C@@H]1OC[C@H]2OC(=O)C=C]

In certain embodiments, the compound of Formula (II) is

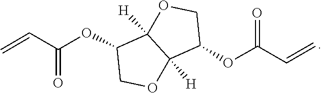 [SMILES: C=CC(=O)O[C@H]1CO[C@H]2[C@@H]1OC[C@@H]2OC(=O)C=C]

As defined herein, R¹ and R² are each independently hydrogen; branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group; or one or both R¹ or R² are each optionally independently joined to linker A together with the intervening atoms to form a linker comprising one or more N-containing heterocycles; or R¹ and R² are joined together with the intervening atoms to form a N-containing heterocycle.

In certain embodiments, R¹ and R² are each independently hydrogen; branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group.

In some embodiments, R¹ is hydrogen.

In certain embodiments, R¹ is branched or unbranched, optionally substituted, cyclic or acyclic aliphatic. In some embodiments, R¹ is branched or unbranched, optionally substituted, cyclic aliphatic. In certain embodiments, R¹ is substituted cyclic aliphatic. In some embodiments, linker is unsubstituted cyclic aliphatic. In certain embodiments, R¹ is branched or unbranched, optionally substituted carbocyclylene. In some embodiments, R¹ is substituted carbocyclylene. In certain embodiments, R¹ is unsubstituted carbocyclylene. In certain embodiments, R¹ is branched or unbranched, optionally substituted C₃-C₈ carbocyclylene. In some embodiments, R¹ is substituted C₃-C₈ carbocyclylene. In certain embodiments, R¹ is unsubstituted C₃-C₈ carbocyclylene. In some embodiments, R¹ is branched or unbranched, optionally substituted, acyclic aliphatic. In some embodiments, R¹ is substituted acyclic aliphatic. In certain embodiments, R¹ is unsubstituted acyclic aliphatic. In some embodiments, R¹ is branched or unbranched, optionally substituted, alkylene. In some embodiments, R¹ is substituted alkylene. In some embodiments, R¹ is unsubstituted alkylene. In some embodiments, R¹ is branched or unbranched, optionally substituted, C₁-C₁₀ alkylene. In some embodiments, R¹ is substituted C₁-C₁₀ alkylene. In some embodiments, R¹ is unsubstituted C₁-C₁₀ alkylene. In some embodiments, R¹ is branched or unbranched, optionally substituted, alkenylene. In some embodiments, R¹ is substituted alkenylene. In some embodiments, R¹ is unsubstituted alkenylene. In some embodiments, R¹ is branched or unbranched, optionally substituted, C₂-C₁₀ alkenylene. In some embodiments, R¹ is substituted C₂-C₁₀ alkenylene. In some embodiments, R¹ is unsubstituted C₂-C₁₀ alkenylene. In some embodiments, R¹ is branched or unbranched, optionally substituted, alkynylene. In some embodiments, R¹ is substituted alkynylene. In some embodiments, R¹ is unsubstituted alkynylene. In some embodiments, R¹ is branched or unbranched, optionally substituted, C₂-C₁₀ alkynylene. In some embodiments, R¹ is substituted C₂-C₁₀ alkynylene. In some embodiments, R¹ is unsubstituted C₂-C₁₀ alkynylene.

In certain embodiments, R¹ is branched or unbranched, optionally substituted, cyclic or acyclic heteroaliphatic. In some embodiments, R¹ is branched or unbranched, optionally substituted, cyclic heteroaliphatic. In certain embodiments, R¹ is substituted cyclic heteroaliphatic. In some embodiments, linker is unsubstituted cyclic heteroaliphatic. In certain embodiments, R¹ is branched or unbranched, optionally substituted heterocyclylene. In some embodiments, R¹ is substituted heterocyclylene. In certain embodiments, R¹ is unsubstituted heterocyclylene. In certain embodiments, R¹ is branched or unbranched, optionally substituted 3-8-membered heterocyclylene. In some embodiments, R¹ is substituted 3-8-membered heterocyclylene. In certain embodiments, R¹ is unsubstituted 3-8-membered heterocyclylene. In some embodiments, R¹ is branched or unbranched, optionally substituted, acyclic heteroaliphatic. In some embodiments, R¹ is substituted acyclic heteroaliphatic. In certain embodiments, R¹ is unsubstituted acyclic heteroaliphatic. In some embodiments, R¹ is branched or unbranched, optionally substituted, heteroalkylene. In some embodiments, R¹ is substituted heteroalkylene. In some embodiments, R¹ is unsubstituted heteroalkylene. In some embodiments, R¹ is branched or unbranched, optionally substituted, C₁-C₁₀ heteroalkylene. In some embodiments, R¹ is substituted C₁-C₁₀ heteroalkylene. In some embodiments, R¹ is unsubstituted C₁-C₁₀ heteroalkylene. In some embodiments, R¹ is branched or unbranched, optionally substituted, heteroalkenylene. In some embodiments, R¹ is substituted heteroalkenylene. In some embodiments, R¹ is unsubstituted heteroalkenylene. In some embodiments, R¹ is branched or unbranched, optionally substituted, C₂-C₁₀ heteroalkenylene. In some embodiments, R¹ is substituted C₂-C₁₀ heteroalkenylene. In some embodiments, R¹ is unsubstituted C₂-C₁₀ heteroalkenylene. In some embodiments, R¹ is branched or unbranched, optionally substituted, heteroalkynylene. In some embodiments, R¹ is substituted heteroalkynylene. In some embodiments, R¹ is unsubstituted heteroalkynylene. In some embodiments, R¹ is branched or unbranched, optionally substituted, C₂-C₁₀ heteroalkynylene. In some embodiments, R¹ is substituted C₂-C₁₀ heteroalkynylene. In some embodiments, R¹ is unsubstituted C₂-C₁₀ heteroalkynylene.

In certain embodiments, R¹ is branched or unbranched, optionally substituted, cyclic or acyclic acyl. In some embodiments, R¹ is substituted cyclic or acyclic acyl. In some embodiments, R¹ is unsubstituted cyclic or acyclic acyl. In certain embodiments, R¹ is branched or unbranched, optionally substituted, cyclic acyl. In some embodiments, R¹ is substituted cyclic acyl. In some embodiments, R¹ is unsubstituted cyclic acyl. In certain embodiments, R¹ is branched or unbranched, optionally substituted, C₅-C₁₀ cyclic acyl. In some embodiments, R¹ is substituted C₅-C₁₀ cyclic acyl. In some embodiments, R¹ is unsubstituted C₅-C₁₀ cyclic acyl. In certain embodiments, R¹ is branched or unbranched, optionally substituted, acyclic acyl. In some embodiments, R¹ is substituted acyclic acyl. In some embodiments, R¹ is unsubstituted acyclic acyl. In certain embodiments, R¹ is branched or unbranched, optionally substituted, C₁-C₁₀ acyclic acyl. In some embodiments, R¹ is substituted C₁-C₁₀ acyclic acyl. In some embodiments, R¹ is unsubstituted C₁-C₁₀ acyclic acyl.

In certain embodiments, R¹ is optionally substituted aryl. In some embodiments, R¹ is substituted aryl. In certain embodiments, R¹ is unsubstituted aryl. In some embodiments, R¹ is optionally substituted C₆-C₁₄ aryl. In certain embodiments, R¹ is substituted C₆-C₁₄ aryl. In some embodiments, R¹ is unsubstituted C₆-C₁₄ aryl.

In certain embodiments, R¹ is optionally substituted heteroaryl. In some embodiments, R¹ is substituted heteroaryl. In certain embodiments, R¹ is unsubstituted heteroaryl. In certain embodiments, R¹ is optionally substituted 5-14-membered heteroaryl. In some embodiments, R¹ is substituted 5-14-membered heteroaryl. In certain embodiments, R¹ is unsubstituted 5-14-membered heteroaryl.

In certain embodiments, R¹ is a nitrogen protecting group. In some embodiments, the nitrogen protecting group is Bn, Boc, Cbz, Fmoc, trifluoroacetyl, triphenylmethyl, acetyl, or Ts.

In some embodiments, R² is hydrogen.

In certain embodiments, R² is branched or unbranched, optionally substituted, cyclic or acyclic aliphatic. In some embodiments, R² is branched or unbranched, optionally substituted, cyclic aliphatic. In certain embodiments, R² is substituted cyclic aliphatic. In some embodiments, linker is unsubstituted cyclic aliphatic. In certain embodiments, R² is branched or unbranched, optionally substituted carbocyclylene. In some embodiments, R² is substituted carbocyclylene. In certain embodiments, R² is unsubstituted carbocyclylene. In certain embodiments, R² is branched or unbranched, optionally substituted C₃-C₈ carbocyclylene. In some embodiments, R² is substituted C₃-C₈ carbocyclylene. In certain embodiments, R² is unsubstituted C₃-C₈ carbocyclylene. In some embodiments, R² is branched or unbranched, optionally substituted, acyclic aliphatic. In some embodiments, R² is substituted acyclic aliphatic. In certain embodiments, R² is unsubstituted acyclic aliphatic. In some embodiments, R² is branched or unbranched, optionally substituted, alkylene. In some embodiments, R² is substituted alkylene. In some embodiments, R² is unsubstituted alkylene. In some embodiments, R² is branched or unbranched, optionally substituted, C₁-C₁₀ alkylene. In some embodiments, R² is substituted C₁-C₁₀ alkylene. In some embodiments, R² is unsubstituted C₁-C₁₀ alkylene. In some embodiments, R² is branched or unbranched, optionally substituted, alkenylene. In some embodiments, R² is substituted alkenylene. In some embodiments, R² is unsubstituted alkenylene. In some embodiments, R² is branched or unbranched, optionally substituted, C₂-C₁₀ alkenylene. In some embodiments, R² is substituted C₂-C₁₀ alkenylene. In some embodiments, R² is unsubstituted C₂-C₁₀ alkenylene. In some embodiments, R² is branched or unbranched, optionally substituted, alkynylene. In some embodiments, R² is substituted alkynylene. In some embodiments, R² is unsubstituted alkynylene. In some embodiments, R² is branched or unbranched, optionally substituted, C₂-C₁₀ alkynylene. In some embodiments, R² is substituted C₂-C₁₀ alkynylene. In some embodiments, R² is unsubstituted C₂-C₁₀ alkynylene.

In certain embodiments, R² is branched or unbranched, optionally substituted, cyclic or acyclic heteroaliphatic. In some embodiments, R² is branched or unbranched, optionally substituted, cyclic heteroaliphatic. In certain embodiments, R² is substituted cyclic heteroaliphatic. In some embodiments, linker is unsubstituted cyclic heteroaliphatic. In certain embodiments, R² is branched or unbranched, optionally substituted heterocyclylene. In some embodiments, R² is substituted heterocyclylene. In certain embodiments, R² is unsubstituted heterocyclylene. In certain embodiments, R² is branched or unbranched, optionally substituted 3-8-membered heterocyclylene. In some embodiments, R² is substituted 3-8-membered heterocyclylene. In certain embodiments, R² is unsubstituted 3-8-membered heterocyclylene. In some embodiments, R² is branched or unbranched, optionally substituted, acyclic heteroaliphatic. In some embodiments, R² is substituted acyclic heteroaliphatic. In certain embodiments, R² is unsubstituted acyclic heteroaliphatic. In some embodiments, R² is branched or unbranched, optionally substituted, heteroalkylene. In some embodiments, R² is substituted heteroalkylene. In some embodiments, R² is unsubstituted heteroalkylene. In some embodiments, R² is branched or unbranched, optionally substituted, C₁-C₁₀ heteroalkylene. In some embodiments, R² is substituted C₁-C₁₀ heteroalkylene. In some embodiments, R² is unsubstituted C₁-C₁₀ heteroalkylene. In some embodiments, R² is branched or unbranched, optionally substituted, heteroalkenylene. In some embodiments, R² is substituted heteroalkenylene. In some embodiments, R² is unsubstituted heteroalkenylene. In some embodiments, R² is branched or unbranched, optionally substituted, C₂-C₁₀ heteroalkenylene. In some embodiments, R² is substituted C₂-C₁₀ heteroalkenylene. In some embodiments, R² is unsubstituted C₂-C₁₀ heteroalkenylene. In some embodiments, R² is branched or unbranched, optionally substituted, heteroalkynylene. In some embodiments, R² is substituted heteroalkynylene. In some embodiments, R² is unsubstituted heteroalkynylene. In some embodiments, R² is branched or unbranched, optionally substituted, C₂-C₁₀ heteroalkynylene. In some embodiments, R² is substituted C₂-C₁₀ heteroalkynylene. In some embodiments, R² is unsubstituted C₂-C₁₀ heteroalkynylene.

In certain embodiments, R² is branched or unbranched, optionally substituted, cyclic or acyclic acyl. In some embodiments, R² is substituted cyclic or acyclic acyl. In some embodiments, R² is unsubstituted cyclic or acyclic acyl. In certain embodiments, R² is branched or unbranched, optionally substituted, cyclic acyl. In some embodiments, R² is substituted cyclic acyl. In some embodiments, R² is unsubstituted cyclic acyl. In certain embodiments, R² is branched or unbranched, optionally substituted, C₅-C₁₀ cyclic acyl. In some embodiments, R² is substituted C₅-C₁₀ cyclic acyl. In some embodiments, R² is unsubstituted C₅-C₁₀ cyclic acyl. In certain embodiments, R² is branched or unbranched, optionally substituted, acyclic acyl. In some embodiments, R² is substituted acyclic acyl. In some embodiments, R² is unsubstituted acyclic acyl. In certain embodiments, R² is branched or unbranched, optionally substituted, C₁-C₁₀ acyclic acyl. In some embodiments, R² is substituted C₁-C₁₀ acyclic acyl. In some embodiments, R² is unsubstituted C₁-C₁₀ acyclic acyl.

In certain embodiments, R² is optionally substituted aryl. In some embodiments, R² is substituted aryl. In certain embodiments, R² is unsubstituted aryl. In some embodiments, R² is optionally substituted C₆-C₁₄ aryl. In certain embodiments, R² is substituted C₆-C₁₄ aryl. In some embodiments, R² is unsubstituted C₆-C₁₄ aryl.

In certain embodiments, R² is optionally substituted heteroaryl. In some embodiments, R² is substituted heteroaryl. In certain embodiments, R² is unsubstituted heteroaryl. In certain embodiments, R² is optionally substituted 5-14-membered heteroaryl. In some embodiments, R² is substituted 5-14-membered heteroaryl. In certain embodiments, R² is unsubstituted 5-14-membered heteroaryl.

In certain embodiments, R² is a nitrogen protecting group. In some embodiments, the nitrogen protecting group is Bn, Boc, Cbz, Fmoc, trifluoroacetyl, triphenylmethyl, acetyl, or Ts.

In some embodiments, one or both R¹ or R² are each independently joined to linker A together with the intervening atoms to form a linker comprising one or more N-containing heterocycles; or R¹ and R² are joined together with the intervening atoms to form a N-containing heterocycle. In certain embodiments, one or both R¹ or R² are each optionally independently joined to linker A together with the intervening atoms to form a linker comprising one or more N-containing heterocycles. In some embodiments, R¹ is joined to linker A together with the intervening atoms to form a linker comprising at least one N-containing heterocycle. In some embodiments, R^{B} is joined to linker A together with the intervening atoms to form a linker comprising at least one N-containing heterocycle. In some embodiments, R¹ and R² are joined together with the intervening atoms to form a N-containing heterocycle. In some embodiments, the N-containing heterocycle is a 3-8-membered N-containing heterocycle. In some embodiments, the N-containing heterocycle is a 5-7-membered N-containing heterocycle. In some embodiments, the N-containing heterocycle is a 5-membered N-containing heterocycle. In some embodiments, the N-containing heterocycle is a 6-membered N-containing heterocycle. In some embodiments, the N-containing heterocycle is a 7-membered N-containing heterocycle.

As defined herein, linker A comprises branched or unbranched, optionally substituted, cyclic or acyclic aliphatic; branched or unbranched, optionally substituted, cyclic or acyclic heteroaliphatic; optionally substituted arylene; or optionally substituted heteroarylene. As defined herein, linker A is branched or unbranched, optionally substituted, cyclic or acyclic aliphatic; branched or unbranched, optionally substituted, cyclic or acyclic heteroaliphatic; optionally substituted arylene; or optionally substituted heteroarylene.

In certain embodiments, linker A is branched or unbranched, optionally substituted, cyclic or acyclic aliphatic. In some embodiments, linker A is branched or unbranched, optionally substituted, cyclic aliphatic. In certain embodiments, linker A is substituted cyclic aliphatic. In some embodiments, linker is unsubstituted cyclic aliphatic. In certain embodiments, linker A is branched or unbranched, optionally substituted carbocyclylene. In some embodiments, linker A is substituted carbocyclylene. In certain embodiments, linker A is unsubstituted carbocyclylene. In certain embodiments, linker A is branched or unbranched, optionally substituted C₃-C₈ carbocyclylene. In some embodiments, linker A is substituted C₃-C₈ carbocyclylene. In certain embodiments, linker A is unsubstituted C₃-C₈ carbocyclylene.

In some embodiments, linker A is branched or unbranched, optionally substituted, acyclic aliphatic. In some embodiments, linker A is substituted acyclic aliphatic. In certain embodiments, linker A is unsubstituted acyclic aliphatic. In some embodiments, linker A is branched or unbranched, optionally substituted, alkylene. In some embodiments, linker A is substituted alkylene. In some embodiments, linker A is unsubstituted alkylene. In some embodiments, linker A is branched or unbranched, optionally substituted, C₁-C₁₀ alkylene. In some embodiments, linker A is substituted C₁-C₁₀ alkylene. In some embodiments, linker A is unsubstituted C₁-C₁₀ alkylene. In some embodiments, linker A is branched or unbranched, optionally substituted, alkenylene. In some embodiments, linker A is substituted alkenylene. In some embodiments, linker A is unsubstituted alkenylene. In some embodiments, linker A is branched or unbranched, optionally substituted, C₂-C₁₀ alkenylene. In some embodiments, linker A is substituted C₂-C₁₀ alkenylene. In some embodiments, linker A is unsubstituted C₂-C₁₀ alkenylene. In some embodiments, linker A is branched or unbranched, optionally substituted, alkynylene. In some embodiments, linker A is substituted alkynylene. In some embodiments, linker A is unsubstituted alkynylene. In some embodiments, linker A is branched or unbranched, optionally substituted, C₂-C₁₀ alkynylene. In some embodiments, linker A is substituted C₂-C₁₀ alkynylene. In some embodiments, linker A is unsubstituted C₂-C₁₀ alkynylene.

In certain embodiments, linker A is branched or unbranched, optionally substituted, cyclic or acyclic heteroaliphatic. In some embodiments, linker A is branched or unbranched, optionally substituted, cyclic heteroaliphatic. In certain embodiments, linker A is substituted cyclic heteroaliphatic. In some embodiments, linker is unsubstituted cyclic heteroaliphatic. In certain embodiments, linker A is branched or unbranched, optionally substituted heterocyclylene. In some embodiments, linker A is substituted heterocyclylene. In certain embodiments, linker A is unsubstituted heterocyclylene. In certain embodiments, linker A is branched or unbranched, optionally substituted 3-8-membered heterocyclylene. In some embodiments, linker A is substituted 3-8-membered heterocyclylene. In certain embodiments, linker A is unsubstituted 3-8-membered heterocyclylene.

In some embodiments, linker A is branched or unbranched, optionally substituted, acyclic heteroaliphatic. In some embodiments, linker A is substituted acyclic heteroaliphatic. In certain embodiments, linker A is unsubstituted acyclic heteroaliphatic. In some embodiments, linker A is branched or unbranched, optionally substituted, heteroalkylene. In some embodiments, linker A is substituted heteroalkylene. In some embodiments, linker A is unsubstituted heteroalkylene. In some embodiments, linker A is branched or unbranched, optionally substituted, C₁-C₁₀ heteroalkylene. In some embodiments, linker A is substituted C₁-C₁₀ heteroalkylene. In some embodiments, linker A is unsubstituted C₁-C₁₀ heteroalkylene. In some embodiments, linker A is branched or unbranched, optionally substituted, heteroalkenylene. In some embodiments, linker A is substituted heteroalkenylene. In some embodiments, linker A is unsubstituted heteroalkenylene. In some embodiments, linker A is branched or unbranched, optionally substituted, C₂-C₁₀ heteroalkenylene. In some embodiments, linker A is substituted C₂-C₁₀ heteroalkenylene. In some embodiments, linker A is unsubstituted C₂-C₁₀ heteroalkenylene. In some embodiments, linker A is branched or unbranched, optionally substituted, heteroalkynylene. In some embodiments, linker A is substituted heteroalkynylene. In some embodiments, linker A is unsubstituted heteroalkynylene. In some embodiments, linker A is branched or unbranched, optionally substituted, C₂-C₁₀ heteroalkynylene. In some embodiments, linker A is substituted C₂-C₁₀ heteroalkynylene. In some embodiments, linker A is unsubstituted C₂-C₁₀ heteroalkynylene.

In certain embodiments, linker A is optionally substituted arylene. In some embodiments, linker A is substituted arylene. In certain embodiments, linker A is unsubstituted arylene. In some embodiments, linker A is optionally substituted C₆-C₁₄ arylene. In certain embodiments, linker A is substituted C₆-C₁₄ arylene. In some embodiments, linker A is unsubstituted C₆-C₁₄ arylene.

In certain embodiments, linker A is optionally substituted heteroarylene. In some embodiments, linker A is substituted heteroarylene. In certain embodiments, linker A is unsubstituted heteroarylene. In certain embodiments, linker A is optionally substituted 5-14-membered heteroarylene. In some embodiments, linker A is substituted 5-14-membered heteroarylene. In certain embodiments, linker A is unsubstituted 5-14-membered heteroarylene.

In certain embodiments,

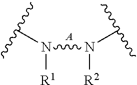

is selected from

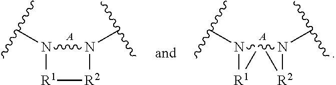

In some embodiments,

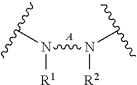

is

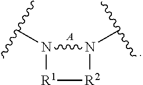

In certain embodiments,

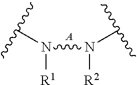

is

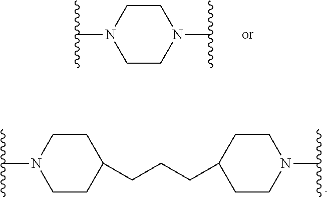 [SMILES: *N1CCN(*)CC1 ; *N1CCC(CCCC2CCN(*)CC2)CC1]

In certain embodiments,

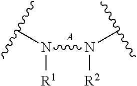

is

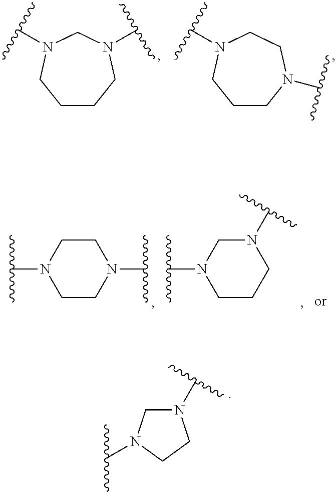

In some embodiments,

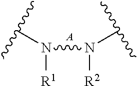

is

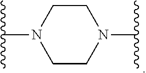 [SMILES: *N1CCN(*)CC1 ; "."]

In certain embodiments,

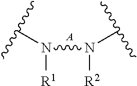

is

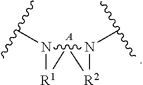

In some embodiments,

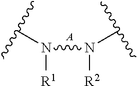

is

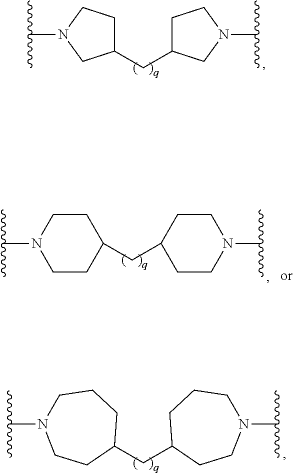

wherein each q is independently 1-10. In certain embodiments, q is 1-6. In some embodiments, q is 1-4. In certain embodiments, q is 1. In some embodiments, q is 2. In certain embodiments, q is 3. In some embodiments, q is 4. In certain embodiments, q is 5. In some embodiments, q is 6. In certain embodiments, q is 7. In some embodiments, q is 8. In certain embodiments, q is 9. In some embodiments, q is 10.

In certain embodiments,

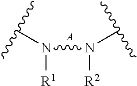

is

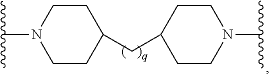

wherein each q is independently 1-10. In certain embodiments, q is 1-6. In some embodiments, q is 1-4. In certain embodiments, q is 1. In some embodiments, q is 2. In certain embodiments, q is 3. In some embodiments, q is 4. In certain embodiments, q is 5. In some embodiments, q is 6. In certain embodiments, q is 7. In some embodiments, q is 8. In certain embodiments, q is 9. In some embodiments, q is 10. In certain embodiments,

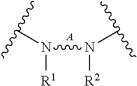

is

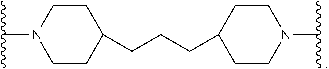 [SMILES: *N1CCC(CCCC2CCN(*)CC2)CC1 ; "."]

As defined herein, R³ is branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group.

In certain embodiments, R³ is branched or unbranched, optionally substituted, cyclic or acyclic aliphatic. In some embodiments, R³ is branched or unbranched, optionally substituted, cyclic aliphatic. In certain embodiments, R³ is substituted cyclic aliphatic. In some embodiments, linker is unsubstituted cyclic aliphatic. In certain embodiments, R³ is branched or unbranched, optionally substituted carbocyclylene. In some embodiments, R³ is substituted carbocyclylene. In certain embodiments, R³ is unsubstituted carbocyclylene. In certain embodiments, R³ is branched or unbranched, optionally substituted C₃-C₈ carbocyclylene. In some embodiments, R³ is substituted C₃-C₈ carbocyclylene. In certain embodiments, R³ is unsubstituted C₃-C₈ carbocyclylene. In some embodiments, R³ is branched or unbranched, optionally substituted, acyclic aliphatic. In some embodiments, R³ is substituted acyclic aliphatic. In certain embodiments, R³ is unsubstituted acyclic aliphatic. In some embodiments, R³ is branched or unbranched, optionally substituted, alkylene. In some embodiments, R³ is substituted alkylene. In some embodiments, R³ is unsubstituted alkylene. In some embodiments, R³ is branched or unbranched, optionally substituted, C₁-C₁₀ alkylene. In some embodiments, R³ is substituted C₁-C₁₀ alkylene. In some embodiments, R³ is unsubstituted C₁-C₁₀ alkylene. In some embodiments, R³ is branched or unbranched, optionally substituted, alkenylene. In some embodiments, R³ is substituted alkenylene. In some embodiments, R³ is unsubstituted alkenylene. In some embodiments, R³ is branched or unbranched, optionally substituted, C₂-C₁₀ alkenylene. In some embodiments, R³ is substituted C₂-C₁₀ alkenylene. In some embodiments, R³ is unsubstituted C₂-C₁₀ alkenylene. In some embodiments, R³ is branched or unbranched, optionally substituted, alkynylene. In some embodiments, R³ is substituted alkynylene. In some embodiments, R³ is unsubstituted alkynylene. In some embodiments, R³ is branched or unbranched, optionally substituted, C₂—C₁₀ alkynylene. In some embodiments, R³ is substituted C₂-C₁₀ alkynylene. In some embodiments, R³ is unsubstituted C₂-C₁₀ alkynylene.

In certain embodiments, R³ is branched or unbranched, optionally substituted, cyclic or acyclic heteroaliphatic. In some embodiments, R³ is branched or unbranched, optionally substituted, cyclic heteroaliphatic. In certain embodiments, R³ is substituted cyclic heteroaliphatic. In some embodiments, linker is unsubstituted cyclic heteroaliphatic. In certain embodiments, R³ is branched or unbranched, optionally substituted heterocyclylene. In some embodiments, R³ is substituted heterocyclylene. In certain embodiments, R³ is unsubstituted heterocyclylene. In certain embodiments, R³ is branched or unbranched, optionally substituted 3-8-membered heterocyclylene. In some embodiments, R³ is substituted 3-8-membered heterocyclylene. In certain embodiments, R³ is unsubstituted 3-8-membered heterocyclylene. In some embodiments, R³ is branched or unbranched, optionally substituted, acyclic heteroaliphatic. In some embodiments, R³ is substituted acyclic heteroaliphatic. In certain embodiments, R³ is unsubstituted acyclic heteroaliphatic. In some embodiments, R³ is branched or unbranched, optionally substituted, heteroalkylene. In some embodiments, R³ is substituted heteroalkylene. In some embodiments, R³ is unsubstituted heteroalkylene. In some embodiments, R³ is branched or unbranched, optionally substituted, C₁-C₁₀ heteroalkylene. In some embodiments, R³ is substituted C₁-C₁₀ heteroalkylene. In some embodiments, R³ is unsubstituted C₁-C₁₀ heteroalkylene. In some embodiments, R³ is branched or unbranched, optionally substituted, heteroalkenylene. In some embodiments, R³ is substituted heteroalkenylene. In some embodiments, R³ is unsubstituted heteroalkenylene. In some embodiments, R³ is branched or unbranched, optionally substituted, C₂-C₁₀ heteroalkenylene. In some embodiments, R³ is substituted C₂-C₁₀ heteroalkenylene. In some embodiments, R³ is unsubstituted C₂-C₁₀ heteroalkenylene. In some embodiments, R³ is branched or unbranched, optionally substituted, heteroalkynylene. In some embodiments, R³ is substituted heteroalkynylene. In some embodiments, R³ is unsubstituted heteroalkynylene. In some embodiments, R³ is branched or unbranched, optionally substituted, C₂-C₁₀ heteroalkynylene. In some embodiments, R³ is substituted C₂-C₁₀ heteroalkynylene. In some embodiments, R³ is unsubstituted C₂-C₁₀ heteroalkynylene.

In certain embodiments, R³ is branched or unbranched, optionally substituted, cyclic or acyclic acyl. In some embodiments, R³ is substituted cyclic or acyclic acyl. In some embodiments, R³ is unsubstituted cyclic or acyclic acyl. In certain embodiments, R³ is branched or unbranched, optionally substituted, cyclic acyl. In some embodiments, R³ is substituted cyclic acyl. In some embodiments, R³ is unsubstituted cyclic acyl. In certain embodiments, R³ is branched or unbranched, optionally substituted, C₅-C₁₀ cyclic acyl. In some embodiments, R³ is substituted C₅-C₁₀ cyclic acyl. In some embodiments, R³ is unsubstituted C₅-C₁₀ cyclic acyl. In certain embodiments, R³ is branched or unbranched, optionally substituted, acyclic acyl. In some embodiments, R³ is substituted acyclic acyl. In some embodiments, R³ is unsubstituted acyclic acyl. In certain embodiments, R³ is branched or unbranched, optionally substituted, C₁-C₁₀ acyclic acyl. In some embodiments, R³ is substituted C₁-C₁₀ acyclic acyl. In some embodiments, R³ is unsubstituted C₁-C₁₀ acyclic acyl.

In certain embodiments, R³ is optionally substituted aryl. In some embodiments, R³ is substituted aryl. In certain embodiments, R³ is unsubstituted aryl. In some embodiments, R³ is optionally substituted C₆-C₁₄ aryl. In certain embodiments, R³ is substituted C₆-C₁₄ aryl. In some embodiments, R³ is unsubstituted C₆-C₁₄ aryl.

In certain embodiments, R³ is optionally substituted heteroaryl. In some embodiments, R³ is substituted heteroaryl. In certain embodiments, R³ is unsubstituted heteroaryl. In certain embodiments, R³ is optionally substituted 5-14-membered heteroaryl. In some embodiments, R³ is substituted 5-14-membered heteroaryl. In certain embodiments, R³ is unsubstituted 5-14-membered heteroaryl.

In certain embodiments, R³ is a nitrogen protecting group. In some embodiments, the nitrogen protecting group is Bn, Boc, Cbz, Fmoc, trifluoroacetyl, triphenylmethyl, acetyl, or Ts.

As defined herein, linker B comprises branched or unbranched, optionally substituted, cyclic or acyclic aliphatic; branched or unbranched, optionally substituted, cyclic or acyclic heteroaliphatic; optionally substituted arylene; or optionally substituted heteroarylene. As defined herein, linker B is branched or unbranched, optionally substituted, cyclic or acyclic aliphatic; branched or unbranched, optionally substituted, cyclic or acyclic heteroaliphatic; optionally substituted arylene; or optionally substituted heteroarylene.

In certain embodiments, linker B is branched or unbranched, optionally substituted, cyclic or acyclic aliphatic. In some embodiments, linker B is branched or unbranched, optionally substituted, cyclic aliphatic. In certain embodiments, linker B is substituted cyclic aliphatic. In some embodiments, linker is unsubstituted cyclic aliphatic. In certain embodiments, linker B is branched or unbranched, optionally substituted carbocyclylene. In some embodiments, linker B is substituted carbocyclylene. In certain embodiments, linker B is unsubstituted carbocyclylene. In certain embodiments, linker B is branched or unbranched, optionally substituted C₃-C₈ carbocyclylene. In some embodiments, linker B is substituted C₃-C₈ carbocyclylene. In certain embodiments, linker B is unsubstituted C₃-C₈ carbocyclylene.

In some embodiments, linker B is branched or unbranched, optionally substituted, acyclic aliphatic. In some embodiments, linker B is substituted acyclic aliphatic. In certain embodiments, linker B is unsubstituted acyclic aliphatic. In some embodiments, linker B is branched or unbranched, optionally substituted, alkylene. In some embodiments, linker B is substituted alkylene. In some embodiments, linker B is unsubstituted alkylene. In some embodiments, linker B is branched or unbranched, optionally substituted, C₁-C₁₀ alkylene. In some embodiments, linker B is substituted C₁-C₁₀ alkylene. In some embodiments, linker B is unsubstituted C₁-C₁₀ alkylene. In some embodiments, linker B is branched or unbranched, optionally substituted, alkenylene. In some embodiments, linker B is substituted alkenylene. In some embodiments, linker B is unsubstituted alkenylene. In some embodiments, linker B is branched or unbranched, optionally substituted, C₂-C₁₀ alkenylene. In some embodiments, linker B is substituted C₂-C₁₀ alkenylene. In some embodiments, linker B is unsubstituted C₂-C₁₀ alkenylene. In some embodiments, linker B is branched or unbranched, optionally substituted, alkynylene. In some embodiments, linker B is substituted alkynylene. In some embodiments, linker B is unsubstituted alkynylene. In some embodiments, linker B is branched or unbranched, optionally substituted, C₂-C₁₀ alkynylene. In some embodiments, linker B is substituted C₂-C₁₀ alkynylene. In some embodiments, linker B is unsubstituted C₂-C₁₀ alkynylene.

In certain embodiments, linker B is branched or unbranched, optionally substituted, cyclic or acyclic heteroaliphatic. In some embodiments, linker B is branched or unbranched, optionally substituted, cyclic heteroaliphatic. In certain embodiments, linker B is substituted cyclic heteroaliphatic. In some embodiments, linker is unsubstituted cyclic heteroaliphatic. In certain embodiments, linker B is branched or unbranched, optionally substituted heterocyclylene. In some embodiments, linker B is substituted heterocyclylene. In certain embodiments, linker B is unsubstituted heterocyclylene. In certain embodiments, linker B is branched or unbranched, optionally substituted 3-8-membered heterocyclylene. In some embodiments, linker B is substituted 3-8-membered heterocyclylene. In certain embodiments, linker B is unsubstituted 3-8-membered heterocyclylene.

In some embodiments, linker B is branched or unbranched, optionally substituted, acyclic heteroaliphatic. In some embodiments, linker B is substituted acyclic heteroaliphatic. In certain embodiments, linker B is unsubstituted acyclic heteroaliphatic. In some embodiments, linker B is branched or unbranched, optionally substituted, heteroalkylene. In some embodiments, linker B is substituted heteroalkylene. In some embodiments, linker B is unsubstituted heteroalkylene. In some embodiments, linker B is branched or unbranched, optionally substituted, C₁-C₁₀ heteroalkylene. In some embodiments, linker B is substituted C₁-C₁₀ heteroalkylene. In some embodiments, linker B is unsubstituted C₁-C₁₀ heteroalkylene. In some embodiments, linker B is branched or unbranched, optionally substituted, heteroalkenylene. In some embodiments, linker B is substituted heteroalkenylene. In some embodiments, linker B is unsubstituted heteroalkenylene. In some embodiments, linker B is branched or unbranched, optionally substituted, C₂-C₁₀ heteroalkenylene. In some embodiments, linker B is substituted C₂-C₁₀ heteroalkenylene. In some embodiments, linker B is unsubstituted C₂-C₁₀ heteroalkenylene. In some embodiments, linker B is branched or unbranched, optionally substituted, heteroalkynylene. In some embodiments, linker B is substituted heteroalkynylene. In some embodiments, linker B is unsubstituted heteroalkynylene. In some embodiments, linker B is branched or unbranched, optionally substituted, C₂-C₁₀ heteroalkynylene. In some embodiments, linker B is substituted C₂-C₁₀ heteroalkynylene. In some embodiments, linker B is unsubstituted C₂-C₁₀ heteroalkynylene.

In certain embodiments, linker B is optionally substituted arylene. In some embodiments, linker B is substituted arylene. In certain embodiments, linker B is unsubstituted arylene. In some embodiments, linker B is optionally substituted C₆-C₁₄ arylene. In certain embodiments, linker B is substituted C₆-C₁₄ arylene. In some embodiments, linker B is unsubstituted C₆-C₁₄ arylene.

In certain embodiments, linker B is optionally substituted heteroarylene. In some embodiments, linker B is substituted heteroarylene. In certain embodiments, linker B is unsubstituted heteroarylene. In certain embodiments, linker B is optionally substituted 5-14-membered heteroarylene. In some embodiments, linker B is substituted 5-14-membered heteroarylene. In certain embodiments, linker B is unsubstituted 5-14-membered heteroarylene.

As defined herein, R⁴ is hydrogen, branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group; or R⁴ is joined to linker B together with the intervening atoms to form a linker comprising a N-containing heterocycle. In certain embodiments, R⁴ is hydrogen, branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group.

In some embodiments, R⁴ is hydrogen.

In certain embodiments, R⁴ is branched or unbranched, optionally substituted, cyclic or acyclic aliphatic. In some embodiments, R⁴ is branched or unbranched, optionally substituted, cyclic aliphatic. In certain embodiments, R⁴ is substituted cyclic aliphatic. In some embodiments, linker is unsubstituted cyclic aliphatic. In certain embodiments, R⁴ is branched or unbranched, optionally substituted carbocyclylene. In some embodiments, R⁴ is substituted carbocyclylene. In certain embodiments, R⁴ is unsubstituted carbocyclylene. In certain embodiments, R⁴ is branched or unbranched, optionally substituted C₃-C₈ carbocyclylene. In some embodiments, R⁴ is substituted C₃-C₈ carbocyclylene. In certain embodiments, R⁴ is unsubstituted C₃-C₈ carbocyclylene. In some embodiments, R⁴ is branched or unbranched, optionally substituted, acyclic aliphatic. In some embodiments, R⁴ is substituted acyclic aliphatic. In certain embodiments, R⁴ is unsubstituted acyclic aliphatic. In some embodiments, R⁴ is branched or unbranched, optionally substituted, alkylene. In some embodiments, R⁴ is substituted alkylene. In some embodiments, R⁴ is unsubstituted alkylene. In some embodiments, R⁴ is branched or unbranched, optionally substituted, C₁-C₁₀ alkylene. In some embodiments, R⁴ is substituted C₁-C₁₀ alkylene. In some embodiments, R⁴ is unsubstituted C₁-C₁₀ alkylene. In some embodiments, R⁴ is branched or unbranched, optionally substituted, alkenylene. In some embodiments, R⁴ is substituted alkenylene. In some embodiments, R⁴ is unsubstituted alkenylene. In some embodiments, R⁴ is branched or unbranched, optionally substituted, C₂-C₁₀ alkenylene. In some embodiments, R⁴ is substituted C₂-C₁₀ alkenylene. In some embodiments, R⁴ is unsubstituted C₂-C₁₀ alkenylene. In some embodiments, R⁴ is branched or unbranched, optionally substituted, alkynylene. In some embodiments, R⁴ is substituted alkynylene. In some embodiments, R⁴ is unsubstituted alkynylene. In some embodiments, R⁴ is branched or unbranched, optionally substituted, C₂-C₁₀ alkynylene. In some embodiments, R⁴ is substituted C₂-C₁₀ alkynylene. In some embodiments, R⁴ is unsubstituted C₂-C₁₀ alkynylene.

In certain embodiments, R⁴ is branched or unbranched, optionally substituted, cyclic or acyclic heteroaliphatic. In some embodiments, R⁴ is branched or unbranched, optionally substituted, cyclic heteroaliphatic. In certain embodiments, R⁴ is substituted cyclic heteroaliphatic. In some embodiments, linker is unsubstituted cyclic heteroaliphatic. In certain embodiments, R⁴ is branched or unbranched, optionally substituted heterocyclylene. In some embodiments, R⁴ is substituted heterocyclylene. In certain embodiments, R⁴ is unsubstituted heterocyclylene. In certain embodiments, R⁴ is branched or unbranched, optionally substituted 3-8-membered heterocyclylene. In some embodiments, R⁴ is substituted 3-8-membered heterocyclylene. In certain embodiments, R⁴ is unsubstituted 3-8-membered heterocyclylene. In some embodiments, R⁴ is branched or unbranched, optionally substituted, acyclic heteroaliphatic. In some embodiments, R⁴ is substituted acyclic heteroaliphatic. In certain embodiments, R⁴ is unsubstituted acyclic heteroaliphatic. In some embodiments, R⁴ is branched or unbranched, optionally substituted, heteroalkylene. In some embodiments, R⁴ is substituted heteroalkylene. In some embodiments, R⁴ is unsubstituted heteroalkylene. In some embodiments, R⁴ is branched or unbranched, optionally substituted, C₁-C₁₀ heteroalkylene. In some embodiments, R⁴ is substituted C₁-C₁₀ heteroalkylene. In some embodiments, R⁴ is unsubstituted C₁-C₁₀ heteroalkylene. In some embodiments, R⁴ is branched or unbranched, optionally substituted, heteroalkenylene. In some embodiments, R⁴ is substituted heteroalkenylene. In some embodiments, R⁴ is unsubstituted heteroalkenylene. In some embodiments, R⁴ is branched or unbranched, optionally substituted, C₂-C₁₀ heteroalkenylene. In some embodiments, R⁴ is substituted C₂-C₁₀ heteroalkenylene. In some embodiments, R⁴ is unsubstituted C₂-C₁₀ heteroalkenylene. In some embodiments, R⁴ is branched or unbranched, optionally substituted, heteroalkynylene. In some embodiments, R⁴ is substituted heteroalkynylene. In some embodiments, R⁴ is unsubstituted heteroalkynylene. In some embodiments, R⁴ is branched or unbranched, optionally substituted, C₂-C₁₀ heteroalkynylene. In some embodiments, R⁴ is substituted C₂-C₁₀ heteroalkynylene. In some embodiments, R⁴ is unsubstituted C₂-C₁₀ heteroalkynylene.

In certain embodiments, R⁴ is branched or unbranched, optionally substituted, cyclic or acyclic acyl. In some embodiments, R⁴ is substituted cyclic or acyclic acyl. In some embodiments, R⁴ is unsubstituted cyclic or acyclic acyl. In certain embodiments, R⁴ is branched or unbranched, optionally substituted, cyclic acyl. In some embodiments, R⁴ is substituted cyclic acyl. In some embodiments, R⁴ is unsubstituted cyclic acyl. In certain embodiments, R⁴ is branched or unbranched, optionally substituted, C₅-C₁₀ cyclic acyl. In some embodiments, R⁴ is substituted C₅-C₁₀ cyclic acyl. In some embodiments, R⁴ is unsubstituted C₅-C₁₀ cyclic acyl. In certain embodiments, R⁴ is branched or unbranched, optionally substituted, acyclic acyl. In some embodiments, R⁴ is substituted acyclic acyl. In some embodiments, R⁴ is unsubstituted acyclic acyl. In certain embodiments, R⁴ is branched or unbranched, optionally substituted, C₁-C₁₀ acyclic acyl. In some embodiments, R⁴ is substituted C₁-C₁₀ acyclic acyl. In some embodiments, R⁴ is unsubstituted C₁-C₁₀ acyclic acyl.

In certain embodiments, R⁴ is optionally substituted aryl. In some embodiments, R⁴ is substituted aryl. In certain embodiments, R⁴ is unsubstituted aryl. In some embodiments, R⁴ is optionally substituted C₆-C₁₄ aryl. In certain embodiments, R⁴ is substituted C₆-C₁₄ aryl. In some embodiments, R⁴ is unsubstituted C₆-C₁₄ aryl.

In certain embodiments, R⁴ is optionally substituted heteroaryl. In some embodiments, R⁴ is substituted heteroaryl. In certain embodiments, R⁴ is unsubstituted heteroaryl. In certain embodiments, R⁴ is optionally substituted 5-14-membered heteroaryl. In some embodiments, R⁴ is substituted 5-14-membered heteroaryl. In certain embodiments, R⁴ is unsubstituted 5-14-membered heteroaryl.

In certain embodiments, R⁴ is a nitrogen protecting group. In some embodiments, the nitrogen protecting group is Bn, Boc, Cbz, Fmoc, trifluoroacetyl, triphenylmethyl, acetyl, or Ts.

In some embodiments, R⁴ is joined to linker B together with the intervening atoms to form a linker comprising a N-containing heterocycle. In some embodiments, the N-containing heterocycle is a 3-8-membered N-containing heterocycle. In some embodiments, the N-containing heterocycle is a 5-7-membered N-containing heterocycle. In some embodiments, the N-containing heterocycle is a 5-membered N-containing heterocycle. In some embodiments, the N-containing heterocycle is a 6-membered N-containing heterocycle. In some embodiments, the N-containing heterocycle is a 7-membered N-containing heterocycle.

In certain embodiments, the one or more compounds of formula (v), (vi), or (vii) are selected from

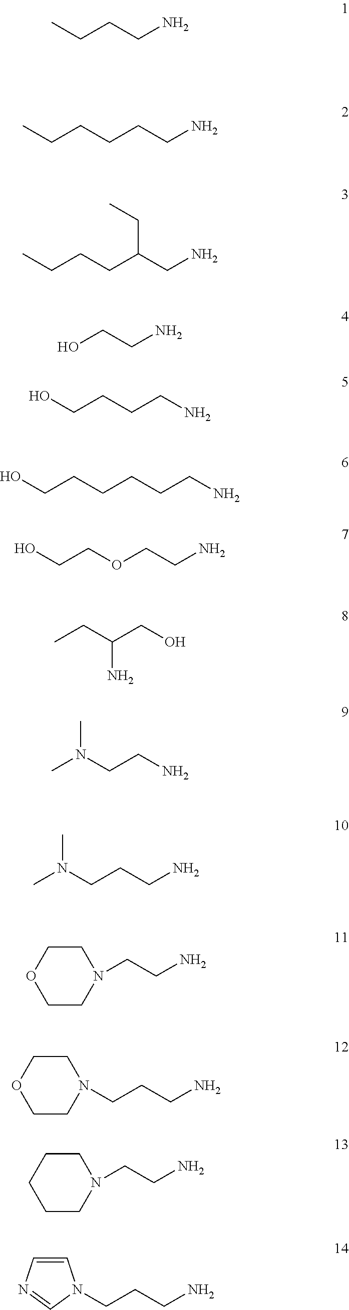

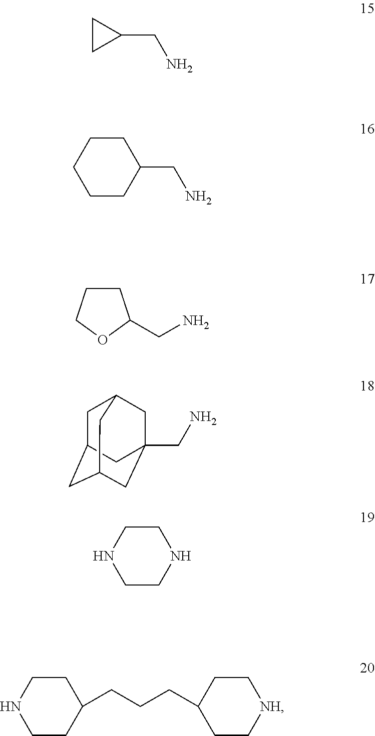

or a salt, isotope, or stereoisomer thereof.

In certain embodiments, one of the one or more compounds of formula (v) is

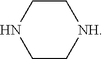

In certain embodiments, one of the one or more compounds of formula (v) is

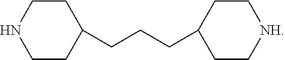 [SMILES: C(CC1CCNCC1)CC1CCNCC1]

In some embodiments the compound is prepared by reacting one or more compounds of Formula (II):

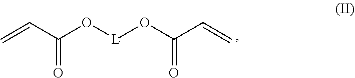

or a salt, isotope, or stereoisomer thereof, with one or more compounds selected from

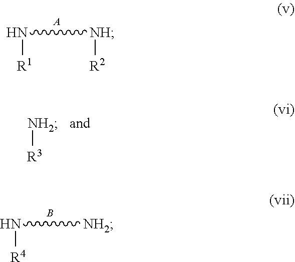

and further reacting the compound of with a compound selected from HOR^{C}, HSR^{C}, or HN(R^{C})₂, wherein each instance of R^{C} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, a nitrogen protecting group when attached to a nitrogen atom, or two instances of R^{C} attached to the same intervening atom are joined together with the intervening atom to form optionally substituted heterocyclyl or optionally substituted heteroaryl.

As defined herein, each instance of R^{C} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, a nitrogen protecting group when attached to a nitrogen atom, or two instances of R^{C} attached to the same intervening atom are joined together with the intervening atom to form optionally substituted heterocyclyl or optionally substituted heteroaryl. In some embodiments, at least one instance of R^{C} is independently hydrogen, optionally substituted alkyl, optionally substituted alkenyl, optionally substituted alkynyl, optionally substituted heteroalkyl, optionally substituted heteroalkenyl, or optionally substituted heteroalkynyl. In some embodiments, at least one instance of R^{C} is optionally substituted carbocyclyl, optionally substituted heterocyclyl, optionally substituted aryl, or optionally substituted heteroaryl. In some embodiments, at least one instance of R^{C} is independently hydrogen, optionally substituted C₁₋₁₀ alkyl, optionally substituted C₁₋₁₀ alkenyl, optionally substituted C₁₋₁₀ alkynyl, optionally substituted C₃₋₁₄ carbocyclyl, or optionally substituted C₆₋₁₄ aryl. In some embodiments, at least one instance of R^{C} is independently hydrogen, optionally substituted C₁₋₁₀ alkyl, or optionally substituted phenyl. In some embodiments, at least one instance of R^{C} is hydrogen, a nitrogen protecting group when attached to a nitrogen atom, an oxygen protecting group when attached to an oxygen atom, or a sulfur protecting group when attached to a sulfur atom. In some embodiments, at least one instance of R^{C} is a nitrogen protecting group when attached to a nitrogen atom, an oxygen protecting group when attached to an oxygen atom, or a sulfur protecting group when attached to a sulfur atom. In some embodiments, two instances of R^{C} attached to the same intervening atom are joined together with the intervening atom to form an optionally substituted, monocyclic, heterocyclic or heteroaryl ring.

### Agents

Agents that are delivered by the systems (e.g., via compounds and particles provided in pharmaceutical compositions, nutraceutical compositions, food products, beverages) described herein may be (e.g., therapeutic or prophylactic), diagnostic, cosmetic, or nutraceutical agents. Any chemical compound to be administered to a subject may be delivered using the compositions, complexes, picoparticles, nanoparticles (e.g., lipid nanoparticles), microparticles, micelles, or liposomes, described herein. In some embodiments, the agent is a small molecule (e.g., an organic molecule, organometallic compound, or inorganic molecule), nucleic acid, protein, peptide, targeting agent, an isotopically labeled chemical compound, vaccine, an immunological agent, an agent useful in bioprocessing (e.g., for intracellular manufacturing of proteins, such as a cell's bioprocessing of a commercially useful chemical or fuel), a vitamin, a mineral, a micronutrient, a biologic, probiotic, polynucleotide, bacteria, cell, or microorganism. Any chemical compound to be administered to a subject may be delivered to the subject using the compositions.

Exemplary agents that may be included in a composition described herein include, but are not limited to, small molecules, organometallic compounds, polynucleotides, proteins, peptides, carbohydrates, monosaccharides, oligosaccharides, polysaccharides, nucleoproteins, mucoproteins, lipoproteins, small molecules linked to proteins, glycoproteins, steroids, nucleotides, oligonucleotides, polynucleotides, nucleosides, antisense oligonucleotides, lipids, hormones, vitamins, minerals, micronutrients, probiotics, biologics, cells, metals, targeting agents, isotopically labeled chemical compounds, drugs (e.g., compounds approved for human or veterinary use by the U.S. Food and Drug Administration as provided in the Code of Federal Regulations), vaccines, immunological agents, agents useful in bioprocessing, bacteria, cell, microorganism and mixtures thereof. The targeting agents are described in more detail herein. In certain embodiments, the agents are nutraceutical agents. In certain embodiments, the agents are pharmaceutical agents (e.g., a therapeutic or prophylactic agent). In certain embodiments, the agent is an antibiotic agent (e.g., an anti-bacterial, anti-viral, or anti-fungal agent), anesthetic, steroidal agent, anti-proliferative agent, anti-inflammatory agent, anti-angiogenesis agent, anti-neoplastic agent, anti-cancer agent, anti-diabetic agent, antigen, vaccine, antibody, decongestant, antihypertensive, sedative, birth control agent, progestational agent, anti-cholinergic, analgesic, immunosuppressant, anti-depressant, anti-psychotic, β-adrenergic blocking agent, diuretic, cardiovascular active agent, vasoactive agent, non-steroidal, nutritional agent, anti-allergic agent, or pain-relieving agent. Vaccines may comprise isolated proteins or peptides, inactivated organisms and viruses, dead organisms and viruses, genetically altered organisms or viruses, and cell extracts. Therapeutic and prophylactic agents may be combined with interleukins, interferon, cytokines, and adjuvants such as cholera toxin, alum, and Freund's adjuvant, etc. In some embodiments, the agent is a cell. In certain embodiments, the agent is a microorganism. In some embodiments, the agent is bacteria. In certain embodiments, the agent is a combination of bacterial strains. In some embodiments, the agent is bacteria or a combination of bacterial strains. In certain embodiments, the bacteria or combination of bacterial strains modify the gut flora.

In certain embodiments, the agent is a vitamin, mineral, micronutrient, biologic, small molecule, probiotic, polynucleotide, bacteria, cell, or microorganism. In some embodiments, the agent is a vitamin, mineral, micronutrient, or probiotic. In certain embodiments, the agent is a vitamin or mineral. In some embodiments, the agent is a vitamin. In certain embodiments, the agent is a mineral. In some embodiments, the agent is a micronutrient. In certain embodiments, the agent is a biologic. In some embodiments, the agent is a small molecule. In certain embodiments, the agent is a probiotic. In some embodiments, the agent is a polynucleotide.

In certain embodiments, the agent is vitamin A, iron, iodine, vitamin B1, vitamin B2 (riboflavin), vitamin B3, vitamin B6, vitamin B7 (niacin), vitamin B9, vitamin B12 (cobalamin), vitamin D (cholecalcifeol), vitamin E (tocopherol), vitamin K1 (phytomenadione), vitamin K3, vitamin C (6-O-palmitoyl-L-ascorbic acid), or zinc. In certain embodiments, the agent is vitamin A, iron, iodine, vitamin B2 (riboflavin), vitamin B7 (niacin), vitamin B12 (cobalamin), vitamin D (cholecalcifeol), vitamin E (tocopherol), vitamin K1 (phytomenadione), vitamin C (6-O-palmitoyl-L-ascorbic acid), or zinc. In some embodiments, the agent is vitamin A, vitamin B2 (riboflavin), vitamin B7 (niacin), vitamin B12 (cobalamin), vitamin D (cholecalcifeol), vitamin E (tocopherol), vitamin K1 (phytomenadione), or vitamin C (6-O-pamlmitoyl-L-ascorbic acid). In certain embodiments, the agent is iron, iodine, or zinc. In some embodiments, the agent is iron or zinc. In some embodiments, the agent is an iron or zinc salt. In some embodiments, the agent is ferrous sulfate or zinc sulfate. In certain embodiments, the agent is vitamin A. In certain embodiments, the agent is vitamin B1. In certain embodiments, the agent is vitamin B2 (riboflavin). In certain embodiments, the agent is vitamin B3. In certain embodiments, the agent is vitamin B6. In certain embodiments, the agent is vitamin B7 (niacin). In certain embodiments, the agent is vitamin B9. In certain embodiments, the agent is vitamin B12 (cobalamin). In certain embodiments, the agent is vitamin D (cholecalcifeol). In certain embodiments, the agent is vitamin E (tocopherol). In certain embodiments, the agent is vitamin K1 (phytomenadione). In certain embodiments, the agent is vitamin K3. In certain embodiments, the agent is vitamin C (6-O-palmitoyl-L-ascorbic acid). In certain embodiments, the agent is iron. In certain embodiments, the agent is ferrous sulfate. In some embodiments, the agent is zinc. In some embodiments, the agent is zinc sulfate. In certain embodiments, the agent is iodine.

In some embodiments, the agent and the compound, or the pharmaceutically acceptable salt, stereoisomer, or isotopically labeled derivative thereof, are not covalently attached. In certain embodiments, the composition encapsulates the agent.

In certain embodiments, an agent to be delivered or used in a composition described herein is a polynucleotide. In certain embodiments, the agent is plasmid DNA (pDNA). In certain embodiments, the agent is single-stranded DNA (ssDNA), double-stranded DNA (dsDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, or viral DNA. In certain embodiments, the agent is RNA. In certain embodiments, the agent is small interfering RNA (siRNA). In certain embodiments, the agent is messenger RNA (mRNA). In certain embodiments, the agent is single-stranded RNA (ssRNA), double-stranded RNA (dsRNA), small interfering RNA (siRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, or viral satellite RNA. In certain embodiments, the agent is an RNA that carries out RNA interference (RNAi). The phenomenon of RNAi is discussed in greater detail, for example, in the following references: Elbashir et al., 2001, Genes Dev., 15:188; Fire et al., 1998, Nature, 391:806; Tabara et al., 1999, Cell, 99:123; Hammond et al., Nature, 2000, 404:293; Zamore et al., 2000, Cell, 101:25; Chakraborty, 2007, Curr. Drug Targets, 8:469; and Morris and Rossi, 2006, Gene Ther., 13:553. In certain embodiments, upon delivery of an RNA into a subject, tissue, or cell, the RNA is able to interfere with the expression of a specific gene in the subject, tissue, or cell. In certain embodiments, the agent is a pDNA, siRNA, mRNA, or a combination thereof.

In certain embodiments, the polynucleotide may be provided as an antisense agent or RNAi. See, e.g., Fire et al., Nature 391:806-811, 1998. Antisense therapy is meant to include, e.g., administration or in situ provision of single- or double-stranded polynucleotides, or derivatives thereof, which specifically hybridize, e.g., bind, under cellular conditions, with cellular mRNA and/or genomic DNA, or mutants thereof, so as to inhibit the expression of the encoded protein, e.g., by inhibiting transcription and/or translation. See, e.g., Crooke, “Molecular mechanisms of action of antisense drugs,” Biochim. Biophys. Acta 1489(1):31-44, 1999; Crooke, “Evaluating the mechanism of action of anti-proliferative antisense drugs,” Antisense Nucleic Acid Drug Dev. 10(2):123-126, discussion 127, 2000; Methods in Enzymology volumes 313-314, 1999. The binding may be by conventional base pair complementarity, or, for example, in the case of binding to DNA duplexes, through specific interactions in the major groove of the double helix (i.e., triple helix formation). See, e.g., Chan et al., J. Mol. Med. 75(4):267-282, 1997.

In some embodiments, pDNA, siRNA, dsRNA, shRNA, miRNA, mRNA, tRNA, asRNA, and/or RNAi can be designed and/or predicted using one or more of a large number of available algorithms. To give but a few examples, the following resources can be utilized to design and/or predict polynucleotides: algorithms found at Alnylum Online; Dharmacon Online; OligoEngine Online; Molecula Online; Ambion Online; BioPredsi Online; RNAi Web Online; Chang Bioscience Online; Invitrogen Online; LentiWeb Online GenScript Online; Protocol Online; Reynolds et al., 2004, Nat. Biotechnol., 22:326; Naito et al., 2006, Nucleic Acids Res., 34:W448; Li et al., 2007, RNA, 13:1765; Yiu et al., 2005, Bioinformatics, 21:144; and Jia et al., 2006, BMC Bioinformatics, 7: 271.

The polynucleotide included in a composition may be of any size or sequence, and they may be single- or double-stranded. In certain embodiments, the polynucleotide includes at least about 30, at least about 100, at least about 300, at least about 1,000, at least about 3,000, or at least about 10,000 base pairs. In certain embodiments, the polynucleotide includes less than about 10,000, less than about 3,000, less than about 1,000, less than about 300, less than about 100, or less than about 30 base pairs. Combinations of the above ranges (e.g., at least about 100 and less than about 1,000) are also within the scope of the invention. The polynucleotide may be provided by any means known in the art. In certain embodiments, the polynucleotide is engineered using recombinant techniques. See, e.g., Ausubel et al., Current Protocols in Molecular Biology (John Wiley & Sons, Inc., New York, 1999); Molecular Cloning: A Laboratory Manual, 2nd Ed., ed. by Sambrook, Fritsch, and Maniatis (Cold Spring Harbor Laboratory Press: 1989). The polynucleotide may also be obtained from natural sources and purified from contaminating components found normally in nature. The polynucleotide may also be chemically synthesized in a laboratory. In certain embodiments, the polynucleotide is synthesized using standard solid phase chemistry. The polynucleotide may be isolated and/or purified. In certain embodiments, the polynucleotide is substantially free of impurities. In certain embodiments, the polynucleotide is at least about 50%, at least about 60%, at least about 70%, at least about 80%, at least about 90%, at least about 95%, or at least about 99% free of impurities.

The polynucleotide may be modified by physical, chemical, and/or biological means. The modifications include methylation, phosphorylation, and end-capping, etc. In certain embodiments, the modifications lead to increased stability of the polynucleotide.

Wherever a polynucleotide is employed in the composition, a derivative of the polynucleotide may also be used. These derivatives include products resulted from modifications of the polynucleotide in the base moieties, sugar moieties, and/or phosphate moieties of the polynucleotide. Modified base moieties include, but are not limited to, 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, 0(6)-methylguanine, and 2-thiocytidine. Modified sugar moieties include, but are not limited to, 2′-fluororibose, ribose, 2′-deoxyribose, 3′-azido-2′,3′-dideoxyribose, 2′,3′-dideoxyribose, arabinose (the 2′-epimer of ribose), acyclic sugars, and hexoses. The nucleosides may be strung together by linkages other than the phosphodiester linkage found in naturally occurring DNA and RNA. Modified linkages include, but are not limited to, phosphorothioate and 5′-N-phosphoramidite linkages. Combinations of the various modifications may be used in a single polynucleotide. These modified polynucleotides may be provided by any means known in the art; however, as will be appreciated by those of skill in the art, the modified polynucleotides may be prepared using synthetic chemistry in vitro.

The polynucleotide described herein may be in any form, such as a circular plasmid, a linearized plasmid, a cosmid, a viral genome, a modified viral genome, and an artificial chromosome.

The polynucleotide described herein may be of any sequence. In certain embodiments, the polynucleotide encodes a protein or peptide. The encoded protein may be an enzyme, structural protein, receptor, soluble receptor, ion channel, active (e.g., pharmaceutically active) protein, cytokine, interleukin, antibody, antibody fragment, antigen, coagulation factor, albumin, growth factor, hormone, and insulin, etc. The polynucleotide may also comprise regulatory regions to control the expression of a gene. These regulatory regions may include, but are not limited to, promoters, enhancer elements, repressor elements, TATA boxes, ribosomal binding sites, and stop sites for transcription, etc. In certain embodiments, the polynucleotide is not intended to encode a protein. For example, the polynucleotide may be used to fix an error in the genome of the cell being transfected.

In certain embodiments, the polynucleotide described herein comprises a sequence encoding an antigenic peptide or protein. A composition containing the polynucleotide can be delivered to a subject to induce an immunologic response sufficient to decrease the chance of a subsequent infection and/or lessen the symptoms associated with such an infection. The polynucleotide of these vaccines may be combined with interleukins, interferon, cytokines, and/or adjuvants described herein.

The antigenic protein or peptides encoded by the polynucleotide may be derived from bacterial organisms, such as Streptococccus pneumoniae, Haemophilus influenzae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorferi, and Camphylobacter jejuni; from viruses, such as smallpox virus, influenza A virus, influenza B virus, respiratory syncytial virus, parainfluenza virus, measles virus, HIV virus, varicella-zoster virus, herpes simplex 1 virus, herpes simplex 2 virus, cytomegalovirus, Epstein-Barr virus, rotavirus, rhinovirus, adenovirus, papillomavirus, poliovirus, mumps virus, rabies virus, rubella virus, coxsackieviruses, equine encephalitis virus, Japanese encephalitis virus, yellow fever virus, Rift Valley fever virus, hepatitis A virus, hepatitis B virus, hepatitis C virus, hepatitis D virus, and hepatitis E virus; and from fungal, protozoan, or parasitic organisms, such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis, and Schistosoma mansoni.

In certain embodiments, the agent in a composition that is delivered to a subject may be a mixture of two or more agents that may be useful as, e.g., combination therapies. The compositions including the two or more agents can be administered to achieve a synergistic effect. In certain embodiments, the compositions including the two or more agents can be administered to improve the activity and/or bioavailability, reduce and/or modify the metabolism, inhibit the excretion, and/or modify the distribution within the body of a subject, of each one of the two or more agents. It will also be appreciated that the therapy employed may achieve a desired effect for the same disorder, and/or it may achieve different effects.

The compositions can be administered concurrently with, prior to, or subsequent to the one or more agents. The two or more agents may be useful for treating and/or preventing a same disease or different diseases. Each one of the agents may be administered at a dose and/or on a time schedule determined for that agent. The agents may also be administered together with each other and/or with the composition described herein in a single dose or administered separately in different doses. The particular combination to employ in a regimen will take into account compatibility of the agents and/or the desired therapeutic and/or prophylactic effect to be achieved. In general, it is expected that the agents utilized in combination be utilized at levels that do not exceed the levels at which they are utilized individually. In some embodiments, the levels utilized in combination will be lower than those utilized individually.

### Targeting Agents

Since it is often desirable to target a particular cell, collection of cells, or tissue, compounds provided herein, and the complexes, liposomes, micelles, and particles (e.g., microparticles and nanoparticles) thereof, may be modified to include targeting moieties. For example, a compound provided herein may include a targeting moiety. A variety of agents or regions that target particular cells are known in the art. See, e.g., Cotten et al., Methods Enzym. 217:618, 1993. The targeting agent may be included throughout a particle of a compound provided herein or may be only on the surface of the particle. The targeting agent may be a protein, peptide, carbohydrate, glycoprotein, lipid, small molecule, or polynucleotide, etc. The targeting agent may be used to target specific cells or tissues or may be used to promote endocytosis or phagocytosis of the particle. Examples of targeting agents include, but are not limited to, antibodies, fragments of antibodies, proteins, peptides, carbohydrates, receptor ligands, sialic acid, and aptamers, etc. If the targeting agent is included throughout a particle, the targeting agent may be included in the mixture that is used to form the particle. If the targeting agent is only on the surface of a particle, the targeting agent may be associated with (e.g., by covalent or non-covalent (e.g., electrostatic, hydrophobic, hydrogen bonding, van der Waals, π-π stacking) interactions) the formed particle using standard chemical techniques.

### Particles, Microparticles, and Nanoparticles

The poly(O-amino esters) provided herein may also be used to form delivery devices. The PAEs provided herein may be used to encapsulate agents. The PAEs provided herein have several properties that make them particularly suitable in the preparation of delivery devices. These include 1) the ability of the polymer to complex and “protect” labile agents; and 2) the ability to stabilize the agent. The encapsulating PAE may be combined with other polymers (e.g., PEG, PLGA) or excipients to form the microparticles or nanoparticles. In certain embodiments, the additional materials are approved by a regulatory agency, such as the U.S. FDA, for human and veterinary use.

In some embodiments, a composition including a compound provided herein and an agent is in the form of a particle. In certain embodiments, the compound provided herein and agent form a complex, and the complex is in the form of a particle. In certain embodiments, the compound provided herein encapsulates the agent and is in the form of a particle. In certain embodiments, the compound provided herein is mixed with the agent, and the mixture is in the form of a particle. In some embodiments, the particle encapsulates the agent.

In certain embodiments, a complex of a compound provided herein and an agent in a composition of is in the form of a particle. In some embodiments, the particle is a nanoparticle or a microparticle. In certain embodiments, the particle is a nanoparticle. In some embodiments, the particle is a microparticle. In some embodiments, the polymers are used to form microparticles containing the agent to be delivered. In certain embodiments, the particle is a microparticle (i.e., particle having a characteristic dimension of less than about 1 millimeter and at least about 1 micrometer, where the characteristic dimension of the particle is the smallest cross-sectional dimension of the particle). In certain embodiments, the diameter of the microparticles ranges from between 500 nm to 50 micrometers, more preferably from 1 micrometer to 20 micrometers, and most preferably from 1 micrometer to 10 micrometers. In some embodiments, the microparticles range from 1-5 micrometers.

The particles may be prepared using any method known in the art, such as precipitation, milling, spray drying, single and double emulsion solvent evaporation, solvent extraction, phase separation, and simple and complex coacervation. In certain embodiments, methods of preparing the particles are the double emulsion process and spray drying. The conditions used in preparing the particles may be altered to yield particles of a desired size or property (e.g., hydrophobicity, hydrophilicity, external morphology, “stickiness”, shape, polydispersity, etc.). The method of preparing the particle and the conditions (e.g., solvent, temperature, concentration, and air flow rate, etc.) used may also depend on the agent being complexed, encapsulated, or mixed, and/or the composition of the matrix.

Methods developed for making particles for delivery of agents that are included in the particles are described in the literature. See, e.g., Doubrow, M., Ed., “Microcapsules and Nanoparticles in Medicine and Pharmacy,” CRC Press, Boca Raton, 1992; Mathiowitz and Langer, J. Controlled Release 5:13-22, 1987; Mathiowitz et al., Reactive Polymers 6:275-283, 1987; Mathiowitz et al., J. Appl. Polymer Sci. 35:755-774, 1988.

If the particles prepared by any of the above methods have a size range outside of the desired range, the particles can be sized, for example, using a sieve. The particles may also be coated. In certain embodiments, the particles are coated with a targeting agent. In certain embodiments, the particles are coated with a surface-altering agent. In some embodiments, the particles are coated to achieve desirable surface properties (e.g., a particular charge).

In certain embodiments, the polydispersity index (PDI, determined by dynamic light scattering) of the particles described herein (e.g., particles included in a composition described herein) is between 0.01 and 0.9, between 0.1 and 0.9, between 0.1 and 0.7, between 0.1 and 0.5, between 0.01 and 0.4, between 0.03 and 0.4, between 0.1 and 0.4, between 0.01 and 0.3, between 0.03 and 0.3, or between 0.1 and 0.3.

In some embodiments, the particle further comprises an excipient. In some embodiments, the particle further comprises one or more of a polysaccharide or derivative thereof, collagen or derivative thereof, hydrolyzed collagen or derivative thereof, or water-soluble synthetic polymer or derivative thereof. In some embodiments, the particle further comprises one or more of hyaluronic acid, starch, dextran, gelatin, polyvinyl alcohol (PVA), alginate, or chitin. In some embodiments, the particle further comprises hyaluronic acid. In some embodiments, the particle further comprises a starch. In some embodiments, the particle further comprises dextran. In some embodiments, the particle further comprises gelatin. In some embodiments, the particle further comprises polyvinyl alcohol (PVA). In some embodiments, the particle further comprises alginate. In some embodiments, the particle further comprises chitin.

In some embodiments, the particle comprises approximately 0.1-90% of an excipient by mass relative to the total mass of the particle. In some embodiments, the particle comprises approximately 0.1-50% of an excipient by mass relative to the total mass of the particle. In some embodiments, the particle comprises approximately 0.1-20% of an excipient by mass relative to the total mass of the particle. In some embodiments, the particle comprises approximately 0.5-10% of an excipient by mass relative to the total mass of the particle. In some embodiments, the particle comprises approximately 1-5% of an excipient by mass relative to the total mass of the particle. In some embodiments, the particle comprises approximately 0.5%, approximately 1%, approximately 1.5%, approximately 2%, approximately 2.5%, approximately 3%, approximately 3.5%, approximately 4%, approximately 5%, approximately 6%, approximately 7%, approximately 8%, approximately 9%, or approximately 10% of an excipient by mass relative to the total mass of the particle.

In some embodiments, the particle comprises approximately 0.1-90% of dextran by mass relative to the total mass of the particle. In some embodiments, the particle comprises approximately 0.1-50% of dextran by mass relative to the total mass of the particle. In some embodiments, the particle comprises approximately 0.1-20% of dextran by mass relative to the total mass of the particle. In some embodiments, the particle comprises approximately 0.5-10% of dextran by mass relative to the total mass of the particle. In some embodiments, the particle comprises approximately 1-5% of dextran by mass relative to the total mass of the particle. In some embodiments, the particle comprises approximately 0.5%, approximately 1%, approximately 1.5%, approximately 2%, approximately 2.5%, approximately 3%, approximately 3.5%, approximately 4%, approximately 5%, approximately 6%, approximately 7%, approximately 8%, approximately 9%, or approximately 10% of dextran by mass relative to the total mass of the particle.

In some embodiments, the particle comprises approximately 0.1-90% of PVA by mass relative to the total mass of the particle. In some embodiments, the particle comprises approximately 0.1-50% of PVA by mass relative to the total mass of the particle. In some embodiments, the particle comprises approximately 0.1-20% of PVA by mass relative to the total mass of the particle. In some embodiments, the particle comprises approximately 0.5-10% of PVA by mass relative to the total mass of the particle. In some embodiments, the particle comprises approximately 1-5% of PVA by mass relative to the total mass of the particle. In some embodiments, the particle comprises approximately 0.5%, approximately 1%, approximately 1.5%, approximately 2%, approximately 2.5%, approximately 3%, approximately 3.5%, approximately 4%, approximately 5%, approximately 6%, approximately 7%, approximately 8%, approximately 9%, or approximately 10% of PVA by mass relative to the total mass of the particle.

In some embodiments, the particle comprises approximately 0.1-90% of an excipient by mass relative to the total mass of the PAE. In some embodiments, the particle comprises approximately 0.1-50% of an excipient by mass relative to the total mass of the PAE. In some embodiments, the particle comprises approximately 0.1-20% of an excipient by mass relative to the total mass of the PAE. In some embodiments, the particle comprises approximately 0.5-10% of an excipient by mass relative to the total mass of the PAE. In some embodiments, the particle comprises approximately 1-5% of an excipient by mass relative to the total mass of the PAE. In some embodiments, the particle comprises approximately 0.5%, approximately 1%, approximately 1.5%, approximately 2%, approximately 2.5%, approximately 3%, approximately 3.5%, approximately 4%, approximately 5%, approximately 6%, approximately 7%, approximately 8%, approximately 9%, or approximately 10% of an excipient by mass relative to the total mass of the PAE.

In some embodiments, the particle comprises approximately 0.1-90% of dextran by mass relative to the total mass of the PAE. In some embodiments, the particle comprises approximately 0.1-50% of dextran by mass relative to the total mass of the PAE. In some embodiments, the particle comprises approximately 0.1-20% of dextran by mass relative to the total mass of the PAE. In some embodiments, the particle comprises approximately 0.5-10% of dextran by mass relative to the total mass of the PAE. In some embodiments, the particle comprises approximately 1-5% of dextran by mass relative to the total mass of the PAE. In some embodiments, the particle comprises approximately 0.5%, approximately 1%, approximately 1.5%, approximately 2%, approximately 2.5%, approximately 3%, approximately 3.5%, approximately 4%, approximately 5%, approximately 6%, approximately 7%, approximately 8%, approximately 9%, or approximately 10% of dextran by mass relative to the total mass of the PAE.

In some embodiments, the particle comprises approximately 0.1-90% of PVA by mass relative to the total mass of the PAE. In some embodiments, the particle comprises approximately 0.1-50% of PVA by mass relative to the total mass of the PAE. In some embodiments, the particle comprises approximately 0.1-20% of PVA by mass relative to the total mass of the PAE. In some embodiments, the particle comprises approximately 0.5-10% of PVA by mass relative to the total mass of the PAE. In some embodiments, the particle comprises approximately 1-5% of PVA by mass relative to the total mass of the PAE. In some embodiments, the particle comprises approximately 0.5%, approximately 1%, approximately 1.5%, approximately 2%, approximately 2.5%, approximately 3%, approximately 3.5%, approximately 4%, approximately 5%, approximately 6%, approximately 7%, approximately 8%, approximately 9%, or approximately 10% of PVA by mass relative to the total mass of the PAE.

In some embodiments, the particle further comprises a stabilizer. In some embodiments, the particle comprises about 0.01-2.0% of a stabilizer relative to the mass of polymer. In some embodiments, the particle comprises about 0.1-1.0% of a stabilizer relative to the mass of polymer. In some embodiments, the particle comprises about 0.3-0.7% of a stabilizer relative to the mass of polymer. In some embodiments, the particle comprises about 0.7% of a stabilizer relative to the mass of polymer. In some embodiments, the particle comprises about 0.6% of a stabilizer relative to the mass of polymer. In some embodiments, the particle comprises about 0.5% of a stabilizer relative to the mass of polymer. In some embodiments, the particle comprises about 0.4% of a stabilizer relative to the mass of polymer. In some embodiments, the particle comprises about 0.3% of a stabilizer relative to the mass of polymer. In some embodiments, the particle further comprises a stabilizer. In some embodiments, the particle comprises about 0.01-2.0% of a stabilizer relative to the mass of PAE. In some embodiments, the particle comprises about 0.1-1.0% of a stabilizer relative to the mass of PAE. In some embodiments, the particle comprises about 0.3-0.7% of a stabilizer relative to the mass of PAE. In some embodiments, the particle comprises about 0.7% of a stabilizer relative to the mass of PAE. In some embodiments, the particle comprises about 0.6% of a stabilizer relative to the mass of PAE. In some embodiments, the particle comprises about 0.5% of a stabilizer relative to the mass of PAE. In some embodiments, the particle comprises about 0.4% of a stabilizer relative to the mass of PAE. In some embodiments, the particle comprises about 0.3% of a stabilizer relative to the mass of PAE.

In some embodiments, the particle further comprises BHT. In some embodiments, the particle comprises about 0.01-2.0% BHT relative to the mass of polymer. In some embodiments, the particle comprises about 0.1-1.0% BHT relative to the mass of polymer. In some embodiments, the particle comprises about 0.3-0.7% BHT relative to the mass of polymer. In some embodiments, the particle comprises about 0.7% BHT relative to the mass of polymer. In some embodiments, the particle comprises about 0.6% BHT relative to the mass of polymer. In some embodiments, the particle comprises about 0.5% BHT relative to the mass of polymer. In some embodiments, the particle comprises about 0.4% BHT relative to the mass of polymer. In some embodiments, the particle comprises about 0.3% BHT relative to the mass of polymer. In some embodiments, the particle comprises about 0.01-2.0% BHT relative to the mass of PAE. In some embodiments, the particle comprises about 0.1-1.0% BHT relative to the mass of PAE. In some embodiments, the particle comprises about 0.3-0.7% BHT relative to the mass of PAE. In some embodiments, the particle comprises about 0.7% BHT relative to the mass of PAE. In some embodiments, the particle comprises about 0.6% BHT relative to the mass of PAE. In some embodiments, the particle comprises about 0.5% BHT relative to the mass of PAE. In some embodiments, the particle comprises about 0.4% BHT relative to the mass of PAE. In some embodiments, the particle comprises about 0.3% BHT relative to the mass of PAE.

### Compositions and Administration

The present disclosure provides compositions (e.g., pharmaceutical compositions, nutraceutical compositions, food products, beverages) comprising a compound provided herein (e.g., a compound of Formula (I)), or a pharmaceutically acceptable salt, stereoisomer, or isotopically labeled derivative thereof, and an agent. In some embodiments, a composition comprises a particle provided herein. In some embodiments, a composition comprises a microparticle provided herein. In some embodiments, a composition further comprises an excipient. In some embodiments, a composition further comprises a pharmaceutically acceptable excipient.

In certain embodiments, the composition is thermally stable, hydrolytically stable, light stable, and/or oxidatively stable. In some embodiments, the composition is thermally stable. In certain embodiments, the composition is hydrolytically stable. In some embodiments, the composition is light stable. In certain embodiments, the composition is oxidatively stable. In some embodiments, the composition improves the thermal stability, hydrolytic stability, light stability, and/or oxidative stability of the agent. In certain embodiments, the composition improves the thermal stability of the agent. In some embodiments, the composition improves the hydrolytic stability of the agent. In certain embodiments, the composition improves the light stability of the agent. In some embodiments, the composition improves the oxidative stability of the agent.

In certain embodiments, the composition degrades under acidic conditions. In some embodiments, the acidic conditions have a pH less than 7.0, less than 6.5, less than 6.0, less than 5.5, less than 5.0, less than 4.5, less than 4.0, less than 3.5, less than 3.0, less than 2.5, less than 2.0, less than 1.5, less than 1.4, less than 1.3, or less than 1.2. In certain embodiments, the acidic conditions have a pH less than 7.0. In some embodiments, the acidic conditions have a pH less than 6.5. In certain embodiments, the acidic conditions have a pH less than 6.0. In some embodiments, the acidic conditions have a pH less than 5.5. In certain embodiments, the acidic conditions have a pH less than 5.0. In some embodiments, the acidic conditions have a pH less than 5.0. In certain embodiments, the acidic conditions have a pH less than 4.5. In some embodiments, the acidic conditions have a pH less than 4.0 In certain embodiments, the acidic conditions have a pH less than 3.5. In some embodiments, the acidic conditions have a pH less than 3.0. In certain embodiments, the acidic conditions have a pH less than 2.5. In some embodiments, the acidic conditions have a pH less than 2.0. In certain embodiments, the acidic conditions have a pH less than 1.5. In some embodiments, the acidic conditions have a pH less than 1.4. In certain embodiments, the acidic conditions have a pH less than 1.3. In some embodiments, the acidic conditions have a pH less than 1.2. In certain embodiments, the acidic conditions have a pH less than 1.1. In some embodiments, the acidic conditions have a pH less than 1.0.

In certain embodiments, degradation of the composition produces one or more natural byproducts. In some embodiments, the natural product is a monosaccharide. In certain embodiments, the natural byproduct is isosorbide. In some embodiments, the natural product is a β-amino acid.

In some embodiments, the composition is a pharmaceutical composition. Pharmaceutical compositions described herein can be prepared by any method known in the art of pharmaceutics. In general, such preparatory methods include bringing a compound, agent, or particle described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents such as calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (II) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound, agent, or particle described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound, agent, or particle in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds, agents, or particles provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound, agent, or particle required to achieve an effective amount will vary from subject to subject, depending, for example, on species, age, and general condition of a subject, severity of the side effects or disorder, identity of the particular compound, agent or particle, mode of administration, and the like. An effective amount may be included in a single dose (e.g., single oral dose) or multiple doses (e.g., multiple oral doses). In certain embodiments, when multiple doses are administered to a subject, any two doses of the multiple doses include different or substantially the same amounts of a compound, agent, or particle described herein. In certain embodiments, when multiple doses are administered to a subject, the frequency of administering the multiple doses to the subject is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject is one dose per day. In certain embodiments, the frequency of administering the multiple doses to the subject is two doses per day. In certain embodiments, the frequency of administering the multiple doses to the subject is three doses per day. In certain embodiments, when multiple doses are administered to a subject, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject. In certain embodiments, the duration between the first dose and last dose of the multiple doses is three months, six months, or one year. In certain embodiments, the duration between the first dose and last dose of the multiple doses is the lifetime of the subject. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound, agent, or particle described herein. In certain embodiments, a dose described herein includes independently between 1 mg and 3 mg, inclusive, of a compound, agent, or particle described herein. In certain embodiments, a dose described herein includes independently between 3 mg and 10 mg, inclusive, of a compound, agent, or particle described herein. In certain embodiments, a dose described herein includes independently between 10 mg and 30 mg, inclusive, of a compound, agent, or particle described herein. In certain embodiments, a dose described herein includes independently between 30 mg and 100 mg, inclusive, of a compound, agent, or particle described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that treat a disease in a subject in need thereof, prevent a disease in a subject in need thereof, or reduce the risk to develop a disease in a subject in need thereof, improve bioavailability, improve safety, reduce drug resistance, reduce and/or modify metabolism, inhibit excretion, and/or modify distribution in a subject. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., lung disease or liver disease). Each additional pharmaceutical agent may be administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

Additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins.

In some embodiments, the composition is a nutraceutical composition. In some embodiments, the nutraceutical composition comprises an agent derived from a food source. In some embodiments, the nutraceutical composition comprises an agent derived from a food source that provides health benefits. In some embodiments, the nutraceutical composition comprises one or more agents selected from a herbal product, vitamin, mineral, fiber, phytonutrient, prebiotic, probiotic, protein, or hydrolyzed protein.

In some embodiments, the composition is a food product. In certain embodiments, the food product is any item that is to be processed, partially processed, or unprocessed for consumption by an animal. In some embodiments, the food product is for consumption by a mammal. In some embodiments, the food product is for consumption by a human. In some embodiments, the food product is a food additive, dietary supplement or food supplement, medical food, infant formula, or geriatric formula. In another aspect, the food product is a food additive. In another aspect, the food product is a dietary supplement. In certain embodiments, the food product is a food supplement. In some embodiments, the food product is a medical food. In some embodiments, the medical food is used for dietary management of a disease with specific nutritional requirements. In some embodiments, the medical food is a formulation for metabolic disorders, a formulation for AIDS, or a formulation for kidney disease. In some embodiments, the medical food is an oral rehydration product or an anti-diarrheal. In some embodiments, the medical food is a liquid. In some embodiments, the medical food is a powdered mix. In certain embodiments, the food product is infant formula. In certain embodiments, the food product is geriatric formula. In some embodiments, the food product is a fortified food. In some embodiments, the fortified food is a condiment, salt, spice, sauce, refined grain or grain product, fat or oil, dehydrated food, freeze-dried food, condensed food, instant food, or snack food. In some embodiments, the fortified food is flour, breakfast cereal, bread, corn meal, pasta, rice, butter, margarine or butter substitute, bouillon cube, powdered soup, salt, snack bar, yogurt, pudding, or baby food.

In some embodiments, the composition is a beverage. In some embodiments, the beverage is a juice, milk, plant-based milk, carbonated beverage, energy beverage, tea, coffee, vitamin water, protein shake, or concentrate.

In some embodiments, the composition is a nutritional supplement. In some embodiments, the nutritional supplement is a protein supplement, a steroid supplement, a caffeine supplement, an amino acid supplement, an herbal supplement, a probiotic, a vitamin supplement, or a mineral supplement. In some embodiments, the nutritional supplement is to manage weight, increase energy, improve sleep, or improve nutrition. In some embodiments, the nutritional supplement is an oral dosage form provided herein.

In some embodiments, the composition comprises one or more of flavoring agent(s), sweetener(s), vitamins, minerals, co-factors, proteins, lipids, peptides, and amino acids.

In certain embodiments, the composition further comprises a sweetener. Sweeteners can be used to improve palatability and are usually classified as natural or artificial. In some embodiments, a sweetener is a natural sweetener or artificial sweetener. Exemplary natural sweeteners include, but are not limited to, dextrose, fructose, glucose, liquid glucose, maltose, rebiana, glycyrrhizin, thaumatin, sorbitol, mannitol, isomalt, glycerol, maltitol, xylitol, and erythritol. Exemplary artificial sweeteners include, but are not limited to, saccharin, cyclamate, aspartame, acesulfame-K, sucralose, alitame, and neotame. In certain embodiments, sucralose is used as a sweetener. In certain embodiments, one or combination of neohespiridin dihydrochalcone, glycerol, and/or sucralose are used as sweeteners. In some embodiments, the concentration of the sweetener in the composition is between 0.01% and 5%, inclusive, by weight. In some embodiments, the concentration of the sweetener in the composition is between 0.01% and 1%, inclusive, by weight. In some embodiments, the concentration of the sweetener in the composition is between 0.5% and 1%, inclusive, by weight. In certain embodiments, the composition further comprises sucralose. In certain embodiments, the composition further comprises sucralose as about 0.01-0.25% based on the dry weight of all the components of the composition.

In certain embodiments, a composition further comprises a colorant. A colorant can be added to enhance the aesthetic appeal of the composition, especially when formulation ingredients or drugs are presented in a non-solution form. Generally, any colorant could be added, such as for example FD&C pigments (for example, blue no 1, blue no 2, red no 3, red no 40, yellow no 5, or yellow no 6). Exemplary colorants include, but are not limited to annatto extract, dehydrated beets (beet powder), canthaxanthin, caramel, β-apo-8′-carotenal, β-carotene, cochineal extract, carmine, sodium copper chlorophyllin, toasted partially defatted cooked cottonseed flour, ferrous gluconate, ferrous lactate, grape color extract, grape skin extract (enocianina), synthetic iron oxide, fruit juice, vegetable juice, carrot oil, paprika, paprika oleoresin, mica-based pearlescent pigments, riboflavin, saffron, spirulina extract, titanium dioxide, tomato lycopene extract; tomato lycopene concentrate, turmeric, turmeric oleoresin, alumina (dried aluminum hydroxide), calcium carbonate, potassium sodium copper chlorophyllin (chlorophyllin-copper complex), dihydroxyacetone, bismuth oxychloride, synthetic iron oxide, ferric ammonium ferrocyanide, ferric ferrocyanide, chromium hydroxide green, chromium oxide greens, guanine, pyrophyllite, mica, talc, aluminum powder, bronze powder, copper powder, zinc oxide, bismuth citrate, disodium EDTA-copper, guaiazulene, henna, lead acetate, pyrophyllite, silver, ultramarines, manganese violet, luminescent zinc sulfide, FD&C Blue No. 1, FD&C Blue No. 2, FD&C Green No. 3, Orange B, Citrus Red No. 2, FD&C Red No. 3, FD&C Red No. 40, FD&C Yellow No. 5, FD&C Yellow No. 6, D&C Blue No. 4, D&C Green No. 5, D&C Green No. 6, D&C Green No. 8, D&C Orange No. 4, D&C Orange No. 5, D&C Orange No. 10, D&C Orange No. 11, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Red No. 39, D&C Violet No. 2, D&C Yellow No. 7, Ext. D&C Yellow No. 7, D&C Yellow No. 8, D&C Yellow No. 10, D&C Yellow No. 11, D&C Black No. 2, D&C Black No. 3, D&C Brown No. 1, and Ext. D&C Violet No. 2. In certain embodiments, a colorant represents 0.001% to about 0.5% based on the weight of all the components of the composition. In some embodiments, the concentration of the colorant in the composition is between 0.001% and 5%, inclusive, by weight. In some embodiments, the concentration of the colorant in the composition is between 0.001% and 1%, inclusive, by weight.

In certain embodiments, a provided composition further comprises a flavoring agent. In certain embodiments, the selection of a suitable flavoring agent to be added depends on the original taste sensation of the composition, including metallic, acidic, alkaline, salty, sweet, bitter and sour taste sensation. Certain flavoring agents, alone or in combination, mask specific taste sensations. For example, metallic taste could be masked with, but not limited to, flavoring agents based on berry fruits, grape, and/or peppermint. For example, acidic taste could be masked with, but not limited to, flavoring agents based on lemon, lime, grapefruit, orange, cherry, and/or strawberry. For example, alkaline taste could be masked with, but not limited to, flavoring agents based on aniseed, caramel, passion fruit, peach and/or banana. For example, salty taste could be masked with, but not limited to, flavoring agents based on butterscotch, caramel, hazelnut, spicy, maple, apricot, apple, peach, vanilla, and/or wintergreen mint. For example, bitter taste could be masked with, but not limited to, flavoring agents based on licorice, passion fruit, coffee, chocolate, peppermint, grapefruit, cherry, peach, raspberry, wild cherry, walnut, mint, and/or anise. For example, sweet taste could be masked with, but not limited to, flavoring agents based on grape, cream, caramel, banana, vanilla and/or fruit berry. For example, sour taste could be masked with, but not limited to, flavoring agents based on citrus flavors, licorice, root, bear and/or raspberry. Flavoring agents can be used alone or in combination and its selection will be dependent also upon the target population and any other substance (e.g., a pharmaceutical or nutraceutical agent) incorporated in the composition. The perception of the flavoring agent changes from individual to individual and also with age: typically a geriatric population will prefer mint or orange flavors whereas younger populations tend to prefer flavors like fruit punch, raspberry, etc. Generally, the amount of flavoring agent needed to mask an unpleasant taste or improve taste overall will depend not only on the composition of the formulation but also on the flavor type and its strength.

In certain embodiments, a flavoring agent is a palatable flavor that has a long shelf life and which does not crystallize or precipitate out of the composition upon storage. In certain embodiments, flavoring agents are natural flavors, derived from various parts of the plants like leaves, fruits and flowers, or synthetic flavor oils or powders. Exemplary flavor oils for use in or as flavoring agents include, but are not limited to, peppermint oil, cinnamon oil, spearmint oil, and oil of nutmeg. Exemplary fruity flavors that for use in or as flavoring agents include, but are not limited to, vanilla, cocoa, coffee, chocolate and citrus. Exemplary fruit essence flavors for use in or as flavoring agents include, but are not limited to, apple, raspberry, cherry, and pineapple. The amount of flavoring agent added can vary with the flavor employed. In some embodiments, the concentration of the flavoring agent in the composition is between about 0% and 5%, by weight. In some embodiments, the concentration of the flavoring agent in the composition is between 0.001% and 5%, inclusive, by weight. In some embodiments, the concentration of the flavoring agent in the composition is between 0.1% and 1%, inclusive, by weight. In some embodiments, the concentration of the flavoring agent in the composition is between 0.5% and 1%, inclusive, by weight.

In certain embodiments, a provided composition further comprises a taste-masking agent. Taste-masking agents can be added to ameliorate the general organoleptic characteristics of the compositions. In certain embodiments, taste-masking agents are used to mask unpleasant taste of some components. The main taste sensations include metallic, acidic, alkaline, salty, sweet, bitter and sour. Exemplary of taste-masking agents include, but are not limited to, menthol, peppermint oil, L-menthol, cyclodextrins, glycerol, maltodextrins, ion-exchange resins, amino acids, gelatin, gelatinized starch, liposomes, lecithin, or lecithin-like substances and salts. The amount of taste-masking added can vary with the taste-masking employed. In certain embodiments, the taste-masking agent comprises about 0% to about 50% based on the dry weight of all the components of the composition. In certain embodiments, the taste-masking agent represents 0% to about 5% based on the dry weight of all the components of the composition.

In another aspect, a provided composition further comprises a cooling agent. Cooling agents may also be added in order to improve the after-taste of the composition. Exemplary cooling agents include, but are not limited to, neohesperidine dihydrochalcone, menthol flavor, L-Menthol and some polyol sugars which are widely used for this purpose. Other components can also be added that should compete with sensory stimuli, such as Cremophor (which is used to coat the surface protein receptors), or saline solutions (e.g., sodium chloride, which competes within channel receptors with the bitter stimuli to reduce the overall perception of bitterness). In certain embodiments, the cooling agents in the composition is one or a combination of neohesperidine dihydrochalcone, menthol, and/or polyol sugar. In certain embodiments, the mucoadhesive composition further comprises cooling agents of about 0% to about 5% based on the weight of all the components of the composition. In certain embodiments, the mucoadhesive composition further comprises cooling agents as about 0.001% to about 2.5% based on the weight of all the components of the composition.

In certain embodiments, a provided composition further comprises one or more preservatives. The preservative employed in the present disclosure can be any preservative, as long as does not negate other desirable properties of the composition. Example of a preservative is an antimicrobial preservative that is used to prevent or inhibit the growth of micro-organisms in the composition. Exemplary preservative agents include, but are not limited to, C₃-C₈ alcohols, phenylethyl alcohol, chlorobutanol, p-hydroxybenzoic, acid esters, benzathonium chloride and benzalkonium chloride, benzoic acid, propyl galate, methylparaben, propylparaben, sorbic acid, sodium benzoate and/or potassium sorbate. The amount of preservative agent added can vary with the preservative agent employed. In certain embodiments, a preservative agent represents about 0% to about 45% based on the weight of all the components of the composition. In certain embodiments, a preservative agent represents about 0% to about 1% (e.g., 0.025% to 0.2%) based on the weight of all the components of the composition.

In certain embodiments, a composition (e.g., pharmaceutical composition, nutraceutical composition, food product, beverage, or nutritional supplement) as provided herein is safe for human use (e.g., administration or consumption).

In some embodiments, any of the pharmaceutical compositions, nutraceutical compositions, food products, beverages, or nutritional supplements described herein comprises about 0.001 mg to about 10,000 mg of a compound, agent, or particle described herein. In some embodiments, any of the pharmaceutical compositions, nutraceutical compositions, food products, beverages, or nutritional supplements described herein comprises about 0.01 mg to about 10,000 mg of a compound, agent, or particle described herein. In some embodiments, any of the pharmaceutical compositions, nutraceutical compositions, food products, beverages, or nutritional supplements described herein comprises about 0.01 mg to about 5,000 mg of a compound, agent, or particle described herein. In some embodiments, any of the pharmaceutical compositions, nutraceutical compositions, food products, beverages, or nutritional supplements described herein comprises about 0.01 mg to about 2,000 mg of a compound, agent, or particle described herein. In some embodiments, any of the pharmaceutical compositions, nutraceutical compositions, food products, beverages, or nutritional supplements described herein comprises about 0.01 mg to about 1,000 mg of a compound, agent, or particle described herein. In some embodiments, any of the pharmaceutical compositions, nutraceutical compositions, food products, beverages, or nutritional supplements described herein comprises about 0.01 mg to about 500 mg of a compound, agent, or particle described herein. In some embodiments, any of the pharmaceutical compositions, nutraceutical compositions, food products, beverages, or nutritional supplements described herein comprises about 0.01 mg to about 200 mg of a compound, agent, or particle described herein. In some embodiments, any of the pharmaceutical compositions, nutraceutical compositions, food products, beverages, or nutritional supplements described herein comprises about 0.01 mg to about 100 mg of a compound, agent, or particle described herein. In some embodiments, any of the pharmaceutical compositions, nutraceutical compositions, food products, beverages, or nutritional supplements described herein comprises about 0.1 mg to about 10,000 mg of a compound, agent, or particle described herein. In some embodiments, any of the pharmaceutical compositions, nutraceutical compositions, food products, beverages, or nutritional supplements described herein comprises about 0.1 mg to about 5,000 mg of a compound, agent, or particle described herein. In some embodiments, any of the pharmaceutical compositions, nutraceutical compositions, food products, beverages, or nutritional supplements described herein comprises about 0.1 mg to about 2,000 mg of a compound, agent, or particle described herein. In some embodiments, any of the pharmaceutical compositions, nutraceutical compositions, food products, beverages, or nutritional supplements described herein comprises about 0.1 mg to about 1,000 mg of a compound, agent, or particle described herein. In some embodiments, any of the pharmaceutical compositions, nutraceutical compositions, food products, beverages, or nutritional supplements described herein comprises about 0.1 mg to about 500 mg of a compound, agent, or particle described herein. In some embodiments, any of the pharmaceutical compositions, nutraceutical compositions, food products, beverages, or nutritional supplements described herein comprises about 0.1 mg to about 200 mg of a compound, agent, or particle described herein. In some embodiments, any of the pharmaceutical compositions, nutraceutical compositions, food products, beverages, or nutritional supplements described herein comprises about 0.1 mg to about 100 mg of a compound, agent, or particle described herein.

In some embodiments, any of the pharmaceutical compositions, nutraceutical compositions, food products, beverages, or nutritional supplements described herein comprises about 0.001 mg to about 0.1 mg. In some embodiments, any of the pharmaceutical compositions, nutraceutical compositions, food products, beverages, or nutritional supplements described herein comprises about 0.01 mg to about 1 mg. In some embodiments, any of the pharmaceutical compositions, nutraceutical compositions, food products, beverages, or nutritional supplements described herein comprises about 0.1 mg to about 10 mg. In some embodiments, any of the pharmaceutical compositions, nutraceutical compositions, food products, beverages, or nutritional supplements described herein comprises about 1 mg to about 100 mg. In some embodiments, any of the pharmaceutical compositions, nutraceutical compositions, food products, beverages, or nutritional supplements described herein comprises about 10 mg to about 1,000 mg. In some embodiments, any of the pharmaceutical compositions, nutraceutical compositions, food products, beverages, or nutritional supplements described herein comprises about 100 mg to about 10,000 mg. In some embodiments, any of the pharmaceutical compositions, nutraceutical compositions, food products, beverages, or nutritional supplements described herein comprises about 0.1-0.15 mg, 0.15-0.2 mg, 0.2-0.25 mg, 0.25-0.3 mg, 0.3-0.35 mg, 0.35-0.4 mg, 0.4-0.45 mg, 0.45-0.5 mg, 0.5-0.6 mg, 0.6-0.7 mg, 0.7-0.8 mg, 0.8-0.9 mg, 0.9-1 mg, 1-1.5 mg, 1.5-2.0 mg, 2-2.5 mg, 2.5-3.0 mg, 3-3.5 mg, 3.5-4.0 mg, 4-4.5 mg, 4.5-5 mg, 5-6 mg, 6-7 mg, 7-8 mg, 8-9 mg, 9-10 mg, 10-12.5 mg, 12.5-15 mg, 15-17.5 mg, 17.5-20 mg, 20-22.5 mg, 22.5-25 mg, 25-30 mg, 30-35 mg, 35-40 mg, 40-45 mg, 45-50 mg, 50-60 mg, 60-70 mg, 70-80 mg, 80-90 mg, 90-100 mg, 100-125 mg, 125-150 mg, 150-175 mg, 175-200 mg, 200-225 mg, 225-250 mg, 250-300 mg, 300-350 mg, 350-400 mg, 400-450 mg, 450-500 mg, 500-600 mg, 600-700 mg, 700-800 mg, 800-900 mg, 900-1,000 mg, 1000-1250 mg, 1250-1500 mg, 1500-1750 mg, 1750-2000 mg, 2000-2250 mg, 2250-2500 mg, 2500-3000 mg, 3000-3500 mg, 3500-4000 mg, 4000-4500 mg, 4500-5000 mg, 5000-6000 mg, 6000-7000 mg, 7000-8000 mg, 8000-9000 mg, or 9000-10000 mg.

In another aspect, any of the pharmaceutical compositions, nutraceutical compositions, food products, beverages, or nutritional supplements described herein comprises about 0.005 mg/mL to about 100 mg/mL of a compound, agent, or particle described herein. In another aspect, any of the pharmaceutical compositions, nutraceutical compositions, food products, beverages, or nutritional supplements described herein comprises about 0.01 mg/mL to about 100 mg/mL of a compound, agent, or particle described herein. In another aspect, any of the pharmaceutical compositions, nutraceutical compositions, food products, beverages, or nutritional supplements described herein comprises about 0.05 mg/mL to about 100 mg/mL of a compound, agent, or particle described herein. In another aspect, any of the pharmaceutical compositions, nutraceutical compositions, food products, beverages, or nutritional supplements described herein comprises about 0.1 mg/mL to about 100 mg/mL of a compound, agent, or particle described herein. In another aspect, any of the pharmaceutical compositions, nutraceutical compositions, food products, beverages, or nutritional supplements described herein comprises about 0.5 mg/mL to about 100 mg/mL of a compound, agent, or particle described herein. In another aspect, any of the pharmaceutical compositions, nutraceutical compositions, food products, beverages, or nutritional supplements described herein comprises about 1 mg/mL to about 100 mg/mL of a compound, agent, or particle described herein.

In another aspect, any of the pharmaceutical compositions, nutraceutical compositions, food products, beverages, or nutritional supplements described herein comprises about 0.005 mg/mL to about 50 mg/mL of a compound, agent, or particle described herein. In another aspect, any of the pharmaceutical compositions, nutraceutical compositions, food products, beverages, or nutritional supplements described herein comprises about 0.01 mg/mL to about 50 mg/mL of a compound, agent, or particle described herein. In another aspect, any of the pharmaceutical compositions, nutraceutical compositions, food products, beverages, or nutritional supplements described herein comprises about 0.05 mg/mL to about 50 mg/mL of a compound, agent, or particle described herein. In another aspect, any of the pharmaceutical compositions, nutraceutical compositions, food products, beverages, or nutritional supplements described herein comprises about 0.1 mg/mL to about 50 mg/mL of a compound, agent, or particle described herein. In another aspect, any of the pharmaceutical compositions, nutraceutical compositions, food products, beverages, or nutritional supplements described herein comprises about 0.5 mg/mL to about 50 mg/mL of a compound, agent, or particle described herein. In another aspect, any of the pharmaceutical compositions, nutraceutical compositions, food products, beverages, or nutritional supplements described herein comprises about 1 mg/mL to about 50 mg/mL of a compound, agent, or particle described herein.

In another aspect, any of the pharmaceutical compositions, nutraceutical compositions, food products, beverages, or nutritional supplements described herein comprises about 0.005 mg/mL to about 25 mg/mL of a compound, agent, or particle described herein. In another aspect, any of the pharmaceutical compositions, nutraceutical compositions, food products, beverages, or nutritional supplements described herein comprises about 0.01 mg/mL to about 25 mg/mL of a compound, agent, or particle described herein. In another aspect, any of the pharmaceutical compositions, nutraceutical compositions, food products, beverages, or nutritional supplements described herein comprises about 0.05 mg/mL to about 25 mg/mL of a compound, agent, or particle described herein. In another aspect, any of the pharmaceutical compositions, nutraceutical compositions, food products, beverages, or nutritional supplements described herein comprises about 0.1 mg/mL to about 25 mg/mL of a compound, agent, or particle described herein. In another aspect, any of the pharmaceutical compositions, nutraceutical compositions, food products, beverages, or nutritional supplements described herein comprises about 0.5 mg/mL to about 25 mg/mL of a compound, agent, or particle described herein. In another aspect, any of the pharmaceutical compositions, nutraceutical compositions, food products, beverages, or nutritional supplements described herein comprises about 1 mg/mL to about 25 mg/mL of a compound, agent, or particle described herein.

In another aspect, any of the pharmaceutical compositions, nutraceutical compositions, food products, beverages, or nutritional supplements described herein comprises about 0.005 mg/mL to about 10 mg/mL of a compound, agent, or particle described herein. In another aspect, any of the pharmaceutical compositions, nutraceutical compositions, food products, beverages, or nutritional supplements described herein comprises about 0.01 mg/mL to about 10 mg/mL of a compound, agent, or particle described herein. In another aspect, any of the pharmaceutical compositions, nutraceutical compositions, food products, beverages, or nutritional supplements described herein comprises about 0.05 mg/mL to about 10 mg/mL of a compound, agent, or particle described herein. In another aspect, any of the pharmaceutical compositions, nutraceutical compositions, food products, beverages, or nutritional supplements described herein comprises about 0.1 mg/mL to about 10 mg/mL of a compound, agent, or particle described herein. In another aspect, any of the pharmaceutical compositions, nutraceutical compositions, food products, beverages, or nutritional supplements described herein comprises about 0.5 mg/mL to about 10 mg/mL of a compound, agent, or particle described herein. In another aspect, any of the pharmaceutical compositions, nutraceutical compositions, food products, beverages, or nutritional supplements described herein comprises about 1 mg/mL to about 10 mg/mL of a compound, agent, or particle described herein.

### Kits

In another aspect, the present disclosure provides a kit comprising a compound provided herein (e.g., a compound of Formula (I)), or a pharmaceutically acceptable salt, stereoisomer, or isotopically labeled derivative thereof; or a pharmaceutical composition thereof; and instructions for using the compound, or pharmaceutically acceptable salt, stereoisomer, or isotopically labeled derivative thereof, or pharmaceutical composition thereof (e.g., for treating and/or preventing a disease or condition in a subject or delivering an agent to a subject).

The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease, disorder, or condition in a subject in need thereof. In certain embodiments, the kits are useful for preventing a disease, disorder, or condition in a subject in need thereof. In certain embodiments, the kits are useful for reducing the risk of developing a disease, disorder, or condition in a subject in need thereof. In some embodiments, the disease, disorder, or condition is a micronutrient deficiency. In certain embodiments, the kits are useful for delivering an agent to a subject. In certain embodiments, the kits are useful for delivering a vitamin to a subject. In certain embodiments, the kits are useful for delivering a mineral to a subject.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease, disorder, or condition in a subject in need thereof. In certain embodiments, the kits and instructions provide for preventing a disease, disorder, or condition in a subject in need thereof. In certain embodiments, the kits and instructions provide for reducing the risk of developing a disease, disorder, or condition in a subject in need thereof. In some embodiments, the disease, disorder, or condition is a micronutrient deficiency. In certain embodiments, the kits and instructions provide for delivering an agent to a subject. In certain embodiments, the kits and instructions provide for delivering a vitamin to a subject. In certain embodiments, the kits are useful for delivering a mineral to a subject. A kit described herein may include one or more additional pharmaceutical agents described herein as a separate composition.

### Methods of Treatment and Uses

Also provided herein are methods for treating and/or preventing a disease, disorder, or condition in a subject, comprising administering to the subject a composition comprising an agent and a compound of Formula (I), or a pharmaceutically acceptable salt, solvate, tautomer, stereoisomer, or isotopically labeled derivative thereof.

In certain embodiments, the disease, disorder, or condition is a micronutrient deficiency, genetic disease, proliferative disease, hematological disease, neurological disease, liver disease, spleen disease, lung disease, painful condition, psychiatric disorder, musculoskeletal disease, metabolic disorder, inflammatory disease, or autoimmune disease. In some embodiments, the disease, disorder, or condition is a micronutrient deficiency. In certain embodiments, the micronutrient deficiency is a vitamin deficiency. In some embodiments, the micronutrient deficiency is a mineral deficiency. In certain embodiments, the micronutrient deficiency is vitamin A deficiency. In some embodiments, the micronutrient deficiency is iron deficiency. In certain embodiments, the micronutrient deficiency is iodine deficiency. In some embodiments, the micronutrient deficiency is vitamin B1 deficiency. In some embodiments, the micronutrient deficiency is vitamin B2 (riboflavin) deficiency. In some embodiments, the micronutrient deficiency is vitamin B3 deficiency. In some embodiments, the micronutrient deficiency is vitamin B6 deficiency. In certain embodiments, the micronutrient deficiency is vitamin B7 (niacin) deficiency. In some embodiments, the micronutrient deficiency is vitamin B9 deficiency. In some embodiments, the micronutrient deficiency is vitamin B12 (cobalamin) deficiency. In certain embodiments, the micronutrient deficiency is vitamin D (cholecalcifeol) deficiency. In some embodiments, the micronutrient deficiency is vitamin E (tocopherol) deficiency. In certain embodiments, the micronutrient deficiency is vitamin K1 (phytomenadione) deficiency. In some embodiments, the micronutrient deficiency is vitamin K3 deficiency. In some embodiments, the micronutrient deficiency is vitamin C (6-O-pamlmitoyl-L-ascorbic acid) deficiency. In certain embodiments, the micronutrient deficiency is zinc deficiency.

### Additional Methods and Uses

Also provided herein is a method of delivering an agent to a subject, comprising administering to the subject a composition provided herein.

In certain embodiments, the agent is any agent provided herein. In some embodiments, the agent is a vitamin, mineral, micronutrient, biologic, small molecule, probiotic, or polynucleotide. In some embodiments, the agent is a vitamin, mineral, micronutrient, or probiotic. In certain embodiments, the agent is a vitamin or mineral. In some embodiments, the agent is a vitamin. In certain embodiments, the agent is a mineral.

In certain embodiments, the composition is administered by any method provide herein. In some embodiments, the composition is administered orally.

Also provided herein is a method of preparing a compound comprising Formula (I):

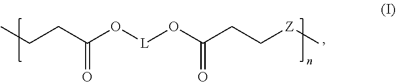

or a pharmaceutically acceptable salt, stereoisomer, or isotopically labeled derivative thereof, the method comprising reacting one or more compounds of Formula (II):

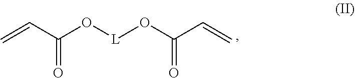

or a salt, isotope, or stereoisomer thereof, with one or more compounds selected from

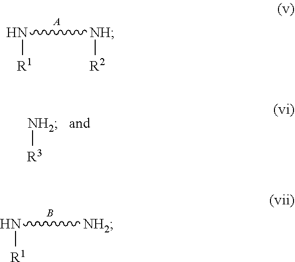

or a salt, isotope, or stereoisomer thereof, wherein:
L is a heterocycle comprising at least one oxygen atom;
linker A is branched or unbranched, optionally substituted, cyclic or acyclic aliphatic; branched or unbranched, optionally substituted, cyclic or acyclic heteroaliphatic; optionally substituted arylene; or optionally substituted heteroarylene;
R¹ and R² are each independently hydrogen; branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group; or
one or both R¹ or R² are each optionally independently joined to linker A together with the intervening atoms to form a linker comprising one or more N-containing heterocycles; or
R¹ and R² are joined together with the intervening atoms to form a N-containing heterocycle;
R³ is branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group;
linker B is branched or unbranched, optionally substituted, cyclic or acyclic aliphatic; branched or unbranched, optionally substituted, cyclic or acyclic heteroaliphatic; optionally substituted arylene; or optionally substituted heteroarylene; and
R⁴ is hydrogen, branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group; or
R⁴ is joined to linker B together with the intervening atoms to form a linker comprising a N-containing heterocycle.

In some embodiments, the method further comprises reacting the compound comprising Formula (I) with one or more compounds selected from HOR^{C}, HSR^{C}, or HN(R^{C})₂. In some embodiments, the method affords a compound of Formula (III), or a pharmaceutically acceptable salt, stereoisomer, or isotopically labeled derivative thereof.

As defined herein, each instance of R^{C} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, a nitrogen protecting group when attached to a nitrogen atom, or two instances of R^{C} attached to the same intervening atom are joined together with the intervening atom to form optionally substituted heterocyclyl or optionally substituted heteroaryl. In some embodiments, at least one instance of R^{C} is independently hydrogen, optionally substituted alkyl, optionally substituted alkenyl, optionally substituted alkynyl, optionally substituted heteroalkyl, optionally substituted heteroalkenyl, or optionally substituted heteroalkynyl. In some embodiments, at least one instance of R^{C} is optionally substituted carbocyclyl, optionally substituted heterocyclyl, optionally substituted aryl, or optionally substituted heteroaryl. In some embodiments, at least one instance of R^{C} is independently hydrogen, optionally substituted C₁₋₁₀ alkyl, optionally substituted C₁₋₁₀ alkenyl, optionally substituted C₁₋₁₀ alkynyl, optionally substituted C₃₋₁₄ carbocyclyl, or optionally substituted C₆₋₁₄ aryl. In some embodiments, at least one instance of R^{C} is independently hydrogen, optionally substituted C₁₋₁₀ alkyl, or optionally substituted phenyl. In some embodiments, at least one instance of R^{C} is hydrogen, a nitrogen protecting group when attached to a nitrogen atom, an oxygen protecting group when attached to an oxygen atom, or a sulfur protecting group when attached to a sulfur atom. In some embodiments, at least one instance of R^{C} is a nitrogen protecting group when attached to a nitrogen atom, an oxygen protecting group when attached to an oxygen atom, or a sulfur protecting group when attached to a sulfur atom. In some embodiments, two instances of R^{C} attached to the same intervening atom are joined together with the intervening atom to form an optionally substituted, monocyclic, heterocyclic or heteroaryl ring.

As defined herein, L is a heterocycle comprising at least one oxygen atom. In certain embodiments, L comprises one oxygen atom. In some embodiments, L comprises at least two oxygen atoms. In certain embodiments, L comprises two oxygen atoms. In some embodiments, L comprises at least three oxygen atoms. In certain embodiments, L comprises three oxygen atoms. In certain embodiments, L comprises a pyran. In certain embodiments, L comprises a furan.

In some embodiments, L is derived from a monosaccharide. In certain embodiments, the monosaccharide is arabinose, lyxose, ribose, xylose, ribulofuranose, xylulose, allose, altrose, galactose, glucose, gulose, idose, mannose, talose, fructose, psicose, sorbose, tagatose, mannoheptulose, and sedoheptulose. In some embodiments, the monosaccharide is allose, altrose, galactose, glucose, gulose, idose, mannose, talose, fructose, psicose, sorbose, tagatose. In certain embodiments, the monosaccharide is glucose or galactose. In some embodiments, the monosaccharide is galactose. In certain embodiments, the monosaccharide is glucose.

In some embodiments, L is an unsubstituted 3-14 membered heterocycle comprising at least one oxygen atom. In certain embodiments, L is a substituted 3-14 membered heterocycle comprising at least one oxygen atom. In certain embodiments, L is optionally substituted, 3- to 7-membered, monocyclic heterocycle, comprising at least one oxygen atom. In certain embodiments, L is substituted, 3- to 7-membered, monocyclic heterocycle, comprising at least one oxygen atom. In certain embodiments, L is unsubstituted, 3- to 7-membered, monocyclic heterocycle, comprising at least one oxygen atom. In certain embodiments, L is optionally substituted, 5- to 6-membered, monocyclic heterocycle, comprising at least one oxygen atom. In certain embodiments, L is substituted, 5- to 5-membered, monocyclic heterocycle, comprising at least one oxygen atom. In certain embodiments, L is unsubstituted, 5- to 6-membered, monocyclic heterocycle, comprising at least one oxygen atom. In certain embodiments, L is optionally substituted, 8- to 10-membered, bicyclic heterocycle, comprising at least one oxygen atom. In certain embodiments, L is substituted, 8- to 10-membered, bicyclic heterocycle, comprising at least one oxygen atom. In certain embodiments, L is unsubstituted, 8- to 10-membered, bicyclic heterocycle, comprising at least one oxygen atom.

In some embodiments, L is substituted with at least one hydroxy or alkoxy substituent. In certain embodiments, L is substituted with —OH, —OR⁵, —OR⁶, or —OR⁷, wherein R⁵, R⁶, and R⁷ are each independently an oxygen protecting group, or wherein R⁵, R⁶, and R⁷ may be joined together with the intervening atoms to form optionally substituted heterocyclyl. In some embodiments, L is substituted with —OH. In certain embodiments, L is substituted with —OR⁵, —OR⁶, or —OR⁷. In some embodiments, L is substituted with —OR⁵. In certain embodiments, L is substituted with —OR⁶. In some embodiments, L is substituted with —OR⁷.

In certain embodiments, L is

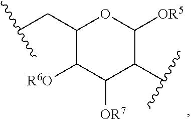

wherein R⁵, R⁶, and R⁷ are each independently hydrogen, C₁₋₆ aliphatic, an oxygen protecting group, or wherein R⁶ and R⁷ may be joined together with the intervening atoms to form optionally substituted heterocyclyl.

In certain embodiments, the compound of Formula (II) is of the formula:

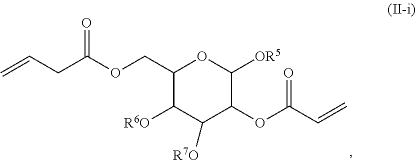

wherein R⁵, R⁶, and R⁷ are each independently hydrogen, C₁₋₆ aliphatic, an oxygen protecting group, or wherein R⁶ and R⁷ may be joined together with the intervening atoms to form optionally substituted heterocyclyl.

As defined herein, R⁵, R⁶, and R⁷ are each independently hydrogen, C₁₋₆, aliphatic, an oxygen protecting group, or wherein R⁶ and R⁷ may be joined together with the intervening atoms to form optionally substituted heterocyclyl. In some embodiments, R⁵, R⁶, and R⁷ are each independently an oxygen protecting group or wherein R⁶ and R⁷ may be joined together with the intervening atoms to form optionally substituted heterocyclyl. In some embodiments, R⁵ is an oxygen protecting group. In some embodiments, R⁵ is C₁₋₆ aliphatic. In some embodiments, R⁵ is hydrogen. In certain embodiments, R⁶ is an oxygen protecting group. In some embodiments, R⁶ is C₁₋₆ aliphatic. In some embodiments, R⁶ is hydrogen. In some embodiments, R⁷ is an oxygen protecting group. In some embodiments, R⁷ is C₁₋₆ aliphatic. In some embodiments, R⁷ is hydrogen. In certain embodiments, the oxygen protecting group is unsubstituted C₁-C₆ alkyl, silyl, TBDPS, TBDMS, TIPS, TES, TMS, MOM, THP, t-Bu, Bn, allyl, acetyl, pivaloyl, or benzoyl. In some embodiments, the oxygen protecting group is unsubstituted C₁-C₆ alkyl.

In certain embodiments, R⁶ and R⁷ are joined together with the intervening atoms to form optionally substituted heterocyclyl. In certain embodiments, R⁶ and R⁷ are joined together with the intervening atoms to form optionally substituted 5-6-membered heterocyclyl. In certain embodiments, R⁶ and R⁷ are joined together with the intervening atoms to form unsubstituted 5-6-membered heterocyclyl. In certain embodiments, R⁶ and R⁷ are joined together with the intervening atoms to form substituted 5-6-membered heterocyclyl. In certain embodiments, R⁶ and R⁷ are joined together with the intervening atoms to form 5-6-membered heterocyclyl substituted with one or more C₁-C₆ alkyl substituents. In some embodiments, R⁶ and R⁷ are joined together with the intervening atoms to form a cyclic ketal. In certain embodiments, R⁶ and R⁷ are joined together with the intervening atoms to form a 1,3-dioxolane. In some embodiments, R⁶ and R⁷ are joined together with the intervening atoms to form 2,2-dimethyl-1,3-dioxolane.

In some embodiments, L is

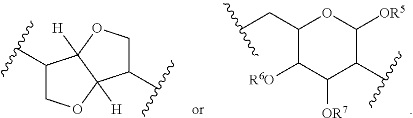

In some embodiments,

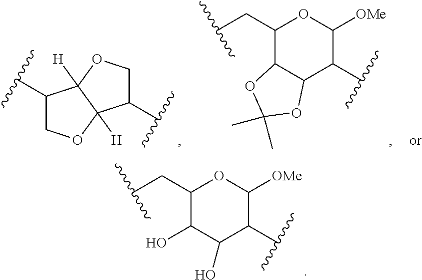

In some embodiments,

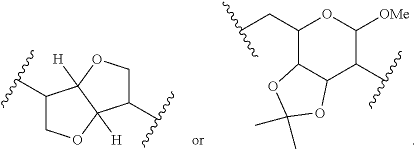 [SMILES: *C1COC2C(*)COC12 ; *CC1OC(OC)C(*)C2OC(C)(C)OC12]

In some embodiments, L is

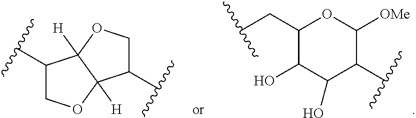 [SMILES: *C1COC2C(*)COC12 ; *CC1OC(OC)C(*)C(O)C1O]

In some embodiments, L is

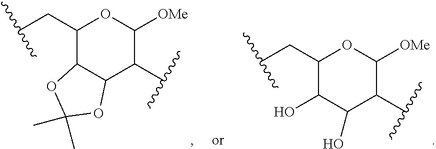 [SMILES: *CC1OC(OC)C(*)C2OC(C)(C)OC12 ; *CC1OC(OC)C(*)C(O)C1O]

In some embodiments, L is

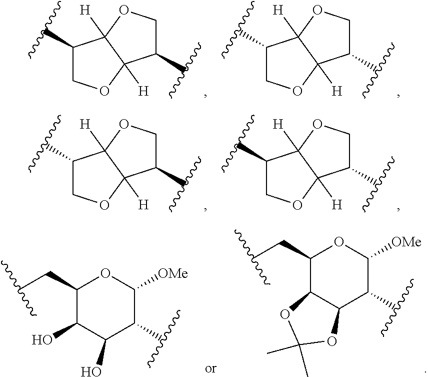

In some embodiments, L is

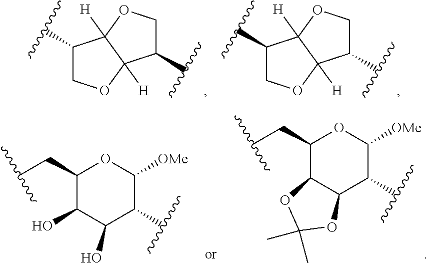

In some embodiments, L is

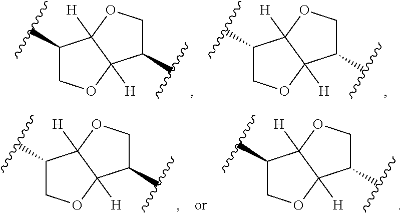

In some embodiments, L is

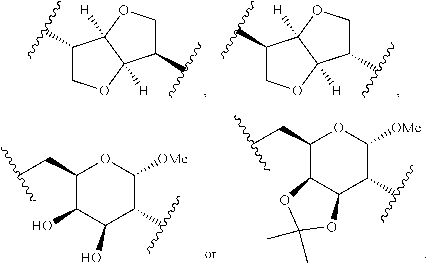

In some embodiments, L is

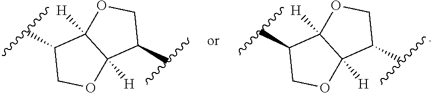 [SMILES: *[C@@H]1CO[C@H]2[C@@H]1OC[C@@H]2* ; *[C@@H]1CO[C@H]2[C@@H]1OC[C@@H]2*]

In some embodiments, L is

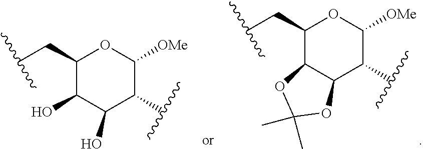 [SMILES: *C[C@H]1O[C@H](OC)[C@H](*)[C@@H](O)[C@H]1O ; *C[C@H]1O[C@H](OC)[C@H](*)[C@H]2OC(C)(C)O[C@H]21]

In certain embodiments, L is

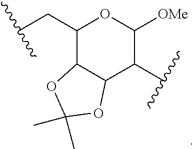 [SMILES: *CC1OC(OC)C(*)C2OC(C)(C)OC12]

In certain embodiments, L is

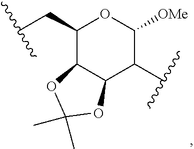 [SMILES: *C[C@H]1O[C@H](OC)C(*)[C@H]2OC(C)(C)O[C@H]21]

In certain embodiments, L is

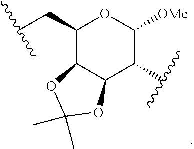 [SMILES: *C[C@H]1O[C@H](OC)[C@H](*)[C@H]2OC(C)(C)O[C@H]21]

In certain embodiments, the compound of Formula (II-i) is:

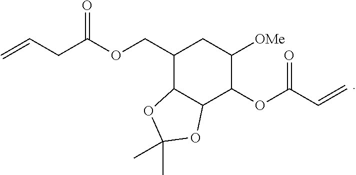 [SMILES: C=CCC(=O)OCC1CC(OC)C(OC(=O)C=C)C2OC(C)(C)OC12 ; "."]

In certain embodiments, the compound of Formula (II-i) is:

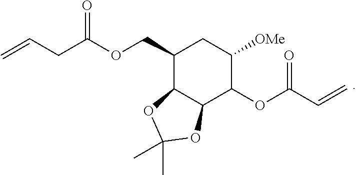 [SMILES: C=CCC(=O)OC[C@H]1C[C@H](OC)C(OC(=O)C=C)[C@H]2OC(C)(C)O[C@@H]12 ; "."]

In certain embodiments, the compound of Formula (II-i) is:

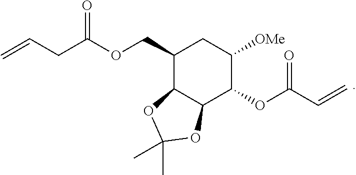 [SMILES: C=CCC(=O)OC[C@H]1C[C@H](OC)[C@H](OC(=O)C=C)[C@H]2OC(C)(C)O[C@@H]12 ; "."]

In certain embodiments, L is

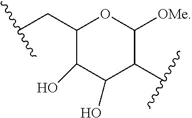 [SMILES: *CC1OC(OC)C(*)C(O)C1O]

In certain embodiments, L is

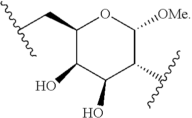 [SMILES: *C[C@H]1O[C@H](OC)[C@H](*)[C@@H](O)[C@H]1O]

In certain embodiments, the compound of Formula (II-i) is:

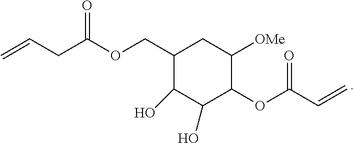 [SMILES: C=CCC(=O)OCC1CC(OC)C(OC(=O)C=C)C(O)C1O ; "."]

In certain embodiments, the compound of Formula (II-i) is:

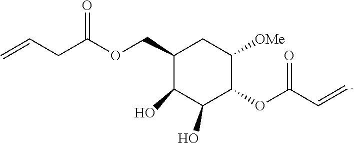 [SMILES: C=CCC(=O)OC[C@H]1C[C@H](OC)[C@H](OC(=O)C=C)[C@@H](O)[C@H]1O ; "."]

In certain embodiments, L is

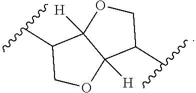 [SMILES: *C1COC2C(*)COC12]

In certain embodiments, L is

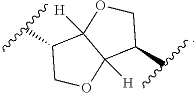 [SMILES: *[C@@H]1COC2C1OC[C@@H]2*]

In some embodiments, L is

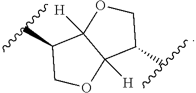 [SMILES: *[C@@H]1COC2C1OC[C@@H]2*]

In some embodiments, L is

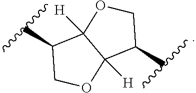 [SMILES: *[C@@H]1COC2C1OC[C@H]2*]

In some embodiments, L is

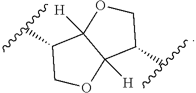 [SMILES: *[C@H]1COC2C1OC[C@@H]2*]

In certain embodiments, L is

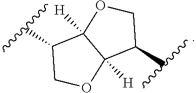 [SMILES: *[C@@H]1CO[C@H]2[C@@H]1OC[C@@H]2*]

In some embodiments, L is

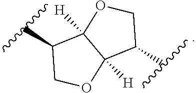 [SMILES: *[C@@H]1CO[C@H]2[C@@H]1OC[C@@H]2*]

In some embodiments, L is

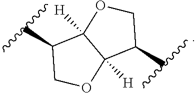 [SMILES: *[C@@H]1CO[C@H]2[C@@H]1OC[C@H]2*]

In some embodiments, L is

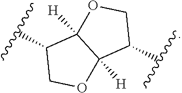 [SMILES: *[C@H]1CO[C@H]2[C@@H]1OC[C@@H]2*]

In certain embodiments, the compound of Formula (II) is

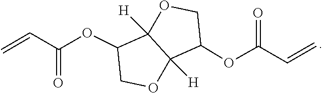 [SMILES: C=CC(=O)OC1COC2C(OC(=O)C=C)COC12]

In certain embodiments, the compound of Formula (II) is

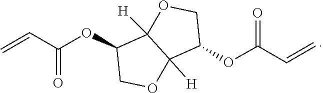 [SMILES: C=CC(=O)O[C@H]1COC2C1OC[C@H]2OC(=O)C=C]

In certain embodiments, the compound of Formula (II) is

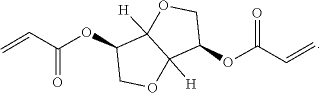 [SMILES: C=CC(=O)O[C@@H]1COC2C1OC[C@H]2OC(=O)C=C]

In certain embodiments, the compound of Formula (II) is

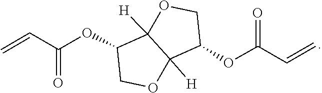 [SMILES: C=CC(=O)O[C@H]1COC2C1OC[C@@H]2OC(=O)C=C]

In certain embodiments, the compound of Formula (II) is

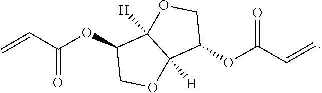 [SMILES: C=CC(=O)O[C@H]1CO[C@H]2[C@@H]1OC[C@H]2OC(=O)C=C]

In certain embodiments, the compound of Formula (II) is

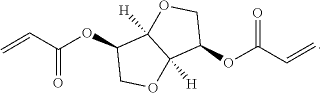 [SMILES: C=CC(=O)O[C@@H]1CO[C@H]2[C@@H]1OC[C@H]2OC(=O)C=C]

In certain embodiments, the compound of Formula (II) is

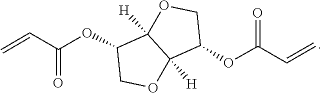 [SMILES: C=CC(=O)O[C@H]1CO[C@H]2[C@@H]1OC[C@@H]2OC(=O)C=C]

As defined herein, R¹ and R² are each independently hydrogen; branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group; or one or both R¹ or R² are each optionally independently joined to linker A together with the intervening atoms to form a linker comprising one or more N-containing heterocycles; or R¹ and R² are joined together with the intervening atoms to form a N-containing heterocycle.

In certain embodiments, R¹ and R² are each independently hydrogen; branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group.

In some embodiments, R¹ is hydrogen.

In certain embodiments, R¹ is branched or unbranched, optionally substituted, cyclic or acyclic aliphatic. In some embodiments, R¹ is branched or unbranched, optionally substituted, cyclic aliphatic. In certain embodiments, R¹ is substituted cyclic aliphatic. In some embodiments, linker is unsubstituted cyclic aliphatic. In certain embodiments, R¹ is branched or unbranched, optionally substituted carbocyclylene. In some embodiments, R¹ is substituted carbocyclylene. In certain embodiments, R¹ is unsubstituted carbocyclylene. In certain embodiments, R¹ is branched or unbranched, optionally substituted C₃-C₈ carbocyclylene. In some embodiments, R¹ is substituted C₃-C₈ carbocyclylene. In certain embodiments, R¹ is unsubstituted C₃-C₈ carbocyclylene. In some embodiments, R¹ is branched or unbranched, optionally substituted, acyclic aliphatic. In some embodiments, R¹ is substituted acyclic aliphatic. In certain embodiments, R¹ is unsubstituted acyclic aliphatic. In some embodiments, R¹ is branched or unbranched, optionally substituted, alkylene. In some embodiments, R¹ is substituted alkylene. In some embodiments, R¹ is unsubstituted alkylene. In some embodiments, R¹ is branched or unbranched, optionally substituted, C₁-C₁₀ alkylene. In some embodiments, R¹ is substituted C₁-C₁₀ alkylene. In some embodiments, R¹ is unsubstituted C₁-C₁₀ alkylene. In some embodiments, R¹ is branched or unbranched, optionally substituted, alkenylene. In some embodiments, R¹ is substituted alkenylene. In some embodiments, R¹ is unsubstituted alkenylene. In some embodiments, R¹ is branched or unbranched, optionally substituted, C₂-C₁₀ alkenylene. In some embodiments, R¹ is substituted C₂-C₁₀ alkenylene. In some embodiments, R¹ is unsubstituted C₂-C₁₀ alkenylene. In some embodiments, R¹ is branched or unbranched, optionally substituted, alkynylene. In some embodiments, R¹ is substituted alkynylene. In some embodiments, R¹ is unsubstituted alkynylene. In some embodiments, R¹ is branched or unbranched, optionally substituted, C₂-C₁₀ alkynylene. In some embodiments, R¹ is substituted C₂-C₁₀ alkynylene. In some embodiments, R¹ is unsubstituted C₂-C₁₀ alkynylene.

In certain embodiments, R¹ is branched or unbranched, optionally substituted, cyclic or acyclic heteroaliphatic. In some embodiments, R¹ is branched or unbranched, optionally substituted, cyclic heteroaliphatic. In certain embodiments, R¹ is substituted cyclic heteroaliphatic. In some embodiments, linker is unsubstituted cyclic heteroaliphatic. In certain embodiments, R¹ is branched or unbranched, optionally substituted heterocyclylene. In some embodiments, R¹ is substituted heterocyclylene. In certain embodiments, R¹ is unsubstituted heterocyclylene. In certain embodiments, R¹ is branched or unbranched, optionally substituted 3-8-membered heterocyclylene. In some embodiments, R¹ is substituted 3-8-membered heterocyclylene. In certain embodiments, R¹ is unsubstituted 3-8-membered heterocyclylene. In some embodiments, R¹ is branched or unbranched, optionally substituted, acyclic heteroaliphatic. In some embodiments, R¹ is substituted acyclic heteroaliphatic. In certain embodiments, R¹ is unsubstituted acyclic heteroaliphatic. In some embodiments, R¹ is branched or unbranched, optionally substituted, heteroalkylene. In some embodiments, R¹ is substituted heteroalkylene. In some embodiments, R¹ is unsubstituted heteroalkylene. In some embodiments, R¹ is branched or unbranched, optionally substituted, C₁-C₁₀ heteroalkylene. In some embodiments, R¹ is substituted C₁-C₁₀ heteroalkylene. In some embodiments, R¹ is unsubstituted C₁-C₁₀ heteroalkylene. In some embodiments, R¹ is branched or unbranched, optionally substituted, heteroalkenylene. In some embodiments, R¹ is substituted heteroalkenylene. In some embodiments, R¹ is unsubstituted heteroalkenylene. In some embodiments, R¹ is branched or unbranched, optionally substituted, C₂-C₁₀ heteroalkenylene. In some embodiments, R¹ is substituted C₂-C₁₀ heteroalkenylene. In some embodiments, R¹ is unsubstituted C₂-C₁₀ heteroalkenylene. In some embodiments, R¹ is branched or unbranched, optionally substituted, heteroalkynylene. In some embodiments, R¹ is substituted heteroalkynylene. In some embodiments, R¹ is unsubstituted heteroalkynylene. In some embodiments, R¹ is branched or unbranched, optionally substituted, C₂-C₁₀ heteroalkynylene. In some embodiments, R¹ is substituted C₂-C₁₀ heteroalkynylene. In some embodiments, R¹ is unsubstituted C₂-C₁₀ heteroalkynylene.

In certain embodiments, R¹ is branched or unbranched, optionally substituted, cyclic or acyclic acyl. In some embodiments, R¹ is substituted cyclic or acyclic acyl. In some embodiments, R¹ is unsubstituted cyclic or acyclic acyl. In certain embodiments, R¹ is branched or unbranched, optionally substituted, cyclic acyl. In some embodiments, R¹ is substituted cyclic acyl. In some embodiments, R¹ is unsubstituted cyclic acyl. In certain embodiments, R¹ is branched or unbranched, optionally substituted, C₅-C₁₀ cyclic acyl. In some embodiments, R¹ is substituted C₅-C₁₀ cyclic acyl. In some embodiments, R¹ is unsubstituted C₅-C₁₀ cyclic acyl. In certain embodiments, R¹ is branched or unbranched, optionally substituted, acyclic acyl. In some embodiments, R¹ is substituted acyclic acyl. In some embodiments, R¹ is unsubstituted acyclic acyl. In certain embodiments, R¹ is branched or unbranched, optionally substituted, C₁-C₁₀ acyclic acyl. In some embodiments, R¹ is substituted C₁-C₁₀ acyclic acyl. In some embodiments, R¹ is unsubstituted C₁-C₁₀ acyclic acyl.

In certain embodiments, R¹ is optionally substituted aryl. In some embodiments, R¹ is substituted aryl. In certain embodiments, R¹ is unsubstituted aryl. In some embodiments, R¹ is optionally substituted C₆-C₁₄ aryl. In certain embodiments, R¹ is substituted C₆-C₁₄ aryl. In some embodiments, R¹ is unsubstituted C₆-C₁₄ aryl.

In certain embodiments, R¹ is optionally substituted heteroaryl. In some embodiments, R¹ is substituted heteroaryl. In certain embodiments, R¹ is unsubstituted heteroaryl. In certain embodiments, R¹ is optionally substituted 5-14-membered heteroaryl. In some embodiments, R¹ is substituted 5-14-membered heteroaryl. In certain embodiments, R¹ is unsubstituted 5-14-membered heteroaryl.

In certain embodiments, R¹ is a nitrogen protecting group. In some embodiments, the nitrogen protecting group is Bn, Boc, Cbz, Fmoc, trifluoroacetyl, triphenylmethyl, acetyl, or Ts.

In some embodiments, R² is hydrogen.

In certain embodiments, R² is branched or unbranched, optionally substituted, cyclic or acyclic aliphatic. In some embodiments, R² is branched or unbranched, optionally substituted, cyclic aliphatic. In certain embodiments, R² is substituted cyclic aliphatic. In some embodiments, linker is unsubstituted cyclic aliphatic. In certain embodiments, R² is branched or unbranched, optionally substituted carbocyclylene. In some embodiments, R² is substituted carbocyclylene. In certain embodiments, R² is unsubstituted carbocyclylene. In certain embodiments, R² is branched or unbranched, optionally substituted C₃-C₈ carbocyclylene. In some embodiments, R² is substituted C₃-C₈ carbocyclylene. In certain embodiments, R² is unsubstituted C₃-C₈ carbocyclylene. In some embodiments, R² is branched or unbranched, optionally substituted, acyclic aliphatic. In some embodiments, R² is substituted acyclic aliphatic. In certain embodiments, R² is unsubstituted acyclic aliphatic. In some embodiments, R² is branched or unbranched, optionally substituted, alkylene. In some embodiments, R² is substituted alkylene. In some embodiments, R² is unsubstituted alkylene. In some embodiments, R² is branched or unbranched, optionally substituted, C₁-C₁₀ alkylene. In some embodiments, R² is substituted C₁-C₁₀ alkylene. In some embodiments, R² is unsubstituted C₁-C₁₀ alkylene. In some embodiments, R² is branched or unbranched, optionally substituted, alkenylene. In some embodiments, R² is substituted alkenylene. In some embodiments, R² is unsubstituted alkenylene. In some embodiments, R² is branched or unbranched, optionally substituted, C₂-C₁₀ alkenylene. In some embodiments, R² is substituted C₂-C₁₀ alkenylene. In some embodiments, R² is unsubstituted C₂-C₁₀ alkenylene. In some embodiments, R² is branched or unbranched, optionally substituted, alkynylene. In some embodiments, R² is substituted alkynylene. In some embodiments, R² is unsubstituted alkynylene. In some embodiments, R² is branched or unbranched, optionally substituted, C₂-C₁₀ alkynylene. In some embodiments, R² is substituted C₂-C₁₀ alkynylene. In some embodiments, R² is unsubstituted C₂-C₁₀ alkynylene.

In certain embodiments, R² is branched or unbranched, optionally substituted, cyclic or acyclic heteroaliphatic. In some embodiments, R² is branched or unbranched, optionally substituted, cyclic heteroaliphatic. In certain embodiments, R² is substituted cyclic heteroaliphatic. In some embodiments, linker is unsubstituted cyclic heteroaliphatic. In certain embodiments, R² is branched or unbranched, optionally substituted heterocyclylene. In some embodiments, R² is substituted heterocyclylene. In certain embodiments, R² is unsubstituted heterocyclylene. In certain embodiments, R² is branched or unbranched, optionally substituted 3-8-membered heterocyclylene. In some embodiments, R² is substituted 3-8-membered heterocyclylene. In certain embodiments, R² is unsubstituted 3-8-membered heterocyclylene. In some embodiments, R² is branched or unbranched, optionally substituted, acyclic heteroaliphatic. In some embodiments, R² is substituted acyclic heteroaliphatic. In certain embodiments, R² is unsubstituted acyclic heteroaliphatic. In some embodiments, R² is branched or unbranched, optionally substituted, heteroalkylene. In some embodiments, R² is substituted heteroalkylene. In some embodiments, R² is unsubstituted heteroalkylene. In some embodiments, R² is branched or unbranched, optionally substituted, C₁-C₁₀ heteroalkylene. In some embodiments, R² is substituted C₁-C₁₀ heteroalkylene. In some embodiments, R² is unsubstituted C₁-C₁₀ heteroalkylene. In some embodiments, R² is branched or unbranched, optionally substituted, heteroalkenylene. In some embodiments, R² is substituted heteroalkenylene. In some embodiments, R² is unsubstituted heteroalkenylene. In some embodiments, R² is branched or unbranched, optionally substituted, C₂-C₁₀ heteroalkenylene. In some embodiments, R² is substituted C₂-C₁₀ heteroalkenylene. In some embodiments, R² is unsubstituted C₂-C₁₀ heteroalkenylene. In some embodiments, R² is branched or unbranched, optionally substituted, heteroalkynylene. In some embodiments, R² is substituted heteroalkynylene. In some embodiments, R² is unsubstituted heteroalkynylene. In some embodiments, R² is branched or unbranched, optionally substituted, C₂-C₁₀ heteroalkynylene. In some embodiments, R² is substituted C₂-C₁₀ heteroalkynylene. In some embodiments, R² is unsubstituted C₂-C₁₀ heteroalkynylene.

In certain embodiments, R² is branched or unbranched, optionally substituted, cyclic or acyclic acyl. In some embodiments, R² is substituted cyclic or acyclic acyl. In some embodiments, R² is unsubstituted cyclic or acyclic acyl. In certain embodiments, R² is branched or unbranched, optionally substituted, cyclic acyl. In some embodiments, R² is substituted cyclic acyl. In some embodiments, R² is unsubstituted cyclic acyl. In certain embodiments, R² is branched or unbranched, optionally substituted, C₅-C₁₀ cyclic acyl. In some embodiments, R² is substituted C₅-C₁₀ cyclic acyl. In some embodiments, R² is unsubstituted C₅-C₁₀ cyclic acyl. In certain embodiments, R² is branched or unbranched, optionally substituted, acyclic acyl. In some embodiments, R² is substituted acyclic acyl. In some embodiments, R² is unsubstituted acyclic acyl. In certain embodiments, R² is branched or unbranched, optionally substituted, C₁-C₁₀ acyclic acyl. In some embodiments, R² is substituted C₁-C₁₀ acyclic acyl. In some embodiments, R² is unsubstituted C₁-C₁₀ acyclic acyl.

In certain embodiments, R² is optionally substituted aryl. In some embodiments, R² is substituted aryl. In certain embodiments, R² is unsubstituted aryl. In some embodiments, R² is optionally substituted C₆-C₁₄ aryl. In certain embodiments, R² is substituted C₆-C₁₄ aryl. In some embodiments, R² is unsubstituted C₆-C₁₄ aryl.

In certain embodiments, R² is optionally substituted heteroaryl. In some embodiments, R² is substituted heteroaryl. In certain embodiments, R² is unsubstituted heteroaryl. In certain embodiments, R² is optionally substituted 5-14-membered heteroaryl. In some embodiments, R² is substituted 5-14-membered heteroaryl. In certain embodiments, R² is unsubstituted 5-14-membered heteroaryl.

In certain embodiments, R² is a nitrogen protecting group. In some embodiments, the nitrogen protecting group is Bn, Boc, Cbz, Fmoc, trifluoroacetyl, triphenylmethyl, acetyl, or Ts.

In some embodiments, one or both R¹ or R² are each independently joined to linker A together with the intervening atoms to form a linker comprising one or more N-containing heterocycles; or R¹ and R² are joined together with the intervening atoms to form a N-containing heterocycle. In certain embodiments, one or both R¹ or R² are each optionally independently joined to linker A together with the intervening atoms to form a linker comprising one or more N-containing heterocycles. In some embodiments, R¹ is joined to linker A together with the intervening atoms to form a linker comprising at least one N-containing heterocycle. In some embodiments, R^{B} is joined to linker A together with the intervening atoms to form a linker comprising at least one N-containing heterocycle. In some embodiments, R¹ and R² are joined together with the intervening atoms to form a N-containing heterocycle. In some embodiments, the N-containing heterocycle is a 3-8-membered N-containing heterocycle. In some embodiments, the N-containing heterocycle is a 5-7-membered N-containing heterocycle. In some embodiments, the N-containing heterocycle is a 5-membered N-containing heterocycle. In some embodiments, the N-containing heterocycle is a 6-membered N-containing heterocycle. In some embodiments, the N-containing heterocycle is a 7-membered N-containing heterocycle.

As defined herein, linker A comprises branched or unbranched, optionally substituted, cyclic or acyclic aliphatic; branched or unbranched, optionally substituted, cyclic or acyclic heteroaliphatic; optionally substituted arylene; or optionally substituted heteroarylene. As defined herein, linker A is branched or unbranched, optionally substituted, cyclic or acyclic aliphatic; branched or unbranched, optionally substituted, cyclic or acyclic heteroaliphatic; optionally substituted arylene; or optionally substituted heteroarylene.

In certain embodiments, linker A is branched or unbranched, optionally substituted, cyclic or acyclic aliphatic. In some embodiments, linker A is branched or unbranched, optionally substituted, cyclic aliphatic. In certain embodiments, linker A is substituted cyclic aliphatic. In some embodiments, linker is unsubstituted cyclic aliphatic. In certain embodiments, linker A is branched or unbranched, optionally substituted carbocyclylene. In some embodiments, linker A is substituted carbocyclylene. In certain embodiments, linker A is unsubstituted carbocyclylene. In certain embodiments, linker A is branched or unbranched, optionally substituted C₃-C₈ carbocyclylene. In some embodiments, linker A is substituted C₃-C₈ carbocyclylene. In certain embodiments, linker A is unsubstituted C₃-C₈ carbocyclylene.

In some embodiments, linker A is branched or unbranched, optionally substituted, acyclic aliphatic. In some embodiments, linker A is substituted acyclic aliphatic. In certain embodiments, linker A is unsubstituted acyclic aliphatic. In some embodiments, linker A is branched or unbranched, optionally substituted, alkylene. In some embodiments, linker A is substituted alkylene. In some embodiments, linker A is unsubstituted alkylene. In some embodiments, linker A is branched or unbranched, optionally substituted, C₁-C₁₀ alkylene. In some embodiments, linker A is substituted C₁-C₁₀ alkylene. In some embodiments, linker A is unsubstituted C₁-C₁₀ alkylene. In some embodiments, linker A is branched or unbranched, optionally substituted, alkenylene. In some embodiments, linker A is substituted alkenylene. In some embodiments, linker A is unsubstituted alkenylene. In some embodiments, linker A is branched or unbranched, optionally substituted, C₂-C₁₀ alkenylene. In some embodiments, linker A is substituted C₂-C₁₀ alkenylene. In some embodiments, linker A is unsubstituted C₂-C₁₀ alkenylene. In some embodiments, linker A is branched or unbranched, optionally substituted, alkynylene. In some embodiments, linker A is substituted alkynylene. In some embodiments, linker A is unsubstituted alkynylene. In some embodiments, linker A is branched or unbranched, optionally substituted, C₂-C₁₀ alkynylene. In some embodiments, linker A is substituted C₂-C₁₀ alkynylene. In some embodiments, linker A is unsubstituted C₂-C₁₀ alkynylene.

In certain embodiments, linker A is branched or unbranched, optionally substituted, cyclic or acyclic heteroaliphatic. In some embodiments, linker A is branched or unbranched, optionally substituted, cyclic heteroaliphatic. In certain embodiments, linker A is substituted cyclic heteroaliphatic. In some embodiments, linker is unsubstituted cyclic heteroaliphatic. In certain embodiments, linker A is branched or unbranched, optionally substituted heterocyclylene. In some embodiments, linker A is substituted heterocyclylene. In certain embodiments, linker A is unsubstituted heterocyclylene. In certain embodiments, linker A is branched or unbranched, optionally substituted 3-8-membered heterocyclylene. In some embodiments, linker A is substituted 3-8-membered heterocyclylene. In certain embodiments, linker A is unsubstituted 3-8-membered heterocyclylene.

In some embodiments, linker A is branched or unbranched, optionally substituted, acyclic heteroaliphatic. In some embodiments, linker A is substituted acyclic heteroaliphatic. In certain embodiments, linker A is unsubstituted acyclic heteroaliphatic. In some embodiments, linker A is branched or unbranched, optionally substituted, heteroalkylene. In some embodiments, linker A is substituted heteroalkylene. In some embodiments, linker A is unsubstituted heteroalkylene. In some embodiments, linker A is branched or unbranched, optionally substituted, C₁-C₁₀ heteroalkylene. In some embodiments, linker A is substituted C₁-C₁₀ heteroalkylene. In some embodiments, linker A is unsubstituted C₁-C₁₀ heteroalkylene. In some embodiments, linker A is branched or unbranched, optionally substituted, heteroalkenylene. In some embodiments, linker A is substituted heteroalkenylene. In some embodiments, linker A is unsubstituted heteroalkenylene. In some embodiments, linker A is branched or unbranched, optionally substituted, C₂-C₁₀ heteroalkenylene. In some embodiments, linker A is substituted C₂-C₁₀ heteroalkenylene. In some embodiments, linker A is unsubstituted C₂-C₁₀ heteroalkenylene. In some embodiments, linker A is branched or unbranched, optionally substituted, heteroalkynylene. In some embodiments, linker A is substituted heteroalkynylene. In some embodiments, linker A is unsubstituted heteroalkynylene. In some embodiments, linker A is branched or unbranched, optionally substituted, C₂-C₁₀ heteroalkynylene. In some embodiments, linker A is substituted C₂-C₁₀ heteroalkynylene. In some embodiments, linker A is unsubstituted C₂-C₁₀ heteroalkynylene.

In certain embodiments, linker A is optionally substituted arylene. In some embodiments, linker A is substituted arylene. In certain embodiments, linker A is unsubstituted arylene. In some embodiments, linker A is optionally substituted C₆-C₁₄ arylene. In certain embodiments, linker A is substituted C₆-C₁₄ arylene. In some embodiments, linker A is unsubstituted C₆-C₁₄ arylene.

In certain embodiments, linker A is optionally substituted heteroarylene. In some embodiments, linker A is substituted heteroarylene. In certain embodiments, linker A is unsubstituted heteroarylene. In certain embodiments, linker A is optionally substituted 5-14-membered heteroarylene. In some embodiments, linker A is substituted 5-14-membered heteroarylene. In certain embodiments, linker A is unsubstituted 5-14-membered heteroarylene.

In certain embodiments,

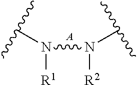

is selected from

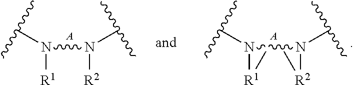

In some embodiments,

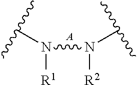

is

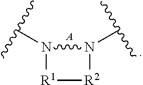

In certain embodiments,

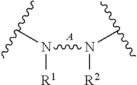

is

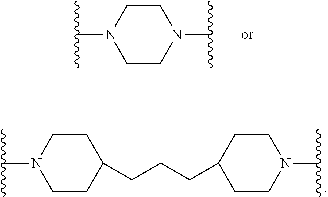 [SMILES: *N1CCN(*)CC1 ; *N1CCC(CCCC2CCN(*)CC2)CC1]

In certain embodiments,

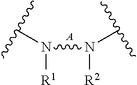

is

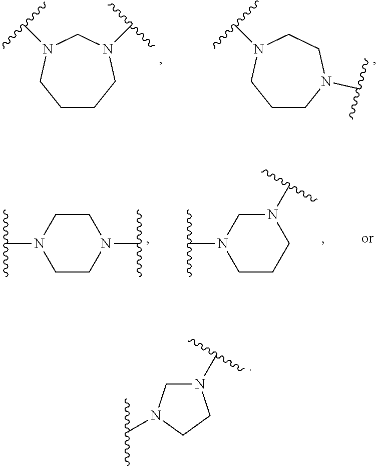 [SMILES: *N1CCCCN(*)C1 ; *N1CCCN(*)CC1 ; *N1CCN(*)CC1 ; *N1CCCN(*)C1 ; *N1CCN(*)C1]

In some embodiments,

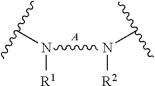

is

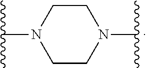 [SMILES: *N1CCN(*)CC1 ; "."]

In certain embodiments,

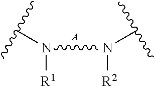

is

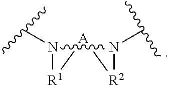

In some embodiments,

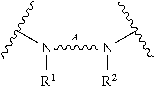

is

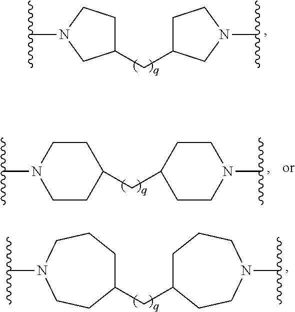

wherein each q is independently 1-10. In certain embodiments, q is 1-6. In some embodiments, q is 1-4. In certain embodiments, q is 1. In some embodiments, q is 2. In certain embodiments, q is 3. In some embodiments, q is 4. In certain embodiments, q is 5. In some embodiments, q is 6. In certain embodiments, q is 7. In some embodiments, q is 8. In certain embodiments, q is 9. In some embodiments, q is 10.

In certain embodiments,

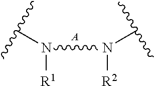

is

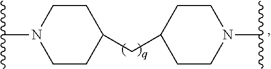

wherein each q is independently 1-10. In certain embodiments, q is 1-6. In some embodiments, q is 1-4. In certain embodiments, q is 1. In some embodiments, q is 2. In certain embodiments, q is 3. In some embodiments, q is 4. In certain embodiments, q is 5. In some embodiments, q is 6. In certain embodiments, q is 7. In some embodiments, q is 8. In certain embodiments, q is 9. In some embodiments, q is 10. In certain embodiments,

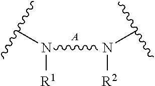

is

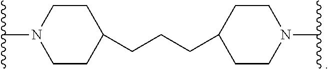 [SMILES: *N1CCC(CCCC2CCN(*)CC2)CC1 ; "."]

As defined herein, R³ is branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group.

In certain embodiments, R³ is branched or unbranched, optionally substituted, cyclic or acyclic aliphatic. In some embodiments, R³ is branched or unbranched, optionally substituted, cyclic aliphatic. In certain embodiments, R³ is substituted cyclic aliphatic. In some embodiments, linker is unsubstituted cyclic aliphatic. In certain embodiments, R³ is branched or unbranched, optionally substituted carbocyclylene. In some embodiments, R³ is substituted carbocyclylene. In certain embodiments, R³ is unsubstituted carbocyclylene. In certain embodiments, R³ is branched or unbranched, optionally substituted C₃-C₈ carbocyclylene. In some embodiments, R³ is substituted C₃-C₈ carbocyclylene. In certain embodiments, R³ is unsubstituted C₃-C₈ carbocyclylene. In some embodiments, R³ is branched or unbranched, optionally substituted, acyclic aliphatic. In some embodiments, R³ is substituted acyclic aliphatic. In certain embodiments, R³ is unsubstituted acyclic aliphatic. In some embodiments, R³ is branched or unbranched, optionally substituted, alkylene. In some embodiments, R³ is substituted alkylene. In some embodiments, R³ is unsubstituted alkylene. In some embodiments, R³ is branched or unbranched, optionally substituted, C₁-C₁₀ alkylene. In some embodiments, R³ is substituted C₁-C₁₀ alkylene. In some embodiments, R³ is unsubstituted C₁-C₁₀ alkylene. In some embodiments, R³ is branched or unbranched, optionally substituted, alkenylene. In some embodiments, R³ is substituted alkenylene. In some embodiments, R³ is unsubstituted alkenylene. In some embodiments, R³ is branched or unbranched, optionally substituted, C₂-C₁₀ alkenylene. In some embodiments, R³ is substituted C₂-C₁₀ alkenylene. In some embodiments, R³ is unsubstituted C₂-C₁₀ alkenylene. In some embodiments, R³ is branched or unbranched, optionally substituted, alkynylene. In some embodiments, R³ is substituted alkynylene. In some embodiments, R³ is unsubstituted alkynylene. In some embodiments, R³ is branched or unbranched, optionally substituted, C₂—C₁₀ alkynylene. In some embodiments, R³ is substituted C₂-C₁₀ alkynylene. In some embodiments, R³ is unsubstituted C₂-C₁₀ alkynylene.

In certain embodiments, R³ is branched or unbranched, optionally substituted, cyclic or acyclic heteroaliphatic. In some embodiments, R³ is branched or unbranched, optionally substituted, cyclic heteroaliphatic. In certain embodiments, R³ is substituted cyclic heteroaliphatic. In some embodiments, linker is unsubstituted cyclic heteroaliphatic. In certain embodiments, R³ is branched or unbranched, optionally substituted heterocyclylene. In some embodiments, R³ is substituted heterocyclylene. In certain embodiments, R³ is unsubstituted heterocyclylene. In certain embodiments, R³ is branched or unbranched, optionally substituted 3-8-membered heterocyclylene. In some embodiments, R³ is substituted 3-8-membered heterocyclylene. In certain embodiments, R³ is unsubstituted 3-8-membered heterocyclylene. In some embodiments, R³ is branched or unbranched, optionally substituted, acyclic heteroaliphatic. In some embodiments, R³ is substituted acyclic heteroaliphatic. In certain embodiments, R³ is unsubstituted acyclic heteroaliphatic. In some embodiments, R³ is branched or unbranched, optionally substituted, heteroalkylene. In some embodiments, R³ is substituted heteroalkylene. In some embodiments, R³ is unsubstituted heteroalkylene. In some embodiments, R³ is branched or unbranched, optionally substituted, C₁-C₁₀ heteroalkylene. In some embodiments, R³ is substituted C₁-C₁₀ heteroalkylene. In some embodiments, R³ is unsubstituted C₁-C₁₀ heteroalkylene. In some embodiments, R³ is branched or unbranched, optionally substituted, heteroalkenylene. In some embodiments, R³ is substituted heteroalkenylene. In some embodiments, R³ is unsubstituted heteroalkenylene. In some embodiments, R³ is branched or unbranched, optionally substituted, C₂-C₁₀ heteroalkenylene. In some embodiments, R³ is substituted C₂-C₁₀ heteroalkenylene. In some embodiments, R³ is unsubstituted C₂-C₁₀ heteroalkenylene. In some embodiments, R³ is branched or unbranched, optionally substituted, heteroalkynylene. In some embodiments, R³ is substituted heteroalkynylene. In some embodiments, R³ is unsubstituted heteroalkynylene. In some embodiments, R³ is branched or unbranched, optionally substituted, C₂-C₁₀ heteroalkynylene. In some embodiments, R³ is substituted C₂-C₁₀ heteroalkynylene. In some embodiments, R³ is unsubstituted C₂-C₁₀ heteroalkynylene.

In certain embodiments, R³ is branched or unbranched, optionally substituted, cyclic or acyclic acyl. In some embodiments, R³ is substituted cyclic or acyclic acyl. In some embodiments, R³ is unsubstituted cyclic or acyclic acyl. In certain embodiments, R³ is branched or unbranched, optionally substituted, cyclic acyl. In some embodiments, R³ is substituted cyclic acyl. In some embodiments, R³ is unsubstituted cyclic acyl. In certain embodiments, R³ is branched or unbranched, optionally substituted, C₅-C₁₀ cyclic acyl. In some embodiments, R³ is substituted C₅-C₁₀ cyclic acyl. In some embodiments, R³ is unsubstituted C₅-C₁₀ cyclic acyl. In certain embodiments, R³ is branched or unbranched, optionally substituted, acyclic acyl. In some embodiments, R³ is substituted acyclic acyl. In some embodiments, R³ is unsubstituted acyclic acyl. In certain embodiments, R³ is branched or unbranched, optionally substituted, C₁-C₁₀ acyclic acyl. In some embodiments, R³ is substituted C₁-C₁₀ acyclic acyl. In some embodiments, R³ is unsubstituted C₁-C₁₀ acyclic acyl.

In certain embodiments, R³ is optionally substituted aryl. In some embodiments, R³ is substituted aryl. In certain embodiments, R³ is unsubstituted aryl. In some embodiments, R³ is optionally substituted C₆-C₁₄ aryl. In certain embodiments, R³ is substituted C₆-C₁₄ aryl. In some embodiments, R³ is unsubstituted C₆-C₁₄ aryl.

In certain embodiments, R³ is optionally substituted heteroaryl. In some embodiments, R³ is substituted heteroaryl. In certain embodiments, R³ is unsubstituted heteroaryl. In certain embodiments, R³ is optionally substituted 5-14-membered heteroaryl. In some embodiments, R³ is substituted 5-14-membered heteroaryl. In certain embodiments, R³ is unsubstituted 5-14-membered heteroaryl.

In certain embodiments, R³ is a nitrogen protecting group. In some embodiments, the nitrogen protecting group is Bn, Boc, Cbz, Fmoc, trifluoroacetyl, triphenylmethyl, acetyl, or Ts.

As defined herein, linker B comprises branched or unbranched, optionally substituted, cyclic or acyclic aliphatic; branched or unbranched, optionally substituted, cyclic or acyclic heteroaliphatic; optionally substituted arylene; or optionally substituted heteroarylene. As defined herein, linker B is branched or unbranched, optionally substituted, cyclic or acyclic aliphatic; branched or unbranched, optionally substituted, cyclic or acyclic heteroaliphatic; optionally substituted arylene; or optionally substituted heteroarylene.

In certain embodiments, linker B is branched or unbranched, optionally substituted, cyclic or acyclic aliphatic. In some embodiments, linker B is branched or unbranched, optionally substituted, cyclic aliphatic. In certain embodiments, linker B is substituted cyclic aliphatic. In some embodiments, linker is unsubstituted cyclic aliphatic. In certain embodiments, linker B is branched or unbranched, optionally substituted carbocyclylene. In some embodiments, linker B is substituted carbocyclylene. In certain embodiments, linker B is unsubstituted carbocyclylene. In certain embodiments, linker B is branched or unbranched, optionally substituted C₃-C₈ carbocyclylene. In some embodiments, linker B is substituted C₃-C₈ carbocyclylene. In certain embodiments, linker B is unsubstituted C₃-C₈ carbocyclylene.

In some embodiments, linker B is branched or unbranched, optionally substituted, acyclic aliphatic. In some embodiments, linker B is substituted acyclic aliphatic. In certain embodiments, linker B is unsubstituted acyclic aliphatic. In some embodiments, linker B is branched or unbranched, optionally substituted, alkylene. In some embodiments, linker B is substituted alkylene. In some embodiments, linker B is unsubstituted alkylene. In some embodiments, linker B is branched or unbranched, optionally substituted, C₁-C₁₀ alkylene. In some embodiments, linker B is substituted C₁-C₁₀ alkylene. In some embodiments, linker B is unsubstituted C₁-C₁₀ alkylene. In some embodiments, linker B is branched or unbranched, optionally substituted, alkenylene. In some embodiments, linker B is substituted alkenylene. In some embodiments, linker B is unsubstituted alkenylene. In some embodiments, linker B is branched or unbranched, optionally substituted, C₂-C₁₀ alkenylene. In some embodiments, linker B is substituted C₂-C₁₀ alkenylene. In some embodiments, linker B is unsubstituted C₂-C₁₀ alkenylene. In some embodiments, linker B is branched or unbranched, optionally substituted, alkynylene. In some embodiments, linker B is substituted alkynylene. In some embodiments, linker B is unsubstituted alkynylene. In some embodiments, linker B is branched or unbranched, optionally substituted, C₂-C₁₀ alkynylene. In some embodiments, linker B is substituted C₂-C₁₀ alkynylene. In some embodiments, linker B is unsubstituted C₂-C₁₀ alkynylene.

In certain embodiments, linker B is branched or unbranched, optionally substituted, cyclic or acyclic heteroaliphatic. In some embodiments, linker B is branched or unbranched, optionally substituted, cyclic heteroaliphatic. In certain embodiments, linker B is substituted cyclic heteroaliphatic. In some embodiments, linker is unsubstituted cyclic heteroaliphatic. In certain embodiments, linker B is branched or unbranched, optionally substituted heterocyclylene. In some embodiments, linker B is substituted heterocyclylene. In certain embodiments, linker B is unsubstituted heterocyclylene. In certain embodiments, linker B is branched or unbranched, optionally substituted 3-8-membered heterocyclylene. In some embodiments, linker B is substituted 3-8-membered heterocyclylene. In certain embodiments, linker B is unsubstituted 3-8-membered heterocyclylene.

In some embodiments, linker B is branched or unbranched, optionally substituted, acyclic heteroaliphatic. In some embodiments, linker B is substituted acyclic heteroaliphatic. In certain embodiments, linker B is unsubstituted acyclic heteroaliphatic. In some embodiments, linker B is branched or unbranched, optionally substituted, heteroalkylene. In some embodiments, linker B is substituted heteroalkylene. In some embodiments, linker B is unsubstituted heteroalkylene. In some embodiments, linker B is branched or unbranched, optionally substituted, C₁-C₁₀ heteroalkylene. In some embodiments, linker B is substituted C₁-C₁₀ heteroalkylene. In some embodiments, linker B is unsubstituted C₁-C₁₀ heteroalkylene. In some embodiments, linker B is branched or unbranched, optionally substituted, heteroalkenylene. In some embodiments, linker B is substituted heteroalkenylene. In some embodiments, linker B is unsubstituted heteroalkenylene. In some embodiments, linker B is branched or unbranched, optionally substituted, C₂-C₁₀ heteroalkenylene. In some embodiments, linker B is substituted C₂-C₁₀ heteroalkenylene. In some embodiments, linker B is unsubstituted C₂-C₁₀ heteroalkenylene. In some embodiments, linker B is branched or unbranched, optionally substituted, heteroalkynylene. In some embodiments, linker B is substituted heteroalkynylene. In some embodiments, linker B is unsubstituted heteroalkynylene. In some embodiments, linker B is branched or unbranched, optionally substituted, C₂-C₁₀ heteroalkynylene. In some embodiments, linker B is substituted C₂-C₁₀ heteroalkynylene. In some embodiments, linker B is unsubstituted C₂-C₁₀ heteroalkynylene.

In certain embodiments, linker B is optionally substituted arylene. In some embodiments, linker B is substituted arylene. In certain embodiments, linker B is unsubstituted arylene. In some embodiments, linker B is optionally substituted C₆-C₁₄ arylene. In certain embodiments, linker B is substituted C₆-C₁₄ arylene. In some embodiments, linker B is unsubstituted C₆-C₁₄ arylene.

In certain embodiments, linker B is optionally substituted heteroarylene. In some embodiments, linker B is substituted heteroarylene. In certain embodiments, linker B is unsubstituted heteroarylene. In certain embodiments, linker B is optionally substituted 5-14-membered heteroarylene. In some embodiments, linker B is substituted 5-14-membered heteroarylene. In certain embodiments, linker B is unsubstituted 5-14-membered heteroarylene.

As defined herein, R⁴ is hydrogen, branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group; or R⁴ is joined to linker B together with the intervening atoms to form a linker comprising a N-containing heterocycle. In certain embodiments, R⁴ is hydrogen, branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group.

In some embodiments, R⁴ is hydrogen.

In certain embodiments, R⁴ is branched or unbranched, optionally substituted, cyclic or acyclic aliphatic. In some embodiments, R⁴ is branched or unbranched, optionally substituted, cyclic aliphatic. In certain embodiments, R⁴ is substituted cyclic aliphatic. In some embodiments, linker is unsubstituted cyclic aliphatic. In certain embodiments, R⁴ is branched or unbranched, optionally substituted carbocyclylene. In some embodiments, R⁴ is substituted carbocyclylene. In certain embodiments, R⁴ is unsubstituted carbocyclylene. In certain embodiments, R⁴ is branched or unbranched, optionally substituted C₃-C₈ carbocyclylene. In some embodiments, R⁴ is substituted C₃-C₈ carbocyclylene. In certain embodiments, R⁴ is unsubstituted C₃-C₈ carbocyclylene. In some embodiments, R⁴ is branched or unbranched, optionally substituted, acyclic aliphatic. In some embodiments, R⁴ is substituted acyclic aliphatic. In certain embodiments, R⁴ is unsubstituted acyclic aliphatic. In some embodiments, R⁴ is branched or unbranched, optionally substituted, alkylene. In some embodiments, R⁴ is substituted alkylene. In some embodiments, R⁴ is unsubstituted alkylene. In some embodiments, R⁴ is branched or unbranched, optionally substituted, C₁-C₁₀ alkylene. In some embodiments, R⁴ is substituted C₁-C₁₀ alkylene. In some embodiments, R⁴ is unsubstituted C₁-C₁₀ alkylene. In some embodiments, R⁴ is branched or unbranched, optionally substituted, alkenylene. In some embodiments, R⁴ is substituted alkenylene. In some embodiments, R⁴ is unsubstituted alkenylene. In some embodiments, R⁴ is branched or unbranched, optionally substituted, C₂-C₁₀ alkenylene. In some embodiments, R⁴ is substituted C₂-C₁₀ alkenylene. In some embodiments, R⁴ is unsubstituted C₂-C₁₀ alkenylene. In some embodiments, R⁴ is branched or unbranched, optionally substituted, alkynylene. In some embodiments, R⁴ is substituted alkynylene. In some embodiments, R⁴ is unsubstituted alkynylene. In some embodiments, R⁴ is branched or unbranched, optionally substituted, C₂-C₁₀ alkynylene. In some embodiments, R⁴ is substituted C₂-C₁₀ alkynylene. In some embodiments, R⁴ is unsubstituted C₂-C₁₀ alkynylene.

In certain embodiments, R⁴ is branched or unbranched, optionally substituted, cyclic or acyclic heteroaliphatic. In some embodiments, R⁴ is branched or unbranched, optionally substituted, cyclic heteroaliphatic. In certain embodiments, R⁴ is substituted cyclic heteroaliphatic. In some embodiments, linker is unsubstituted cyclic heteroaliphatic. In certain embodiments, R⁴ is branched or unbranched, optionally substituted heterocyclylene. In some embodiments, R⁴ is substituted heterocyclylene. In certain embodiments, R⁴ is unsubstituted heterocyclylene. In certain embodiments, R⁴ is branched or unbranched, optionally substituted 3-8-membered heterocyclylene. In some embodiments, R⁴ is substituted 3-8-membered heterocyclylene. In certain embodiments, R⁴ is unsubstituted 3-8-membered heterocyclylene. In some embodiments, R⁴ is branched or unbranched, optionally substituted, acyclic heteroaliphatic. In some embodiments, R⁴ is substituted acyclic heteroaliphatic. In certain embodiments, R⁴ is unsubstituted acyclic heteroaliphatic. In some embodiments, R⁴ is branched or unbranched, optionally substituted, heteroalkylene. In some embodiments, R⁴ is substituted heteroalkylene. In some embodiments, R⁴ is unsubstituted heteroalkylene. In some embodiments, R⁴ is branched or unbranched, optionally substituted, C₁-C₁₀ heteroalkylene. In some embodiments, R⁴ is substituted C₁-C₁₀ heteroalkylene. In some embodiments, R⁴ is unsubstituted C₁-C₁₀ heteroalkylene. In some embodiments, R⁴ is branched or unbranched, optionally substituted, heteroalkenylene. In some embodiments, R⁴ is substituted heteroalkenylene. In some embodiments, R⁴ is unsubstituted heteroalkenylene. In some embodiments, R⁴ is branched or unbranched, optionally substituted, C₂-C₁₀ heteroalkenylene. In some embodiments, R⁴ is substituted C₂-C₁₀ heteroalkenylene. In some embodiments, R⁴ is unsubstituted C₂-C₁₀ heteroalkenylene. In some embodiments, R⁴ is branched or unbranched, optionally substituted, heteroalkynylene. In some embodiments, R⁴ is substituted heteroalkynylene. In some embodiments, R⁴ is unsubstituted heteroalkynylene. In some embodiments, R⁴ is branched or unbranched, optionally substituted, C₂-C₁₀ heteroalkynylene. In some embodiments, R⁴ is substituted C₂-C₁₀ heteroalkynylene. In some embodiments, R⁴ is unsubstituted C₂-C₁₀ heteroalkynylene.

In certain embodiments, R⁴ is branched or unbranched, optionally substituted, cyclic or acyclic acyl. In some embodiments, R⁴ is substituted cyclic or acyclic acyl. In some embodiments, R⁴ is unsubstituted cyclic or acyclic acyl. In certain embodiments, R⁴ is branched or unbranched, optionally substituted, cyclic acyl. In some embodiments, R⁴ is substituted cyclic acyl. In some embodiments, R⁴ is unsubstituted cyclic acyl. In certain embodiments, R⁴ is branched or unbranched, optionally substituted, C₅-C₁₀ cyclic acyl. In some embodiments, R⁴ is substituted C₅-C₁₀ cyclic acyl. In some embodiments, R⁴ is unsubstituted C₅-C₁₀ cyclic acyl. In certain embodiments, R⁴ is branched or unbranched, optionally substituted, acyclic acyl. In some embodiments, R⁴ is substituted acyclic acyl. In some embodiments, R⁴ is unsubstituted acyclic acyl. In certain embodiments, R⁴ is branched or unbranched, optionally substituted, C₁-C₁₀ acyclic acyl. In some embodiments, R⁴ is substituted C₁-C₁₀ acyclic acyl. In some embodiments, R⁴ is unsubstituted C₁-C₁₀ acyclic acyl.

In certain embodiments, R⁴ is optionally substituted aryl. In some embodiments, R⁴ is substituted aryl. In certain embodiments, R⁴ is unsubstituted aryl. In some embodiments, R⁴ is optionally substituted C₆-C₁₄ aryl. In certain embodiments, R⁴ is substituted C₆-C₁₄ aryl. In some embodiments, R⁴ is unsubstituted C₆-C₁₄ aryl.

In certain embodiments, R⁴ is optionally substituted heteroaryl. In some embodiments, R⁴ is substituted heteroaryl. In certain embodiments, R⁴ is unsubstituted heteroaryl. In certain embodiments, R⁴ is optionally substituted 5-14-membered heteroaryl. In some embodiments, R⁴ is substituted 5-14-membered heteroaryl. In certain embodiments, R⁴ is unsubstituted 5-14-membered heteroaryl.

In certain embodiments, R⁴ is a nitrogen protecting group. In some embodiments, the nitrogen protecting group is Bn, Boc, Cbz, Fmoc, trifluoroacetyl, triphenylmethyl, acetyl, or Ts.

In some embodiments, R⁴ is joined to linker B together with the intervening atoms to form a linker comprising a N-containing heterocycle. In some embodiments, the N-containing heterocycle is a 3-8-membered N-containing heterocycle. In some embodiments, the N-containing heterocycle is a 5-7-membered N-containing heterocycle. In some embodiments, the N-containing heterocycle is a 5-membered N-containing heterocycle. In some embodiments, the N-containing heterocycle is a 6-membered N-containing heterocycle.

In some embodiments, the N-containing heterocycle is a 7-membered N-containing heterocycle.

In certain embodiments, the one or more compounds of formula (v), (vi), or (vii) are selected from

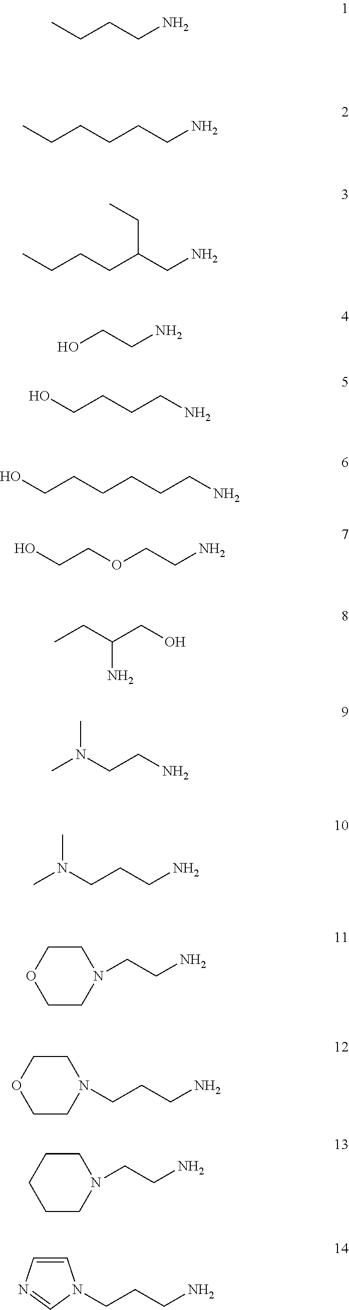

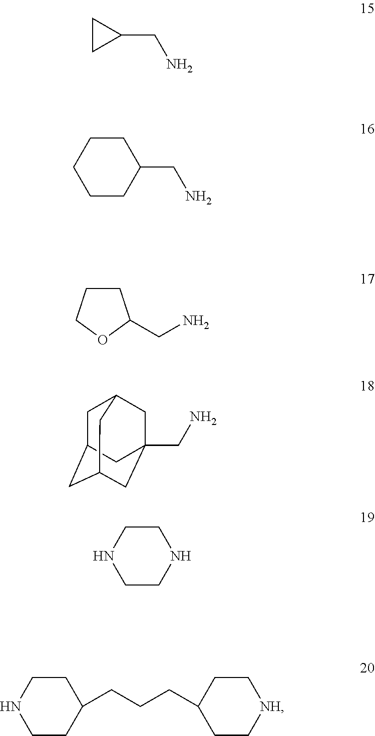

or a salt, isotope, or stereoisomer thereof.

In certain embodiments, one of the one or more compounds of formula (v) is H N NH

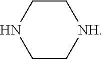

In certain embodiments, one of the one or more compounds of formula (v) is

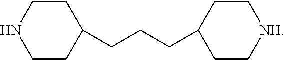 [SMILES: C(CC1CCNCC1)CC1CCNCC1]

### EXAMPLES

### Example 1

To protect Vitamin A through storage, transport, and food preparation an encapsulation strategy was developed that results in natural degradation products, and therefore may have stronger resonance amongst food producers who were reticent to commercialize products with BMC, which is considered a microplastic.

A natural product-based polymer, poly(O-amino ester) (PAE), was developed with similar micronutrient encapsulation, protection, and release properties as BMC. Unlike BMC, PAE degrades into two natural product-like small molecules under boiling water condition, which circumvents potential issues regarding microplastics. Five compositions of PAE were evaluated and achieved over 80% vitamin A (VA) recovery after a two-hour boiling, with over 90% of VA released after 10-minutes and over 99% of VA released after 30-minutes of treatment with simulated gastric fluid (SGF), and >46% recovery after long-term storage (4 months) under heated (40° C.) and humidified (75%) conditions. The MPs achieved encapsulation, robust stabilization, and efficiency release of multiple micronutrients with various water or oil solubility using the PAE MPs.

PAE-based MPs are fully degradable in boiling water, affording two natural product-based small molecules. PAE degraded into isosorbide and a beta amino acid. Isosorbide is classified as a sugar derivative and approved by the FDA as a GRAS material. There have been several examples of beta amino acid usage in food industry (e.g., beta-alanine (CarnoSyn®) and p-aminobenzoic acid). The following studies characterized the PAE-based micronutrient delivery platform.

### Characterization of PAE MP Physiochemical Properties and PAE Encapsulation and Protection Mechanisms

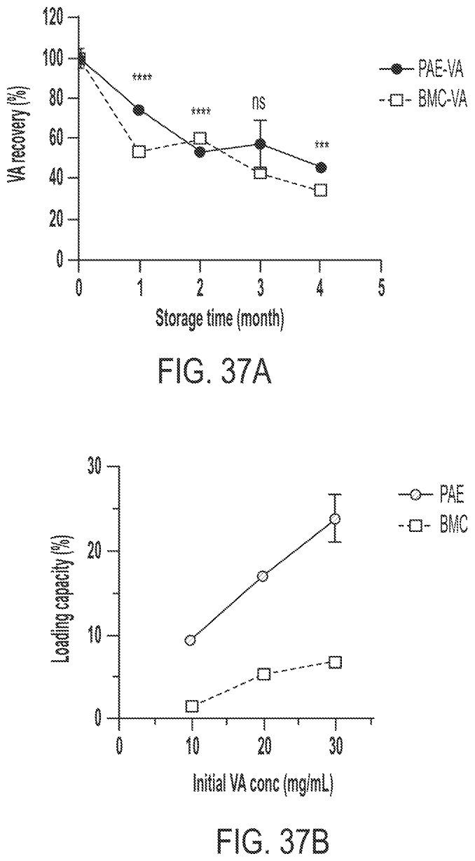

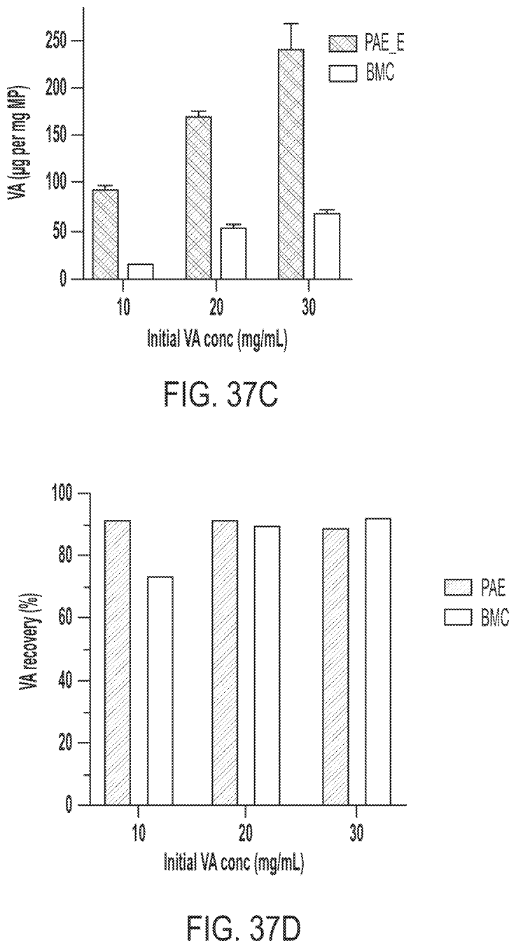

The encapsulation and protection efficiency of VA depended mainly on the physicochemical properties of PAE and PAE MP, which included (1) molecular weight (MW), hydrophobicity, chain-end functionality, glass and melting temperatures; and (2) VA distribution across MP, surface smoothness, and porosity. VA stability experiments showed that the increase in PAE hydrophobicity, which corresponds to the high ratio of 4,4′-trimethylenedipiperidine (TDP) in PAE composition, can significantly improve VA recovery under boiling water condition. Results regarding VA encapsulation and protection mechanism suggest that hydrophobic interactions between PAE and VA molecules drive high VA encapsulation and recovery. Therefore, the effect of MW, polymer compositions, functionality of polymer chain end, and hydrophobicity on VA-PAE MP stability were determined using gel permeation chromatograph (GPC), nuclear magnetic resonance (NMR), differential scanning calorimetry (DSC), and water contact angle. PAE encapsulation (e.g., loading capacity of VA, FIG. 37C) and protection properties (e.g., VA recovery after 2-hour boiling in water, FIG. 37D) were compared to that of BMC. Properties were determined using scanning electron microscope (SEM) and multiphoton excitation fluorescence microscopy.

### Stabilization and Controlled Release of VA

To evaluate the effects on VA stabilization, VA (10% wt) was encapsulated with MPs fabricated from PAE_C, PAE_D, or PAE_E. The resulting MPs were tested in boiling water for 2 hours. All three forms of PAE significantly enhanced the stability of VA by 7 to 14-fold (44%-83% recovery with encapsulation vs 6% recovery without encapsulation). Increased hydrophobicity of PAE provided improved stabilization ability with PAE_E showing the highest recovery of VA (83%). Without wishing to be bound by theory, the correlation between PAE hydrophobicity and stabilization ability could be attributed to the elevated molecular interactions between PAE and VA since the latter is also hydrophobic. On the other hand, SEM images showed that the rigidity of PAE_C, PAE_D, and PAE_E microparticle structure increased with hydrophobicity, which could also contribute to PAE_E providing highest protection for VA at the microscopic level. A time-course experiment further supported the stabilization ability of PAE_E encapsulating VA, as over 90% of VA was recovered after boiling for 0.5 h, in contrast to the unencapsulated VA where less than 20% were recovered. Therefore, PAE_E was selected as the most promising candidate to proceed with further characterization.

Protection efficiency of VA by PAE_E MPs against environmental oxidation was assessed. BHT is a free radical scavenger commonly used as food additives to protect against oxidation. For comparison, MPs were synthesized by adding 0.5% wt BHT to the PAE_E MP formulation. The results showed that no significant differences were observed for VA recovery between PAE_E MPs with and without BHT addition. These results demonstrated that PAE_E MPs provided high protection against oxidation such that no antioxidant additive is necessary to achieve high VA recovery.

The effect of % VA loading on its protection efficiency was investigated. Consistent VA recovery rate (>80%) after 2-hour boiling was observed across varied VA loadings (5% wt to 30% wt) in PAE_E MPs. The PAE MPs exhibit high loading capacity and protection efficiency independent of the loading quantity.

Given the high stabilization of VA by PAE_E MP, release of VA was evaluated under three conditions: room-temperature water, boiling water, and SGF at 37° C., which simulates human stomach environment. Controlled release is defined as release under only the SGF condition. PAE_E-VA MP samples were incubated under each condition for different time points. No significant VA release was observed under room-temperature or boiling water conditions up to 2 hours. By contrast, VA was rapidly released in SGF at 37° C. where over 90% of encapsulated VA was detected in the supernatant after 10-minute treatment. PAE_E MPs dissolved quickly under these conditions but not the other two water conditions. The results suggest that upon oral consumption, the pH-sensitivity of PAE_E material will allow quick dissociation between the microparticles and VA molecules, which may afford high bioavailability in human digestion system.

### Degradation

While PAE MP showed high VA recovery after cooking, PAE degradation profile, as determined by NMR, showed that less than 10% of polymer was left in boiled products after two hours with no polymer detected after four hours. The degradation rate of PAE polymer characterized by NMR did not quantify amount of oligomer.

GPC was used to further characterize the degradation rate, which is part of protection mechanism study. The protection mechanism of PAE MP during boiling and room temperature was investigated to explain how PAE can render high protection level of VA while undergoing degradation using GPC and SEM. Since the polymer is almost fully degraded during boiling there could be a potentially added safety benefit because little polymer is left to be ingested.

### Assessing the Long-Term Stability of VA-PAE MP

The large-scale implementation of the developed food fortification technology in the targeted regions is limited by local cooking practices and long-term storage, which negatively impact VA bioactivity. In the following studies, the MP samples were exposed to open air with controlled temperature and humidity without sealing. Testing was conducted for the improvement of VA stability under an accelerated storage condition (40° C., 75% humidity). A nine-month storage stability experiment of the PAE-VA MP was conducted under these conditions. At the end of each month, the stored MPs were evaluated for the recovery of VA (i) directly after storage and (ii) with an additional step of boiling in water for 2 hours. Compared to unencapsulated VA, which showed complete degradation within the first month of storage, PAE_E MP retained 75% VA recovery after one month of storage. While the overall recovery percentage of VA declined over the storage period, PAE_E MP still retained 46% VA recovery after four months of storage. Nonetheless, these recovery levels may still be beneficial to patients with micronutrient deficiencies.

Since PAE_E MP provided sufficient protection against oxidation without the necessity for antioxidant additives, the persistence of this protection efficiency was evaluated over storage periods. 0.5% of butylated hydroxytoluene was added to another set of formulations to investigate the effect of adding antioxidant on the long-term stability of PAE-VA MP. For bench-scale production, VA palmitate itself without BHT was used. No significant difference in VA recovery was observed between PAE_E MP with and without BHT additives, demonstrating the robust protection of PAE_E MP against oxidation. For example, the first and second month time points revealed that both formulations exhibited comparable protection efficiency as BMC under the same storage condition.

Different loading percentages of VA were evaluated for effect on recovery over the storage period. The PAE_E MP encapsulation effect was extended over a 4-month storage period. No significant difference was observed between the VA recovery rate from 10% wt and 15% wt VA loading encapsulation.

Given the tolerance of PAE_E MP to micronutrient loading, its ability to stabilize multiple encapsulated micronutrients was evaluated under long-term storage conditions. To optimize VA protection by PAE MP, vitamin E (VE) is an alternative to BHT. VE is a natural micronutrient and can serve as an antioxidant. PAE-VA MP was modified to increase VE loading ratio up to 10% to substitute BHT and confer better stability during long-term storage. The hydrophobic nature of VE molecule enabled easy and straightforward encapsulation by PAE along with VA. For PAE_E MPs encapsulated with 10% wt VA and 10% wt vitamin E (VE), 27% of VA was recovered after 4 months of storage.

PAE_E was evaluated for protection of VA under boiling conditions after the long-term storage. Compared to unencapsulated VA which did not survive boiling conditions, PAE_E MP retained 43% VA recovery after 1-month storage followed by 2-hour of boiling. Consistent with the general decline of VA recovery rate with prolonged storage periods, VA recovery also presented a decreasing trend when tested after storage followed by boiling conditions. BHT addition, increased VA loading, and VE co-encapsulation with VA were tolerated when measuring VA recovery after storage followed by boiling conditions. The protection efficiency of PAE_E MP was calculated as the ratio between the recovered VA before and after boiling conditions. The protection efficiency is indicative of whether PAE_E MP lost its protective ability against boiling conditions during the storage period. The protection efficiency of PAE_E MP declined from 80% to 40% over 4-moth storage, compared to >80% at starting point of the storage.

A mixture of starch with MP improves long-term stability of VA by absorbing water vapor, creating a relatively dry environment for MP sample under high humidity conditions. These formulations undergo long-term storage studies (9-12 months) alone and with the other MNs included in the bouillon cube alongside Vitamin A, such as B9, B12, and iodine. Moreover, the impact of diamino acid on food matrix components is assessed.

### Evaluation of Other Micronutrients Encapsulation and Protection by PAE MP

As high loading and enhanced protection of VA using the PAE-based platform described herein was achieved, the PAE_E MP was evaluated for encapsulation of other vital micronutrients for stabilization and controlled release. Hydrophobic micronutrients, such as vitamin C (VC), vitamin D (VD), iron, B2, B9, B12, K1, K3, niacin, zinc, iodine and vitamin E (VE), were individually encapsulated into PAE MP to test their boiling stability. A two-step oil/water emulsion method was applied to hydrophilic micronutrient protection, in which different types of excipients are needed to form smaller microparticles, followed by encapsulation.

VD and VE (10% wt) were encapsulated with PAE_E MP. Like VA, VD and VE are both oil-soluble vitamins. VD is thermally labile, while VE is thermally stable. After incubating at boiling water for 2 hours, over 80% of VD and VE were recovered, compared to less than 5% recovery of free-form VD. In the controlled release experiment by treating the MP with SGF, both micronutrients were released with comparable efficiency (>80% release) as VA.

Next, PAE_E MP was evaluated for encapsulation and stabilization of hydrophilic micronutrients, such as vitamin C palmitate (VCP). VCP is a fat-soluble ester form of vitamin C (VC; ascorbic acid) which retains the biological functions of VC. Using the same fabrication method, VCP was encapsulated with PAE_E MP. After 2-hour boiling, over 46% of VCP was recovered, compared to no detectable recovery of free-form VCP 30 minutes after boiling condition.

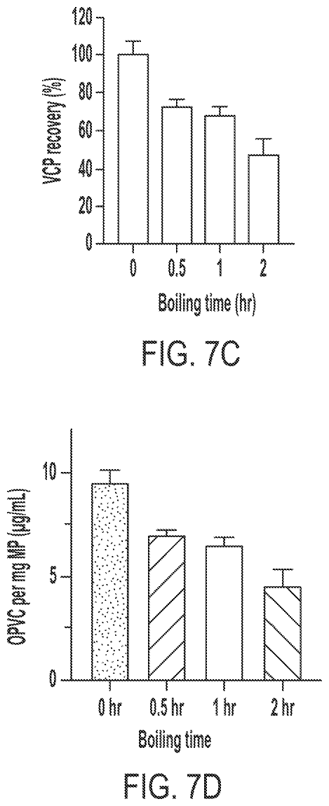

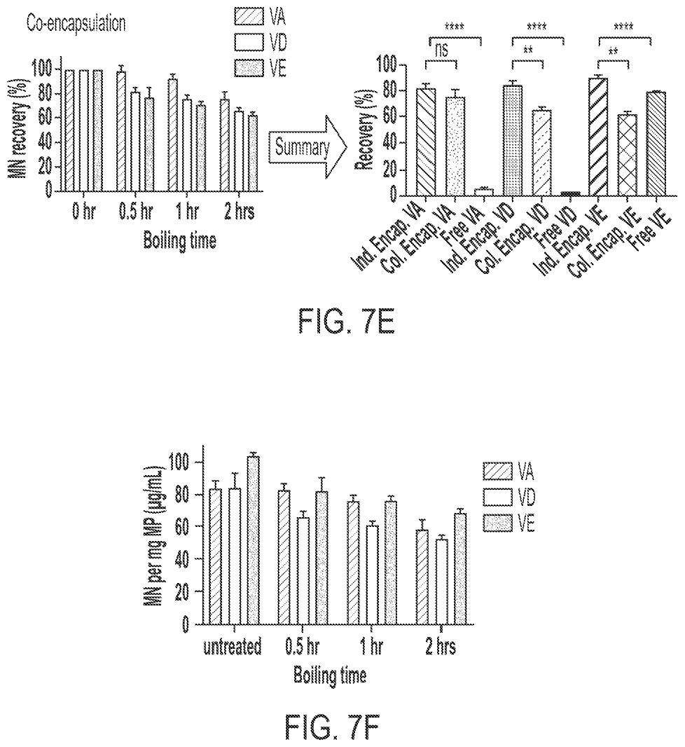

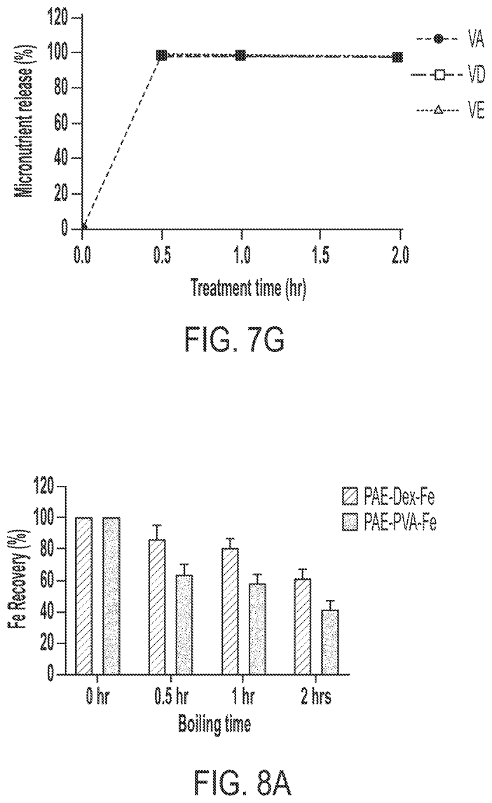

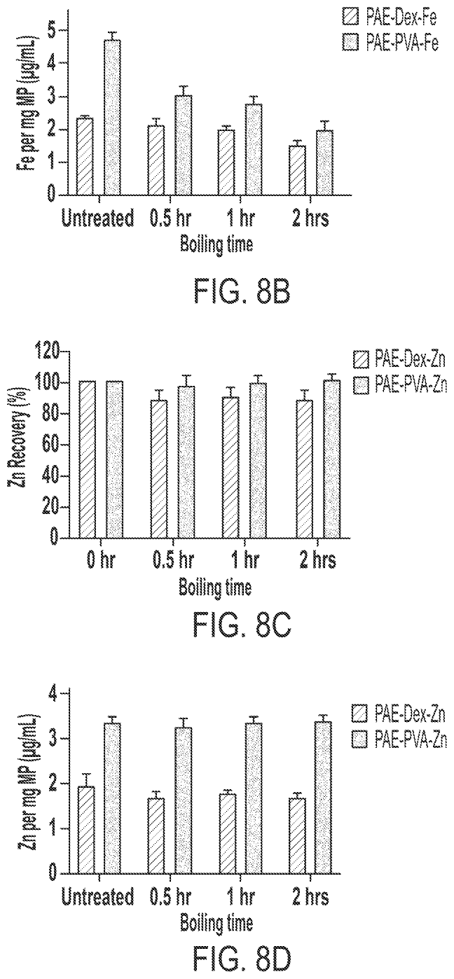

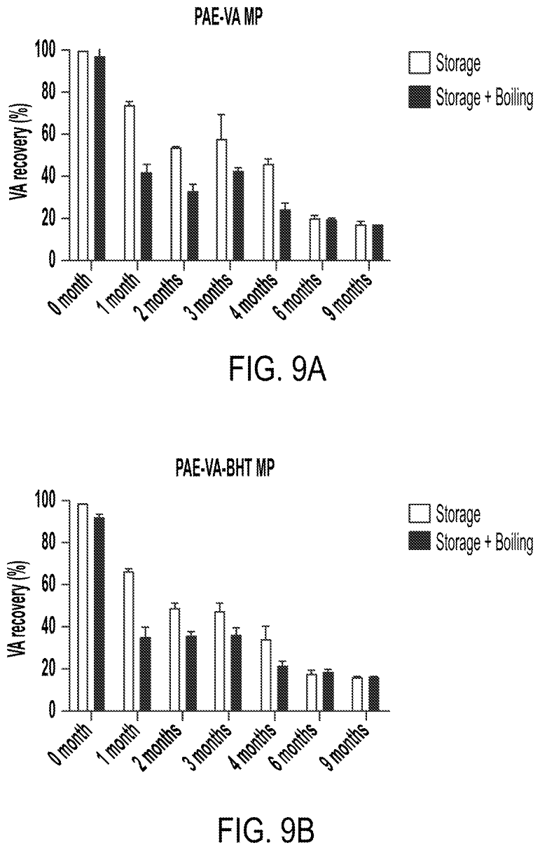

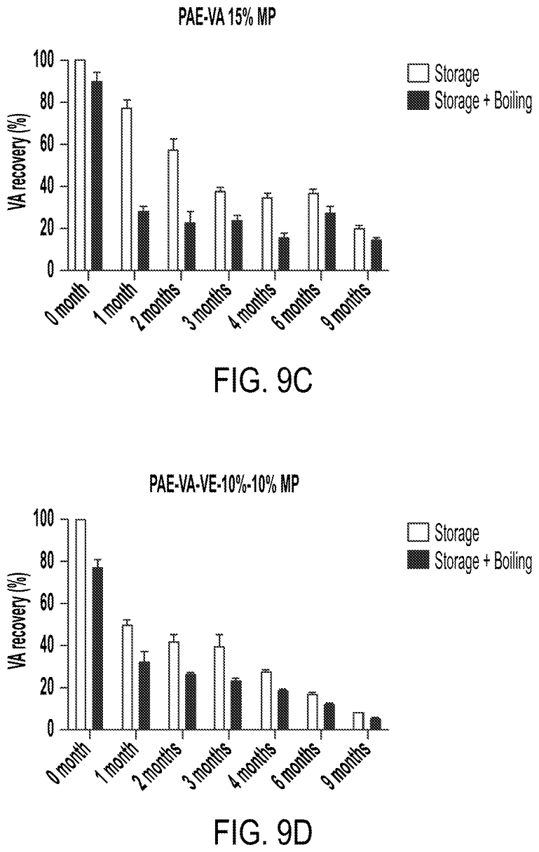

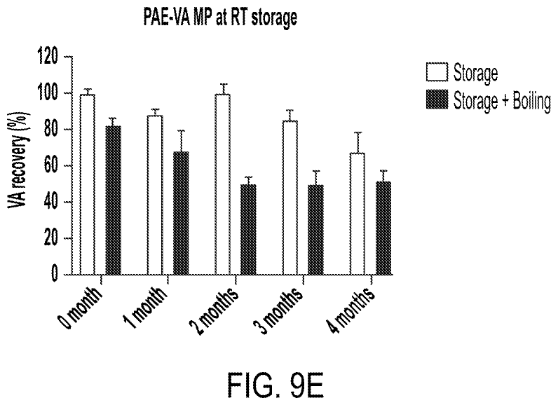

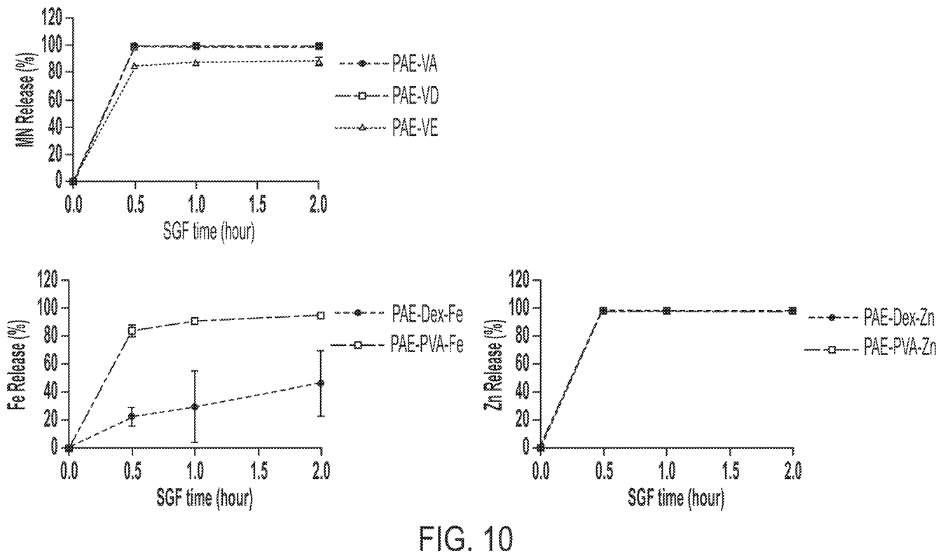

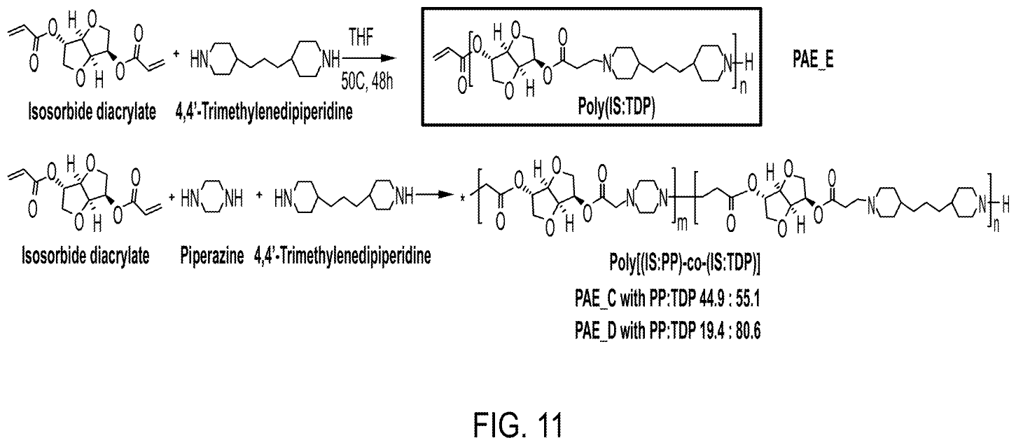

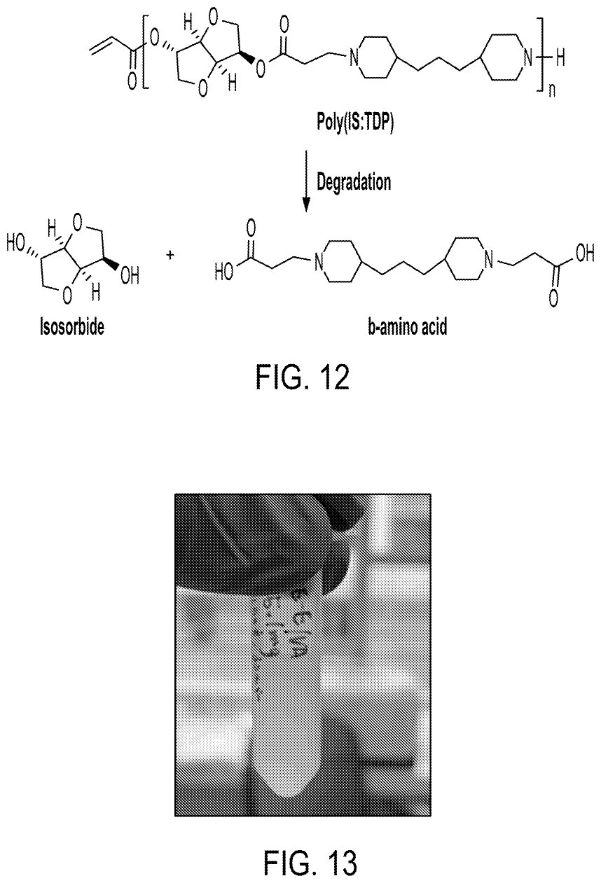

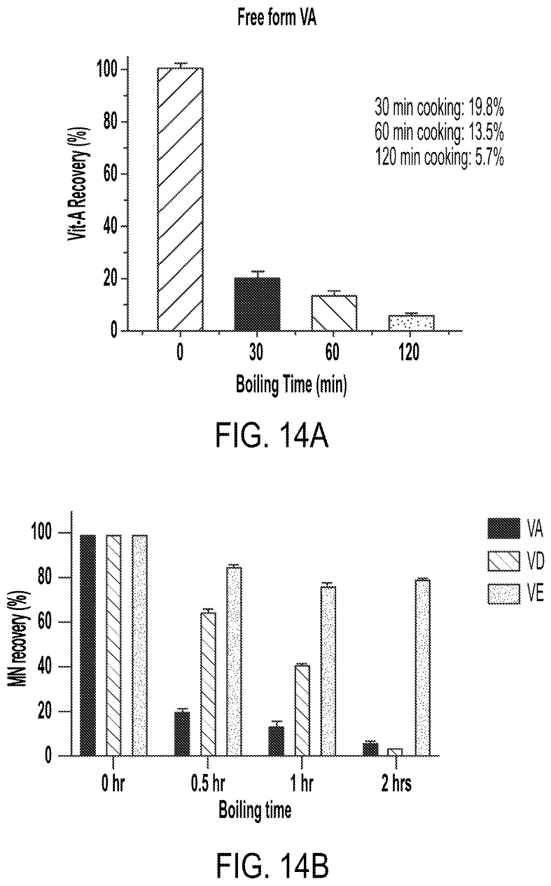

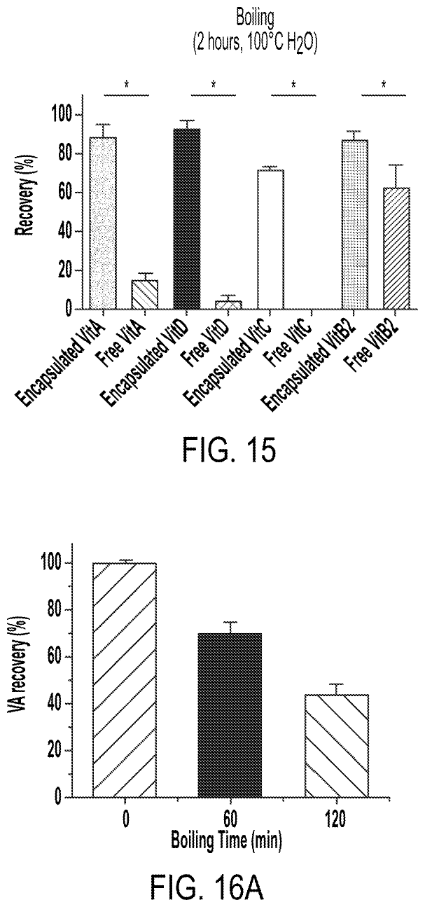

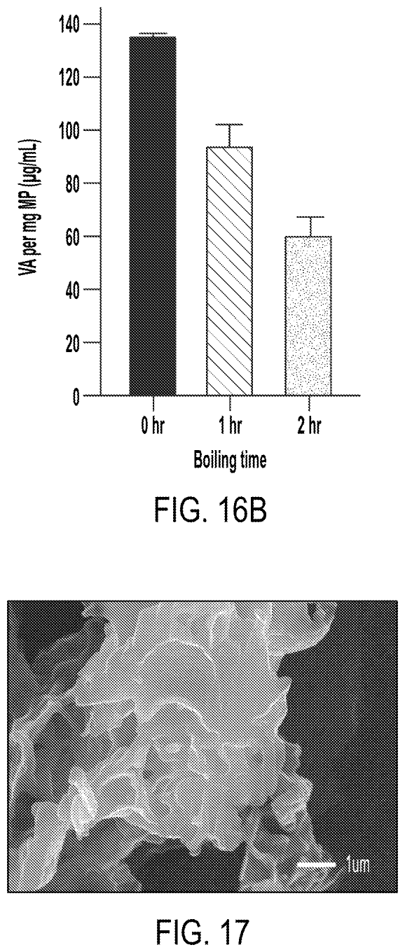

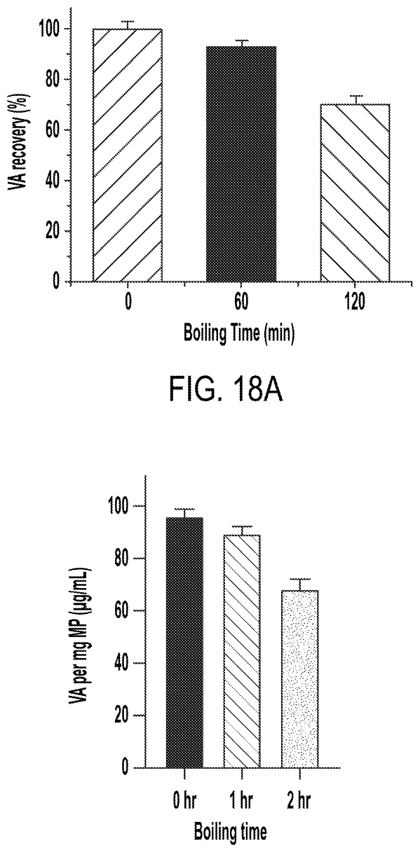

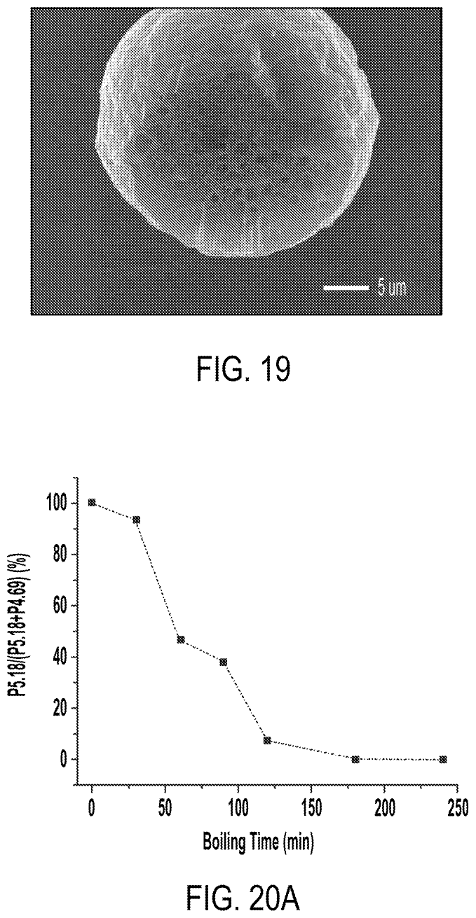

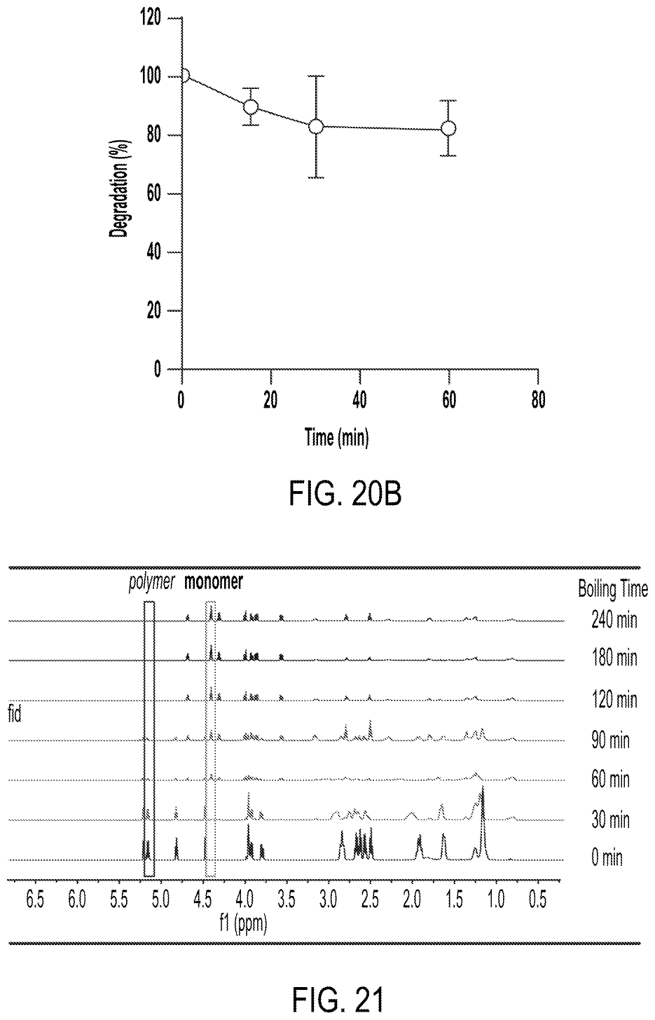

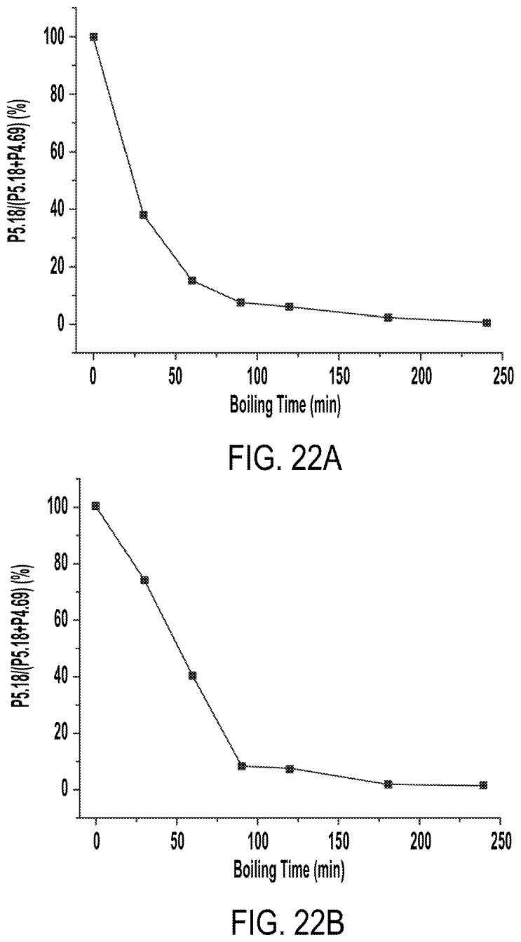

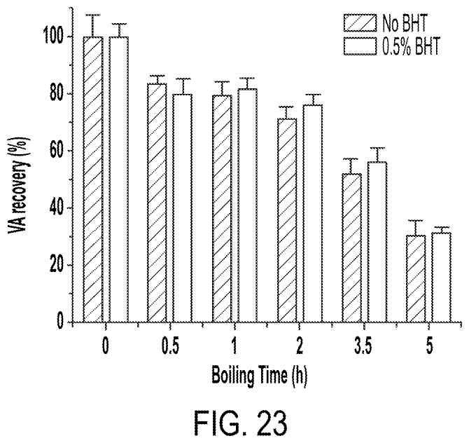

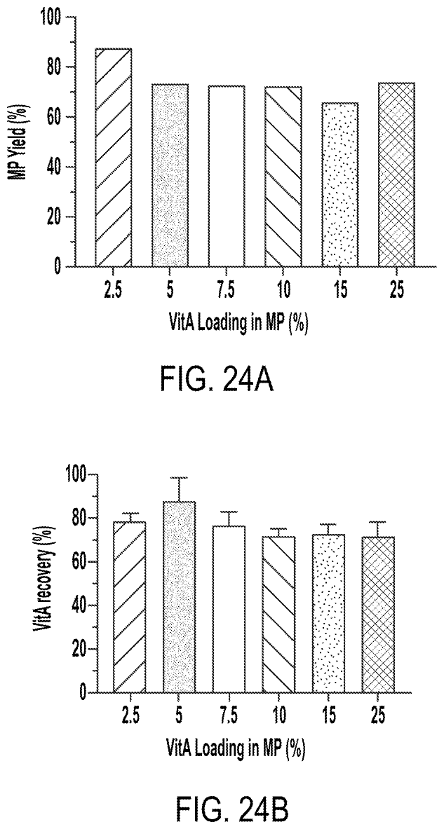

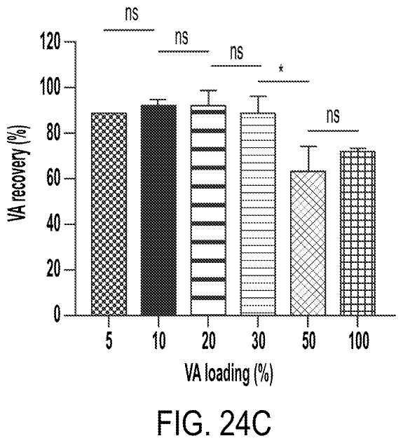

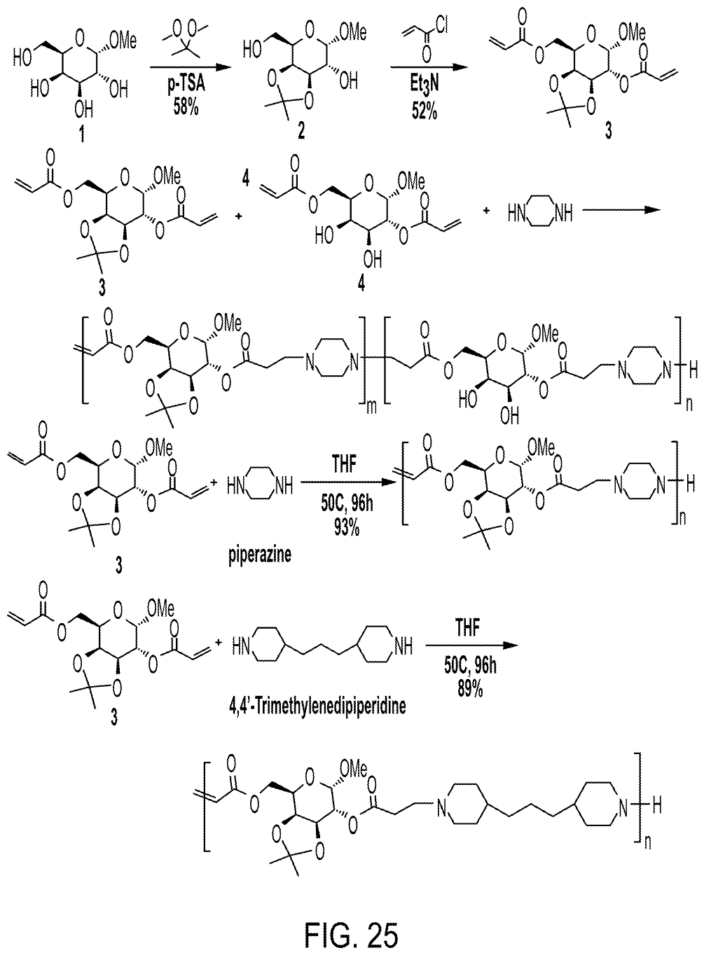

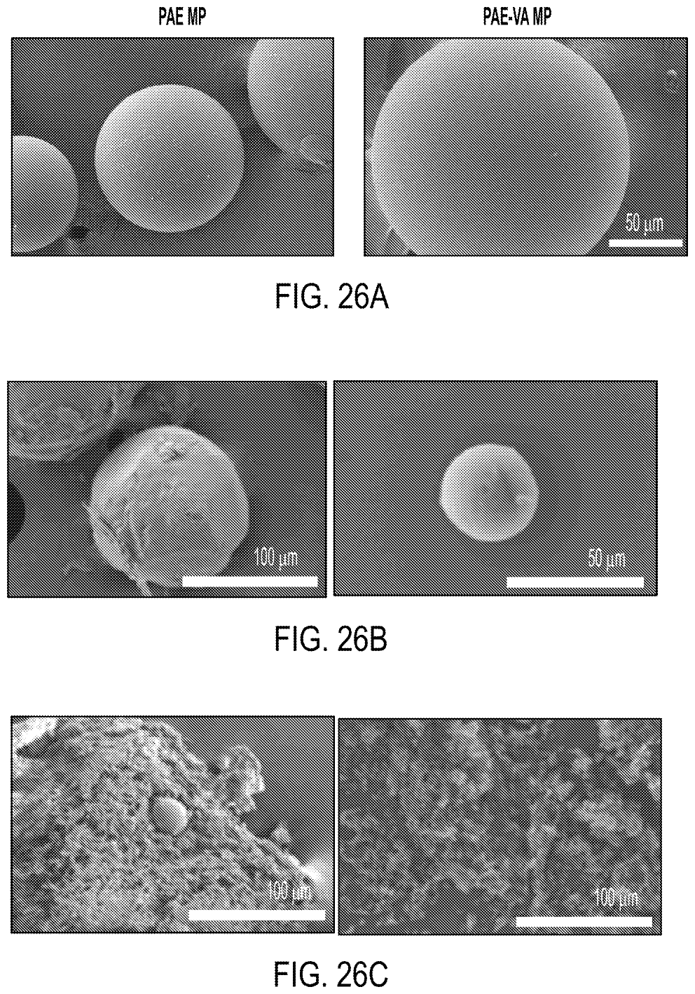

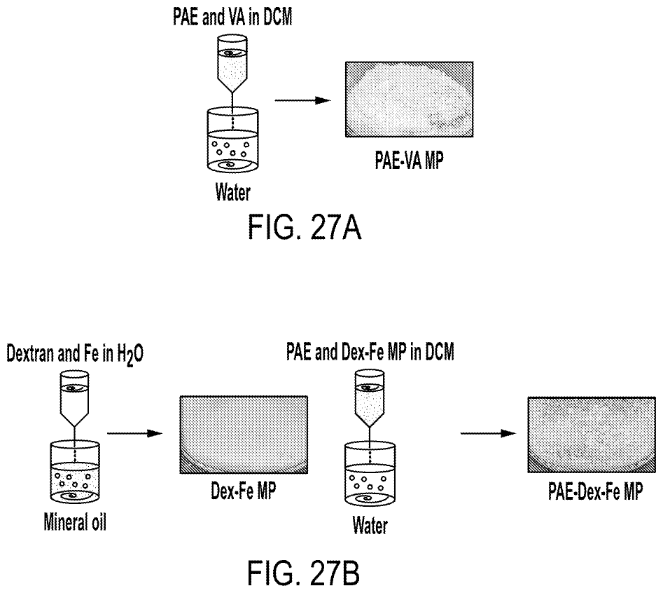

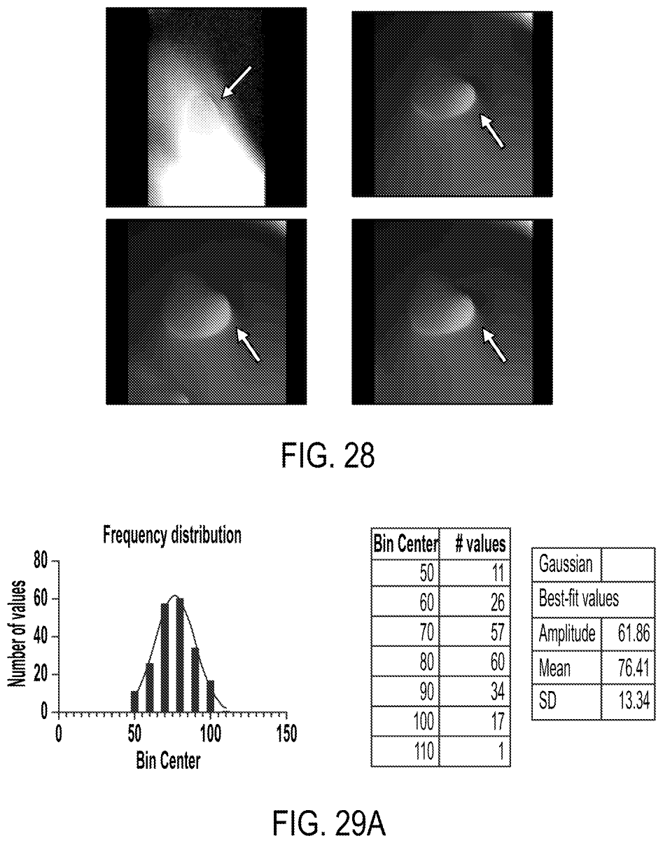

VA, VD, and VE were collectively encapsulated into PAE_E MPs, followed by 2-hour boiling or SGF treatment. The three micronutrients were effectively stabilized and efficiently released under this collective form (FIGS. 7E-7G).

The diversity of micronutrients encapsulated by PAE_E MP was expanded beyond vitamins to include metals like iron (as ferrous sulfate) and zinc (as zinc sulfate). A similar method was applied to encapsulated hydrophilic micronutrients (e.g., ferrous sulfate or zinc sulfate) by PAE. Other excipients, such as dextran (Dex) and poly(vinyl alcohol) (PVA), were tested as an alternative to hyaluronic acid, which has high cost and processing challenged due to viscosity usage. The results showed that the PAE_E MPs successfully encapsulated each of these two micronutrients. After 2-hour boiling in water, retention of these two micronutrients was observed with >99% recovery for Zn and >60% recovery for Fe. Under SGF conditions, both micronutrients were rapidly released after 30 minutes.

Additionally, PAE MPs are tested in different food matrices such as liquids and oil. The stability of PAE-MPs after cooking is carried out in water. Possible reactivity of diamino acids with food components depends mainly on the food matrix and may affect long-term stability of PAE MP. The interaction of diamino acids with food matrix is tested with the banana milk experiment. PAE MP is co-loaded with VA and iron, heated and mixed with the banana milk—polyphenol rich. Any change in the banana milk color over time indicates cross reaction.

Assess VA Absorption from PAE-Based Microparticles in Clinical Trials

Necessarily, the bioavailability of VA from PAELE particles in humans needs to be measured and evaluated for food fortification determination. Isotopically labeled VA in PAE_E particles is added to Maggi bouillon cubes for subject consumption, and blood draws are taken for retinyl palmitate analysis from plasma. Bioequivalence testing is used to analyze the raw data and determine bioequivalence of VA absorption from particles as compared to free uncooked VA (the highest potential level of absorption) as well as to free cooked VA. The absorption levels of VA from PAEE particles and BMC particles is compared. Additionally, this bioavailability study includes PAE MPs together with the other MNs, such as iron, B9, B12, zinc, and iodine. Toxicity data may be included in the bioavailability protocol approval.

### Example 2. Poly(β-Amino Ester) Microparticles for Micronutrient Fortification

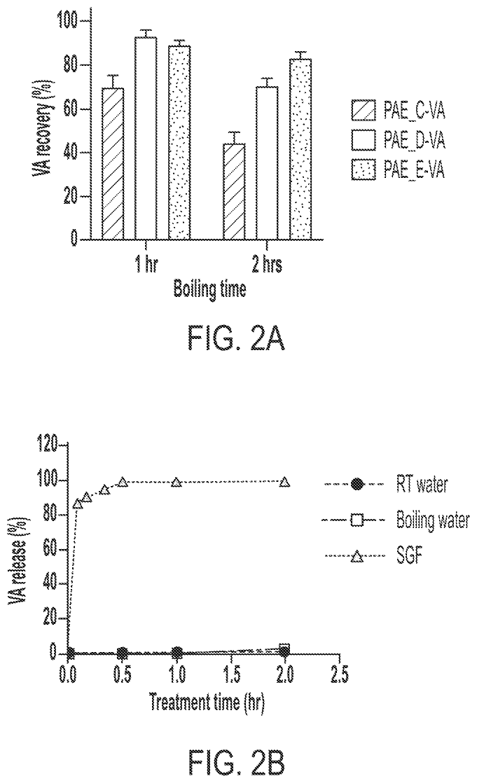

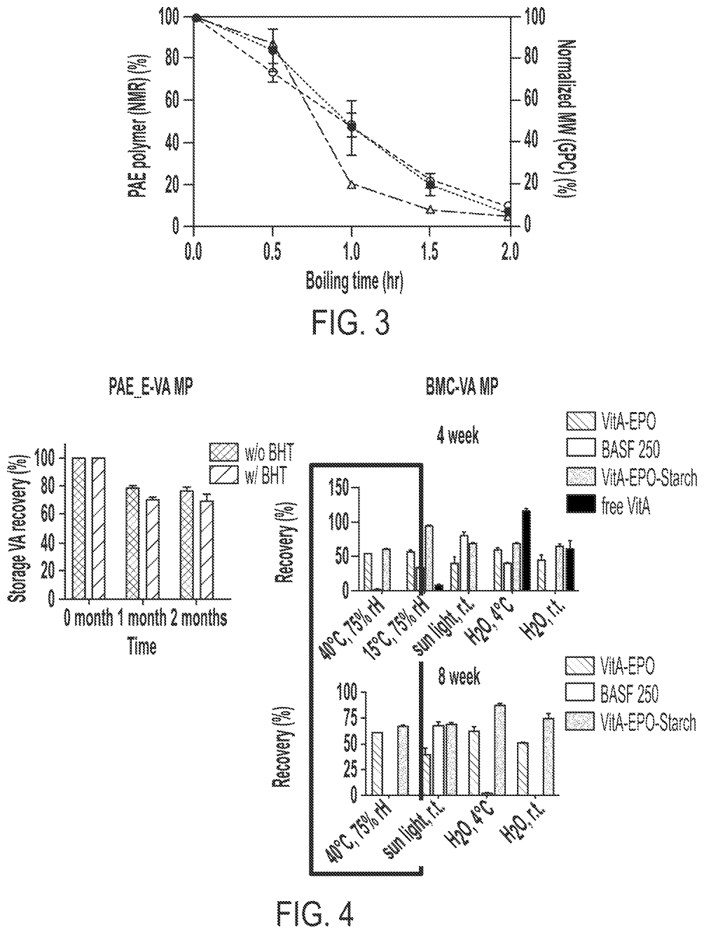

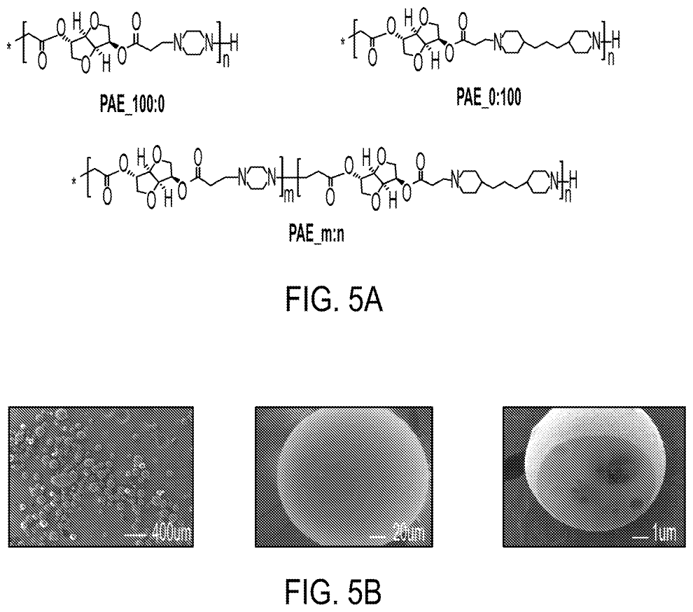

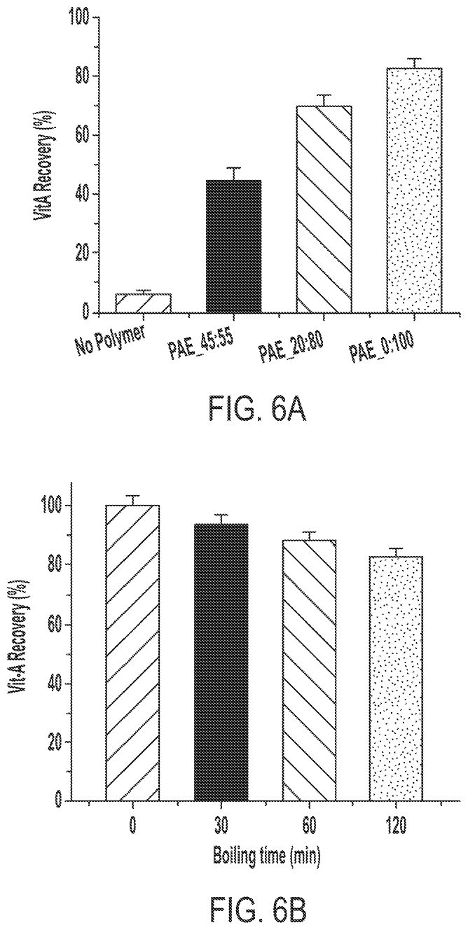

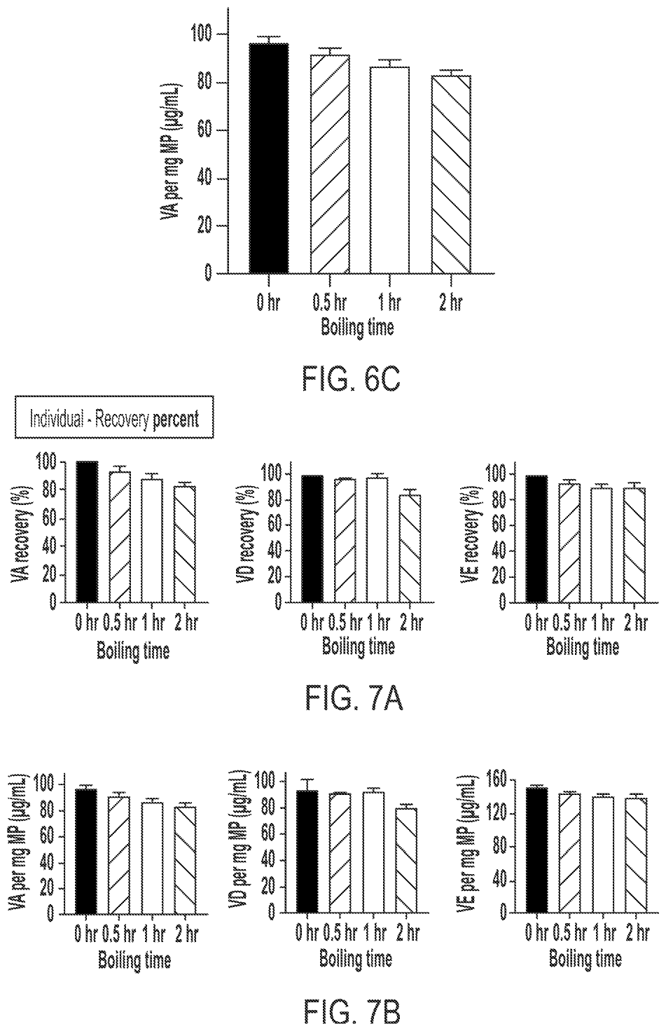

Various biodegradable and pH-responsive polymeric microparticles (MPs) capable of stabilizing micronutrients under simulated cooking condition and releasing them in simulated gastric fluid (SGF) have been developed. The MPs comprise sugar-based poly(p-amino esters) (PAE): poly[(isosorbide diacrylate)-co-piperazine] (PAE_100:0), poly[(isosorbide diacrylate)-co-(4,4′-trimethylenedipiperidine)] (PAE_0:100), and their terpolymers with different composition (PAE_m:n)(FIG. 5A). Micronutrients were encapsulated in PAE MPs using either oil-in-water or water-in-oil-in-water emulsion process, followed by centrifugation to remove unencapsulated micronutrients. The micronutrient-containing MPs were then dried by lyophilization and obtained in high yields (FIG. 5B). Encapsulation of micronutrients in PAE MPs stabilized them under simulated cooking condition, in water at 100° C. for up to 2 hours. Depending on the polymer composition, PAE MPs exhibited different degrees of protection of vitamin A in 100° C. water at 120 minutes (FIG. 6A). In particular, PAE_0:100 MPs were able to achieve vitamin A retention (>80% at 120 minutes) in 100° C. or RT water (FIG. 6B and FIG. 6C). Due to their pH-responsiveness, these PAE MPs quickly dissolved in 37° C. SGF and released the encapsulated vitamin A rapidly (>80% at 30 minutes) (FIG. 2B).

After exposure to boiling water for two hours, over 80% of VA and VD were recovered from individually encapsulated PAE microparticles, compared to less than 10% recovery from the free form micronutrients (FIG. 7). The boiling stability of VE was also improved from 80% of the free form to over 90% (FIG. 7). When collectively encapsulated in PAE microparticles, VD and VE showed decreased boiling stability to slightly below 80% under the same condition. The change in protection, compared to the individual encapsulation potentially resulted from increased micronutrient-to-PAE ratio.

Two excipients, dextran (Dex) and polyvinyl alcohol (PVA) were individually used to fabricate the first-step microparticles for Fe and Zn. Upon encapsulation by PAE to form the second-step microparticles, PAE-Dex-Fe and PAE-PVA-Zn performed best, with recoveries of 61.26% and 101.99% after two-hour boiling in water, respectively.

As a pH responsive polymer, PAE dissolves in acidic aqueous solution. After 30-minute treatment of SGF under 37° C., over 99% of micronutrients were effectively released from the PAE microparticles (FIG. 10).

Under the accelerated storage condition at 40° C. and 75% humidity, 74.46% of VA was recovered from the PAE-VA MP sample after one month. Compared to complete degradation of the free form under the same conditions, PAE microparticles showed effective protection for VA.

### Example 3. Synthesis of Isosorbide Diacrylate (2,5-Di-O-Acryloyl-1,4:3,6-Dianhydro-D-Glucitol)

Isosorbide (10 g, 68.4 mmol) and 4-dimethylaminopyridine (0.836 g, 6.8 mmol) were dissolved in 200 mL of anhydrous dichloromethane in a 500 mL round bottom flask. Then trimethylamine (17.310 g, 171.0 mmol) was added, and the reaction mixture was cooled to 0° C. and vigorously stirred. Acryloyl chloride (15.48 g, 166.0 mmol) was dissolved in 60 mL anhydrous dichloromethane and added slowly via a dropping funnel while warming up to room temperature. The reaction mixture was stirred for additional 24 hours after complete addition. Then the reaction mixture was washed with 2 times of 200 mL of 1 M hydrochloric acid followed by 2 times of 200 mL of saturated sodium chloride solution. The organic layer was dried over sodium sulfate, concentrated under reduced pressure, and then flash column chromatography was performed using dichloromethane and ethyl acetate as solvents. The pure product (15.65 g, 90% yield) was received as yellowish solid after removal of the solvent under reduced pressure.

### Example 4. Synthesis of PAE

PAE were synthesized with different ratios of TDP and piperazine (PAE_A to PAE_E) with piperazine ratio changing from 100% to 0% and fully characterized by GPC for molecular weight, DSC for glass transition temperature, water contact angle for hydrophobicity, and NMR for structure. The poly(β-amino ester) was synthesized according to the methods reported by Langer et al. with minor change. (13) Briefly, to synthesize poly[(isosorbide diacrylate)-co-piperazine] (PAE_A), isosorbide diacrylate (1.00 g, 3.93 mmol) and piperazine (0.34 g, 3.93 mmol) were dissolved in THE (5 mL) and reacted at 50° C. for 48 hours. The mixture was then cooled to room temperature and diluted to 0.05 mg mL⁻¹ with THF. To end-cap PAE_A, piperazine (1.00 g, 11.61 mmol) was added and reacted for 24 hours. After that, the product was precipitated into hexane and dried under vacuum overnight. LC-MS was used to detect any unreacted residual monomers in the PAE microparticle final product, and NMR was used to detect residual solvent.

To compare the effect of hydrophobicity on micronutrient protection efficiency, poly[(isosorbide diacrylate)-co-4,4′-trimethylenedipiperidine] (PAE_E) and terpolymers with different compositions were also synthesized by using different monomer feed ratios of piperazine to 4,4′-trimethylenedipiperidine. Addition of piperazine or TDP after polymerization ensures that isosorbide diacrylate is the limiting reagent in the reaction, i.e., any remaining C═C bond in polymer chains are end-capped by piperazine or TDP.

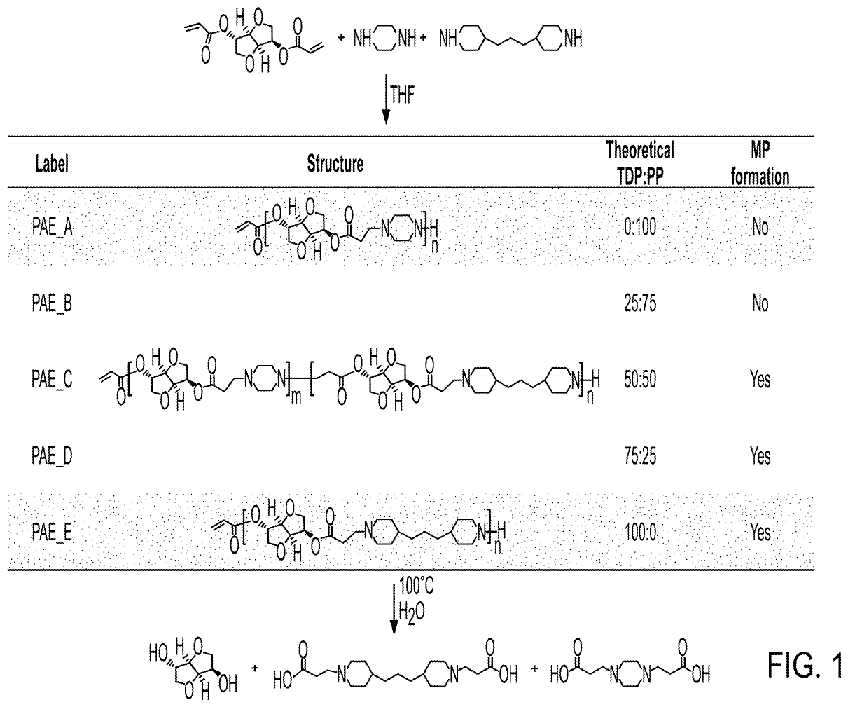

The polymer labels corresponding to monomer feed ratios are presented in FIG. 1. Polymers with the lower feed ratio of piperazine to 4,4′-trimethylenedipiperidine, were expected to exhibit higher hydrophobicity.

### Example 5. Microparticle Fabrication and Characterization

For hydrophobic micronutrients, PAE MPs were prepared by a modified oil-water emulsion method by which the PAE polymer was dissolved in an organic phase (dichloromethane) and then dispersed into an aqueous phase (water) under stirring condition. (2) For the organic phase, 200 mg PAE and 20 mg hydrophobic micronutrients (e.g., VA) were dissolved in 2 mL dichloromethane. The resulting organic phase was then emulsified in 40 mL of 1% polyvinyl alcohol solution with a stirring rate at 300 rpm for 2 hours. The obtained emulsion was then added into 150 mL deionized water with stirring at 500 rpm for 10 min to solidify the MPs. MPs in the emulsion were settled by gravity and thoroughly washed with deionized water via centrifugation for three times. The final dry MPs were obtained by lyophilization. Polymers with higher hydrophobicity, PAE_C, PAE_D, and PAE_E, formed solid, collectable microparticle-structure materials.

The final PAE microparticle products obtained from the emulsion processes were powder-like solid materials. The microparticles exhibited spherical, homogeneous structure with smooth surface and a size range of 100 to 200 m, depending on encapsulated micronutrients.

To encapsulate vitamin A (VA; retinyl palmitate), both VA and PAE were dissolved in the organic phase and the above emulsion procedure was performed. Notably, the addition of VA (10% wt) did not interfere with the formation of solid microparticle-structure materials for PAE_C, PAE_D, or PAE_E.

The encapsulation process was modified for metal salts that did not dissolve in the organic phase. A reverse emulsion method was used to fabricate a pre-MP sample. Specifically, the metal salt (ferrous sulfate or zinc sulfate) was dissolved in water phase along with an excipient (polyvinyl alcohol (PVA) or dextran) and then dispersed into mineral oil, followed by washing and collection. The resulting pre-MPs became more soluble in the organic phase and thus were compatible with the original workflow for MP encapsulation.

Scanning electron microscopy (SEM) was used to characterize the shape and surface properties of the PAE microparticles. Spherical structure and smooth surface are two key features of MPs to present as free-flowing powders for high compatibility with industrial food processing and vital for robust encapsulation. As illustrated by the SEM images, PAE_C afforded solid materials that were amorphous with few spherical structures. As hydrophobicity increased from PAE_C to PAE_D, spherical-like structures were observed, though with significant defects on the surface. Fortuitously, the PAE with highest hydrophobicity (PAE_E) afforded MPs with clear spherical structures and smooth surface.

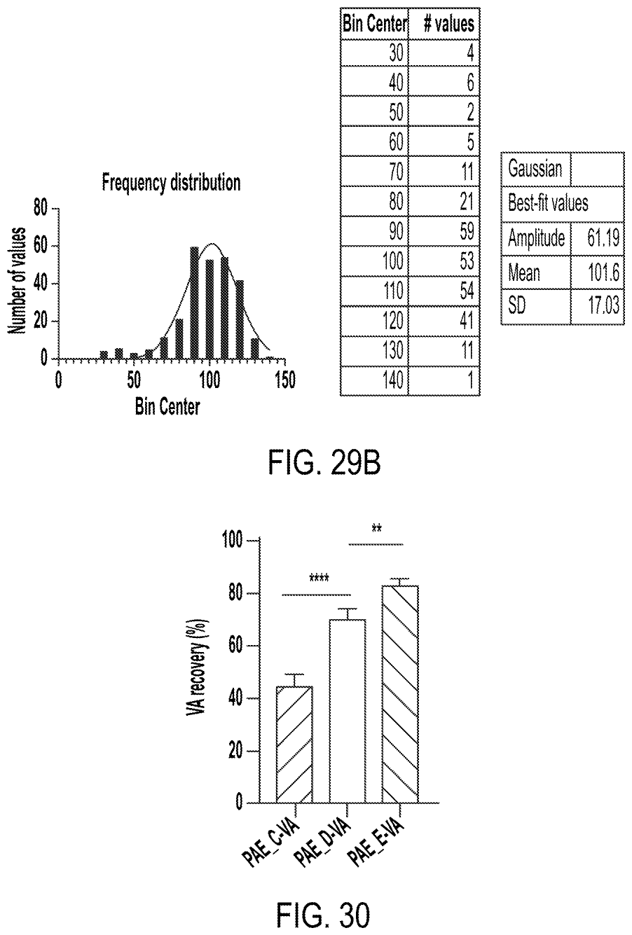

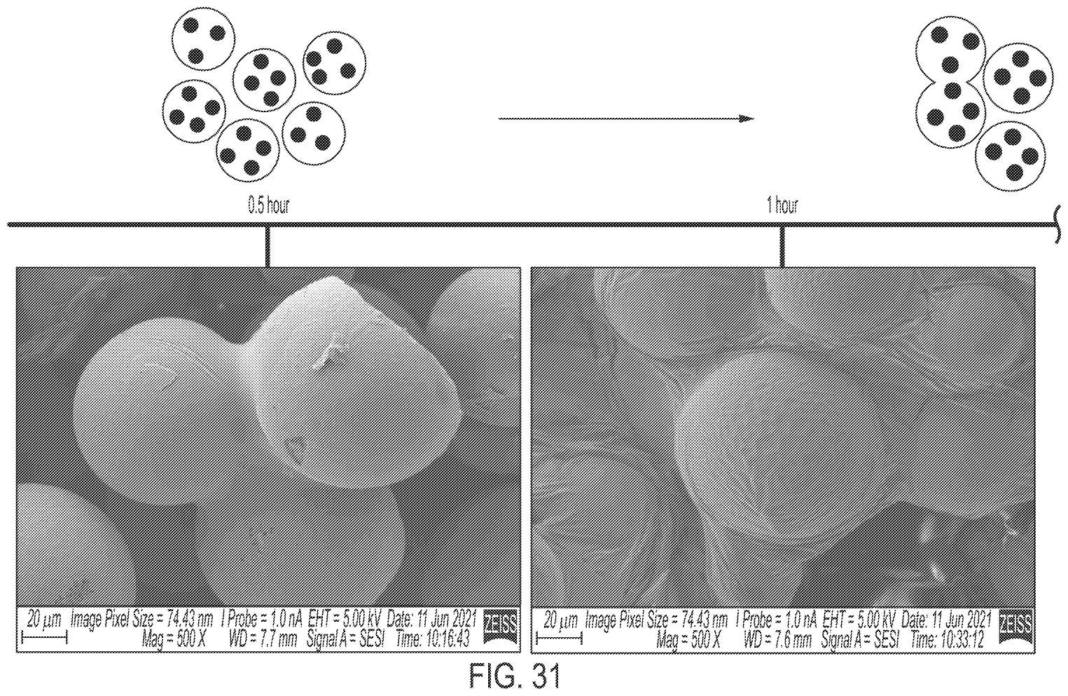

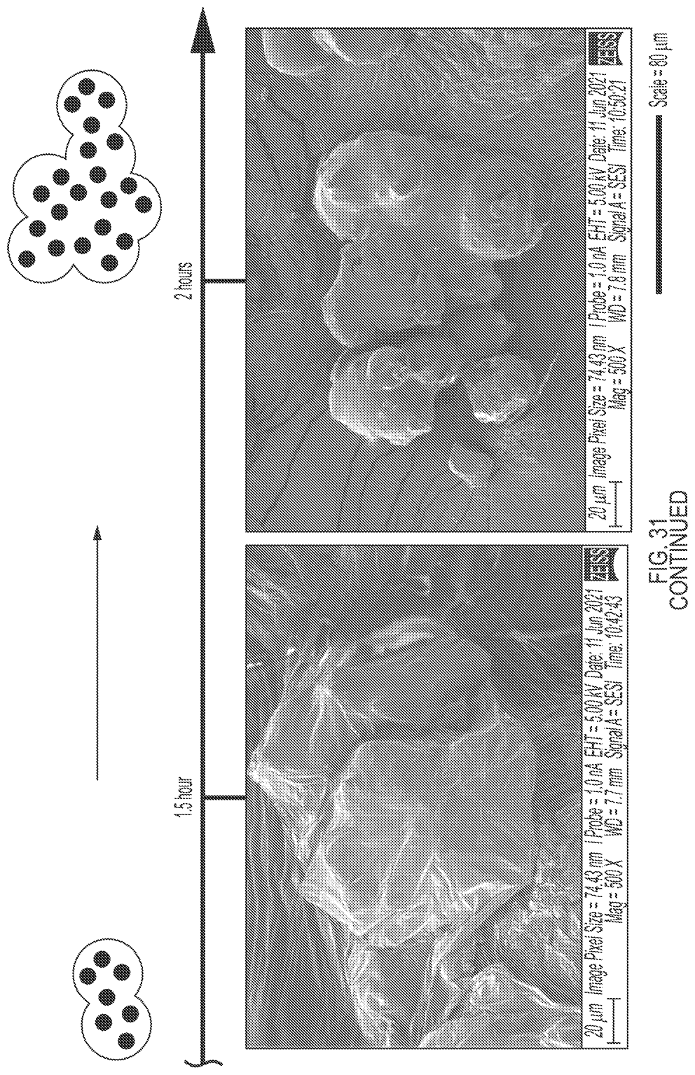

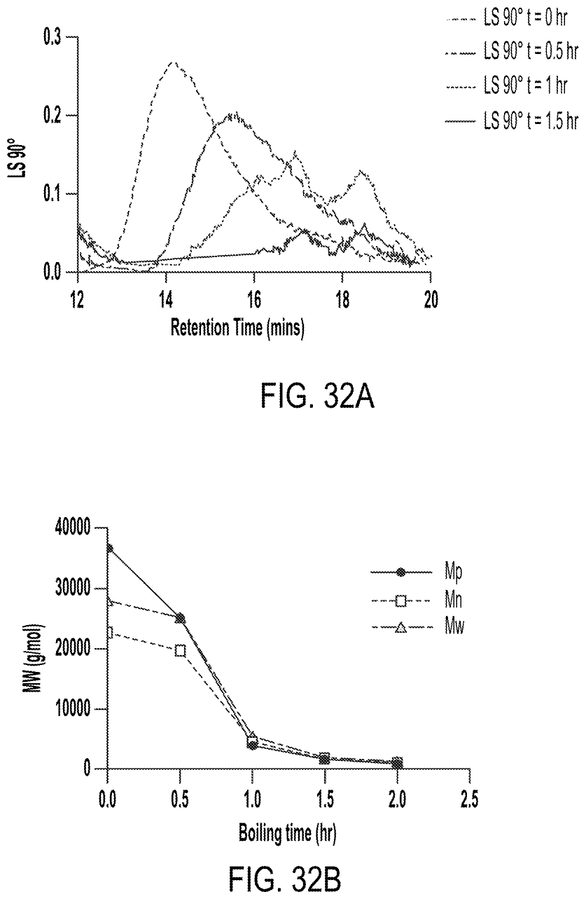

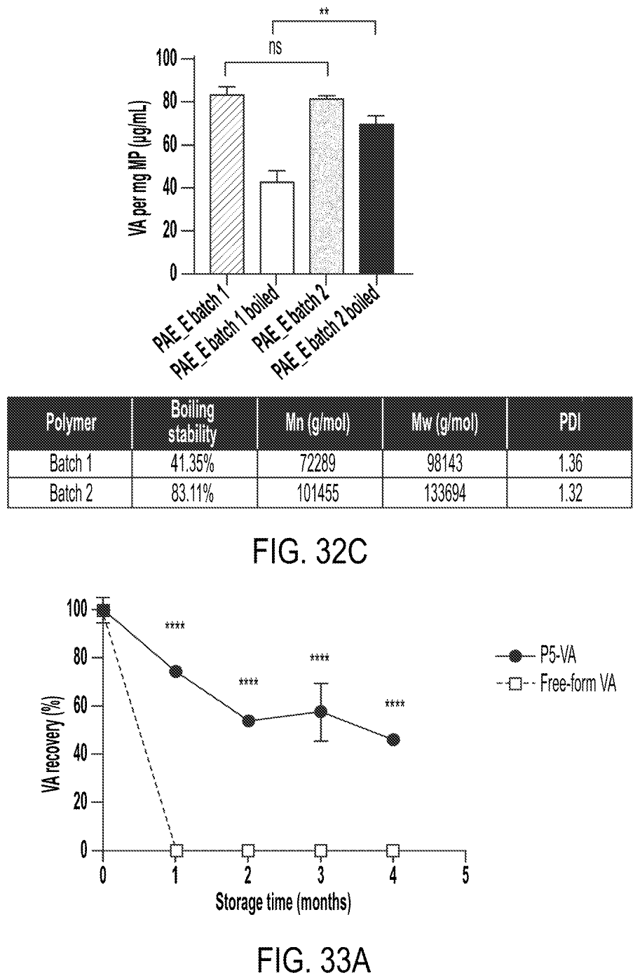

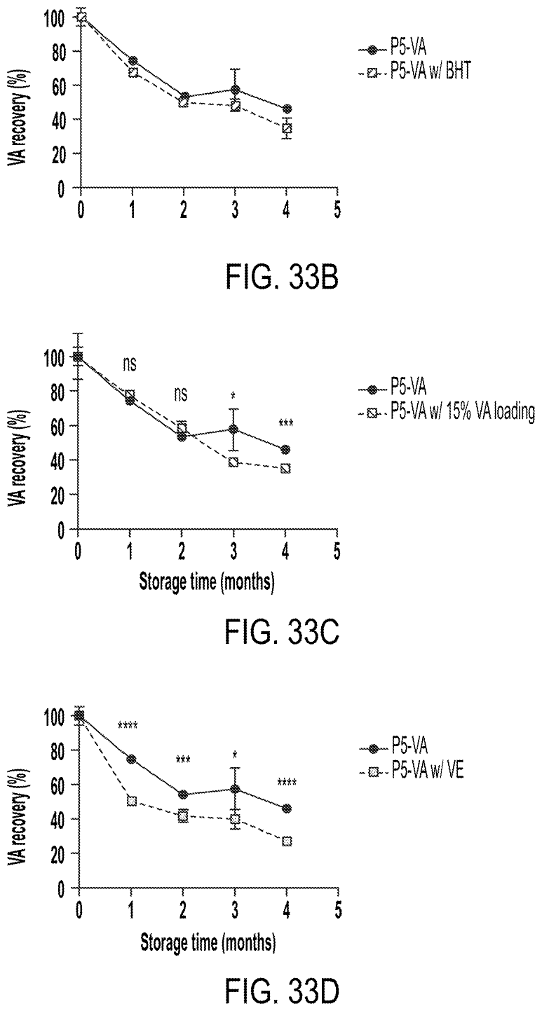

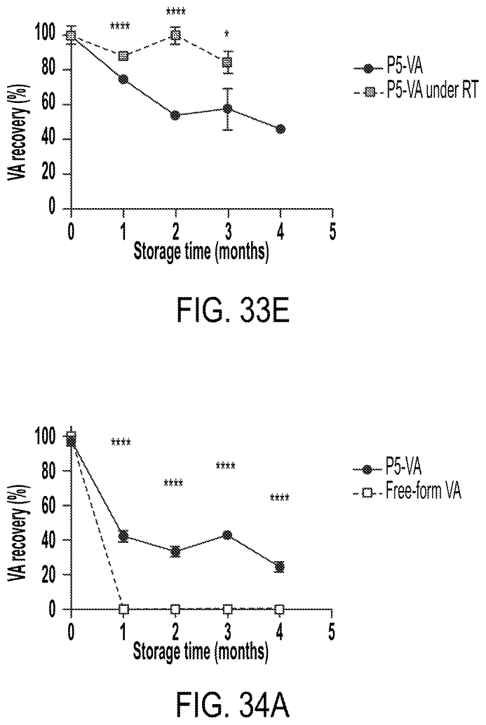

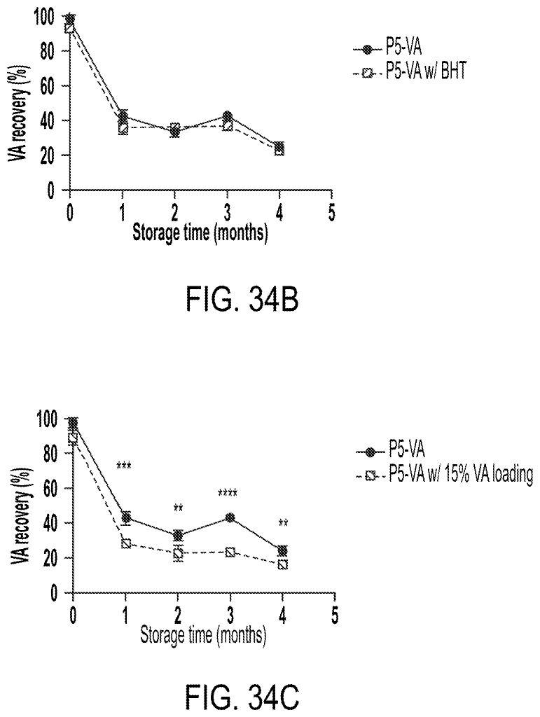

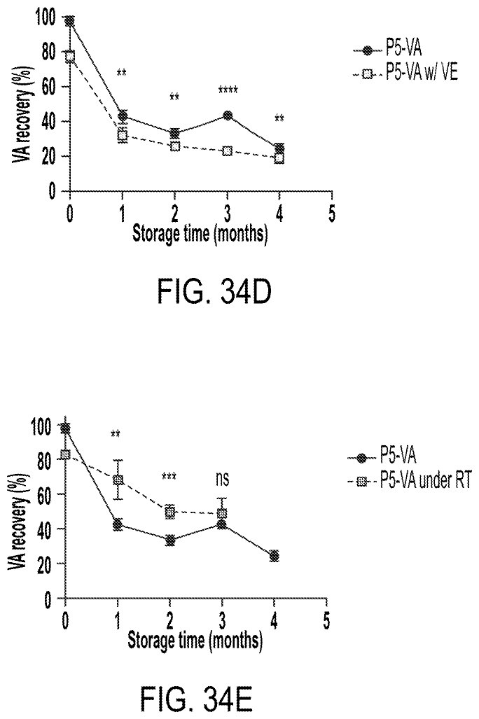

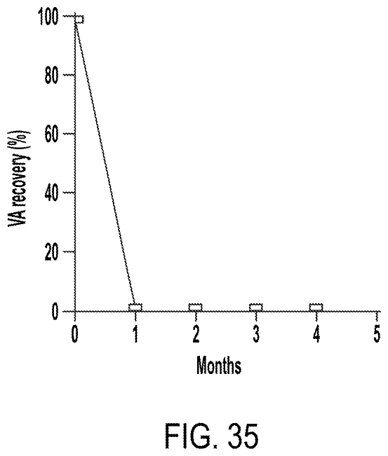

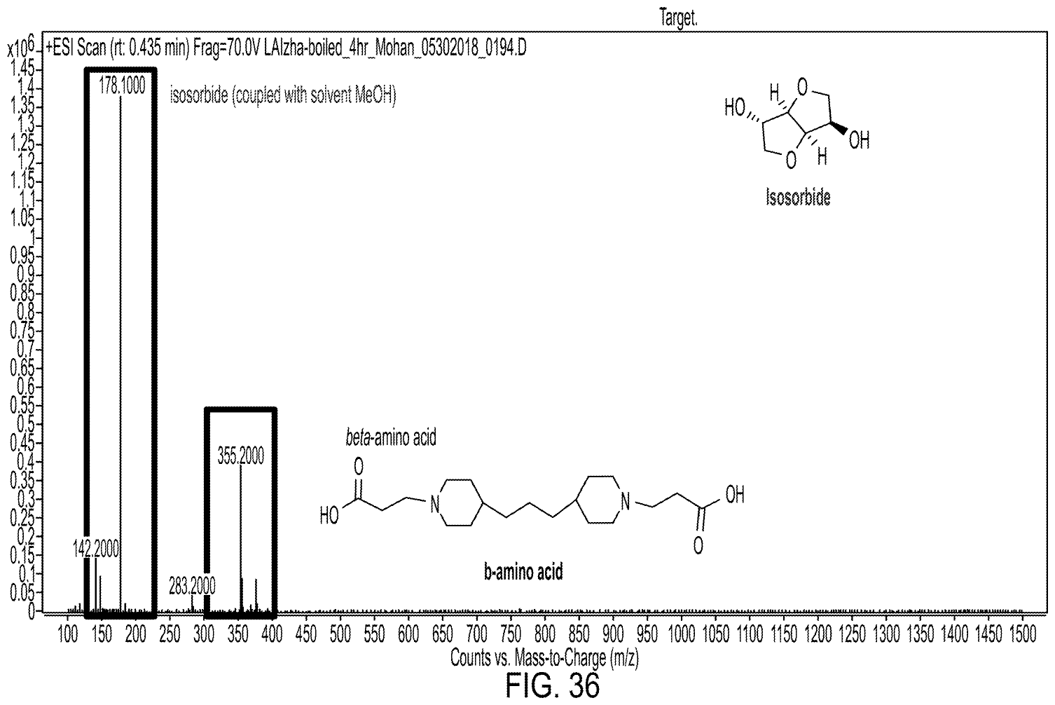

Comparison of MPs formed by each PAE with and without VA encapsulation showed no significant differences in their surface and shape properties. SEM analysis was used to determine the particle size distribution of the PAE_E MPs with and without VA encapsulation (FIGS. 29A-29B). Multi-photon fluorescence microscopy revealed homogenous distribution of VA across the PAE_E-VA MPs.

### Example 6. Characterization of Micronutrient Loading, Stability and Release

The boiling stability of micronutrients in PAE MP was studied in water at 100° C. At each time point, samples were lyophilized to obtain the boiled product.

The release profiles of micronutrients from PAE MP were studied in simulated gastric fluid (SGF) at 37° C. The SGF-treated samples were centrifuged at 15000 rpm for 10 min at each time point. The supernatant was then removed, and the pellet was lyophilized to obtain the SGF-treated product.

High-performance liquid chromatography (HPLC), inductively coupled plasma-optical emission spectrometer (ICP-OES), and colorimetric assay kits were used for micronutrient quantification. Vitamins A, D, E, B2, B9, and B12 were analyzed via high-performance liquid chromatography (Agilent 1100; Agilent Technologies) using a C-18 column (Acclaim Polar Advantage II, 3 μm, 4.6 mm×150 mm) and were detected by a photodiode detector at 325, 265, 290, 265, 286, and 230 nm, respectively. Iron, zinc, and vitamin C were analyzed using BioVision colorimetric assay kits.

### REFERENCES

1. Bailey R L, West K P Jr, Black R E. The epidemiology of global micronutrient deficiencies. Ann Nutr Metab. 2015; 66 Suppl 2:22-33.
2. Bhutta Z A, Salam R A. Global nutrition epidemiology and trends. Ann Nutr Metab. 2012; 61 Suppl 1:19-27.
3. Levinson, F J, Bassett, L. Malnutrition is still a major contributor to child deaths. Washington, DC. Population Reference Bureau, 2007.
4. Black R. Micronutrient deficiency—an underlying cause of morbidity and mortality. Bull World Health Organ. 2003; 81(2):79.
5. UNICEF. The State of the World's Children 2019. Children, Food and Nutrition: Growing well in a changing world. UNICEF, New York, 2019.
6. Datenblatt. “Isosorbide”. Acros. Retrieved 6 Jan. 2013.
7. Datenblatt. “D-Glucose” (PDF). Carl Roth [de]. Retrieved 24 Aug. 2010.
8. Feng X., et al., Thermal analysis characterization of isosorbide-containing thermosets, Isosorbide epoxy as BPA replacement for thermosets industry J. Therm. Anal. Calorim., 109, 1267-1275 (2012), doi:10.1007/s10973-012-2581-2
9. Lynn D. M., Langer R., Degradable poly(beta-amino esters): Synthesis, characterization, and self-assembly with plasmid DNA. J Am Chem Soc 122, 10761-10768 (2000).
10. T. Kemala, E. Budianto, B. Soegiyono, Preparation and characterization of microspheres based on blend of poly(lactic acid) and poly(epsilon-caprolactone) with poly(vinyl alcohol) as emulsifier. Arab J Chem 5, 103-108 (2012).
11. Andersson, M., Karumbunathan, V., Zimmermann, M. B. Global iodine status in 2011 and trends over the past decade. The Journal of Nutrition 142(4), 744-750 (2012).
12. Black, R. E. Global distribution and disease burden related to micronutrient deficiencies International Nutrition: Achieving Millennium Goals and Beyond 78, 21-28 (2014): Karger Publishers.
13. D. M. Lynn, R. Langer. Degradable poly(beta-amino esters): Synthesis, characterization, and self-assembly with plasmid DNA. J. Am. Chem. Soc. 122, 10761-10768 (2000).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound of Formula (I): 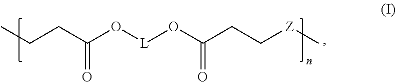
or a pharmaceutically acceptable salt, stereoisomer, or isotopically labeled derivative thereof, wherein:
L is a heterocycle comprising at least one oxygen atom;
each Z is independently of Formula (i), (ii), (iii), or (iv): 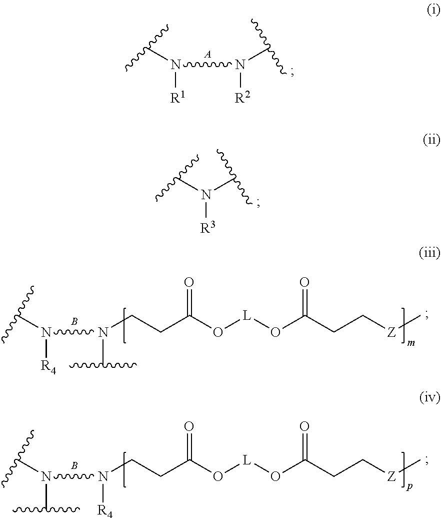
linker A is branched or unbranched, optionally substituted, cyclic or acyclic aliphatic; branched or unbranched, optionally substituted, cyclic or acyclic heteroaliphatic; optionally substituted arylene; or optionally substituted heteroarylene;
R¹ and R² are each independently hydrogen; branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group; or
one or both R¹ or R² are each optionally independently joined to linker A together with the intervening atoms to form a linker comprising one or more N-containing heterocycles; or
R¹ and R² are joined together with the intervening atoms to form a N-containing heterocycle;
R³ is branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group;
linker B is branched or unbranched, optionally substituted, cyclic or acyclic aliphatic; branched or unbranched, optionally substituted, cyclic or acyclic heteroaliphatic; optionally substituted arylene; or optionally substituted heteroarylene;
R⁴ is hydrogen, branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group; or
R⁴ is joined to linker B together with the intervening atoms to form a linker comprising a N-containing heterocycle; and
m, n, and p are each independently an integer between 1 and 10,000.

2. A compound of Formula (III): 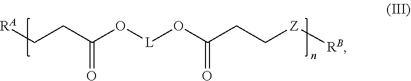
or a pharmaceutically acceptable salt, stereoisomer, or isotopically labeled derivative thereof, wherein:
L is a heterocycle comprising at least one oxygen atom;
each Z is independently of Formula (i), (ii), (iii), or (iv): 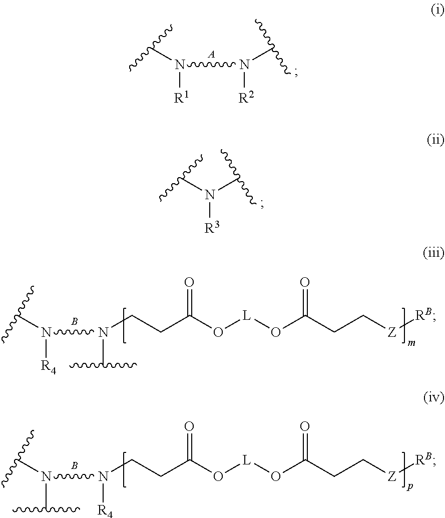
linker A is branched or unbranched, optionally substituted, cyclic or acyclic aliphatic; branched or unbranched, optionally substituted, cyclic or acyclic heteroaliphatic; optionally substituted arylene; or optionally substituted heteroarylene;
R¹ and R² are each independently hydrogen; branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group; or
one or both R¹ or R² are each optionally independently joined to linker A together with the intervening atoms to form a linker comprising one or more N-containing heterocycles; or
R¹ and R² are joined together with the intervening atoms to form a N-containing heterocycle;
R³ is branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group;
linker B is branched or unbranched, optionally substituted, cyclic or acyclic aliphatic; branched or unbranched, optionally substituted, cyclic or acyclic heteroaliphatic; optionally substituted arylene; or optionally substituted heteroarylene;
R⁴ is hydrogen, branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group; or
R⁴ is joined to linker B together with the intervening atoms to form a linker comprising a N-containing heterocycle;
m, n, and p are each independently an integer between 1 and 10,000;
each instance of R^{A} is independently —OR^{C}, —SR^{C}, —N(R^{C})₂, —ZR^{C}, or 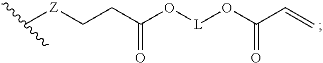
each instance of R^{B} is independently 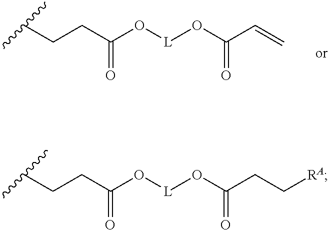
and
each instance of R^{C} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, a nitrogen protecting group when attached to a nitrogen atom, or two instances of R^{C} attached to the same intervening atom are joined together with the intervening atom to form optionally substituted heterocyclyl or optionally substituted heteroaryl.

3. The compound of claim 1 or 2, or a pharmaceutically acceptable salt, stereoisomer, or isotopically labeled derivative thereof, wherein L is 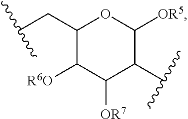
and
the compound of Formula I is of Formula (I-A) 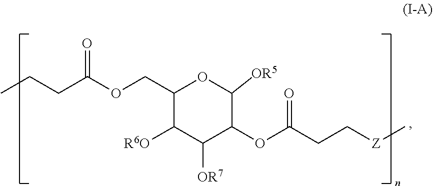
wherein R⁵, R⁶, and R⁷ are each independently hydrogen, C₁₋₆ alkyl, an oxygen protecting group, or wherein R⁶ and R⁷ may be joined together with the intervening atoms to form optionally substituted heterocyclyl.

4. The compound of claim 3, or a pharmaceutically acceptable salt, stereoisomer, or isotopically labeled derivative thereof, wherein L is 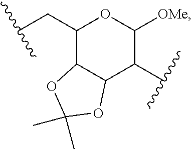 [SMILES: *CC1OC(OC)C(*)C2OC(C)(C)OC12]
and
the compound of Formula (I-A) is of Formula (I-A-iii)

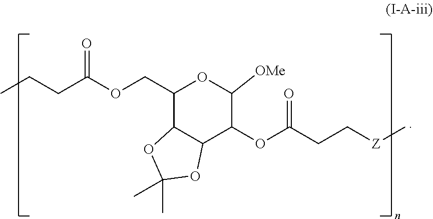

5. The compound of claim 4, or a pharmaceutically acceptable salt, stereoisomer, or isotopically labeled derivative thereof, wherein L is 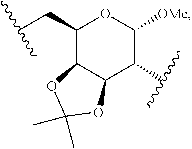 [SMILES: *C[C@H]1O[C@H](OC)[C@H](*)[C@H]2OC(C)(C)O[C@H]21]
and
the compound of Formula (I-A) is of Formula (I-A-ii)

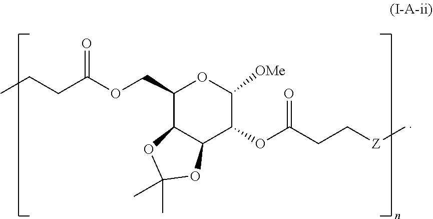

6. The compound of claim 3, wherein L is 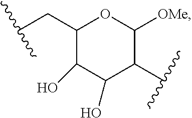 [SMILES: *CC1OC(OC)C(*)C(O)C1O]
and
the compound of Formula (I-A) is of Formula (I-A-v)

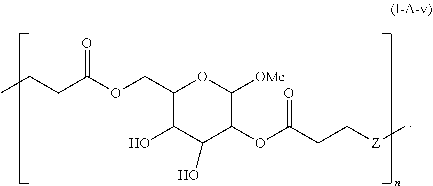

7. The compound of claim 6, wherein L is 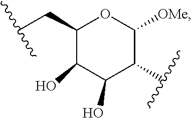 [SMILES: *C[C@H]1O[C@H](OC)[C@H](*)[C@@H](O)[C@H]1O]
and
the compound of Formula (I-A) is of Formula (I-A-iv)

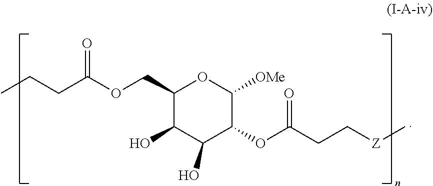

8. The compound of claim 3, wherein L is 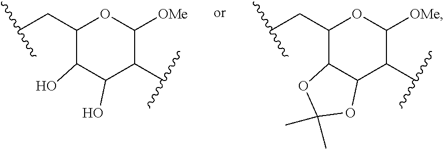 [SMILES: *CC1OC(OC)C(*)C(O)C1O ; "or" ; *CC1OC(OC)C(*)C2OC(C)(C)OC12]
and
the compound of Formula (I-A) is of Formula (I-A-vii) 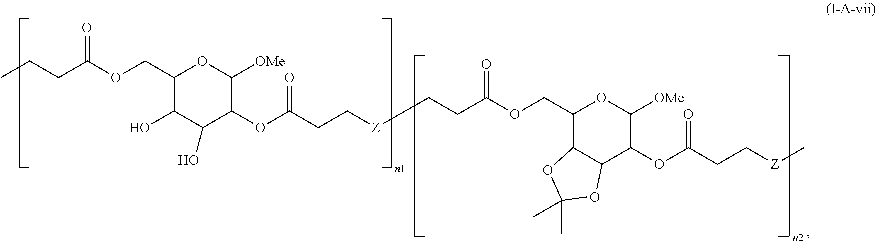
wherein the sum of n1 and n2 is n.

9. The compound of claim 8 wherein L is 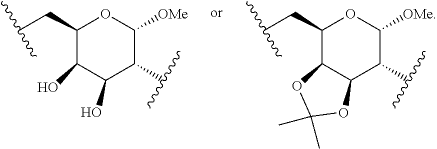 [SMILES: *C[C@H]1O[C@H](OC)[C@H](*)[C@@H](O)[C@H]1O ; "or" ; *C[C@H]1O[C@H](OC)[C@H](*)[C@H]2OC(C)(C)O[C@H]21]
and
the compound of Formula (I-A) is of Formula (I-A-vi) 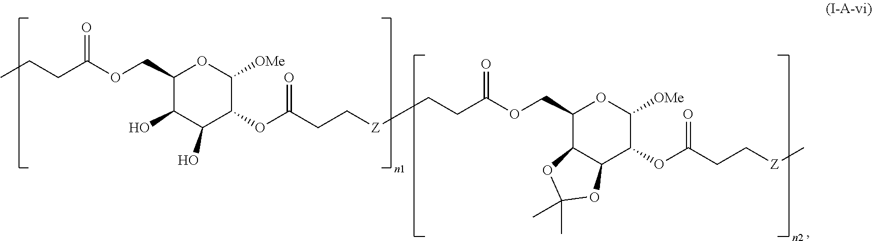
wherein the sum of n1 and n2 is n.

10. The compound of claim 1 or 2, or a pharmaceutically acceptable salt, stereoisomer, or isotopically labeled derivative thereof, wherein L is 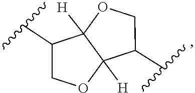 [SMILES: *C1COC2C(*)COC12]
and
the compound of Formula (I) is of Formula (I-B):

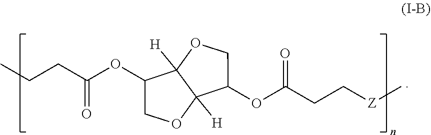

11. The compound of claim 10, or a pharmaceutically acceptable salt, stereoisomer, or isotopically labeled derivative thereof, wherein the compound of Formula (I) is of Formula (I-B-i) or Formula (I-B-ii): 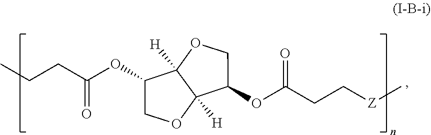
wherein L is 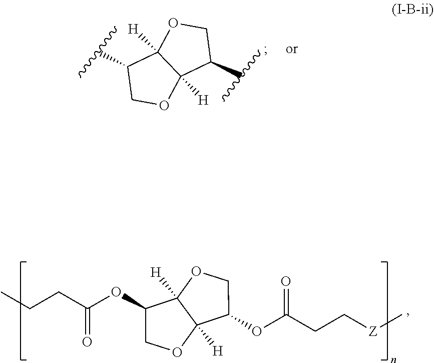
wherein L is

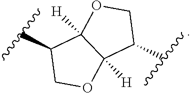 [SMILES: *[C@@H]1CO[C@H]2[C@@H]1OC[C@@H]2*]

12. The compound of any one of claims 1-11, or a pharmaceutically acceptable salt, stereoisomer, or isotopically labeled derivative thereof, wherein n is an integer between 3 and 10,000.

13. The compound of claim 12, or a pharmaceutically acceptable salt, stereoisomer, or isotopically labeled derivative thereof, wherein n is an integer between 10 and 500.

14. The compound of any one of claims 1-13, or a pharmaceutically acceptable salt, stereoisomer, or isotopically labeled derivative thereof, wherein each Z is independently of Formula (i) or (ii).

15. The compound of any one of claims 1-14, or a pharmaceutically acceptable salt, stereoisomer, or isotopically labeled derivative thereof, wherein each Z is independently of Formula (i).

16. The compound of claim 15, or a pharmaceutically acceptable salt, stereoisomer, or isotopically labeled derivative thereof, wherein one or both R¹ or R² are each independently joined to linker A together with the intervening atoms to form a linker comprising one or more N-containing heterocycles; or R¹ and R² are joined together with the intervening atoms to form a N-containing heterocycle.

17. The compound of claim 16, or a pharmaceutically acceptable salt, stereoisomer, or isotopically labeled derivative thereof, wherein the N-containing heterocycle is a 6-membered N-containing heterocycle.

18. The compound of any one of claims 1, 2, or 4-17, or a pharmaceutically acceptable salt, stereoisomer, or isotopically labeled derivative thereof, wherein L is 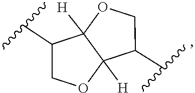 [SMILES: *C1COC2C(*)COC12]
and
the compound of Formula (I) is of Formula (I-B-iii): 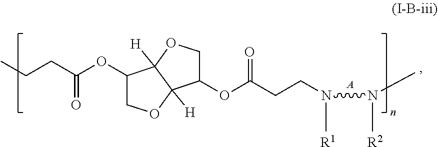
wherein each 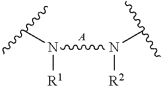
is independently selected from

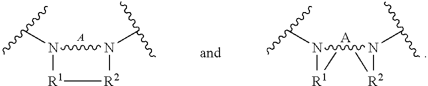

19. The compound of any one of claims 1-18, or a pharmaceutically acceptable salt, stereoisomer, or isotopically labeled derivative thereof, wherein 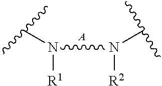
is

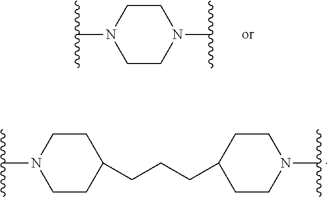 [SMILES: *N1CCN(*)CC1 ; *N1CCC(CCCC2CCN(*)CC2)CC1]

20. The compound of claim 19, or a pharmaceutically acceptable salt, stereoisomer, or isotopically labeled derivative thereof, wherein approximately 0-10%, approximately 5-15%, approximately 10-20%, approximately 15-25%, approximately 20-30%, approximately 25-35%, approximately 30-40%, approximately 35-45%, approximately 40-50%, approximately 45-55%, approximately 50-60%, approximately 55-65%, approximately 60-70%, approximately 65-75%, approximately 70-80%, approximately 75-85%, approximately 80-90%, approximately 85-95%, or approximately 90-100% of Z is

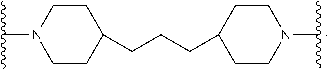 [SMILES: *N1CCC(CCCC2CCN(*)CC2)CC1]

21. The compound of claim 19 or 20, or a pharmaceutically acceptable salt, stereoisomer, or isotopically labeled derivative thereof, wherein approximately 0-10%, approximately 5-15%, approximately 10-20%, approximately 15-25%, approximately 20-30%, approximately 25-35%, approximately 30-40%, approximately 35-45%, approximately 40-50%, approximately 45-55%, approximately 50-60%, approximately 55-65%, approximately 60-70%, approximately 65-75%, approximately 70-80%, approximately 75-85%, approximately 80-90%, approximately 85-95%, or approximately 90-100% of Z is

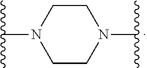 [SMILES: *N1CCN(*)CC1 ; "."]

22. The compound of claim 20, or a pharmaceutically acceptable salt, stereoisomer, or isotopically labeled derivative thereof, wherein Z is

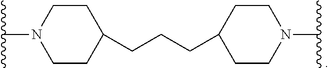 [SMILES: *N1CCC(CCCC2CCN(*)CC2)CC1 ; "."]

23. The compound of claim 19, or a pharmaceutically acceptable salt, stereoisomer, or isotopically labeled derivative thereof, wherein approximately 15-30% of Z is 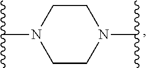 [SMILES: *N1CCN(*)CC1 ; ","]
and approximately 70-85% of Z is

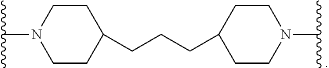 [SMILES: *N1CCC(CCCC2CCN(*)CC2)CC1 ; "."]

24. The compound of claim 19, or a pharmaceutically acceptable salt, stereoisomer, or isotopically labeled derivative thereof, wherein approximately 40-55% of Z is 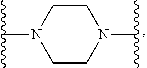 [SMILES: *N1CCN(*)CC1 ; ","]
and approximately 45-60% of Z is

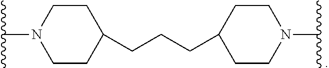 [SMILES: *N1CCC(CCCC2CCN(*)CC2)CC1 ; "."]

25. The compound of claim 19, or a pharmaceutically acceptable salt, stereoisomer, or isotopically labeled derivative thereof, wherein approximately 70-85% of Z is 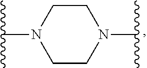 [SMILES: *N1CCN(*)CC1 ; ","]
and approximately 15-30% of Z is

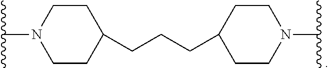 [SMILES: *N1CCC(CCCC2CCN(*)CC2)CC1 ; "."]

26. The compound of claim 19, or a pharmaceutically acceptable salt, stereoisomer, or isotopically labeled derivative thereof, wherein Z is

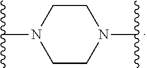 [SMILES: *N1CCN(*)CC1 ; "."]

27. The compound of any one of claims 2-26, wherein each instance of R^{A} is independently —OR^{C}, —SR^{C}, —N(R^{C})₂, or —ZR^{C}

28. The compound of any one of claims 2-26, wherein each instance of R^{A} is independently —ZR^{C} or

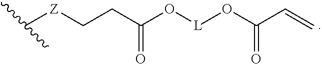

29. The compound of any one of claims 2-28, wherein at least one instance of R^{B} is

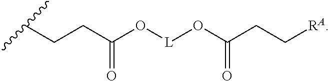

30. The compound of any one of claims 2-29, wherein at least one instance of R^{B} is

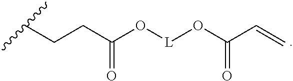

31. A composition comprising a compound of any one of claims 1-30, or a pharmaceutically acceptable salt, stereoisomer, or isotopically labeled derivative thereof, and an agent.

32. The composition of claim 31, wherein the agent is a vitamin, mineral, micronutrient, biologic, small molecule, probiotic, polynucleotide, bacteria, cell, or microorganism.

33. The composition of claim 32, wherein the agent is a vitamin or mineral.

34. The composition of claim 33, wherein the agent is vitamin A, iron, iodine, vitamin B2 (riboflavin), vitamin B7 (niacin), vitamin b12 (cobalamin), vitamin D (cholecalcifeol), vitamin E (tocopherol), vitamin K1 (phytomenadione), vitamin C (6-O-palmitoyl-L-ascorbic acid), or zinc.

35. The composition of claim 34, wherein the agent is vitamin A, vitamin D (cholecalcifeol), vitamin E (tocopherol), or vitamin C (6-O-palmitoyl-L-ascorbic acid).

36. The composition of claim 33, wherein the agent is ferrous sulfate or zinc sulfate.

37. The composition of any one of claims 31-36, wherein the composition encapsulates the agent.

38. The composition of any one of claims 31-37, wherein the composition is in the form of a microparticle or nanoparticle.

39. The composition of any one of claims 31-38, wherein the agent and the compound are not covalently attached.

40. The composition of any one of claims 31-39, wherein the composition is thermally stable, hydrolytically stable, light stable, and/or oxidatively stable.

41. The composition of any one of claims 31-40, wherein the composition improves the thermal stability, hydrolytic stability, light stability, and/or oxidative stability of the agent.

42. The composition of any one of claims 31-41, wherein the composition degrades under acidic conditions.

43. The composition of claim 42, wherein the acidic conditions have pH less than 7.0.

44. The composition of claim 43, wherein the acid conditions have pH of about 1.2.

45. The composition of any one of claims 42-44, wherein degradation under acidic conditions releases the agent.

46. The composition of any one of claims 31-45, wherein degradation of the composition produces one or more natural byproducts.

47. The composition of claim 46, wherein the natural byproduct is isosorbide.

48. The composition of claim 46 or 47, wherein the natural byproduct is a p-amino acid.

49. The composition of any one of claims 31-48, wherein the composition further comprises an excipient.

50. The composition of claim 49, wherein the excipient is a polysaccharide or derivative thereof, collagen or derivative thereof, hydrolyzed collagen or derivative thereof, or water-soluble synthetic polymer or derivative thereof.

51. The composition of claim 49 or 50, wherein the excipient is dextran.

52. The composition of claim 49 or 50, wherein the excipient is polyvinyl alcohol (PVA).

53. The composition of any one of claims 49-52, wherein the composition comprises about 1-5% of the excipient by weight relative to the total mass of the particle.

54. The composition of any one of claims 31-52, wherein the excipient is a stabilizer.

55. The composition of claim 54, wherein the stabilizer is BHT.

56. The composition of claim 54 or 55, wherein the composition comprises about 0.5% of the stabilizer relative to the mass of polymer.

57. A pharmaceutical composition comprising a compound of any one of claims 1-30 or a composition of any one of claims 31-56.

58. A nutraceutical composition comprising a compound of any one of claims 1-30 or a composition of any one of claims 31-56.

59. A food product comprising a compound of any one of claims 1-30 or a composition of any one of claims 31-56.

60. A beverage comprising a compound of any one of claims 1-30 or a composition of any one of claims 31-56.

61. A nutritional supplement comprising a compound of any one of claims 1-30 or a composition of any one of claims 31-56.

62. The composition of any one of claims 57-61, wherein the composition further comprises an excipient.

63. A kit comprising:
a compound of any one of claims 1-30, or a pharmaceutically acceptable salt, stereoisomer, or isotopically labeled derivative thereof; or a pharmaceutical composition of any one of claims 31-56; and
instructions for using the compound, or pharmaceutically acceptable salt, stereoisomer, or isotopically labeled derivative thereof, or pharmaceutical composition.

64. A method of delivering an agent to a subject, comprising administering to the subject a composition of any one of claims 31-56.

65. A method of preparing a compound comprising Formula (I), or a pharmaceutically acceptable salt, stereoisomer, or isotopically labeled derivative thereof, the method comprising reacting one or more compounds of Formula (II): 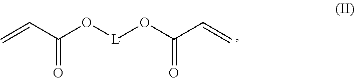
or a salt, isotope, or stereoisomer thereof, with one or more compounds selected from 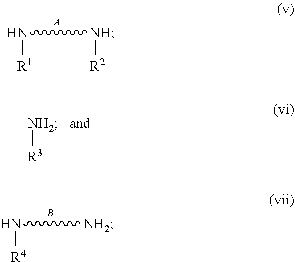
or a salt, isotope, or stereoisomer thereof, wherein:
L is a heterocycle comprising at least one oxygen atom;
linker A is branched or unbranched, optionally substituted, cyclic or acyclic aliphatic; branched or unbranched, optionally substituted, cyclic or acyclic heteroaliphatic; optionally substituted arylene; or optionally substituted heteroarylene;
R¹ and R² are each independently hydrogen; branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group; or
one or both R¹ or R² are each optionally independently joined to linker A together with the intervening atoms to form a linker comprising one or more N-containing heterocycles; or
R¹ and R² are joined together with the intervening atoms to form a N-containing heterocycle;
R³ is branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group;
linker B is branched or unbranched, optionally substituted, cyclic or acyclic aliphatic; branched or unbranched, optionally substituted, cyclic or acyclic heteroaliphatic; optionally substituted arylene; or optionally substituted heteroarylene; and
R⁴ is hydrogen, branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group; or
R⁴ is joined to linker B together with the intervening atoms to form a linker comprising a N-containing heterocycle.

66. The method of claim 65, wherein the method further comprises reacting the compound with one or more compounds selected from HOR^{C}, HSR^{C}, or HN(R^{C})₂, wherein each instance of R^{C} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, a nitrogen protecting group when attached to a nitrogen atom, or two instances of R^{C} attached to the same intervening atom are joined together with the intervening atom to form optionally substituted heterocyclyl or optionally substituted heteroaryl.

67. The method of claim 65 or 66, wherein the compound of Formula (II) is of the formula: 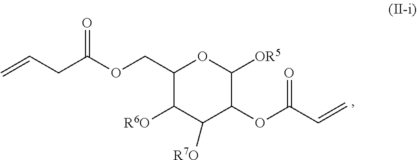
wherein R⁵, R⁶, and R⁷ are each independently an oxygen protecting group, or wherein R⁶ and R⁷ may be joined together with the intervening atoms to form optionally substituted heterocyclyl.

68. The method of claim 67, wherein the compound of Formula (II-i) is

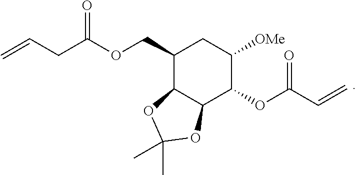 [SMILES: C=CCC(=O)OC[C@H]1C[C@H](OC)[C@H](OC(=O)C=C)[C@H]2OC(C)(C)O[C@@H]12 ; "."]

69. The method of claim 65 or 66, wherein the compound of Formula (II) is

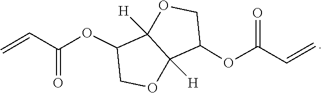 [SMILES: C=CC(=O)OC1COC2C(OC(=O)C=C)COC12]

70. The method of any one of claims 65-69, wherein one of the one or more compounds of
formula (v) is

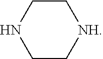

71. The method of any one of claims 65-70, wherein one of the one or more compounds of formula (v) is

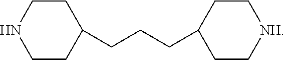 [SMILES: C(CC1CCNCC1)CC1CCNCC1]

72. A compound prepared by reacting one or more compounds of Formula (II): 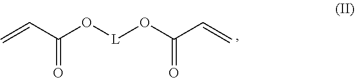
or a salt, isotope, or stereoisomer thereof, with one or more compounds selected from 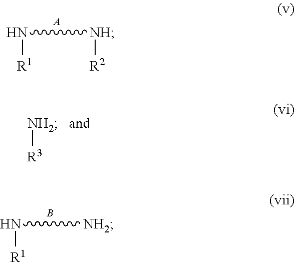
or a salt, isotope, or stereoisomer thereof, wherein:
L is a heterocycle comprising at least one oxygen atom;
linker A is branched or unbranched, optionally substituted, cyclic or acyclic aliphatic; branched or unbranched, optionally substituted, cyclic or acyclic heteroaliphatic; optionally substituted arylene; or optionally substituted heteroarylene;
R¹ and R² are each independently hydrogen; branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group; or
one or both R¹ or R² are each optionally independently joined to linker A together with the intervening atoms to form a linker comprising one or more N-containing heterocycles; or
R¹ and R² are joined together with the intervening atoms to form a N-containing heterocycle;
R³ is branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group;
linker B is branched or unbranched, optionally substituted, cyclic or acyclic aliphatic; branched or unbranched, optionally substituted, cyclic or acyclic heteroaliphatic; optionally substituted arylene; or optionally substituted heteroarylene; and
R⁴ is hydrogen, branched or unbranched, optionally substituted, cyclic or acyclic aliphatic; branched or unbranched, optionally substituted, cyclic or acyclic heteroaliphatic; branched or unbranched, optionally substituted, cyclic or acyclic acyl; optionally substituted aryl; optionally substituted heteroaryl; or a nitrogen protecting group; or
R⁴ is joined to linker B together with the intervening atoms to form a linker comprising a N-containing heterocycle.

73. The compound of claim 72, wherein the compound of Formula (II) is of the formula: 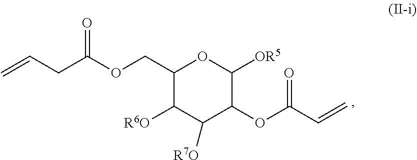
wherein R⁵, R⁶, and R⁷ are each independently an oxygen protecting group, or wherein R⁶ and R⁷ may be joined together with the intervening atoms to form optionally substituted heterocyclyl.

74. The compound of claim 73, wherein the compound of Formula (II-i) is

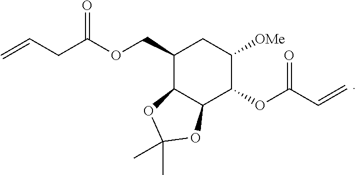 [SMILES: C=CCC(=O)OC[C@H]1C[C@H](OC)[C@H](OC(=O)C=C)[C@H]2OC(C)(C)O[C@@H]12 ; "."]

75. The compound of claim 73, wherein the compound of Formula (II-i) is

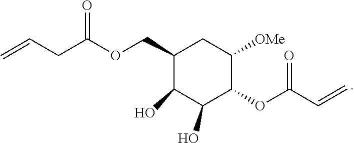 [SMILES: C=CCC(=O)OC[C@H]1C[C@H](OC)[C@H](OC(=O)C=C)[C@@H](O)[C@H]1O ; "."]

76. The compound of claim 73, wherein the compound of Formula (II) is

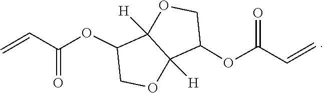 [SMILES: C=CC(=O)OC1COC2C(OC(=O)C=C)COC12]

77. The compound of any one of claims 72-76, wherein one of the one or more compounds of formula (v) is

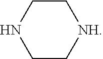

78. The compound of any one of claims 72-77, wherein one of the one or more compounds of formula (v) is

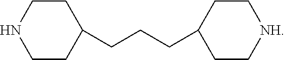 [SMILES: C(CC1CCNCC1)CC1CCNCC1]

79. A method of treating or preventing a disease, disorder, or condition in a subject, comprising administering to the subject a composition of any one of claims 31-56.

80. The method of claim 79, wherein the disease, disorder, or condition is a micronutrient deficiency, genetic disease, proliferative disease, hematological disease, neurological disease, liver disease, spleen disease, lung disease, painful condition, psychiatric disorder, musculoskeletal disease, metabolic disorder, inflammatory disease, or autoimmune disease.

81. The method of claim 80 wherein the disease, disorder, or condition is a micronutrient deficiency.

82. The method of claim 81, wherein the micronutrient deficiency is a vitamin or mineral deficiency.

83. The method of claim 81 or 82, wherein the micronutrient deficiency is vitamin A deficiency.

84. The method of claim 81 or 82, wherein the micronutrient deficiency is vitamin D deficiency.

85. The method of claim 81 or 82, wherein the micronutrient deficiency is vitamin E deficiency.

86. The method of claim 81 or 82, wherein the micronutrient deficiency is iron deficiency.

87. The method of claim 81 or 82, wherein the micronutrient deficiency is zinc deficiency.
